(19) United States
(12) Patent Application Publication
(10) Pub. No.: US 2026/0042747 A1
(43) Pub. Date: Feb. 12, 2026

(54) KIT INHIBITORS, COMPOUNDS, PHARMACEUTICAL COMPOSITIONS, AND METHODS OF USE THEREOF

(71) Applicant: Alivexis, Inc., Tokyo (JP)

(72) Inventors: Taisuke TAKAHASHI, Tokyo (JP); Takafumi SHIMIZU, Tokyo (JP); Yoh TERADA, Tokyo (JP); Takeo URAKAMI, Tokyo (JP); William SINKO, Kent County, DE (US); Ramulu PODDUTOORI, Karnataka (IN); Venkateshwar Rao GUMMADI, Karnataka (IN)

(73) Assignee: Alivexis, Inc., Tokyo (JP)

(21) Appl. No.: 19/099,662

(22) PCT Filed: Jul. 31, 2023
(86) PCT No.: PCT/JP2023/027945

§ 371 Date: Jan. 29, 2025

Related U.S. Application Data
(60) Provisional application No. 63/370,035, filed on Aug. 1, 2022.

(51) Int. Cl. C07D 401/14; A61K 31/4184; A61K 31/427; A61K 31/437; A61K 31/4439; A61K 31/444; A61K 31/4545; A61K 31/496; A61K 31/497; A61K 31/506; A61P 35/00; C07B 59/00; C07D 235/26; C07D 401/12; C07D 403/12; C07D 403/14; C07D 405/14; C07D 417/12; C07D 471/04

(52) U.S. Cl. CPC C07D 401/14; A61K 31/4184; A61K 31/427; A61K 31/437; A61K 31/4439; A61K 31/444; A61K 31/4545; A61K 31/496; A61K 31/497; A61K 31/506; A61P 35/00; C07B 59/002; C07D 235/26; C07D 401/12; C07D 403/12; C07D 403/14; C07D 405/14; C07D 417/12; C07D 471/04

(57) ABSTRACT

Compounds and compositions that are useful in inhibiting a receptor tyrosine kinase, KIT. The compounds and compositions provided herein are useful for the prevention or treatment of one or more KIT mediated diseases or conditions, such as cancers, autoimmune diseases, allergic diseases, inflammatory diseases, fibrosis, metabolic disorders, and neurodegenerative diseases.

### TECHNICAL FIELD

This application is directed to inhibitors of a receptor tyrosine kinase, KIT (α-KIT), and methods for their use, such as to prevent or treat one or more KIT mediated diseases or conditions.

### BACKGROUND ART

Protein phosphorylation is an important cellular regulatory mechanism as many enzymes and receptors are activated/deactivated by phosphorylation and dephospho-rylation events, by means of kinases and phosphatases. Receptor tyrosine kinases (RTKs) play an important role in a variety of cellular processes including growth, motility, differentiation, and metabolism. KIT (c-KIT) is a tyrosine kinase receptor that acts as a facultative onco-protein and regulates the development and function of multiple distinct cell lineages. These include hematopoietic progenitors, melanocytes, germ cells, and mast cells (MCs), which are all significantly affected by loss-of-function mutations of KIT or its cytokine ligand, stem cell factor (SCF) (Valent et al., Theranostics 2020, 10(23), 10743).

Abnormalities in the expression and function of KIT are associated with several human diseases. KIT activation has been implicated in various human cancers, including mastocytosis, germ cell tumors, small-cell lung carcinoma, gastrointestinal stromal tumors (GIST), acute myelogenous leukemia, neuroblastoma, melanoma, ovarian carcinoma, and breast carcinoma (Turner et al., Blood 1992, 80: 374-381; Heinrich et al., J Clin Oncol 2002, 20: 1692-1703). KIT activation in these tumors is generally accomplished by one or more of three mechanisms: autocrine and/or paracrine stimulation by SCF, cross-activation by other kinases, and/or the acquisition of activating mutations (Hirota et al., Science (Wash DC) 1998, 279: 577-580; Heinrich et al. 2002). Pharmacological inhibition of KIT or SCF with small molecule inhibitors or specific antibodies can also affect the function of healthy and car-cinogenic cells expressing KIT receptor (Valent et al., J Allergy Clin Immunol 2022, 149:1866-74). Indeed, several kinase inhibitors are approved for the treatment of GIST and other malignancies with KIT activation in both humans and non-human mammals.

It has become evident that KIT and its ligand participate in the development and function of multiple distinct cell lineages. These include cells in parts of the central nervous system, the interstitial cells of Cajal in the gut, taste cells, as well as several hematopoietic cell lineages in addition to MCs, including dendritic cells, eosinophils, and ILC2s (Tsai M et al., J. Allergy Clin. Immunol. 2022, 149:1845-54). While most lineages lose KIT as they differentiate, MCs are one of the few cell types that retain KIT expression even after differentiation into mature cells (Lennartsson J et al., Stem Cells 2005, 23:16-43).

KIT and its ligand SCF play a dominant role in the development and maturation of tissue MCs. In contrast to other myeloid cells, MCs are long-lived. This is because the development of fully mature MCs from stem cells through pluripotent and committed MC progenitor cells may take several months. In addition, some mature tissue MCs survive months or perhaps years when residing within local tissue microen-vironments (Cerny-Reiterer S et al., Oncotarget 2015, 6:3071-84). At maturation, MCs reside in local tissue sites at which SCF and other growth factors and cytokines are expressed, supporting long-term survival as well as effector cell functions.

As multifunctional cells of the innate immune system, MCs contribute to adaptive immune responses, and they play a role in allergic and other inflammatory reactions, as well. MCs express high-affinity receptors for IgE and produce numerous biologically active substances, some of which are stored in cytoplasmic granules for rapid release (Valent et al. 2020). MCs can be activated by many different stimuli, including allergens through IgE and IgE receptors; by IgG immune complexes; by complement and cytokines; by toxins, venom, and bacterial byproducts, as well as by certain medications (Valent et al. 2020). MC activation occurs in a number of different disease states and pathologies. Clinical symptoms may be severe, resulting in challenges to everyday life. In patients with allergic (atopic) disorders and in those with mastocytosis, MC activation may be substantial and systemic, even resulting in overt anaphylaxis.

Imatinib is a small molecule TKI which blocks KIT and other kinases including ABL1, ABL2, PDGFRs, and many more. Initially developed as a targeted cancer drug, Imatinib is approved by both the US Food and Drug Administration and European Medicines Agency for the treatment of systemic mastocytosis (SM), where the numbers of MCs are excessively elevated. The rationale for the use of imatinib in SM came from several studies. In one report, patients with chronic myeloid leukemia were treated with imatinib for several years. During this therapy, the number of MCs in the bone marrow were found to have decreased after 12 months; and after 24 months, MCs were almost completely absent in bone marrow sections (Cerny-Reiterer S et al. 2015). Simultaneously, serum tryptase levels, a MC activation marker, decreased to the point where they were low to undetectable (Cerny-Reiterer S et al. 2015). In a different study, 62 patients with poorly controlled or uncontrolled severe asthma who had airway hyperresponsiveness despite receiving maximal medical therapy were treated with imatinib (400 mg per day orally) for 24 weeks. Therapy with imatinib was found to reduce airway hyperresponsiveness and bronchoalveolar lavage tryptase levels more effectively in these patients than in the placebo control (Cahill K N et al., N Engl J Med 2017, 376:1911-20). Taken together, these data suggest that therapy with KIT-targeting drugs may lead to partial or even complete eradication of the MC lineage in patients with various MC diseases. In addition, some of these TKIs can also suppress IgE-dependent activation of MCs and basophils (Valent et al. 2022). Therefore, highly specific KIT inhibitors may offer a new, safer treatment option for patients with severe MC diseases.

### SUMMARY OF INVENTION

Provided herein, inter alia, are compounds and compositions comprising such compounds that are useful for inhibiting KIT. In particular, disclosed herein are methods inhibiting the activity of KIT for the prevention or treatment of KIT mediated diseases or conditions associated with improperly regulated kinase signal transduction of MCs such as cancers, autoimmune diseases, allergic diseases, inflammatory diseases, fibrosis, metabolic disorders, and neurodegenerative diseases by administering to a subject a therapeutically effective amount of one or more compounds or compositions provided herein.

In one embodiment, provided herein is a pharmaceutical composition comprising a pharmaceutically acceptable carrier or diluent and a compound represented by the following Formula (I):

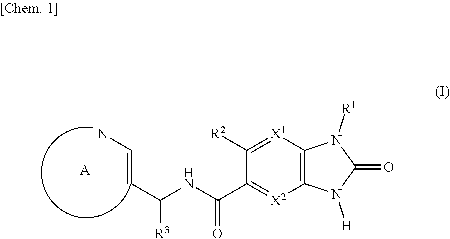

or a pharmaceutically acceptable salt thereof, wherein:
R¹ is hydrogen or C₁₋₃-alkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen or C₁₋₄-alkyl,
a ring A:

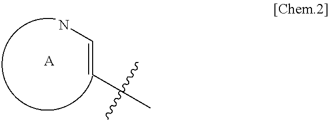

is a group of the formula:

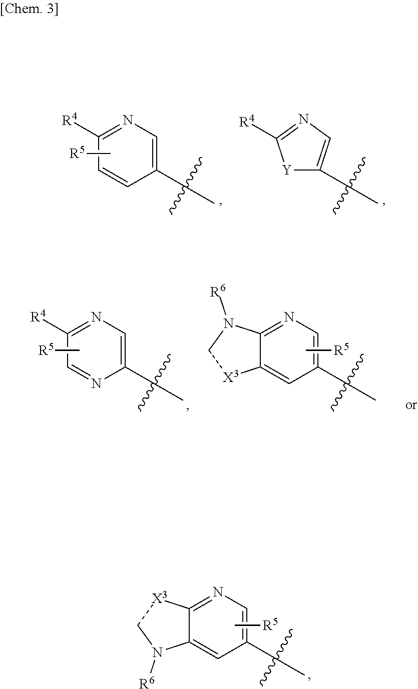

the wavy line indicates a point of attachment to the rest of molecule,
R⁴ is —N(R^{A})(R^{B}), —O—R^{A}, or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino.

In another embodiment, provided herein is a compound represented by the following Formula (Ia):

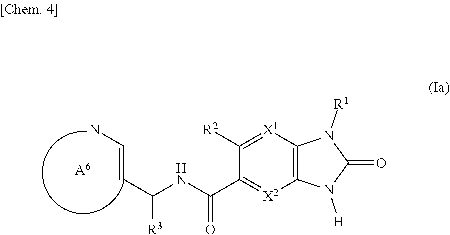

or a pharmaceutically acceptable salt thereof, wherein:
R¹ is hydrogen or C₁₋₃-alkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen or C₁₋₄-alkyl,
a ring A¹:

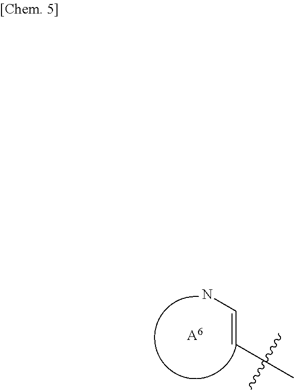

is a group of the formula:

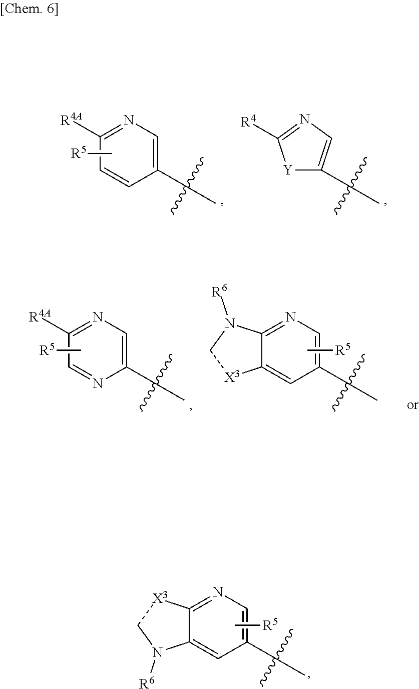

the wavy line indicates a point of attachment to the rest of molecule,
R⁴ is —N(R^{A})(R^{B}), —O—R^{A}, or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R^{4A} is —N(R^{A})(R^{B}), or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
provided that the compound is not

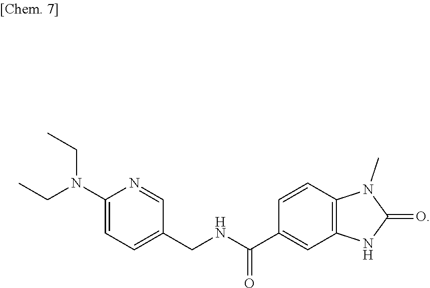 [SMILES: CCN(CC)c1ccc(CNC(=O)c2ccc3c(c2)[nH]c(=O)n3C)cn1]

Another embodiment comprises method for preventing or treating a KIT mediated disease or condition in a subject by administering to the subject a therapeutically effective amount of one or more of the disclosed compounds, or a pharmaceutically acceptable salt thereof, or a pharmaceutical composition comprising the compound(s).

Also provided herein is use of one or more of the disclosed compounds, or a pharmaceutically acceptable salt thereof, for the preparation of a medicament for the prevention or treatment of a KIT mediated disease or condition.

In another embodiment, provided herein is one or more of the disclosed compounds, or a pharmaceutically acceptable salt thereof, or a pharmaceutical composition comprising the disclosed compound(s) for use in preventing or treating a KIT mediated disease or condition.

### DESCRIPTION OF EMBODIMENTS

### Definitions

The term “alkyl” used alone or as part of a larger moiety, such as “haloalkyl”, “cycloalkyl”, “alkylamino”, and the like, means saturated aliphatic straight-chain or branched monovalent hydrocarbon radical. Unless otherwise specified, an alkyl group typically has 1 to 4 carbon atoms, i.e., C₁₋₄-alkyl. As used herein, a “C₁₋₄-alkyl” group means a radical having from 1 to 4 carbon atoms in a linear or branched arrangement, and includes methyl, ethyl, propyl, isopropyl, butyl, isobutyl, sec-butyl and tert-butyl.

The term “deuteroalkyl” means an alkyl having one or more deuterium atoms. For example, the term “C₁₋₃-deuteroalkyl” means a radical having from 1 to 3 carbon atoms, and includes mono, di, and trideuteromethyl.

The term “alkylamino” means amino substituted with one or more alkyl groups. For example, “mono- or di-(C₁₋₄-alkyl)amino” includes methylamino, ethylamino, isopropylamino, dimethylamino, diethylamino, (ethyl)(methyl)amino, (isopropyl)(methyl)amino and (tert-butyl)(methyl)amino.

The term “haloalkyl” means alkyl substituted with one or more halogen atoms. For example, “C₁₋₄-haloalkyl” includes fluoromethyl, difluoromethyl, trifluoromethyl, chloromethyl, dichloromethyl, bromomethyl, fluoroethyl, difluoroethyl, dichloroethyl and chloropropyl. In one aspect, an example of C₁₋₄-haloalkyl includes difluoromethyl or trifluoromethyl, and in another aspect, an example of C₁₋₄-haloalkyl includes trifluoromethyl.

The term “halogen” or “halo” means fluorine or fluoro (F), chlorine or chloro (Cl), bromine or bromo (Br), or iodine or iodo (I).

The term “aryl” means a monocyclic or bicyclic aromatic hydrocarbon radical such as phenyl, naphthyl, anthracenyl, 1,2-dihydronaphthyl, 1,2,3,4-tetrahydronaphthyl, fluorenyl, indanyl and indenyl. In one aspect, an example of aryl includes phenyl or naphthyl, and in another aspect, an example of aryl includes phenyl.

The term “cycloalkyl” means a 3-12 membered saturated aliphatic cyclic hydrocarbon radical. It can be monocyclic, bicyclic (e.g., a bridged, spirofused, or fused bicyclic ring), or tricyclic. For example, monocyclic C₃₋₆-cycloalkyl means a radical having from 3 to 6 carbon atoms arranged in a monocyclic ring. For example, “C₃₋₆-cycloalkyl” includes, but is not limited to, cyclopropyl, cyclobutyl, cyclopentyl, cyclohexyl, bicyclo[1.1.1]pentyl, bicyclo[3.1.0]hexyl, and spiro[2.3]hexyl.

The term “heteroaryl” refers to monocyclic or bicyclic aromatic ring radical having at least one (typically 1 to 4, more typically 1 or 2) heteroatoms (e.g., oxygen, nitrogen, or sulfur). Examples of such heteroaryl groups include pyrrolyl, pyrazolyl, imidazolyl, triazolyl, tetrazolyl, furyl, oxazolyl, isoxazolyl, oxadiazolyl, thienyl, thiazolyl, isothiazolyl, thiadiazolyl, pyridyl, pyridazinyl, pyrimidinyl, pyrazyl, indolyl, isoindolyl, benzofuryl, benzothienyl, indazolyl, benzimidazolyl, benzoxazolyl, ben-zothiazolyl, quinolyl, isoquinolyl, cinnolinyl, quinazolinyl, quinoxalyl, pyrolopyridyl, and imidazolopyridyl.

The term “heterocyclyl” refers to a monocyclic or bicyclic non-aromatic ring radical having at least one (typically 1 to 4, more typically 1 or 2) heteroatoms (e.g., oxygen, nitrogen, or sulfur). Each heteroatom is independently selected from nitrogen, quaternary nitrogen, oxidized nitrogen (e.g., NO); oxygen; and sulfur, including sulfoxide and sulfone. Some of bonds that constitute the heterocyclyl may be unsaturated bonds. A part of the bicyclic heterocyclyl may have aromaticity. Examples thereof include aspects in a state where the monocyclic heterocyclyl having a 5- and 6-membered ring is fused with a benzene ring, a pyrrole ring, a furan ring, a thiophene ring, a pyrazole ring, an imidazole ring, an oxazole ring, a thiazole ring, or a pyridine ring, and a state where at least a partially unsaturated hydrocarbon ring radical having a 5- and 6-membered ring is fused with a pyrrole ring, a furan ring, a thiophene ring, a pyrazole ring, an imidazole ring, an oxazole ring, a thiazole ring, or a pyridine ring. Examples of such heterocyclyl groups include aziridinyl, azetidinyl, pyrrolidinyl, piperidinyl, dihydropyridyl, tetrahydropyridyl, dihydropyridazinyl, tetrahydropy-ridazinyl, dihydropyrimidinyl, tetrahydropyrimidinyl, dihydropyrazinyl, tetrahy-dropyrazinyl, oxetanyl, tetrahydrofuryl, dihydrofuryl, tetrahydropyranyl, dihy-dropyranyl, tetrahydrothienyl, tetrahydrothiopyranyl, dihydrothiopyranyl, piperazinyl, morpholinyl, thiomorpholinyl, dihydroindolyl, dihydroisoindolyl, dihydrobenzofuryl, dihydroisobenzofuryl, tetrahydrobenzoxazolyl, dihydrofuropyridyl, dihydropyrazolo-morpholinyl, pyridinodioxanyl, dihydroazabenzofuryl, dihydroazaisobenzofuryl, dihy-droazaindolyl, 2-oxaspiro[3.5]nonyl, and 2-oxaspiro[3.3]heptyl.

Examples of “N-containing heterocyclic ring” formed by taken together R^{A} and R^{B} at the —N(R^{A})(R^{B}) with the nitrogen atom include azetidine, pyrrolidine, pyrazole, imidazole, triazole, tetrazole, piperidine, dihydropyridine, tetrahydropyridine, piperazine, morpholine, and thiomorpholine.

If a group is described as being “substituted”, a non-hydrogen substituent is in the place of a hydrogen substituent on a carbon, sulfur or nitrogen of the substituent. Thus, for example, a substituted alkyl is an alkyl wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent can be identical or different (unless otherwise stated).

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. If the compound contains a disubstituted cycloalkyl, the cycloalkyl substituent may have a cis- or trans-configuration. The term “geometric isomer” refers to compounds having at least one double bond, wherein the double bond(s) may exist in cis (also referred to as syn or entgegen (E)) or trans (also referred to as anti or zusammen (Z)) forms as well as mixtures thereof. When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99% or 99.9% pure by weight. Geometric isomeric purity is determined by dividing the weight of the named or depicted geometric isomer in the mixture by the total weight of all of the geometric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diastereomeric forms (e.g., diastereomerically pure, diastereomerically enriched and equimolar mixtures of one or more diastereomers (e.g., racemic mixtures) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enan-tiomerically-pure intermediates, reagents, and catalysts by well-known asymmetric synthetic methods.

When a compound is designated by a name or structure that indicates a single enantiomer, unless indicated othelwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

It is to be understood that when a compound herein is represented by a structural formula or designated by a chemical name herein, all other tautomeric forms which may exist for the compound are encompassed by the structural formula.

As used herein, the term “pharmaceutically acceptable salt” refers to pharmaceutical salts that are, within the scope of sound medical judgment, suitable for use in contact with the tissues of humans and lower animals without undue toxicity, irritation, and allergic response, and are commensurate with a reasonable benefit/risk ratio. Pharmaceutically acceptable salts are well known in the art. For example, S. M. Berge, et al. describes pharmacologically acceptable salts in J. Pharm. Sci., 1977, 66:1-19.

Included in the present teachings are pharmaceutically acceptable salts of the compounds disclosed herein. Compounds having basic amine groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, nitric, and sulfuric acids) and of organic acids (such as, e.g., acetic, trifluoroacetic, benzene-sulfonic, benzoic, methanesulfonic, and p-toluenesulfonic acids). Compounds with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Furthermore, the present invention also includes various hydrates or solvates, and any of crystalline polymorphs of the compound of Formula (I) and a pharmaceutically acceptable salt thereof. In addition, the present invention also includes compounds labeled with various radioactive or non-radioactive isotopes.

It is to be noted that the compound of Formulas (Ia) and (Ib) is included within the scope of the compound of Formula (I), and accordingly suitable examples or embodiments of the compound of Formulas (Ia) and (Ib) are to be referred to those as ex-emplified for the compound of Formula (I).

### <Compositions and Compounds of the Invention>

In a first embodiment, the invention provides a pharmaceutical composition comprising a pharmaceutically acceptable carrier or diluent and a compound represented by the following Formula (I).

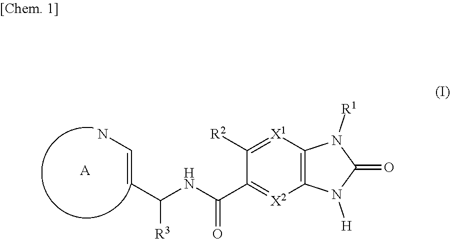

or a pharmaceutically acceptable salt thereof, wherein:
R¹ is hydrogen, C₁₋₃-alkyl, or C₁₋₃-deuteroalkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen, aryl, carboxy, —CO—O—(C₁₋₄-alkyl), or C₁₋₄-alkyl which may be substituted with hydroxy,
a ring A:

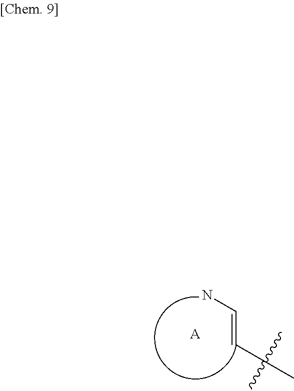

is a group o the formula:

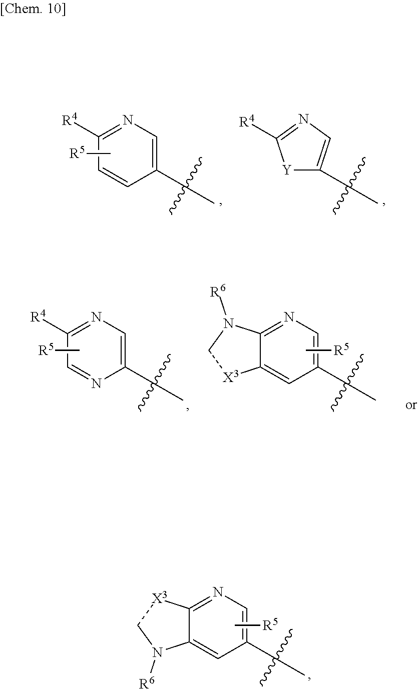

the wavy line indicates a point of attachment to the rest of molecule,
R⁴ is —N(R^{A})(R^{B}), —O—R^{A}, or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, hydroxy, —O—(C₁₋₄-alkyl), C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, —O—(C₁₋₄-haloalkyl), amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino.

In a 2^{nd} embodiment, the invention provides a pharmaceutical composition according to the 1^{st} embodiment, which is a pharmaceutical composition comprising a pharmaceutically acceptable carrier or diluent and a compound represented by the following Formula (I).

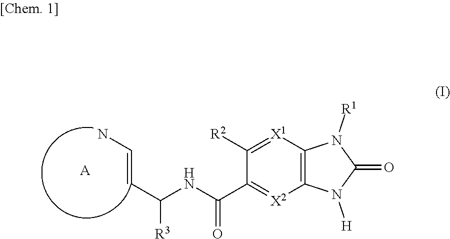

or a pharmaceutically acceptable salt thereof, wherein:
R¹ is hydrogen or C₁₋₃-alkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen or C₁₋₄-alkyl,
a ring A:

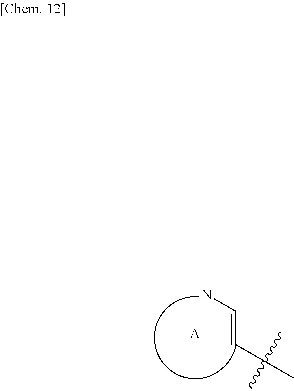

is a group of the formula:

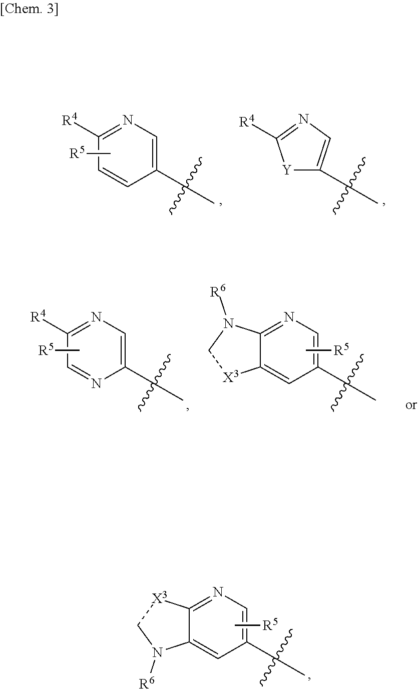

the wavy line indicates a point of attachment to the rest of molecule,
R⁴ is —N(R^{A})(R^{B}), —R^{A}, or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino.

In a 3^{rd} embodiment, the invention provides a pharmaceutical composition according to the 2^{nd} embodiment, wherein R¹ is hydrogen, methyl, ethyl, or isopropyl, and R³ is hydrogen or methyl.

In a 4^{th} embodiment, the invention provides a pharmaceutical composition according to the 3^{rd} embodiment, wherein the ring A:

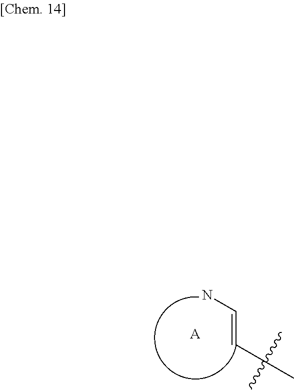

is a group of the formula:

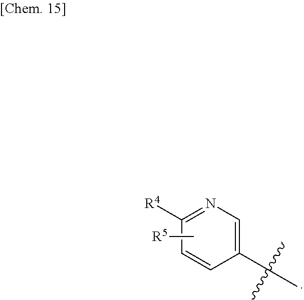

In a 5^{th} embodiment, the invention provides a pharmaceutical composition according to the 1^{st} to 4^{th} embodiments, wherein R⁴ is —N(R^{A})(R^{B}), or —O—R^{A}, and R^{s} is hydrogen, in which R^{A} is C₁₋₄-alkyl optionally substituted with one or more groups selected from the group consisting of halogen, C₃₋₆-cycloalkyl, phenyl, tetrahydropyranyl, piperidyl, piperazinyl, pyridyl, pyrimidyl, and indazolyl, in which said C₃₋₆-cycloalkyl, phenyl, tetrahydropyranyl, piperidyl, piperazinyl, pyridyl, pyrimidyl, and indazolyl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; phenyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with tetrahydropyranyl, piperidyl, or piperazinyl, each of which may be further substituted with halogen, cyano, hydroxy, C₁₋₄-alkyl, —O—(C₁₋₄-alkyl), C₁₋₄-haloalkyl, amino, or mono- or di-(C₁₋₄-alkyl)amino; tetrahydropyranyl, piperidyl, or piperazinyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or pyridyl, pyrimidyl, or indazolyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and R^{B} is methyl.

In a 6^{th} embodiment, the invention provides a pharmaceutical composition according to the 5^{th} embodiment, wherein R^{A} is isopropyl; C₃₋₆-cycloalkyl substituted with one or more groups selected from the group consisting of halogen, cyano, C₁₋₄-alkyl, and C₁₋₄-haloalkyl; phenyl substituted with one or more groups selected from the group consisting of halogen, cyano, C₁₋₄-alkyl, and C₁₋₄-haloalkyl; or pyridyl substituted with one or more groups selected from the group consisting of halogen, cyano, C₁₋₄-alkyl, and C₁₋₄-haloalkyl.

In a 7^{th} embodiment, the invention provides a pharmaceutical composition according to the 1^{st} embodiment, wherein the compound is selected from the group consisting of
N-((6-(2-Fluorophenoxy)pyridin-3-yl)methyl)-1-methyl-2-oxo-2,3-dihydro-1H-ben zimidazole-5-carboxamide,
N-((6-(4-Fluorophenoxy)pyridin-3-yl)methyl)-1-isopropyl-2-oxo-2,3-dihydro-1H-b enzimidazole-5-carboxamide,
6-Fluoro-N-((6-(2-fluorophenoxy)pyridin-3-yl)methyl)-1-methyl-2-oxo-2,3-dihydro-1H-benzimidazole-5-carboxamide,
N-((6-(2,4-Difluorophenoxy)pyridin-3-yl)methyl)-1-methyl-2-oxo-2,3-dihydro-1H-benzimidazole-5-carboxamide,
N-((2-(4-Fluorophenoxy)thiazol-5-yl)methyl)-1-methyl-2-oxo-2,3-dihydro-1H-benz imidazole-5-carboxamide,
N-(1-(6-(4-Fluorophenoxy)pyridin-3-yl)ethyl)-1-methyl-2-oxo-2,3-dihydro-1H-ben zimidazole-5-carboxamide,
(S)—N-(1-(6-(3,4-Difluorophenoxy)pyridin-3-yl)ethyl)-1-methyl-2-oxo-2,3-dihydro-1H-benzimidazole-5-carboxamide,
N-((6-((4-Fluorophenyl)amino)pyridin-3-yl)methyl)-2-oxo-2,3-dihydro-1H-benzimid azole-5-carboxamide,
N-((6-((4-Fluorophenyl)(methyl)amino)pyridin-3-yl)methyl)-1-methyl-2-oxo-2,3-dih ydro-1H-benzimidazole-5-carboxamide,
N-((6-((4,4-Difluorocyclohexyl)(methyl)amino)pyridin-3-yl)methyl)-1-methyl-2-oxo-2,3-dihydro-1H-benzimidazole-5-carboxamide,
N-((6-(Isopropyl(methyl)amino)pyridin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyridin-3-yl)methyl)-2-oxo-2,3-dihydro-1H-benzimidazole-5-carboxamide,
(S)—N-(1-(6-((4-Fluorophenyl)(methyl)amino)pyridin-3-yl)ethyl)-1-methyl-2-oxo-2,3-dihydro-1H-benzimidazole-5-carboxamide,
(S)-1-Methyl-N-((6-(2-methylpyrrolidin-1-yl)pyridin-3-yl)methyl)-2-oxo-2,3-dihydro-1H-benzimidazole-5-carboxamide,
N-((6-(1-(4-Fluorophenyl)ethyl)pyridin-3-yl)methyl)-1-methyl-2-oxo-2,3-dihydro-1H-benzimidazole-5-carboxamide,
N-((6-(Fluoro(4-fluorophenyl)methyl)pyridin-3-yl)methyl)-1-methyl-2-oxo-2,3-dihyd ro-1H-benzimidazole-5-carboxamide,
N-((1-Isopropyl-2,3-dihydro-1H-pyrrolo[2,3-b]pyridin-5-yl)methyl)-1-methyl-2-oxo-2,3-dihydro-1H-benzimidazole-5-carboxamide,
N-((1-(4,4-difluorocyclohexyl)-2,3-dihydro-1H-pyrrolo[2,3-b]pyridin-5-yl)methyl)-1-methyl-2-oxo-2,3-dihydro-1H-benzimidazole-5-carboxamide,
N-((1-(2-Fluorophenyl)-2,3-dihydro-1H-pyrrolo[2,3-b]pyridin-5-yl)methyl)-1-methyl-2-oxo-2,3-dihydro-1H-benzimidazole-5-carboxamide,
1-Methyl-2-oxo-N-((1-phenyl-1H-pyrrolo[2,3-b]pyridin-5-yl)methyl)-2,3-dihydro-1H-benzimidazole-5-carboxamide,
N-((3-(2,6-Difluorophenyl)-3H-imidazo[4,5-b]pyridin-6-yl)methyl)-1-methyl-2-oxo-2,3-dihydro-1H-benzimidazole-5-carboxamide,
N-((6-(3-Cyanophenoxy)pyridin-3-yl)methyl)-1-methyl-2-oxo-2,3-dihydro-1H-benzi midazole-5-carboxamide,
N-((6-(Cyclopropyl(4-fluorophenyl)amino)pyridin-3-yl)methyl)-1-methyl-2-oxo-2,3-dihydro-1H-benzimidazole-5-carboxamide,
1-Methyl-N-((6-(methyl(1-methylpiperidin-4-yl)amino)pyridin-3-yl)methyl)-2-oxo-2,3-dihydro-1H-benzimidazole-5-carboxamide,
N-((2-((4,4-Difluorocyclohexyl)amino)thiazol-5-yl)methyl)-1-methyl-2-oxo-2,3-dihy dro-1H-benzimidazole-5-carboxamide,
1-Methyl-N-((6-(methyl(3-((4-methylpiperazin-1-yl)methyl)phenyl)amino)pyridin-3-yl)methyl)-2-oxo-2,3-dihydro-1H-benzimidazole-5-carboxamide,
N-(1-(6-(2,4-Difluorophenoxy)pyridin-3-yl)ethyl)-1-methyl-2-oxo-2,3-dihydro-1H-be nzimidazole-5-carboxamide,
N-((6-((4,4-Difluorocyclohexyl)(methyl)amino)pyrazin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyrazin-3-yl)methyl)-2-oxo-2,3-dihydro-1H-benzimidazole-5-carboxamide,
1-Methyl-N-((6-(methyl(spiro[2.3]hexan-5-yl)amino)pyridin-3-yl)methyl)-2-oxo-2,3-dihydro-1H-benzimidazole-5-carboxamide,
(S)—N-((6-((3,3-Difluorocyclopentyl)(methyl)amino)pyridin-3-yl)methyl)-1-methyl-2-oxo-2,3-dihydro-1H-benzimidazole-5-carboxamide,
(R)—N-((6-((3,3-Difluorocyclopentyl)(methyl)amino)pyridin-3-yl)methyl)-1-methyl-2-oxo-2,3-dihydro-1H-benzimidazole-5-carboxamide,
N-((6-((3-(Difluoromethoxy)cyclobutyl)(methyl)amino)pyridin-3-yl)methyl)-1-methyl-2-oxo-2,3-dihydro-1H-benzimidazole-5-carboxamide and its cis- and trans-isomers,
1-(Trideuteromethyl)-N-((6-(methyl(cis-3-(trifluoromethyl)cyclobutyl)amino)pyridin-3-yl)methyl)-2-oxo-2,3-dihydro-1H-benzimidazole-5-carboxamide,
6-Fluoro-N-((6-(methyl(cis-3-(trifluoromethyl)cyclobutyl)amino)pyridin-3-yl)methyl)-2-oxo-2,3-dihydro-1H-benzimidazole-5-carboxamide,
1-Methyl-N-((6-(methyl(3-(trifluoromethoxy)cyclobutyl)amino)pyridin-3-yl)methyl)-2-oxo-2,3-dihydro-1H-benzimidazole-5-carboxamide and its cis- and trans-isomers,
N-((6-(((1S,3R)-3-Fluorocyclopentyl)(methyl)amino)pyridin-3-yl)methyl)-1-methyl-2-oxo-2,3-dihydro-1H-benzimidazole-5-carboxamide,
N-((6-(((1R,3S)-3-Fluorocyclopentyl)(methyl)amino)pyridin-3-yl)methyl)-1-methyl-2-oxo-2,3-dihydro-1H-benzimidazole-5-carboxamide,
N-((6-((6,6-Difluorobicyclo[3.1.0]hexan-3-yl)(methyl)amino)pyridin-3-yl)methyl)-1-methyl-2-oxo-2,3-dihydro-1H-benzimidazole-5-carboxamide, and
N-((6-((4,4-Difluorocyclohexyl)(methyl)amino)pyridin-3-yl)methyl)-6-fluoro-1-methyl-2-oxo-2,3-dihydro-1H-benzimidazole-5-carboxamide,
or a pharmaceutically acceptable salt thereof.

In an 8^{th} embodiment, the invention provides a compound represented by the following Formula (Ia):

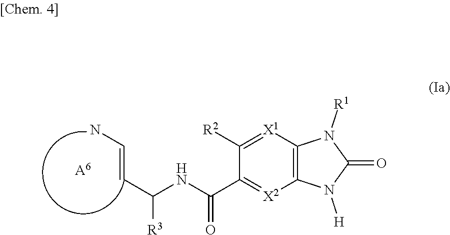

or a pharmaceutically acceptable salt thereof, wherein:
R¹ is hydrogen, C₁₋₃-alkyl, or C₁₋₃-deuteroalkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen, aryl, carboxy, —CO—O—(C₁₋₄-alkyl), or C₁₋₄-alkyl which may be substituted with hydroxy,
a ring A¹:

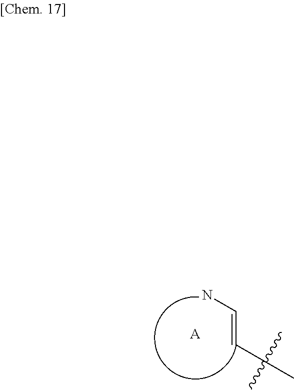

is a group of the formula:

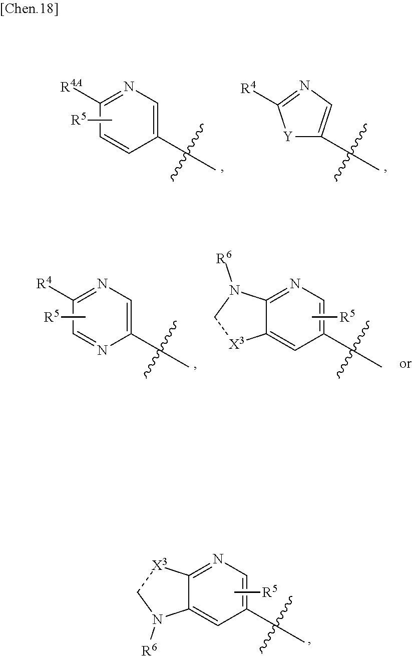

the wavy line indicates a point of attachment to the rest of molecule,
R⁴ is —N(R^{A})(R^{B}), —O—R^{A}, or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
R^{4A} is —N(R^{A})(R^{B}), or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, hydroxy, —O—(C₁₋₄-alkyl), C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, —O—(C₁₋₄-haloalkyl), amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
provided that the compound is not

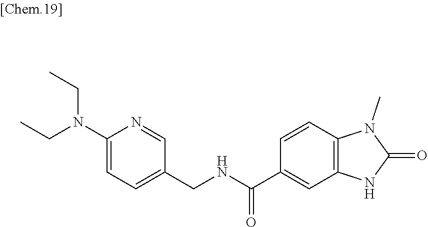 [SMILES: CCN(CC)c1ccc(CNC(=O)c2ccc3c(c2)[nH]c(=O)n3C)cn1]

In a 9^{th} embodiment, the invention provides a compound according to the 8^{th} embodiment, which is a compound represented by the following Formula (Ia):

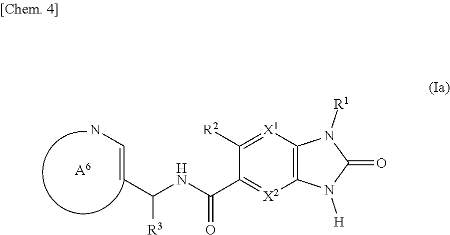

or a pharmaceutically acceptable salt thereof, wherein:
R¹ is hydrogen or C₁₋₃-alkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen or C₁₋₄-alkyl,
a ring A¹:

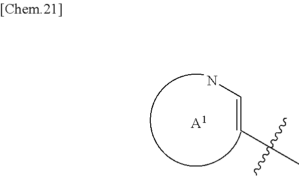

is a group of the formula:

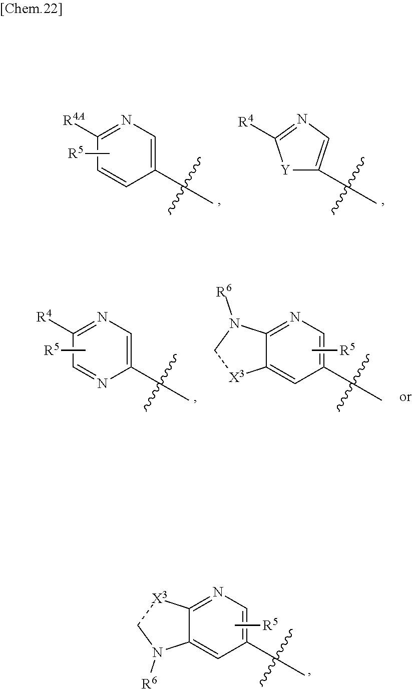

the wavy line indicates a point of attachment to the rest of molecule,
R⁴ is —N(R^{A})(R^{B}), —O—R^{A}, or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R^{4A} is —N(R^{A})(R^{B}), or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
provided that the compound is not

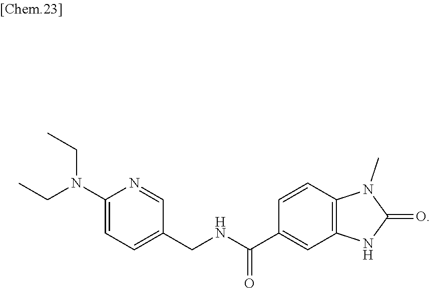 [SMILES: CCN(CC)c1ccc(CNC(=O)c2ccc3c(c2)[nH]c(=O)n3C)cn1]

In a 10^{th} embodiment, the invention provides a compound according to the 9^{th} embodiment, or a pharmaceutically acceptable salt thereof, wherein R¹ is hydrogen, methyl, ethyl, or isopropyl, and R³ is hydrogen or methyl.

In an 11^{th} embodiment, the invention provides a compound according to the 10^{th} embodiment, or a pharmaceutically acceptable salt thereof, wherein X¹ and X² are each independently C—R⁷, in which R⁷ is hydrogen or halogen.

In a 12^{th} embodiment, the invention provides a compound according to the 11^{th} embodiment, or a pharmaceutically acceptable salt thereof, wherein the ring A¹:

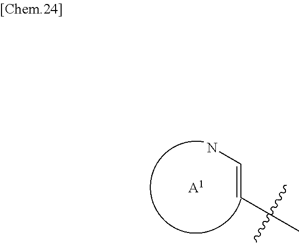

is a group of the formula:

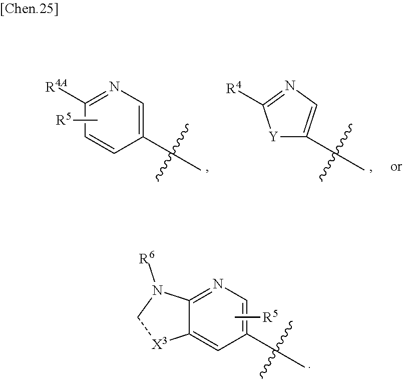

In a 13^{th} embodiment, the invention provides a compound according to the 8^{th} to 12^{th} embodiments, or a pharmaceutically acceptable salt thereof, wherein R⁴ and R^{4A} are each —N(R^{A})(R^{B}), and R⁵ is hydrogen, and R⁶ is R^{A}, in which R^{A} is C₁₋₄-alkyl optionally substituted with one or more groups selected from the group consisting of halogen, C₃₋₆-cycloalkyl, phenyl, tetrahydropyranyl, piperidyl, piperazinyl, pyridyl, pyrimidyl, and indazolyl, in which said C₃₋₆-cycloalkyl, phenyl, tetrahydropyranyl, piperidyl, piperazinyl, pyridyl, pyrimidyl, and indazolyl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; phenyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with tetrahydropyranyl, piperidyl, or piperazinyl, each of which may be further substituted with halogen, cyano, hydroxy, C₁₋₄-alkyl, —O—(C₁₋₄-alkyl), C₁₋₄-haloalkyl, amino, or mono- or di-(C₁₋₄-alkyl)amino; tetrahydropyranyl, piperidyl, or piperazinyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or pyridyl, pyrimidyl, or indazolyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and R^{B} is methyl.

In a 14^{th} embodiment, the invention provides a compound according to the 13^{th} embodiment, or a pharmaceutically acceptable salt thereof, wherein R^{A} is isopropyl; C₃₋₆-cycloalkyl substituted with one or more groups selected from the group consisting of halogen, cyano, C₁₋₄-alkyl, and C₁₋₄-haloalkyl; phenyl substituted with one or more groups selected from the group consisting of halogen, cyano, C₁₋₄-alkyl, and C₁₋₄-haloalkyl; or pyridyl substituted with one or more groups selected from the group consisting of halogen, cyano, C₁₋₄-alkyl, and C₁₋₄-haloalkyl.

In a 15^{th} embodiment, the invention provides a compound according to the 8^{th} embodiment, or a pharmaceutically acceptable salt thereof, wherein:
R¹ is hydrogen, C₁₋₃-alkyl, or C₁₋₃-deuteroalkyl,
R² is hydrogen or halogen,
R³ is hydrogen or C₁₋₄-alkyl,
a ring A¹;

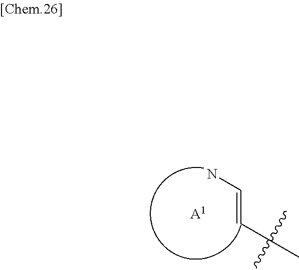

is a group of the formula:

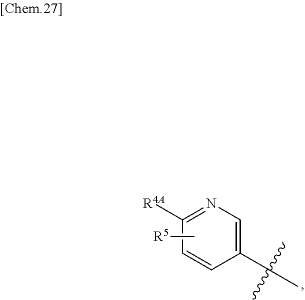

the wavy line indicates a point of attachment to the rest of molecule,
R^{4A} is —N(R^{A})(R^{B}),
R⁵ is hydrogen or halogen,
X¹ and X² are each independently N or C—R⁷,
R⁷ is hydrogen or halogen,
R^{A} is C₃₋₆-cycloalkyl substituted with one or more groups selected from the group consisting of halogen, C₁₋₄-haloalkyl, and —O—(C₁₋₄-haloalkyl), and
R^{B} is hydrogen or C₁₋₄-alkyl.

In a 16^{th} embodiment, the invention provides a compound according to the 8^{th} embodiment, wherein the compound is selected from the group consisting of
N-((2-(4-Fluorophenoxy)thiazol-5-yl)methyl)-1-methyl-2-oxo-2,3-dihydro-1H-benz imidazole-5-carboxamide,
N-((6-((4-Fluorophenyl)amino)pyridin-3-yl)methyl)-2-oxo-2,3-dihydro-1H-benzimi dazole-5-carboxamide,
N-((6-((4-Fluorophenyl)(methyl)amino)pyridin-3-yl)methyl)-1-methyl-2-oxo-2,3-dih ydro-1H-benzimidazole-5-carboxamide,
N-((6-((4,4-Difluorocyclohexyl)(methyl)amino)pyridin-3-yl)methyl)-1-methyl-2-oxo-2,3-dihydro-1H-benzimidazole-5-carboxamide,
N-((6-(Isopropyl(methyl)amino)pyridin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyridin-3-yl)methyl)-2-oxo-2,3-dihydro-1H-benzimidazole-5-carboxamide,
(S)—N-(1-(6-((4-Fluorophenyl)(methyl)amino)pyridin-3-yl)ethyl)-1-methyl-2-oxo-2,3-dihydro-1H-benzimidazole-5-carboxamide,
(S)-1-Methyl-N-((6-(2-methylpyrrolidin-1-yl)pyridin-3-yl)methyl)-2-oxo-2,3-dihydro-1H-benzimidazole-5-carboxamide,
N-((6-(1-(4-Fluorophenyl)ethyl)pyridin-3-yl)methyl)-1-methyl-2-oxo-2,3-dihydro-1H-benzimidazole-5-carboxamide,
N-((6-(Fluoro(4-fluorophenyl)methyl)pyridin-3-yl)methyl)-1-methyl-2-oxo-2,3-dihyd ro-1H-benzimidazole-5-carboxamide,
N-((1-Isopropyl-2,3-dihydro-1H-pyrrolo[2,3-b]pyridin-5-yl)methyl)-1-methyl-2-oxo-2,3-dihydro-1H-benzimidazole-5-carboxamide,
N-((1-(4,4-difluorocyclohexyl)-2,3-dihydro-1H-pyrrolo[2,3-b]pyridin-5-yl)methyl)-1-methyl-2-oxo-2,3-dihydro-1H-benzimidazole-5-carboxamide,
N-((1-(2-Fluorophenyl)-2,3-dihydro-1H-pyrrolo[2,3-b]pyridin-5-yl)methyl)-1-methyl-2-oxo-2,3-dihydro-1H-benzimidazole-5-carboxamide,
1-Methyl-2-oxo-N-((1-phenyl-1H-pyrrolo[2,3-b]pyridin-5-yl)methyl)-2,3-dihydro-1H-benzimidazole-5-carboxamide,
N-((3-(2,6-Difluorophenyl)-3H-imidazo[4,5-b]pyridin-6-yl)methyl)-1-methyl-2-oxo-2,3-dihydro-1H-benzimidazole-5-carboxamide,
N-((6-(Cyclopropyl(4-fluorophenyl)amino)pyridin-3-yl)methyl)-1-methyl-2-oxo-2,3-dihydro-1H-benzimidazole-5-carboxamide,
1-Methyl-N-((6-(methyl(1-methylpiperidin-4-yl)amino)pyridin-3-yl)methyl)-2-oxo-2, 3-dihydro-1H-benzimidazole-5-carboxamide,
N-((2-((4,4-Difluorocyclohexyl)amino)thiazol-5-yl)methyl)-1-methyl-2-oxo-2,3-dihy dro-1H-benzimidazole-5-carboxamide,
1-Methyl-N-((6-(methyl(3-((4-methylpiperazin-1-yl)methyl)phenyl)amino)pyridin-3-yl)methyl)-2-oxo-2,3-dihydro-1H-benzimidazole-5-carboxamide,
N-((6-((4,4-Difluorocyclohexyl)(methyl)amino)pyrazin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyrazin-3-yl)methyl)-2-oxo-2,3-dihydro-1H-benzimidazole-5-carboxamide,
1-Methyl-N-((6-(methyl(spiro[2.3]hexan-5-yl)amino)pyridin-3-yl)methyl)-2-oxo-2,3-dihydro-1H-benzimidazole-5-carboxamide,
(S)—N-((6-((3,3-Difluorocyclopentyl)(methyl)amino)pyridin-3-yl)methyl)-1-methyl-2-oxo-2,3-dihydro-1H-benzimidazole-5-carboxamide,
(R)—N-((6-((3,3-Difluorocyclopentyl)(methyl)amino)pyridin-3-yl)methyl)-1-methyl-2-oxo-2,3-dihydro-1H-benzimidazole-5-carboxamide,
N-((6-((3-(Difluoromethoxy)cyclobutyl)(methyl)amino)pyridin-3-yl)methyl)-1-methyl-2-oxo-2,3-dihydro-1H-benzimidazole-5-carboxamide and its cis- and trans-isomers,
1-(Trideuteromethyl)-N-((6-(methyl(cis-3-(trifluoromethyl)cyclobutyl)amino)pyridin-3-yl)methyl)-2-oxo-2,3-dihydro-1H-benzimidazole-5-carboxamide,
6-Fluoro-N-((6-(methyl(cis-3-(trifluoromethyl)cyclobutyl)amino)pyridin-3-yl)methyl)-2-oxo-2,3-dihydro-1H-benzimidazole-5-carboxamide,
1-Methyl-N-((6-(methyl(3-(trifluoromethoxy)cyclobutyl)amino)pyridin-3-yl)methyl)-2-oxo-2,3-dihydro-1H-benzimidazole-5-carboxamide and its cis- and trans-isomers,
N-((6-(((1S,3R)-3-Fluorocyclopentyl)(methyl)amino)pyridin-3-yl)methyl)-1-methyl-2-oxo-2,3-dihydro-1H-benzimidazole-5-carboxamide,
N-((6-(((1R,3S)-3-Fluorocyclopentyl)(methyl)amino)pyridin-3-yl)methyl)-1-methyl-2-oxo-2,3-dihydro-1H-benzimidazole-5-carboxamide,
N-((6-((6,6-Difluorobicyclo[3.1.0]hexan-3-yl)(methyl)amino)pyridin-3-yl)methyl)-1-methyl-2-oxo-2,3-dihydro-1H-benzimidazole-5-carboxamide, and
N-((6-((4,4-Difluorocyclohexyl)(methyl)amino)pyridin-3-yl)methyl)-6-fluoro-1-meth yl-2-oxo-2,3-dihydro-1H-benzimidazole-5-carboxamide,
or a pharmaceutically acceptable salt thereof.

In a 17^{th} embodiment, the invention provides a compound represented by the following Formula (Ib):

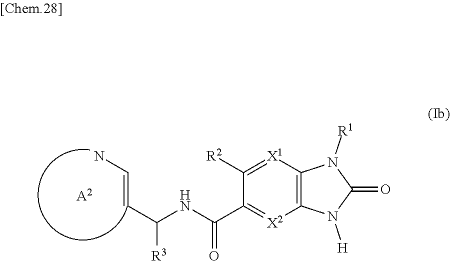

or a pharmaceutically acceptable salt thereof, wherein:
R¹ is hydrogen, C₁₋₃-alkyl, or C₁₋₃-deuteroalkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen, aryl, carboxy, —CO—O—(C₁₋₄-alkyl), or C₁₋₄-alkyl which may be substituted with hydroxy,
a ring A²:

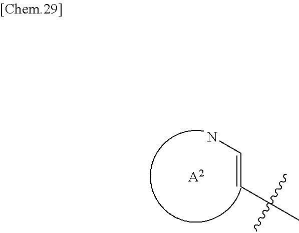

is a group of the formula:

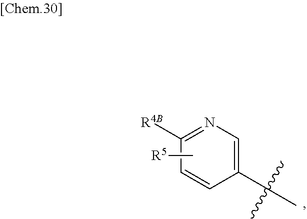

the wavy line indicates a point of attachment to the rest of molecule,
R^{4B} is —O—R^{C},
R⁵ is hydrogen, halogen, or C₁₋₄-alkyl,
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R^{C} is C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino.

In an 18^{th} embodiment, the invention provides a compound according to the 17^{th} embodiment, or a pharmaceutically acceptable salt thereof, wherein:
R¹ is hydrogen, C₁₋₃-alkyl, or C₁₋₃-deuteroalkyl,
R² is hydrogen or halogen,
R³ is hydrogen or C₁₋₄-alkyl,
R⁵ is hydrogen or halogen,
R⁷ is hydrogen or halogen, and
R^{C} is C₃₋₆-cycloalkyl substituted with one or more groups selected from the group consisting of halogen, C₁₋₄-haloalkyl, and —O—(C₁₋₄-haloalkyl).

In certain embodiments, the invention is any one of the compounds depicted in the exemplification section of the instant application; and pharmaceutically acceptable salts as well as the neutral forms of these compounds also are included in the invention. Specifically, the invention is any one of the compounds depicted in Examples 1-147; and pharmaceutically acceptable salts as well as the neutral forms of these compounds also are included in the invention. In preferred embodiments, the invention is any one of Compounds 1-147; and pharmaceutically acceptable salts as well as the neutral forms of these compounds also are included in the invention.

### <Methods of Preparing Compounds of the Invention>

Methods of preparing compounds of Formula (I) are disclosed. In general, a compound of Formula (I) or a pharmaceutically acceptable salt thereof may be prepared by reacting a compound of Formula (I

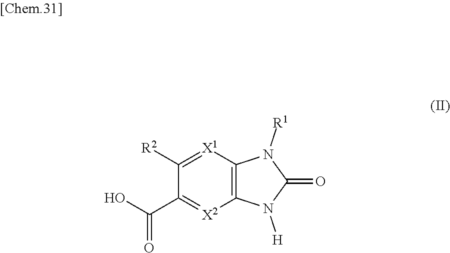

or its salt with a compound of Formula (III)

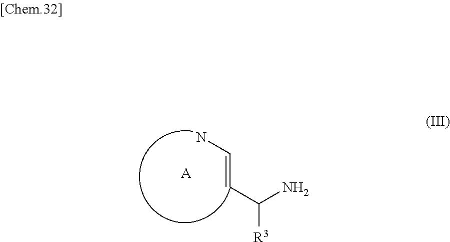

or its salt.

Suitable salts of the compounds (II) and (III) can be referred to the salts as ex-emplified for the compound (I).

The reaction is usually carried out in a presence of a conventional condensing agent such as 1-ethyl-3-(3-dimethylaminopropyl)carbodiimide (EDC) or its hydrochloride, 1-[bis(dimethylamino)methylene]-1H-1,2,3-triazolo[4,5-b]pyridinium 3-oxide hexafluorophosphate (HATU), benzotriazol-1-yloxytripyrrolidinophosphonium hexafluorophosphate (PyBOP), or propanephosphonic acid anhydride (T₃P), in a conventional solvent, such as methylene chloride, chloroform, pyridine, dioxane, tetrahydrofuran, or N,N-dimethylformamide.

The reaction temperature is not critical and the reaction can be carried out under cooling, at room temperature, or under heating.

This reaction is preferably carried out in the presence of a conventional inorganic base or in the presence of a conventional organic base.

The compounds obtained by the above process can be isolated and purified by a conventional method such as pulverization, recrystallization, chromatography, and re-precipitation.

Detailed synthetic protocols for preparing exemplary compounds of Formula (I) and their intermediate compounds can be referred to Examples and Preparations below or similar manners thereto or conventional manners.

### <Pharmaceutical Compositions>

The compounds of Formula (I) disclosed therein are KIT inhibitors. The pharmaceutical composition of the present invention comprises one or more KIT inhibitors, or a pharmaceutically acceptable salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions of the present teachings optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### <Methods of Treatment>

Methods of preventing or treating a KIT mediated disease or condition, es-pecially, the KIT mediated disease or condition associated with improperly regulated kinase signal transduction of mast cells, in a subject are disclosed. The methods can include administering to the subject a therapeutically effective amount of one or more compounds of Formula (I) or compositions provided herein.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

In one embodiment, the KIT mediated disease or condition is a cancer. Examples of cancers include, but are not limited to, mastocytosis, mastocytoma, solid tumor, gastrointestinal stromal tumor (“GIST”), small cell lung cancer, non-small cell lung cancer, acute myelocytic leukemia, acute lymphocytic leukemia, myelodysplastic syndrome, chronic myelogenous leukemia, colorectal carcinoma, gastric carcinoma, testicular cancer, glioblastoma, astrocytoma, melanoma, mast cell tumor, neuroblastoma, sarcoma, and seminoma.

In other embodiments, the KIT mediated disease or condition is an autoimmune disease. Examples of autoimmune diseases include, but are not limited to, multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, and proliferative glomerulonephritis.

In some embodiments, the KIT mediated disease or condition is an allergic disease. Examples of allergic diseases include, but are not limited to, asthma, severe asthma, allergic rhinitis, chronic rhinitis, allergic sinusitis, anaphylactic syndrome, urticaria, food allergy, seasonal allergy, angioedema, atopic dermatitis, allergic contact dermatitis, erythema nodosum, erythema multiforme, cutaneous necrotizing venulitis, insect bite skin inflammation, and blood sucking parasitic infestation.

In other embodiments, the KIT mediated disease or condition is an inflammatory disease. Examples of inflammatory diseases include, but are not limited to, rheumatoid arthritis, conjunctivitis, rheumatoid spondylitis, osteoarthritis, gouty arthritis and other arthritic conditions, inflammatory bowel diseases (IBD), irritable bowel syndrome (IBS), and eosinophilic esophagitis.

In yet other embodiments, the KIT mediated disease or condition is fibrosis. Examples of fibrosis includes, but are not limited to, pulmonary fibrosis, hepatic fibrosis, cardiac fibrosis, and myelofibrosis.

In still other embodiments, the KIT mediated disease or condition is a metabolic disorder. Examples of metabolic disorders include, but are not limited to, diabetes mellitus and its chronic complications; obesity; type I diabetes or type II diabetes; hyperlipidemias and dyslipidemias; atherosclerosis; hypertension; and cardiovascular disease.

In other embodiments, the KIT mediated disease or condition is a neurodegenerative disease. Examples of neurodegenerative diseases include, but are not limited to, Alzheimer's disease, Parkinson's disease, Huntington's disease, the prion diseases, motor neuron disease (MND), and amyotrophic lateral sclerosis (ALS).

In yet other embodiments, the KIT mediated disease or condition includes bone loss, tumor angiogenesis, interstitial cystitis, pulmonary arterial hypertension (PAH), and primary pulmonary hypertension (PPH).

### <Methods of Administration and Dosage Forms>

The precise amount of compound administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57^{th} ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, in-traarticular (in the joints), intravenous, intramuscular, intratumoral, intradermal, intraperitoneal, subcutaneous, orally, topically, intrathecally, inhalationally, trans-dermally, rectally, and the like. Administration techniques that can be employed with the agents and methods described herein are found in e.g., Goodman and Gilman, The Pharmacological Basis of Therapeutics, current ed., Pergamon; and Remington's Pharmaceutical Sciences (current edition), Mack Publishing Co., Easton, Pa.

In addition, the compounds of Formula (I) can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds described herein will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition. In some embodiments, the compounds of Formula (I) can be used in combination with other agents known to have beneficial activity with the compounds of Formula (I) for treating or preventing the KIT mediated diseases or conditions. For example, the compounds of Formula (I) can be administered alone or in combination with one or more other anticancer agents, immunomodulatory agents, antiallergic agents, anti-inflammatory agents, antifibrotic agents, antimetabolites, and anti-neurodegenerative disease agents for treating or preventing the KIT mediated diseases or conditions.

The particular mode of administration and the dosage regimen will be selected by the attending clinician, taking into account the particulars of the case (e.g., the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for preventing or treating a KIT mediated diseases or conditions using the compounds of Formula (I) for guidance.

The compounds or the corresponding pharmaceutical compositions taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intra-pulmonary, intrathecal, rectal and topical modes of administration. Parenteral administration can be by continuous infusion over a selected period of time.

The pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for oral or intravenous administration.

Typically, for oral therapeutic administration, a compound of the present teachings may be incorporated with excipient and used in the form of ingestible tablets, buccal tablets, troches, capsules, elixirs, suspensions, syrups, wafers, and the like.

Typically for parenteral administration, solutions of a compound of the present teachings can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound described herein for the extemporaneous preparation of sterile injectable solutions or dispersions are appropriate.

### EXAMPLES

### Synthetic Preparation of Compound Embodiments

### Abbreviations

TABLE 1

| | |
|---|---|
| Boc | tert-butyloxycarbonyl |
| bs | broad singlet |
| CDI | 1,1′-carbonyldiimidazole |
| d | doublet |
| DAST | (diethylamino)sulfur trifluoride |
| DBU | 1,8-diazabicyclo[5.4.0]undec-7-ene |
| DCM | dichloromethane |
| dd | double doublet |
| DIPEA | diisopropylethylamine |
| DMAP | 4-(dimethylamino)pyridine |
| DMF | N,N-dimethylformamide |
| DMSO | dimethyl sulfoxide |
| DPPA | diphenylphosphoryl azide |
| EDC | 1-ethyl-3-(3-dimethylaminopropyl)carbodiimide |
| EtOAc | ethyl acetate |
| EtOH | ethanol |
| h | hour(s) |
| HATU | 1-[bis(dimethylamino)methylene]-1H-1,2,3-triazolo[4,5- |
| | b]pyridinium 3-oxide hexafluorophosphate |
| HOAt | 1-hydroxy-7-azabenzotriazole |
| HOBt | 1-hydroxybenzotriazole |
| HPLC | high performance liquid chromatography |
| KOtBu | potassium tert-butoxide |
| LC-MS | liquid chromatography-mass spectrometry |
| m | multiplet |
| M | molar |
| MeOH | methanol |
| min | minutes |
| MHz | megahertz |
| N | normal acid or base |
| NMR | nuclear magnetic resolution |
| Pd₂(dba)₃ | tris(dibenzylideneacetone)dipalladium(0) |
| PTSA | p-toluenesulfonic acid |
| PyBOP | benzotriazol-1-yloxytripyrrolidinophosphonium |
| | hexafluorophosphate |
| RT | room temperature |
| s | singlet |
| t | triplet |
| t-BuOH | tert-butyl alcohol |
| T₃P | propanephosphonic acid anhydride |
| TFA | trifluoroacetic acid |
| THF | tetrahydrofuran |
| Xphos | 2-dicyclohexylphosphino-2′,4′,6′-triisopropylbiphenyl |
| Xantphos | 4,5-bis(diphenylphosphino)-9,9-dimethylxanthene |

### Example-1

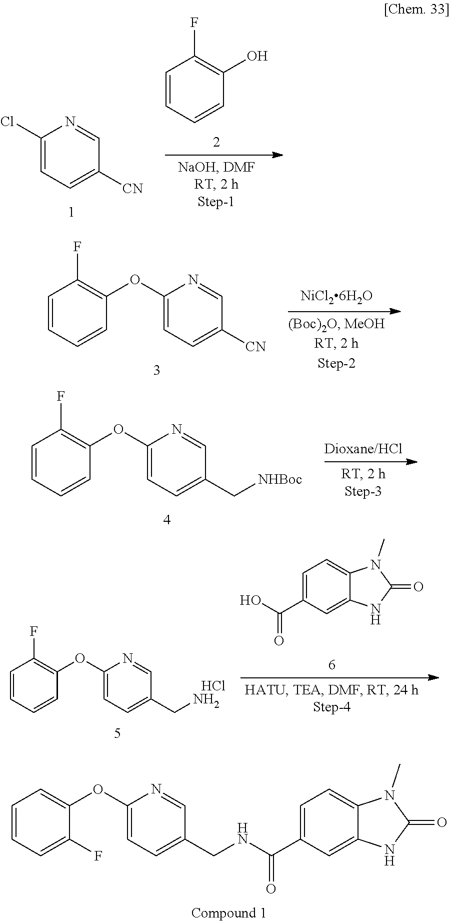

### Step-1: 6-(2-Fluorophenoxy)nicotinonitrile

To a stirred solution of 2-fluorophenol (9 g, 80.28 mmol) in DMF (90 mL) was added NaOH (3.53 g, 88.31 mmol) followed by 6-chloronicotinonitrile (11.12 g, 80.28 mmol) and stirred at RT for 2 h. The reaction mixture was poured into ice cold water and stirred for another 2 h. The solid separated was collected by filtration and dried to get the title compound (10 g, 58.1%). LC-MS: 215 [M+H]⁺

### Step-2: tert-Butyl ((6-(2-fluorophenoxy)pyridin-3-yl)methyl)carbamate

To a solution of 6-(2-fluorophenoxy)nicotinonitrile (3 g, 14 mmol) in MeOH (40 mL) was added NiCl₂·6H₂O (1.66 g, 7 mmol) followed by dropwise addition of di-tert-butyl dicarbonate (4.58 g, 21 mmol) at 0° C. and stirred for 5 min. Then NaBH₄ (1.06 g, 28.01 mmol) was added in portions thereto by maintaining same temperature. After stirring for 2 h at RT, the reaction mass was concentrated to remove the solvent, diluted with EtOAc and filtered on celite bed. The filtrate was washed with water, dried over anhydrous Na₂SO₄ and concentrated to afford the title compound (3.5 g, 78.6%). LC-MS: 319 [M+H]⁺

### Step-3: (6-(2-Fluorophenoxy)pyridin-3-yl)methanamine hydrochloride

To a solution of tert-butyl ((6-(2-fluorophenoxy)pyridin-3-yl)methyl)carbamate (3 g, 9.4 mmol) in 1,4-dioxane (5 ml) was added 4 N HCl in 1,4-dioxane (20 mL) and stirred for 2 h at RT. The reaction mixture was then concentrated, added diethyl ether thereto, and the solid was collected by filtration to get the title compound (2.35 g, 97.9%). LC-MS: 219 [M+H]⁺

### Step-4: N-((6-(2-Fluorophenoxy)pyridin-3-yl)methyl)-1-methyl-2-oxo-2,3-dihydro-1H-benzimid azole-5-carboxamide (Compound 1)

To a solution of 1-methyl-2-oxo-2,3-dihydro-1H-benzimidazole-5-carboxylic acid (2 g, 10.4 mmol) in DMF (30 mL) were added HATU (4.99 g, 13.11 mmol) and triethylamine (5.53 g, 54.64 mmol), and stirred at RT for 10 min. Then (6-(2-fluorophenoxy)pyridin-3-yl)methanamine hydrochloride (2.38 g, 9.34 mmol) was added thereto and the mixture was stirred at RT for 24 h. The reaction mixture was poured into ice cold water and extracted with EtOAc. The separated organic layer washed with brine, dried over anhydrous Na₂SO₄ and concentrated to get a crude compound. The crude compound was purified by combi flash column chromatography using 10% MeOH in DCM as an eluent to afford the title compound (0.4 g, 10.9%). LC-MS: 393 [M+H]⁺; ¹H-NMR (400 MHz, DMSO-D₆) δ 11.11 (s, 1H), 8.95 (t, 1H), 8.07 (s, 1H), 7.88 (d, 1H), 7.63 (d, 1H), 7.53 (s, 1H), 7.4-7.2 (m, 4H), 7.2-7.1 (dd, 2H), 4.45-4.43 (d, 2H), 3.33 (s, 3H).

### Example-2

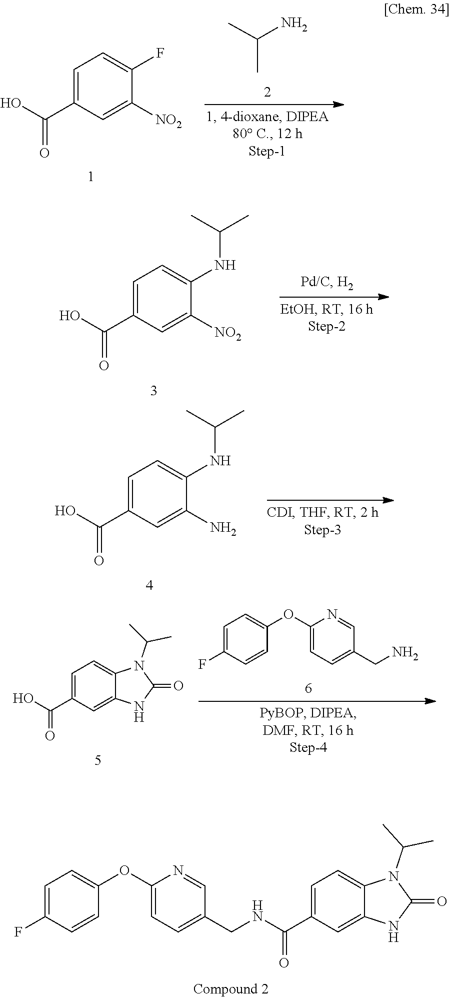

### Step-1: 4-(Isopropylamino)-3-nitrobenzoic acid

To a solution of 4-fluoro-3-nitrobenzoic acid (1 g, 5.4 mmol) in 1,4-dioxane (10 ml) was added DIPEA (2.09 g, 16.2 mmol) followed by propan-2-amine (0.32 g, 5.4 mmol) and stirred at 80° C. for 12 h. Then the reaction mixture was cooled to RT, acidified with citric acid, and poured into ice cold water. The solid separated was collected by filtration and dried to get the title compound (1 g, 10.9%). LC-MS: 223 [M−H]⁻

### Step-2: 3-Amino-4-(isopropylamino)benzoic acid

To a solution of 4-(isopropylamino)-3-nitrobenzoic acid (0.7 g, 3.12 mmol) in EtOH (5 ml) was added Pd/C (0.99 g, 9.3 mmol) under nitrogen atmosphere at RT and the reaction mixture was stirred for 16 h under hydrogen bladder pressure. The reaction mass was filtered through celite, and the filtrate was concentrated to get the title compound (0.45 g, 74.2%). LC-MS: 195 [M+H]⁺; ¹H-NMR (400 MHz, DMSO-D₆) δ 12.7 (s, 1H), 7.18-7.13 (m, 2H), 6.42-6.40 (d, 1H), 4.84-4.82 (d, 2H), 4.6 (s, 1H), 3.64-3.62 (m, 1H), 1.17-1.16 (d, 6H).

### Step-3: 1-Isopropyl-2-oxo-2,3-dihydro-1H-benzimidazole-5-carboxylic acid

To a solution of 3-amino-4-(isopropylamino)benzoic acid (0.2 g, 1.03 mmol) in THF (8 ml) was added CDI (0.33 g, 2.06 mmol) and stirred at RT for 2 h. The reaction mixture was poured into ice cold water and acidified with 1 N HCl, which was extracted with EtOAc, washed with brine, dried over anhydrous Na₂SO₄ and concentrated to get the title compound (0.2 g, 88.4%). LC-MS: 221 [M+H]⁺

### Step-4: N-((6-(4-Fluorophenoxy)pyridin-3-yl)methyl)-1-isopropyl-2-oxo-2,3-dihydro-1H-benzim idazole-5-carboxamide (Compound 2)

To a stirred solution of 1-isopropyl-2-oxo-2,3-dihydro-1H-benzimidazole-5-carboxylic acid (0.1 g, 0.45 mmol) in DMF (5 mL) was added DIPEA (0.17 g, 1.36 mmol) followed by PyBOP (0.35 g, 0.68 mmol) and stirred at RT for 10 min. Then (6-(4-fluorophenoxy)pyridin-3-yl)methanamine (0.11 g, 0.49 mmol) was added thereto and the mixture was stirred at RT for 16 h. The reaction mixture was poured into ice cold water, extracted with EtOAc, washed with brine, dried over anhydrous Na₂SO₄ and concentrated to get a crude compound. The crude compound was purified by preparative TLC, using 5% MeOH in DCM as an eluent to afford the title compound (0.04 g, 21.1%). LC-MS: 421 [M+H]⁺; ¹H-NMR (400 MHz, DMSO-D₆) δ 11.07 (s, 1H), 8.95 (t, 1H), 8.08 (s, 1H), 7.8-7.78 (d, 2H), 7.59-7.57 (d, 1H), 7.5 (s, 1H), 7.33-7.31 (d, 1H), 7.25-7.21 (m, 2H), 7.16-7.13 (m, 2H), 7.01-6.99 (d, 1H), 4.42-4.40 (d, 2H), 1.45 (d, 6H).

### Example-3

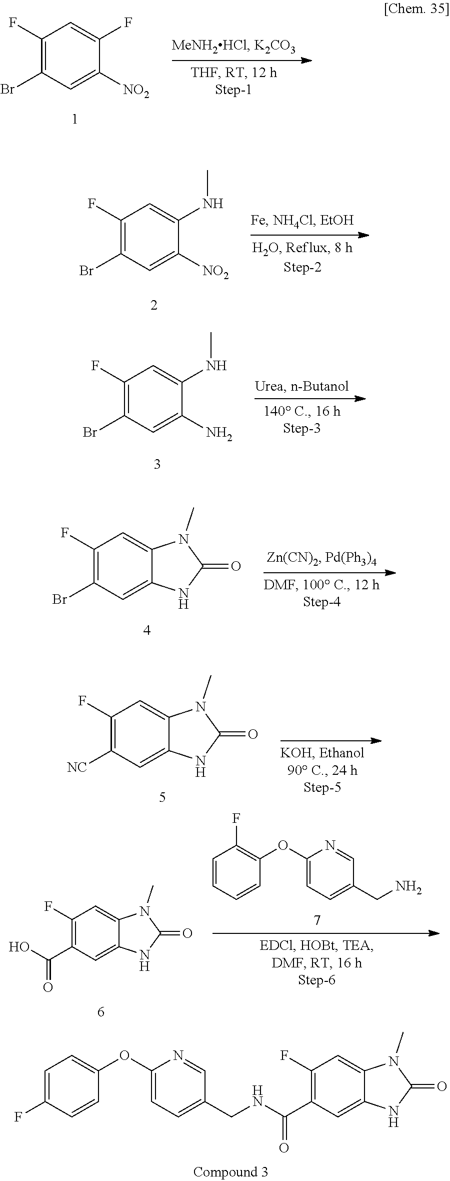

### Step-1: 4-Bromo-5-fluoro-N-methyl-2-nitroaniline

To a stirred solution 1-bromo-2,4-difluoro-5-nitrobenzene (4 g, 16.80 mmol) in THF (40 mL) was added K₂CO₃ (5.8 g, 42.01 mmol) followed by methylamine hydrochloride (1.36 g, 20.16 mmol) and stirred at RT for 12 h. The reaction mixture was concentrated and poured into ice cold water, which was extracted with EtOAc, washed with brine, dried over anhydrous Na₂SO₄ and concentrated to get a crude compound. This was purified by combi flash column chromatography using 10% EtOAc in hexane as an eluent to afford the title compound (2.7 g, 64.6%). ¹H-NMR (400 MHz, DMSO-D₆) δ 8.35-8.31 (m, 2H), 7.01-6.98 (d, 1H) 2.92-2.91 (d, 3H).

### Step-2: 4-Bromo-5-fluoro-N¹-methylbenzene-1,2-diamine

To a stirred solution of 4-bromo-5-fluoro-N-methyl-2-nitroaniline (2 g, 8.03 mmol) in THF (20 mL) and EtOH (20 ml) was added ammonium chloride (2.14 g, 40.15 mmol) followed by iron powder (2.24 g, 40.15 mmol) and stirred at 80° C. for 8 h. The reaction mixture was filtered on celite bed and the filtrate was poured into ice cold water, extracted with EtOAc, washed with brine, dried over anhydrous Na₂SO₄ and concentrated to get the title compound (1.5 g, 85.2%). LC-MS: 219 [M+H]⁺

### Step-3: 5-Bromo-6-fluoro-1-methyl-1,3-dihydro-2H-benzimidazol-2-one

To a solution of 4-bromo-5-fluoro-N¹-methylbenzene-1,2-diamine (0.04 g, 0.16 mmol) in n-butanol (5 ml) was added urea (1.31 g, 21.91 mmol) and stirred at 140° C. for 16 h. The reaction mixture was cooled to RT and acidified with 1 N HCl and concentrated. The crude material was stirred with diethyl ether and collected by filtration to get the title compound (0.02 g, 51%). LC-MS: 245 [M+H]⁺

### Step-4: 6-Fluoro-1-methyl-2-oxo-2,3-dihydro-1H-benzimidazole-5-carbonitrile

To a solution of 5-bromo-6-fluoro-1-methyl-1,3-dihydro-2H-benzimidazol-2-one (0.25 g, 1.02 mmol) in DMF (5 ml) was added zinc cyanide (0.24 g, 2.04 mmol) followed by tetrakis(triphenylphosphine)palladium(0) (0.24 g, 0.2 mmol) and stirred at 100° C. for 12 h. The reaction mixture was poured into ice cold water, extracted with 10% MeOH in DCM, washed with brine, dried over anhydrous Na₂SO₄ and concentrated to get a crude compound. This was purified by combi flash column chromatography using 5% MeOH in DCM as an eluent to get the title compound (0.15 g, 77.3%). LC-MS: 190 [M−H]⁻; ¹H-NMR (400 MHz, DMSO-D₆) δ 11.36 (s, 1H), 7.42-7.40 (s, 1H), 7.38-7.36 (s, 1H), 3.3-3.5 (s, 3H).

### Step-5: 6-Fluoro-1-methyl-2-oxo-2,3-dihydro-1H-benzimidazole-5-carboxylic acid

To a solution of 6-fluoro-1-methyl-2-oxo-2,3-dihydro-1H-benzimidazole-5-carbonitrile (0.15 g, 0.78 mmol) in water (3 ml) and EtOH (3 ml) was added KOH (0.88 g, 15.7 mmol) and stirred at 90° C. for 24 h. Then the reaction mixture was concentrated, diluted with water and acidified with 1 N HCl. The precipitate formed was collected by filtration and dried to get the title compound (0.12 g, 73%). LC-MS: 209 [M−H]⁻; ¹H-NMR (400 MHz, DMSO-D₆) δ 12.87 (s, 1H), 11.36 (s, 1H), 7.46-7.45 (d, 1H), 6.87-6.84 (d, 1H), 3.26-3.25 (s, 3H).

### Step-6: 6-Fluoro-N-((6-(2-fluorophenoxy)pyridin-3-yl)methyl)-1-methyl-2-oxo-2,3-dihydro-1H-benzimidazole-5-carboxamide trifluoroacetate (Compound 3)

To a solution of 6-fluoro-1-methyl-2-oxo-2,3-dihydro-1H-benzimidazole-5-carboxylic acid (0.06 g, 0.29 mmol) in DMF (2 mL) were added EDC·HCl (0.066 g, 0.34 mmol) and HOBt (0.047 g, 0.34 mmol), and stirred at RT for 10 min. Then (6-(2-fluorophenoxy)pyridin-3-yl)methanamine (0.05 g, 0.23 mmol) was added thereto followed by triethylamine (0.087 g, 0.85 mmol) and the mixture was stirred at RT for 16 h. The reaction mixture was poured into ice cold water, extracted with EtOAc, dried over anhydrous Na₂SO₄ and concentrated to get a crude compound. This was purified by preparative HPLC using method A to afford the title compound as TFA salt (0.022 g, 23.4%) LC-MS: 411 [M+H]⁺; ¹H-NMR (400 MHz, DMSO-D₆) δ 11.04 (s, 1H), 8.67-8.66 (d, 1H), 8.04-8.03 (d, 1H), 7.84-7.81 (dd, 1H), 7.3-7.0 (m, 7H), 4.42-4.40 (d, 2H), 3.3-3.28 (s, 3H).

### Example-4

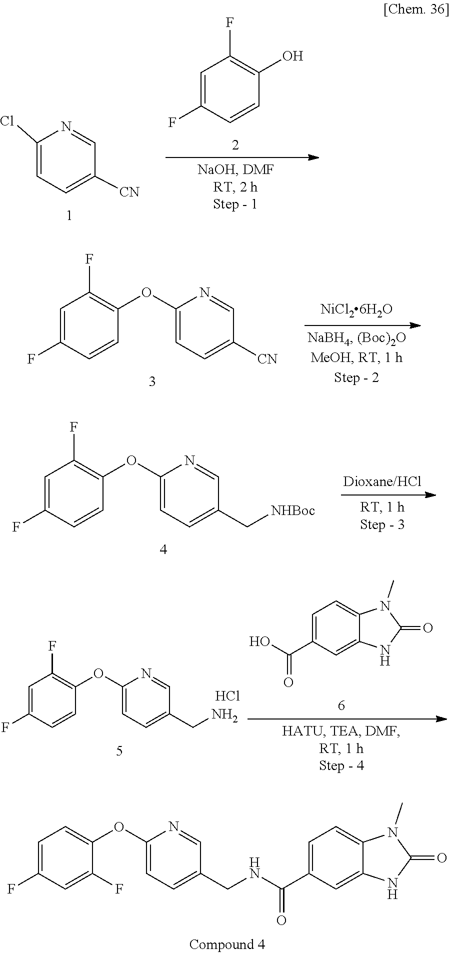

### Step-1: 6-(2,4-Difluorophenoxy)nicotinonitrile

To a stirred solution of 2,4-difluorophenol (0.93 g, 7.21 mmol) in DMF (3 mL) was added NaOH (0.43 g, 10.82 mmol) followed by 6-chloronicotinonitrile (1 g, 7.21 mmol) and stirred at RT for 2 h. The reaction mixture was poured into ice cold water and stirred for another 2 h, and the solid separated was collected by filtration and dried. This was purified by combi flash column chromatography using 20% EtOAc in hexane as an eluent to afford the title compound (0.8 g, 48%). ¹H-NMR (400 MHz, DMSO-D₆) δ 8.65-8.65 (d, 1H), 8.39-8.36 (dd, 1H), 7.52-7.44 (m, 2H), 7.4-7.38 (d, 1H), 7.2-7.15 (m, 1H).

### Step-2: tert-Butyl ((6-(2,4-difluorophenoxy)pyridin-3-yl)methyl)carbamate

To a solution of 6-(2,4-difluorophenoxy)nicotinonitrile (0.4 g, 1.72 mmol) in MeOH (5 mL) was added NiCl₂·6H₂O (0.21 g, 0.86 mmol) followed by dropwise addition of di-tert-butyl dicarbonate (0.75 g, 3.44 mmol) at 0° C. and stirred for 5 min. Then NaBH₄ (0.098 g, 2.58 mmol) was added in portions thereto by maintaining the same temperature. After stirring for 1 h at RT, the reaction mass was concentrated to remove the solvent, diluted with EtOAc and filtered on celite bed. The filtrate was washed with water, dried over anhydrous Na₂SO₄ and concentrated to afford the title compound (0.55 g, 95%). LC-MS: 337 [M+H]⁺

### Step-3: (6-(2,4-Difluorophenoxy)pyridin-3-yl)methanamine hydrochloride

To a solution of tert-butyl ((6-(2,4-difluorophenoxy)pyridin-3-yl)methyl)carbamate (3.4 g, 10.1 mmol) in 1,4-dioxane (10 mL) was added 4 N HCl in 1,4-dioxane (30 mL) and stirred for 1 h at RT. The reaction mixture was then concentrated, added diethyl ether thereto and the solid was collected by filtration to get the title compound (2.7 g, 98.9%). LC-MS: 237 [M+H]⁺

### Step-4: N-((6-(2,4-Difluorophenoxy)pyridin-3-yl)methyl)-1-methyl-2-oxo-2,3-dihydro-1H-benzi midazole-5-carboxamide (Compound 4)

To a solution of 1-methyl-2-oxo-2,3-dihydro-1H-benzimidazole-5-carboxylic acid (1.2 g, 6.2 mmol) in DMF (5 mL) were added HATU (2.84 g, 7.49 mmol) and triethylamine (1.89 g, 18.73 mmol), and stirred at RT for 10 min. Then (6-(2,4-difluorophenoxy)pyridin-3-yl)methanamine hydrochloride (1.03 g, 3.77 mmol) was added thereto and the mixture was stirred at RT for 2 h. The reaction mixture was added into ice cold water, and the precipitate formed was collected by filtration and dried. This was further purified by combi flash column chromatography using 5% MeOH in DCM as an eluent to afford the title compound (0.87 g, 56%). LC-MS: 411 [M+H]⁺; ¹H-NMR (400 MHz, DMSO-D₆) δ 11.05 (s, 1H), 8.92-8.89 (t, 1H), 8.01-8.0 (s, 1H), 7.80-7.78 (dd, 1H), 7.6-7.58 (d, 1H), 7.47 (s, 1H), 7.3 (m, 2H), 7.13-7.06 (m, 3H), 4.38-4.37 (d, 2H) 3.29-3.27 (s, 3H).

### Example-5

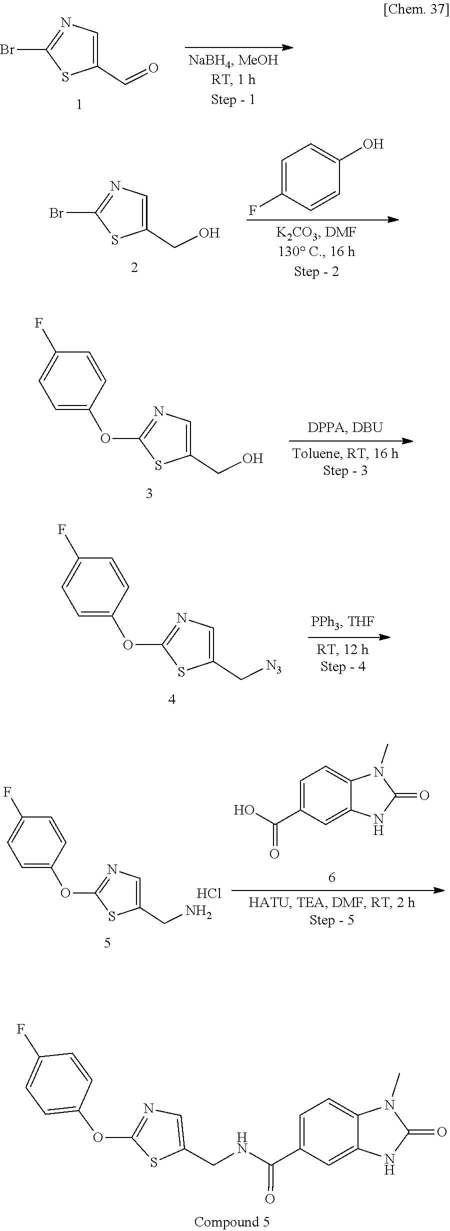

### Step-1: (2-Bromothiazol-5-yl)methanol

To a solution of 2-bromothiazole-5-carbaldehyde (2 g, 10.41 mmol) in MeOH (20 mL) was added NaBH₄ (0.59 g 15.62 mmol) and stirred at RT for 1 h. The reaction mass was concentrated to remove the solvent and the residue was dissolved in EtOAc, washed with water, dried over anhydrous Na₂SO₄ and concentrated to get the title compound. (2 g, 99%). LC-MS: 194 [M+H]⁺

### Step-2: (2-(4-Fluorophenoxy)thiazol-5-yl)methanol

To a stirred solution of 4-fluorophenol (1.38 g, 12.36 mmol) in DMF (20 mL) was added K₂CO₃ (2.13 g, 15.46 mmol) and stirred at 130° C. for 30 min. The reaction mass was cooled to 80° C., then (2-bromothiazol-5-yl)methanol (2.13 g, 15.46 mmol) was added thereto and stirred at 130° C. for 16 h. The reaction mixture was poured into ice cold water, diluted with EtOAc, washed with water, dried over anhydrous Na₂SO₄ and concentrated. The crude residue was purified by combi flash column chromatography using 20% EtOAc in hexane as an eluent to afford the title compound (1 g, 35.9%). LC-MS: 226 [M+H]⁺

### Step-3: 5-(Azidomethyl)-2-(4-fluorophenoxy)thiazole

To a stirred solution of (2-(4-fluorophenoxy)thiazol-5-yl)methanol (0.1 g, 0.44 mmol) in DCM (3 mL) and toluene (3 mL) was added DPPA (0.24 g, 0.88 mmol) followed by DBU (0.13 g, 0.88 mmol) and stirred at 0° C. for 2 h, then stirred at RT for 16 h. The reaction mixture was poured into ice cold water, extracted with DCM, dried over anhydrous Na₂SO₄ and concentrated to obtain a residue. This was purified by combi flash column chromatography using 10% EtOAc in hexane as an eluent to afford the title compound (0.08 g, 72.7%). LC-MS: 251 [M+H]⁺; ¹H-NMR (400 MHz, DMSO-D₆) δ 7.45-7.41 (m, 1H), 7.3-7.2 (m, 4H), 4.49 (s, 2H).

### Step-4 (2-(4-Fluorophenoxy)thiazol-5-yl)methanamine hydrochloride

To a solution of 5-(azidomethyl)-2-(4-fluorophenoxy)thiazole (0.04 g, 0.16 mmol) in THF (1 mL) and water (1 mL), was added PPh₃ (0.063 g, 0.24 mmol) stirred for 12 h at RT. Then 4 N HCl in 1,4-dioxane (1 mL) was added to the reaction mixture at 0° C. and stirred for 1 h. The reaction mass was concentrated, added diethyl ether thereto and the solid was collected by filtration to get the title compound (0.04 g, 96%). LC-MS: 225 [M+H]⁺

### Step-5: N-((2-(4-Fluorophenoxy)thiazol-5-yl)methyl)-1-methyl-2-oxo-2,3-dihydro-1H-benzimida zole-5-carboxamide trifluoroacetate (Compound 5)

To a solution of 1-methyl-2-oxo-2,3-dihydro-1H-benzimidazole-5-carboxylic acid (0.25 g, 1.3 mmol) in DMF (10 mL) were added HATU (0.59 g, 1.56 mmol) and triethylamine (0.65 g, 6.5 mmol), and stirred at RT for 10 min. Then (2-(4-fluorophenoxy)thiazol-5-yl)methanamine hydrochloride (0.27 g, 1.04 mmol) was added thereto and the mixture was stirred at RT for 2 h. The reaction mixture was poured into ice cold water, extracted with EtOAc, washed with brine, dried over anhydrous Na₂SO₄ and concentrated to get a crude compound. This was purified by preparative HPLC using method A to afford the title compound as TFA salt (0.045 g, 63.3%), LC-MS: 399 [M+H]⁺; ¹H-NMR (400 MHz, DMSO-D₆) δ 11.1 (s, 1H), 9.05 (m, 1H), 7.60-7.58 (d, 1H), 7.41 (s, 1H), 7.41-7.36 (m, 2H), 7.32-7.26 (m, 2H), 7.18-7.13 (m, 2H), 4.47-4.46 (d, 2H), 3.46 (s, 3H).

### Example-6

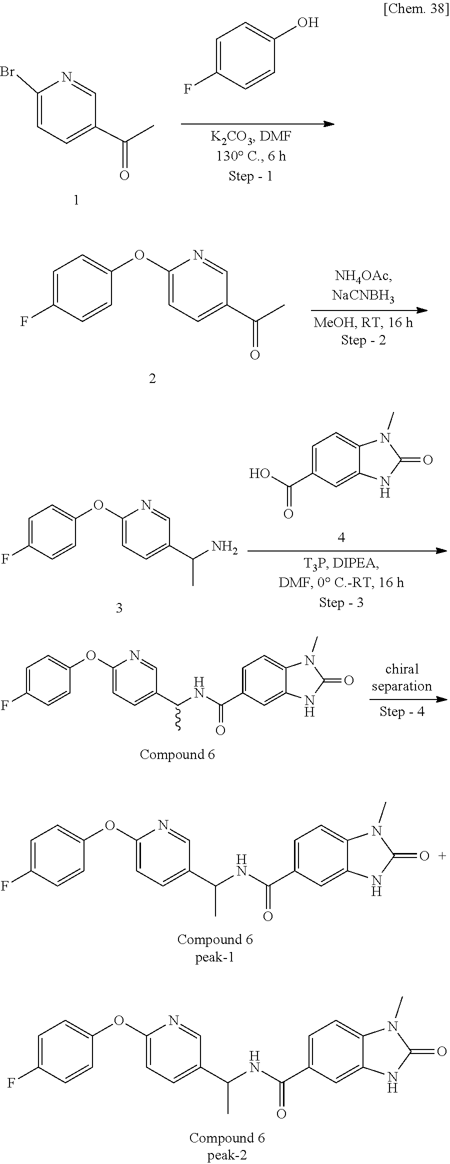

### Step-1: 1-(6-(4-Fluorophenoxy)pyridin-3-yl)ethan-1-one

To a stirred solution of 1-(6-bromopyridin-3-yl)ethan-1-one (2.67 g, 13.39 mmol) in DMF (30 mL) was added K₂CO₃ (5.54 g, 40.17 mmol) followed by 4-fluorophenol (1.5 g, 13.39 mmol) and stirred at 130° C. for 6 h. The reaction mixture was poured into ice cold water, the solid separated was collected by filtration and dried to afford the title compound (3.0 g, 86.5%). LC-MS: 232 [M+H]⁺

### Step-2: 1-(6-(4-Fluorophenoxy)pyridin-3-yl)ethan-1-amine

To a stirred solution of 1-(6-(4-fluorophenoxy)pyridin-3-yl)ethan-1-one (1 g, 4.32 mmol) in MeOH (25 mL) was added ammonium acetate (3.33 g, 43.29 mmol) followed by sodium cyanoborohydride (0.82 g, 12.98 mmol) and stirred at RT for 16 h. The reaction mixture was concentrated to remove the solvent, and the residue was diluted with water and basified with sodium hydroxide until pH-12, which was then extracted with EtOAc. The organic layer was dried over anhydrous Na₂SO₄ and concentrated to afford the title compound (0.9 g, 89.64%). LC-MS: 233 [M+H]⁺

### Step-3: N-(1-(6-(4-Fluorophenoxy)pyridin-3-yl)ethyl)-1-methyl-2-oxo-2,3-dihydro-1H-benzimid azole-5-carboxamide (Compound 6)

To a solution of 1-methyl-2-oxo-2,3-dihydro-1H-benzimidazole-5-carboxylic acid (0.15 g, 0.78 mmol) in DMF (5 mL) was added T₃P (0.745 g, 2.34 mmol) and stirred at RT for 10 min. Then 1-(6-(4-fluorophenoxy)pyridin-3-yl)ethan-1-amine (0.36 g, 1.56 mmol) was added thereto followed by DIPEA (0.30 g, 2.34 mmol) and the mixture was stirred at RT for 16 h. The reaction mixture was added into ice cold water, extracted with ethyl acetate, dried over anhydrous Na₂SO₄ and concentrated to get a crude product. This was purified by combi flash column chromatography using 4% MeOH in DCM as an eluent to afford the title compound (0.14 g, 24.6%). LC-MS: 407 [M+H]⁺.

### Step-4: Optical isomers of N-(1-(6-(4-Fluorophenoxy)pyridin-3-yl)ethyl)-1-methyl-2-oxo-2,3-dihydro-1H-benzimid azole-5-carboxamide (Compound 6 Peak 1 and Peak 2)

The obtained Compound 6 was further submitted to chiral separation to obtain two optical isomers of the compound as Compound 6 Peak 1 (0.025 g, 4.38%) and Compound 6 Peak 2 (0.025 g, 4.38%). (Preparative HPLC method: Column—Chiralpak IA (250×20×5 μm); Eluent-A—0.1% DEA in HEXANE, B—IPA:DCM (90:10)%; ISOCRATIC: A:B(60:40); Flow: 15 mL/min)

Compound 6 Peak 1: LC-MS: 407 [M+H]+; ¹H-NMR (400 MHz, DMSO-D6) δ 11.07 (s, 1H), 8.8 (d, 1H), 8.1 (s, 1H), 7.85 (dd, 1H), 7.6 (dd, 1H), 7.5 (s, 1H), 7.21-7.12 (m, 5H), 7.0 (d, 1H), 5.14 (t, 1H), 3.3 (s, 3H), 1.48 (d, 3H); Chiral HPLC RRT: 5.85.

Compound 6 Peak 2: LC-MS: 407 [M+H]+; ¹H-NMR (400 MHz, DMSO-D6) δ 11.07 (s, 1H), 8.8 (d, 1H), 8.1 (s, 1H), 7.85 (dd, 1H), 7.6 (dd, 1H), 7.5 (s, 1H), 7.21-7.12 (m, 5H), 7.0 (d, 1H), 5.14 (t, 1H), 3.3 (s, 3H), 1.48 (d, 3H); Chiral HPLC RRT: 9.16.

### Example-7

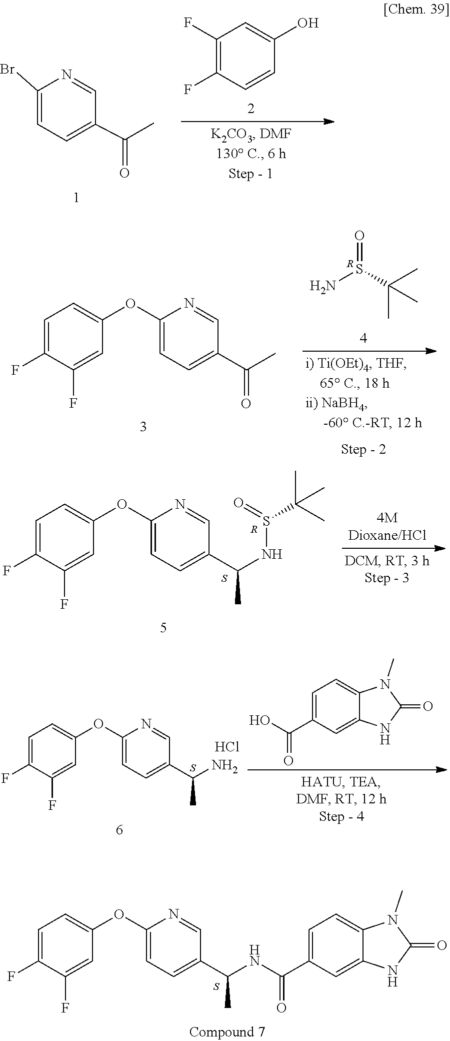

### Step-1: 1-(6-(3,4-Difluorophenoxy)pyridin-3-yl)ethan-1-one

To a stirred solution of 3,4-difluorophenol (1.61 g, 12.49 mmol) in DMF (3 mL) was added Cs₂CO₃ (16.27 g, 49.99 mmol) followed by 1-(6-bromopyridin-3-yl)ethan-1-one (2.5 g, 12.49 mmol) and stirred at 130° C. for 6 h. The reaction mixture was poured into ice cold water, extracted with EtOAc, washed with brine, dried over anhydrous Na₂SO₄ and concentrated to get a crude compound. The compound was purified by combi flash column chromatography using 30% EtOAc in hexane as an eluent to afford the title compound (2 g, 64.3%). LC-MS: 250 [M+H]⁺

### Step-2: (R)—N—((S)-1-(6-(3,4-Difluorophenoxy)pyridin-3-yl)ethyl)-2-methylpropane-2-sulfina mide

To a solution of 1-(6-(3,4-difluorophenoxy)pyridin-3-yl)ethan-1-one (1 g, 4.04 mmol) in THF (15 ml) were added titanium tetraethoxide (1.83 g, 8.02 mmol) and (R)-2-methylpropane-2-sulfinamide (0.48 g, 4.01 mmol) under inert atmosphere. The reaction mass was heated to reflux for 18 h. The reaction mass was cooled to −60° C., NaBH₄ (0.60 g, 16.05 mmol) was added thereto and stirred for 30 min at the same temperature, then stirred at RT for 12 h. The reaction mass was poured into ice cold water, extracted with EtOAc, dried over anhydrous Na₂SO₄ and concentrated to get the title compound. (1.4 g, 97%) LC-MS: 355 [M+H]⁺

### Step-3: (S)-1-(6-(3,4-Difluorophenoxy)pyridin-3-yl)ethan-1-amine hydrochloride

To a solution of (R)—N—((S)-1-(6-(3,4-difluorophenoxy)pyridin-3-yl)ethyl)-2-methylpropane-2-sulfinam ide (1.4 g, 3.95 mmol) in DCM (10 mL) was added 4 N HCl in 1,4-dioxane (8 mL) and stirred at RT for 3 h. The reaction mass was then concentrated, diethyl ether was added thereto and the precipitate was collected by filtration to get the title compound (0.95 g, 96%). LC−MS: 249 [M−H]⁻

### Step-4: (S)—N-(1-(6-(3,4-Difluorophenoxy)pyridin-3-yl)ethyl)-1-methyl-2-oxo-2,3-dihydro-1H-benzimidazole-5-carboxamide (Compound 7)

To a solution of 1-methyl-2-oxo-2,3-dihydro-1H-benzimidazole-5-carboxylic acid (0.15 g, 0.78 mmol) in DMF (8 mL) were added HATU (0.35 g, 0.93 mmol) and triethylamine (0.39 g, 3.9 mmol), and stirred at RT for 10 min. Then (S)-1-(6-(3,4-difluorophenoxy)pyridin-3-yl)ethan-1-amine hydrochloride (0.22 g, 0.78 mmol) was added thereto and the mixture was stirred at RT for 12 h. The reaction mixture was poured into ice cold water, extracted with EtOAc, dried over anhydrous Na₂SO₄ and concentrated to get a crude compound. This was purified by combi flash column chromatography using 100% EtOAc as an eluent to afford the title compound (0.05 g, 15%). LC-MS: 425 [M+H]⁺; ¹H-NMR (400 MHz, DMSO-D₆) δ 11.11 (s, 1H), 8.78-8.76 (d, 1H), 8.19 (d, 1H), 7.95-7.92 (dd, 1H), 7.68-7.66 (d, 1H), 7.55-7.46 (m, 2H), 7.4-7.3 (m, 1H), 7.19-7.17 (d, 1H), 7.1-7.07 (d, 1H), 7.04-7.01 (d, 1H), 5.22-5.18 (m, 2H), 3.36-3.34 (s, 3H), 1.53-1.51 (d, 2H).

### Example-8

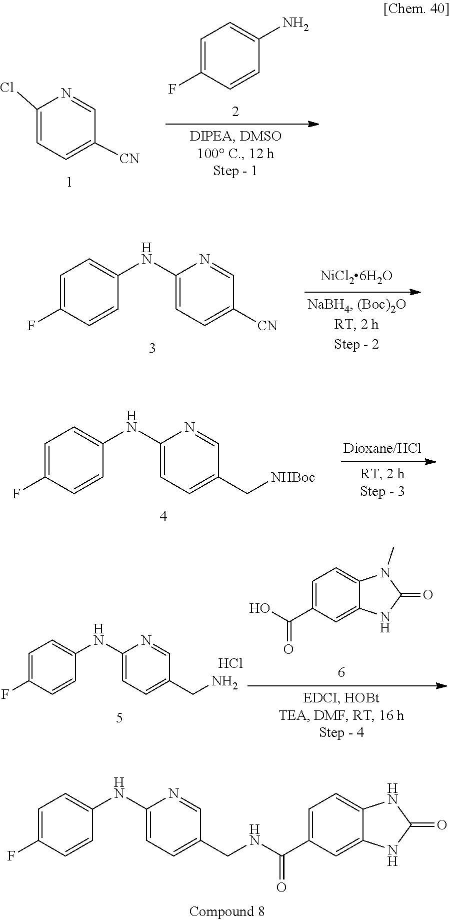

### Step-1: 6-((4-Fluorophenyl)amino)nicotinonitrile

To a stirred solution of 6-chloronicotinonitrile (0.686 g, 4.95 mmol) in DMSO (5 mL) was added DIPEA (1.454 g, 11.25 mmol) followed by 4-fluoroaniline (0.5 g, 4.5 mmol) and stirred at 100° C. for 12 h. The reaction mixture was concentrated to remove DIPEA then poured into ice cold water, and the solid thrown out was collected by filtration and dried. The crude residue was purified by combi flash column chromatography using 20% EtOAc in hexane as an eluent to afford the pure title compound (0.5 g, 52.11%). LC-MS: 214.05 [M+H]⁺

### Step-2: tert-butyl ((6-((4-Fluorophenyl)amino)pyridin-3-yl)methyl)carbamate

A solution of 6-((4-fluorophenyl)amino)nicotinonitrile (0.4 g, 1.88 mmol) in MeOH (8 mL) was cooled to 0° C., NiCl₂·6H₂O (0.22 g, 0.93 mmol) was added thereto followed by dropwise addition of di-tert-butyl dicarbonate (0.61 g, 2.81 mmol) and stirred for 5 min. Then NaBH₄ (0.14 g, 3.75 mmol) was added in portions thereto by maintaining the same temperature. After stirring for 2 h at RT, the reaction mass was concentrated to remove solvent. The residue was diluted with EtOAc, washed with water, dried over anhydrous Na₂SO₄ and concentrated to afford the title compound (0.5 g crude). LC-MS: 318 [M+H]⁺

### Step-3: 5-(Aminomethyl)-N-(4-fluorophenyl)pyridin-2-amine hydrochloride

To a solution of tert-butyl ((6-((4-fluorophenyl)amino)pyridin-3-yl)methyl)carbamate (0.4 g, 1.26 mmol) in 1,4-dioxane (2 mL) was added 4 N HCl in 1,4-dioxane (4 mL) and stirred for 2 h at RT. The reaction mass was concentrated, added diethyl ether thereto and the solid was collected by filtration to get the title compound (0.4 g). LC-MS: 218 [M+H]⁺

### Step-4: N-((6-((4-Fluorophenyl)amino)pyridin-3-yl)methyl)-2-oxo-2,3-dihydro-1H-benzimidazol e-5-carboxamide trifluoroacetate (Compound 8)

To a solution of 2-oxo-2,3-dihydro-1H-benzimidazole-5-carboxylic acid (0.18 g, 1.01 mmol) in DMF (6 mL) were added EDC·HCl (0.23 g, 1.21 mmol) and HOBt (0.16 g, 1.21 mmol) and stirred at RT for 10 min. Then 5-(aminomethyl)-N-(4-fluorophenyl)pyridin-2-amine hydrochloride (0.22 g, 0.86 mmol) followed by triethylamine (0.51 g, 5.05 mmol) was added thereto and the mixture was stirred at RT for 16 h. The reaction mixture was added into ice cold water, the precipitate formed was collected by filtration and dried. The crude residue was further purified by preparative HPLC using method A to afford the title compound as a TFA salt (0.07 g, 16.5%). LC-MS: 378 [M+H]⁺

¹H-NMR (400 MHz, DMSO-D₆) δ 10.85 (d, 2H), 9.5 (bs, 1H), 8.85 (m, 1H), 8.0 (s, 1H), 7.7-7.55 (m, 3H), 7.1 (d, 1H), 7.4 (s, 1H), 7.1 (t, 2H), 6.9 (d, 1H), 6.8 (d, 1H), 4.3 (d, 2H).

### Example-9

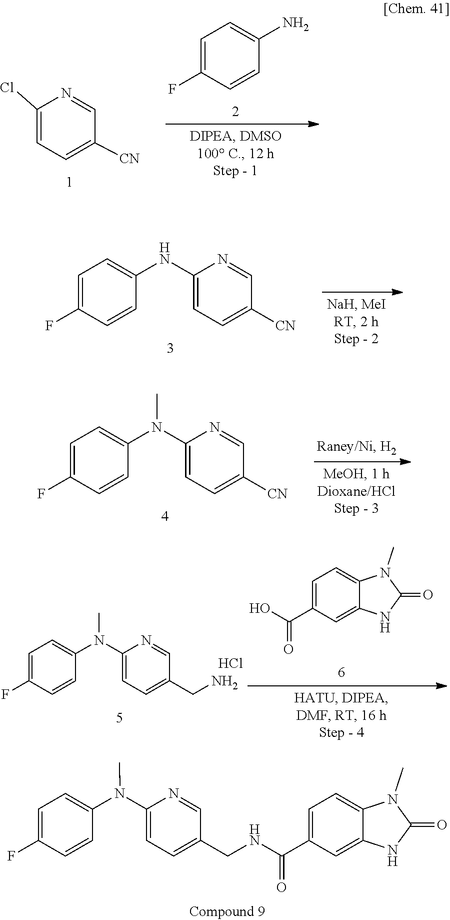

### Step-1: 6-((4-Fluorophenyl)amino)nicotinonitrile

To a stirred solution of 6-chloronicotinonitrile (13.7 g, 98.99 mmol) in DMSO (100 mL) was added DIPEA (29.08 g, 225.0 mmol) followed by 4-fluoroaniline (10 g, 89.99 mmol) and stirred at 100° C. for 12 h. The reaction mixture was concentrated to remove DIPEA then poured into ice cold water, and the solid separated was collected by filtration and dried. This was purified by combi flash column chromatography using 20% EtOAc in hexane as an eluent to afford the title compound (9.0 g, 46.91%). LC-MS: 214 [M+H]⁺

### Step-2: 6-((4-Fluorophenyl)(methyl)amino)nicotinonitrile

To a stirred solution of 6-((4-fluorophenyl)amino)nicotinonitrile (9 g, 42.21 mmol) in DMF (90 mL) was added NaH (60% dispersion in mineral oil) (1.07 g, 24.45 mmol) followed by dropwise addition of iodomethane (6.59 g, 46.4 mmol) at 0° C. and stirred at RT for 2 h. The reaction mixture was poured into ice cold water, and the solid separated was collected by filtration and dried. This was purified by combi flash column chromatography using 20% EtOAc in hexane as an eluent to afford the title compound (8 g, 83.40%). LC-MS: 228 [M+H]⁺

### Step-3: 5-(Aminomethyl)-N-(4-fluorophenyl)-N-methylpyridin-2-amine hydrochloride

A solution of 6-((4-fluorophenyl)(methyl)amino)nicotinonitrile (2.5 g, 11.00 mmol) in MeOH (25 mL) was cooled to 0° C., 7 N ammonia in MeOH (2.5 mL) was added thereto followed by the addition of Raney nickel (2.5 g) and stirred under hydrogen bladder pressure for 1 h at RT. The reaction mass was filtered through celite and the filtrate was concentrated. The material was taken in 1,4-dioxane (10 mL) and 4 N HCl in 1,4-dioxane (10 mL) was added thereto at 0° C. and stirred for 2 h at RT. The reaction mass was concentrated, washed with diethyl ether and collected by filtration to afford the title compound (2.3 g, 78.2%). LC-MS: 232 [M+H]⁺

### Step-4: N-((6-((4-Fluorophenyl)(methyl)amino)pyridin-3-yl)methyl)-1-methyl-2-oxo-2,3-dihydro-1H-benzimidazole-5-carboxamide trifluoroacetate (Compound 9)

To a solution of 1-methyl-2-oxo-2,3-dihydro-1H-benzimidazole-5-carboxylic acid (1.12 g, 5.83 mmol) in DMF (12 mL) was added HATU (2.66 g, 6.99 mmol) and stirred at RT for 10 min. Then 5-(aminomethyl)-N-(4-fluorophenyl)-N-methylpyridin-2-amine hydrochloride (1.6 g, 5.94 mmol) followed by DIPEA (2.95 g, 22.82 mmol) was added thereto, and the mixture was stirred at RT for 16 h. The reaction mixture was added into ice cold water, and the precipitate formed was collected by filtration and dried. This was further purified by preparative HPLC using method A to afford the title compound as a TFA salt (0.35 g, 11.40%). LC-MS: 406 [M+H]⁺; ¹H-NMR (400 MHz, DMSO-D₆) δ 11.05 (s, 1H), 8.9 (bs, 1H), 8 (s, 1H), 7.6 (d, 2H), 7.5 (s, 1H), 7.25-7.4 (m, 4H), 7.15 (d, 1H), 6.65 (bs, 1H), 4.3 (d, 2H), 3.4 (s, 3H), 3.3 (s, 3H).

### Example-10

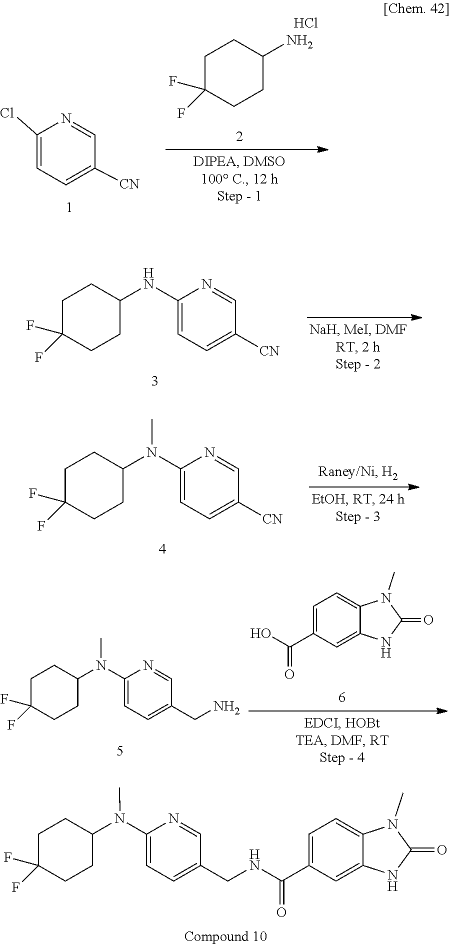

### Step-1: 6-((4,4-Difluorocyclohexyl)amino)nicotinonitrile

To a stirred solution of 6-chloronicotinonitrile (4.5 g, 32.47 mmol) in DMSO (100 mL) was added DIPEA (20.98 g, 162.39 mmol) followed by 4,4-difluorocyclohexan-1-amine hydrochloride (5.57 g, 32.47 mmol) and stirred at 100° C. for 12 h. The reaction mixture was concentrated to remove DIPEA then poured into ice cold water, and the solid separated was collected by filtration and dried. This was purified by combi flash column chromatography using 20% EtOAc in hexane as an eluent to afford the title compound (5.2 g, 67.48%). LC-MS: 236 [M−H]⁻.

### Step-2: 6-((4,4-Difluorocyclohexyl)(methyl)amino)nicotinonitrile

To a stirred solution of 6-((4,4-difluorocyclohexyl)amino)nicotinonitrile (5.2 g, 21.9 mmol) in DMF (30 mL) was added NaH (60% dispersion in mineral oil) (0.876 g, 21.9 mmol) followed by dropwise addition of iodomethane (3.80 g, 26.74 mmol) at 0° C. and stirred at RT for 2 h. The reaction mixture was poured into ice cold water, and the solid separated was collected by filtration and dried. This was purified by combi flash column chromatography using 20% EtOAc in hexane as an eluent to afford the title compound (5 g, 90.9%). LC-MS: 252 [M+H]⁺

### Step-3: 5-(Aminomethyl)-N-(4,4-difluorocyclohexyl)-N-methylpyridin-2-amine

To a solution of 6-((4,4-difluorocyclohexyl)(methyl)amino)nicotinonitrile (2 g, 7.95 mmol) in EtOH (40 mL) was added Raney nickel (2 g) and stirred under hydrogen bladder pressure for 24 h at RT. The reaction mass was filtered through celite and the filtrate was concentrated to afford the title compound (1.8 g, 88.6%). LC-MS: 256 [M+H]⁺

### Step-4: N-((6-((4,4-Difluorocyclohexyl)(methyl)amino)pyridin-3-yl)methyl)-1-methyl-2-oxo-2,3-dihydro-1H-benzimidazole-5-carboxamide (Compound 10)

To a solution of 1-methyl-2-oxo-2,3-dihydro-1H-benzimidazole-5-carboxylic acid (1.5 g, 7.84 mmol) in DMF (15 mL) were added EDC·HCl (1.52 g, 7.92 mmol) and HOBt (0.80 g, 5.92 mmol), and stirred at RT for 10 min. Then 5-(aminomethyl)-N-(4,4-difluorocyclohexyl)-N-methylpyridin-2-amine (1.52 g, 5.96 mmol) followed by triethylamine (3.57 g, 35.25 mmol) was added thereto and the mixture was stirred at RT for 16 h. The reaction mixture was poured into ice cold water, and the precipitate formed was collected by filtration and dried. This was purified by combi flash column chromatography using 5% MeOH in DCM as an eluent to afford the title compound (0.6 g, 23.78%). LC-MS: 430 [M+H]⁺; ¹H-NMR (400 MHz, DMSO-D₆) δ 11.0 (s, 1H), 8.8 (m, 1H), 8 (d, 1H), 7.6 (d, 1H), 7.45 (m, 2H), 7.1 (d, 1H), 6.6 (d, 1H), 4.6 (m, 1H), 4.35 (d, 2H), 3.25 (s, 3H), 2.85 (s, 3H), 2.0-2.1 (m, 3H), 1.95 (m, 1H), 1.7 (m, 2H), 1.6 (d, 2H).

### Example-11

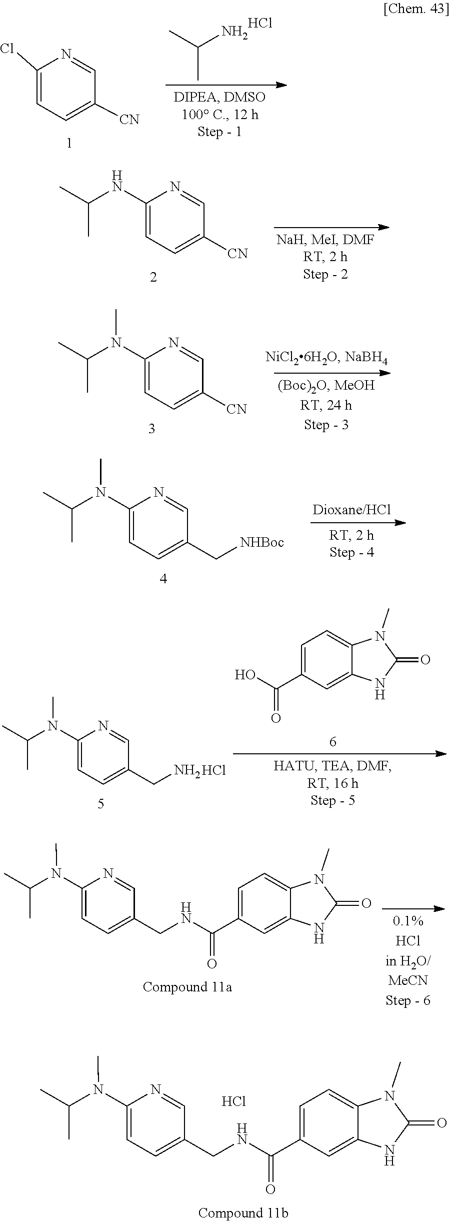

### Step-1: 6-(Isopropylamino)nicotinonitrile

To a stirred solution of 6-chloronicotinonitrile (3.5 g, 25.26 mmol) in DMSO (35 mL) was added DIPEA (16.3 g, 126.3 mmol) followed by propan-2-amine hydrochloride (4.83 g, 50.52 mmol) and stirred at 100° C. for 12 h. The reaction mixture was concentrated to remove DIPEA then poured into ice cold water, and the solid separated was collected by filtration and dried. This was purified by combi flash column chromatography using 20% EtOAc in hexane as an eluent to afford the title compound (2.8 g, 68.8%). LC-MS: 162 [M+H]⁺

### Step-2: 6-(Isopropyl(methyl)amino)nicotinonitrile

To a stirred solution of 6-(isopropylamino)nicotinonitrile (2.8 g, 17.36 mmol) in DMF (28 mL) was added NaH (60% dispersion in mineral oil)(1.04 g, 26.04 mmol) followed by dropwise addition of iodomethane (2.95 g, 20.8 mmol) at 0° C. and stirred at RT for 2 h. The reaction mixture was poured into ice cold water, and the solid separated was collected by filtration and dried. This was purified by combi flash column chromatography using 20% EtOAc in hexane as an eluent to afford the title compound (2.5 g, 68.8%). LC-MS: 176 [M+H]⁺

### Step-3: tert-Butyl ((6-(isopropyl(methyl)amino)pyridin-3-yl)methyl)carbamate

A solution of 6-(isopropyl(methyl)amino)nicotinonitrile (2.5 g, 14.26 mmol) in MeOH (500 mL) was cooled to 0° C., added NiCl₂·6H₂O (0.51 g, 2.13 mmol) followed by dropwise addition of di-tert-butyl dicarbonate (6.22 g, 28.53 mmol) and stirred for 5 min. Then NaBH₄ (0.81 g, 21.39 mmol) was added in portions thereto by maintaining the same temperature. After stirring for 1 h at RT, the reaction mass was concentrated to remove MeOH. The residue was diluted with EtOAc, washed with water, dried over anhydrous Na₂SO₄ and concentrated. The crude compound was purified by combi flash chromatography using 20% EtOAc in hexane as an eluent to afford the title compound (1.6 g, 40.9%). LC-MS: 280 [M+H]⁺

### Step-4: 5-(Aminomethyl)-N-isopropyl-N-methylpyridin-2-amine hydrochloride

To a solution of tert-butyl ((6-(isopropyl(methyl)amino)pyridin-3-yl)methyl)carbamate (1.6 g, 5.7 mmol) in 1,4-dioxane (10 mL) was added 4 N HCl in 1,4-dioxane (10 mL) and stirred for 2 h at RT. The reaction mixture was then concentrated, diethyl ether was added thereto, and the obtained residue was collected by filtration to get the crude title compound (1.6 g). LC-MS: 180 [M+H]⁺

### Step-5: N-((6-(Isopropyl(methyl)amino)pyridin-3-yl)methyl)-1-methyl-2-oxo-2,3-dihydro-1H-be nzimidazole-5-carboxamide trifluoroacetate (Compound 11a)

To a solution of 1-methyl-2-oxo-2,3-dihydro-1H-benzimidazole-5-carboxylic acid (1.78 g, 9.26 mmol) in DMF (25 mL) were added EDC·HCl (1.77 g, 9.26 mmol) and HOBt (0.63 g, 4.63 mmol), and stirred at RT for 10 min. Then 5-(aminomethyl)-N-isopropyl-N-methylpyridin-2-amine hydrochloride (1.6 g, 7.41 mmol) followed by triethylamine (5.61 g, 55.56 mmol) was added thereto and the mixture was stirred at RT for 16 h. The reaction mixture was added into ice cold water, and precipitate formed was collected by filtration and washed with water and dried. This was further purified by preparative HPLC using method A to afford the title compound as a TFA salt (0.7 g, 20.2%). LC-MS: 354 [M+H]⁺

### Step-6: N-((6-(Isopropyl(methyl)amino)pyridin-3-yl)methyl)-1-methyl-2-oxo-2,3-dihydro-1H-be nzimidazole-5-carboxamide hydrochloride (Compound 11b)

N-((6-(isopropyl(methyl)amino)pyridin-3-yl)methyl)-1-methyl-2-oxo-2,3-dihydr o-1H-benzimidazole-5-carboxamide trifluoroacetate was dissolved in the mixture of 0.1% HCl in water:acetonitrile (60:40) (700 mL) and concentrated completely at RT to afford the title compound as a HCl salt (0.476 g, 81.8%). LC-MS: 354 [M+H]⁺; ¹H-NMR (400 MHz, CD₃OD) δ 8.06-8.03 (dd, 1H), 7.88 (m, 1H), 7.69-7.67 (dd, 1H), 7.61 (d, 1H), 7.38-7.35 (d, 1H), 7.21-7.19 (d, 1H), 4.49 (s, 2H), 4.42 (m, 1H), 3.43 (S, 3H), 3.11 (s, 3H), 1.35 (d, 6H).

### Example-12

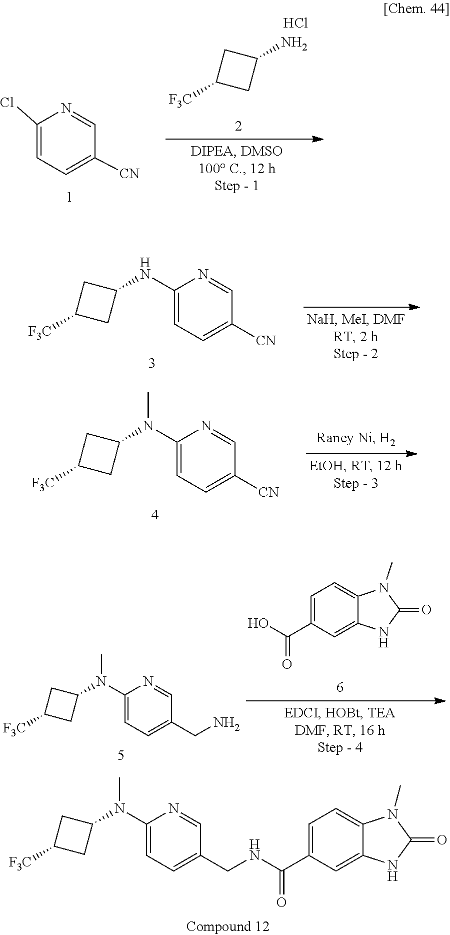

### Step-1: 6-((cis-3-(Trifluoromethyl)cyclobutyl)amino)nicotinonitrile

To a stirred solution of 6-chloronicotinonitrile (1.42 g, 10.24 mmol) in DMSO (20 mL) was added DIPEA (7.95 g, 61.49 mmol) followed by cis-3-(trifluoromethyl)cyclobutan-1-amine hydrochloride (2.00 g, 11.40 mmol) and stirred at 100° C. for 12 h. The reaction mixture was concentrated to remove DIPEA then poured into ice cold water, and the solid separated was collected by filtration and dried. This was purified by combi flash column chromatography using 20% EtOAc in hexane as an eluent to afford the title compound (2.32 g, 93.9%). LC-MS: 242 [M+H]⁺.

### Step-2: 6-(methyl(cis-3-(Trifluoromethyl)cyclobutyl)amino)nicotinonitrile

To a stirred solution of 6-((cis-3-(trifluoromethyl)cyclobutyl)amino)nicotinonitrile (2.32 g, 9.62 mmol) in DMF (25 mL) was added NaH (60% dispersion in mineral oil) (0.38 g, 9.6 mmol) followed by dropwise addition of iodomethane (1.64 g, 11.54 mmol) at 0° C. and stirred at RT for 2 h. The reaction mixture was poured into ice cold water, and the solid separated was collected by filtration and dried to afford the title compound (2.4 g, 97%). LC-MS: 256 [M+H]⁺

### Step-3: 5-(Aminomethyl)-N-methyl-N-(cis-3-(trifluoromethyl)cyclobutyl)pyridin-2-amine

To a solution of 6-(methyl(cis-3-(trifluoromethyl)cyclobutyl)amino)nicotinonitrile (2.4 g, 9.40 mmol) in EtOH (25 mL) was added 7 N ammonia in MeOH (2.3 mL) followed by the addition of Raney nickel (2.4 g) and stirred under hydrogen bladder pressure for 12 h at RT. The reaction mass was filtered through celite and the filtrate was concentrated to afford the title compound (2.3 g, 94.6%). LC-MS: 260 [M+H]⁺

### Step-4: 1-Methyl-N-((6-(methyl(cis-3-(trifluoromethyl)cyclobutyl)amino)pyridin-3-yl)methyl)-2-oxo-2,3-dihydro-1H-benzimidazole-5-carboxamide (Compound 12)

To a solution of 1-methyl-2-oxo-2,3-dihydro-1H-benzimidazole-5-carboxylic acid (1.45 g, 7.54 mmol) in DMF (25 mL) was added EDC·HCl (1.45 g, 7.54 mmol) and HOBt (0.54 g, 3.99 mmol), and stirred at RT for 10 min. Then 5-(aminomethyl)-N-methyl-N-(cis-3-(trifluoromethyl)cyclobutyl)pyridin-2-amine (2.3 g, 8.87 mmol) was added thereto followed by triethylamine (4.48 g, 44.35 mmol) and the mixture was stirred at RT for 16 h. The reaction mixture was poured into ice cold water, and the precipitate formed was collected by filtration and dried. This was further washed with diethyl ether (3×25 mL) followed by pentane (25 mL) to afford the title compound (1.28 g, 39.26%). LC-MS: 434 [M+H]⁺

¹H-NMR (400 MHz, DMSO-D₆) δ 11.1 (s, 1H), 8.8 (t, 1H), 8.05 (s, 1H), 7.6 (d, 1H), 7.5 (m, 2H), 7.1 (d, 1H), 6.6 (d, 1H), 4.9 (t, 1H), 4.3 (d, 2H), 3.3 (s, 3H), 2.9 (m, 4H), 2.35 (m, 2H), 2.2 (m, 2H).

### Example-13

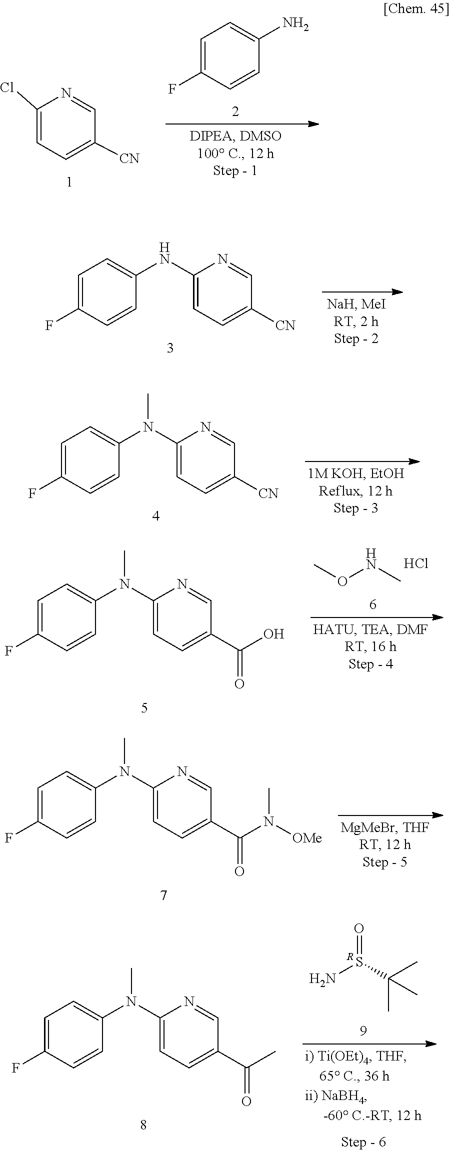

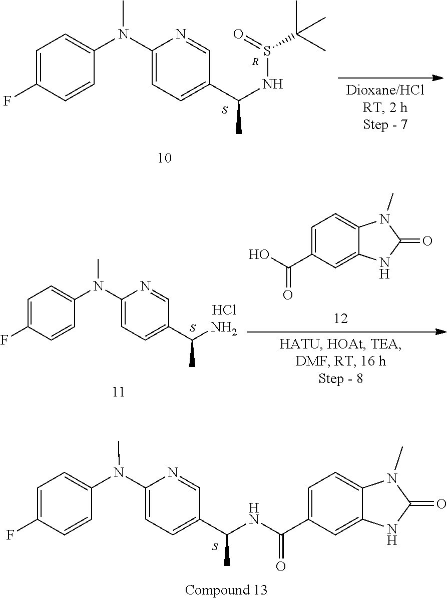

### Step-1: 6-((4-Fluorophenyl)amino)nicotinonitrile

To a stirred solution of 6-chloronicotinonitrile (6.85 g, 49.4 mmol) in DMSO (40 mL) was added DIPEA (14.54 g, 112.49 mmol) followed by 4-fluoroaniline (5 g, 44.99 mmol) and stirred at 100° C. for 12 h. The reaction mixture concentrated to remove DIPEA then poured into ice cold water, and the solid separated was collected by filtration and dried to afford the title compound (9.5 g, 90.2%). LC-MS: 214.1 [M+H]⁺

### Step-2: 6-((4-Fluorophenyl)(methyl)amino)nicotinonitrile

To a stirred solution of 6-((4-fluorophenyl)amino)nicotinonitrile (5 g, 23.449 mmol) in DMF (30 mL) was added NaH (60% dispersion in mineral oil) (0.916 g, 22.990 mmol) followed by dropwise addition of iodomethane (3.99 g, 28.14 mmol) at 0° C. and stirred at RT for 2 h. The reaction mixture poured into ice cold water, and the solid separated was collected by filtration and dried. This crude residue was purified by combi flash column chromatography using 20% EtOAc in hexane as an eluent to afford the pure title compound (2.5 g, 46.92%). LC-MS: 228.0 [M+H]⁺

### Step-3: 6-((4-Fluorophenyl)(methyl)amino)nicotinic acid

To a stirred solution of 6-((4-fluorophenyl)(methyl)amino)nicotinonitrile (2 g, 8.80 mmol) in ethanol (10 mL) and water (10 mL) was added KOH (0.99 g, 17.60 mmol) and heated to reflux for 12 h. The reaction mixture was concentrated to remove EtOH then pH of the reaction mixture was adjusted to 2 using 1 N HCl. The solid precipitated was collected by filtration and dried to afford the title compound (1.7 g, 78.7%). LC-MS: 245 [M−H]⁻

### Step-4: 6-((4-Fluorophenyl)(methyl)amino)-N-methoxy-N-methylnicotinamide

To a solution of 6-((4-fluorophenyl)(methyl)amino)nicotinic acid (2 g, 8.12 mmol) in DMF (15 mL) was added HATU (4.63 g, 12.18 mmol) and stirred at RT for 10 min. Then N,O-dimethylhydroxylamine hydrochloride (0.95 g, 9.74 mmol) was added thereto followed by DIPEA (2.46 g, 19.03 mmol) and the mixture was stirred at RT for 16 h. The reaction mixture was poured into ice cold water, extracted with EtOAc and dried over anhydrous Na₂SO₄ and concentrated to afford title compound (2.7 g), which was taken further without purification. LC-MS: 290 [M+H]⁺

### Step-5: 1-(6-((4-Fluorophenyl)(methyl)amino)pyridin-3-yl)ethan-1-one

To a stirred solution of 6-((4-fluorophenyl)(methyl)amino)-N-methoxy-N-methylnicotinamide (2.7 g, 9.33 mmol) in THF (15 mL) was added methyl magnesium bromide (2.22 g, 18.66 mmol) at 0° C. and the mixture was stirred at RT for 12 h. The reaction mixture was quenched with saturated ammonium chloride, diluted with water and extracted with DCM. This was purified by combi flash column chromatography using 20% EtOAc in hexane as an eluent to afford the title compound (1.85 g, 81.5%). LC-MS: 245 [M+H]⁺

### Step-6:(R)—N—((S)-1-(6-((4-Fluorophenyl)(methyl)amino)pyridin-3-yl)ethyl)-2-methy lpropane-2-sulfinamide

To a stirred solution of 1-(6-((4-fluorophenyl)(methyl)amino)pyridin-3-yl)ethan-1-one (1.0 g, 4.09 mmol) in THF (20 mL) was added titanium tetraethoxide (1.86 g, 8.18 mmol) at 0° C. followed by (R)-2-methylpropane-2-sulfinamide (0.50 g, 4.09 mmol) and the mixture was refluxed for 36 h. The reaction mixture was cooled to −60° C., added sodium borohydride (0.62 g, 16.37 mmol) and stirred for 0.5 h at the same temperature. Further the reaction mixture was warmed to RT and stirred for 12 h. The reaction mixture was filtered, and the filtrate was diluted with water and extracted with EtOAc, dried over anhydrous Na₂SO₄ and concentrated under reduced pressure to obtain a crude residue. This was purified by combi flash column chromatography using 30% EtOAc in hexane as an eluent to afford the title compound (1.1 g, 76.9%). LC-MS: 350 [M+H]⁺

### Step-7: (S)-5-(1-Aminoethyl)-N-(4-fluorophenyl)-N-methylpyridin-2-amine hydrochloride

To a solution of (R)—N—((S)-1-(6-((4-fluorophenyl)(methyl)amino)pyridin-3-yl)ethyl)-2-methylpropane-2-sulfinamide (1.2 g, 3.43 mmol) in 1,4-dioxane (5 mL) was added 4 N HCl in 1,4-dioxane (10 mL) and stirred for 2 h at RT. The reaction mass was concentrated, added diethyl ether and the solid was collected by filtration to get the title compound (1.1 g) which was taken further as such. LC-MS: 246 [M+H]⁺

### Step-8: (S)—N-(1-(6-((4-Fluorophenyl)(methyl)amino)pyridin-3-yl)ethyl)-1-methyl-2-oxo-2,3-d ihydro-1H-benzimidazole-5-carboxamide (Compound 13)

To a solution of 1-methyl-2-oxo-2,3-dihydro-1H-benzimidazole-5-carboxylic acid (0.25 g, 1.3 mmol) in DMF (7 mL) was added HATU (0.59 g, 1.56 mmol) and HOAt (0.21 g, 1.56 mmol), and stirred at RT for 10 min. Then (S)-5-(1-aminoethyl)-N-(4-fluorophenyl)-N-methylpyridin-2-amine hydrochloride (0.29 g, 1.04 mmol) followed by triethylamine (0.66 g, 6.5 mmol) was added thereto and the mixture was stirred at RT for 16 h. The reaction mixture was diluted with water, extracted with EtOAc, dried over anhydrous Na₂SO₄ and concentrated to obtain a crude residue. This was purified by combi flash column chromatography using 10% MeOH in DCM as an eluent to afford the title compound (0.01 g, 1.83%). LC-MS: 420 [M+H]⁺; ¹H-NMR (400 MHz, CD₃OD) δ 8.15 (d, 1H), 7.65 (dd, 1H), 7.55 (d, 1H), 7.5 (dd, 1H), 7.25 (m, 2H), 7.15 (m, 3H), 6.5 (d, 1H), 5.1-5.2 (q, 1H), 3.2-3.5 (m, 8H), 1.5 (d, 3H).

### Example-14

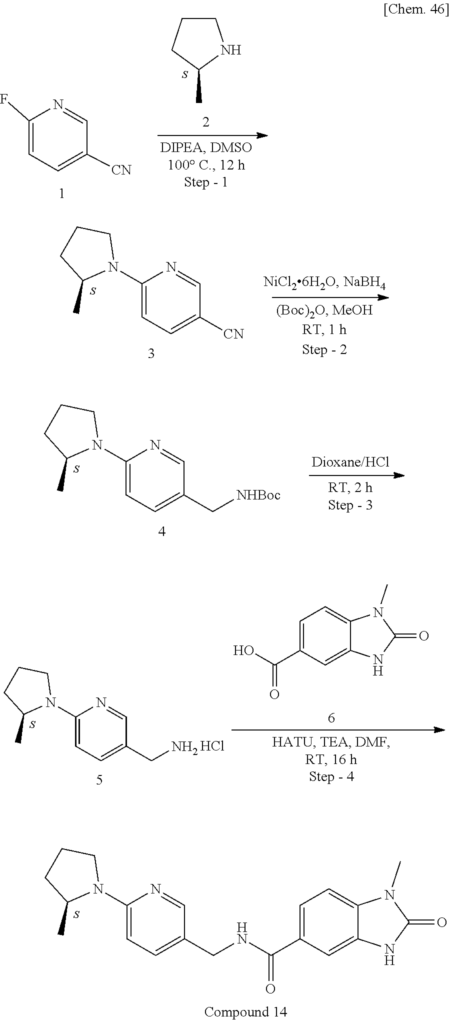

### Step-1: (S)-6-(2-Methylpyrrolidin-1-yl)nicotinonitrile

To a stirred solution of 6-fluoronicotinonitrile (2.2 g, 18.02 mmol) in acetonitrile (10 mL) was added DIPEA (13.97 g, 108.1 mmol) followed by (S)-2-methylpyrrolidine (2.41 g, 28.3 mmol) and stirred at 100° C. for 12 h. The reaction mixture was concentrated to remove DIPEA then poured into ice cold water, and the solid separated was collected by filtration and dried. This was purified by combi flash column chromatography using 20% EtOAc in hexane as an eluent to afford the title compound (3.3 g, 97.82%). LC-MS: 188 [M+H]⁺

### Step-2: tert-Butyl (S)-((6-(2-methylpyrrolidin-1-yl)pyridin-3-yl)methyl)carbamate

A solution of (S)-6-(2-methylpyrrolidin-1-yl)nicotinonitrile (1.75 g, 9.35 mmol) in MeOH (350 mL) was cooled to 0° C., NiCl₂·6H₂O (0.22 g, 0.93 mmol) was added thereto followed by dropwise addition of di-tert-butyl dicarbonate (4.08 g, 18.69 mmol) and stirred for 5 min. Then NaBH₄ (2.48 g, 65.42 mmol) was added in portions thereto by maintaining the same temperature. After stirring for 1 h at RT, the reaction mass was concentrated to remove the solvent. The residue diluted with EtOAc, washed with water, dried over anhydrous Na₂SO₄ and concentrated. The crude compound was purified by combi flash chromatography using 20% EtOAc in hexane as an eluent to afford the title compound (2.2 g, 80.8%). LC-MS: 292 [M+H]⁺

### Step-3: (S)-(6-(2-Methylpyrrolidin-1-yl)pyridin-3-yl)methanamine hydrochloride

To a solution of tert-butyl (S)-((6-(2-methylpyrrolidin-1-yl)pyridin-3-yl)methyl)carbamate (1.9 g, 6.52 mmol) in 1,4-dioxane (15 mL) was added 4 N HCl in 1,4-dioxane (20 mL) and stirred for 2 h at RT. The reaction mixture was concentrated, added diethyl ether and the solid was collected by filtration to get the title compound (1.3 g, 87.8%). LC-MS: 192 [M+H]⁺

### Step-4: (S)-1-Methyl-N-((6-(2-methylpyrrolidin-1-yl)pyridin-3-yl)methyl)-2-oxo-2,3-dihydro-1H-benzimidazole-5-carboxamide (Compound 14)

To a solution of 1-methyl-2-oxo-2,3-dihydro-1H-benzimidazole-5-carboxylic acid (1.09 g, 5.65 mmol) in DMF (15 mL) were added EDC·HCl (1.08 g, 5.64 mmol) and HOBt (0.51 g, 3.7 mmol), and stirred at RT for 10 min. Then (S)-(6-(2-methylpyrrolidin-1-yl)pyridin-3-yl)methanamine hydrochloride (1.2 g, 5.27 mmol) followed by triethylamine (4.44 g, 43.91 mmol) was added thereto and the mixture was stirred at RT for 16 h. The reaction mixture was diluted with water and extracted with 5% MeOH in DCM and concentrated under reduced pressure. The crude residue was further washed with 1:1 solution of DCM and diethyl ether (3×25 mL) to afford the title compound (0.71 g, 37.1%). LC-MS: 366 [M+H]⁺; ¹H-NMR (400 MHz, CD₃OD) δ 11.05 (s, 1H), 8.8 (t, 1H), 8.0 (d, 1H), 7.6 (dd, 1H), 7.5 (d, 1H), 7.45 (dd, 2H), 7.1 (d, 1H), 6.4 (d, 1H), 4.3 (d, 2H), 4.1 (m, 1H), 3.4 (m, 2H), 3.3 (s, 3H), 3.2 (m, 2H), 1.8-2.1 (m, 3H), 1.1 (m, 1H).

### Example-15

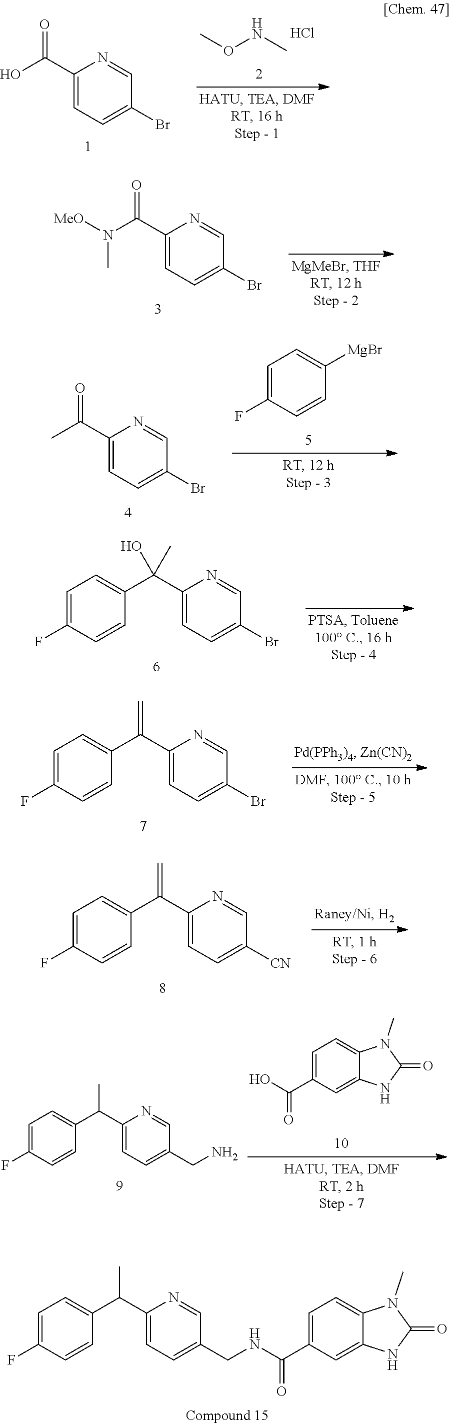

### Step-1: 5-Bromo-N-methoxy-N-methylpicolinamide

To a solution of 5-bromopicolinic acid (4.0 g, 19.8 mmol) in DMF (20 mL) was added HATU (6.78 g, 17.82 mmol) and stirred at RT for 10 min. Then N,O-dimethylhydroxylamine hydrochloride (2.32 g, 23.76 mmol) was added thereto followed by triethylamine (7.51 g, 74.25 mmol) and the mixture was stirred at RT for 2 h. The reaction mixture was added into ice cold water and extracted with EtOAc, dried over anhydrous Na₂SO₄ and concentrated to obtain a residue. This was purified by combi flash column chromatography using 20% EtOAc in hexane as an eluent to afford the title compound (3.8 g, 84.45%). LC-MS: 245 [M+H]⁺

### Step-2: 1-(5-Bromopyridin-2-yl)ethan-1-one

To a stirred solution of 5-bromo-N-methoxy-N-methylpicolinamide (3.8 g, 15.5 mmol) in THF (40 mL) was added methyl magnesium bromide (2.77 g, 23.25 mmol) at 0° C. and stirred at RT for 12 h. The reaction mixture was quenched by saturated solution of ammonium chloride and extracted with DCM, dried over anhydrous Na₂SO 4 and concentrated to get the title compound (2.8 g, 90.3%). LC-MS: 200 [M+H]⁺

### Step-3: 1-(5-Bromopyridin-2-yl)-1-(4-fluorophenyl)ethan-1-ol

To a stirred solution of 1-(5-bromopyridin-2-yl)ethan-1-one (2.8 g, 14.45 mmol) in THF (25 mL) was added (4-fluorophenyl)magnesium bromide (4.32 g, 21.67 mmol) at 0° C. and stirred at RT for 12 h. The reaction mixture was quenched by saturated solution of ammonium chloride, extracted with DCM, dried over anhydrous Na₂SO₄ and concentrated to obtain a residue. This was purified by combi flash column chromatography using 30% EtOAc in hexane as an eluent to afford the title compound (1.5 g, 35.12%). LC-MS: 296 [M+H]⁺

### Step-4: 5-Bromo-2-(1-(4-fluorophenyl)vinyl)pyridine

To a solution of 1-(5-bromopyridin-2-yl)-1-(4-fluorophenyl)ethan-1-ol (1.5 g, 5.07 mmol) in toluene (10 mL) was added PTSA (0.096 g, 0.50 mmol) at 0° C. and stirred at 100° C. for 16 h. The reaction mixture was quenched with water, extracted with EtOAc, dried over anhydrous Na₂SO₄ and concentrated to obtain a residue. This was purified by combi flash column chromatography using 10% EtOAc in hexane as an eluent to afford the title compound (0.9 g, 69.23%). LC-MS: 280 [M+H]⁺

### Step-5: 6-(1-(4-Fluorophenyl)vinyl)nicotinonitrile

To a solution of 5-bromo-2-(1-(4-fluorophenyl)vinyl)pyridine (0.7 g, 2.51 mmol) in DMF (5 mL) was added zinc cyanide (0.36 g, 3.02 mmol). The reaction mixture was degassed for 15 mins with argon, tetrakis(triphenylphosphine)palladium(0) (0.29 g, 0.25 mmol) was added thereto and heated at 120° C. for 10 h. The reaction mixture was quenched with KMnO₄ solution and EtOAc was added thereto. The organic layer was separated, dried over anhydrous Na₂SO₄ and concentrated to obtain a residue. This was purified by combi flash column chromatography using 30% EtOAc in hexane as an eluent to afford the title compound (0.45 g, 79.73%). LC-MS: 225 [M+H]⁺

### Step-6: (6-(1-(4-Fluorophenyl)ethyl)pyridin-3-yl)methanamine

To a stirred solution of 6-(1-(4-fluorophenyl)vinyl)nicotinonitrile (0.35 g, 1.56 mmol) in MeOH (8 mL) was added Raney nickel (0.092 g, 1.56 mmol) followed by methanolic ammonia (0.027 g, 1.56 mmol) and stirred at RT for 1 h under hydrogen bladder pressure. The reaction mass was filtered through celite and the filtrate concentrated to afford the title compound (0.21 g, 58.4%). LC-MS: 231 [M+H]⁺

### Step-7: N-((6-(1-(4-Fluorophenyl)ethyl)pyridin-3-yl)methyl)-1-methyl-2-oxo-2,3-dihydro-1H-be nzimidazole-5-carboxamide (Compound 15)

To a solution of 1-methyl-2-oxo-2,3-dihydro-1H-benzimidazole-5-carboxylic acid (0.10 g, 0.52 mmol) in DMF (6 mL) was added HATU (0.24 g, 0.62 mmol) and stirred at RT for 10 min. Then (6-(1-(4-fluorophenyl)ethyl)pyridin-3-yl)methanamine (0.11 g, 0.47 mmol) was added thereto followed by triethylamine (0.26 g, 2.6 mmol) and the mixture was stirred at RT for 2 h. The reaction mixture was added into ice cold water, extracted with EtOAc, dried over anhydrous Na₂SO₄ and concentrated to obtain a residue. This was purified by prep HPLC using method A to afford the title compound (0.007 g, 3.33%). LC-MS: 405 [M+H]⁺; ¹H-NMR (400 MHz, DMSO-D₆) δ 11.08 (bs, 1H), 8.93 (t, 1H), 8.45 (bs, 1H), 7.62-7.60 (d, 2H), 7.49 (s, 1H), 7.33-7.30 (t, 2H), 7.25-7.23 (d, 1H), 7.14-7.12 (d, 1H), 7.09-7.05 (t, 2H), 4.42-4.40 (d, 2H), 4.26-4.25 (q, 1H), 3.29 (s, 3H), 1.57-1.56 (d, 3H).

### Example-16

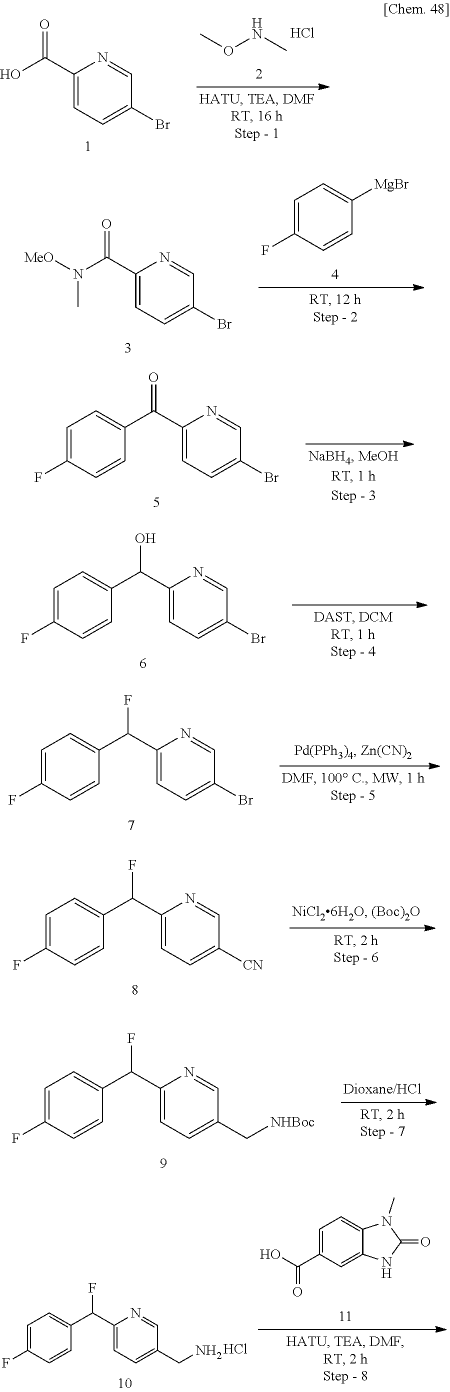

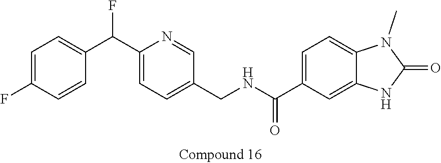 [SMILES: Cn1c(=O)[nH]c2cc(C(=O)NCc3ccc(C(F)c4ccc(F)cc4)nc3)ccc21]

### Step-1: 5-Bromo-N-methoxy-N-methylpicolinamide

To a solution of 5-bromopicolinic acid (2.0 g, 9.9 mmol) in DMF (20 mL) was added HATU (4.52 g, 11.88 mmol) and stirred at RT for 10 min. Then N,O-dimethylhydroxylamine hydrochloride (1.16 g, 11.88 mmol) was added thereto followed by triethylamine (3.00 g, 29.69 mmol) and the mixture was stirred at RT for 16 h. The reaction mixture was added into ice cold water, extracted with EtOAc, dried over anhydrous Na₂SO₄ and concentrated to obtain a residue. This was purified by combi flash column chromatography using 20% EtOAc in hexane as an eluent to afford the title compound (0.75 g, 30.91%). LC-MS: 245 [M+H]⁺

### Step-2: (5-Bromopyridin-2-yl)(4-fluorophenyl)methanone

To a stirred solution of 5-bromo-N-methoxy-N-methylpicolinamide (0.75 g, 3.06 mmol) in THF (10 mL) was added (4-fluorophenyl)magnesium bromide (1.22 g, 6.12 mmol) and stirred at RT for 1 h. The reaction mixture was quenched by ammonium chloride, extracted with EtOAc, dried over anhydrous Na₂SO₄ and concentrated to obtain a residue. This was purified by combi flash column chromatography using 20% EtOAc in hexane as an eluent to afford the title compound (0.68 g, 79.34%). LC-MS: 280 [M+H]⁺

### Step-3: (5-Bromopyridin-2-yl)(4-fluorophenyl)methanol

To a stirred solution of (5-bromopyridin-2-yl)(4-fluorophenyl)methanone (0.68 g, 2.42 mmol) in MeOH (5 mL) was added NaBH₄ (0.14 g, 3.65 mmol) at 0° C. and stirred at RT for 1 h. The reaction mass was concentrated, diluted with EtOAc and water. The organic layers were dried over anhydrous Na₂SO₄ and concentrated to obtain a residue. This was purified by combi flash column chromatography using 30% EtOAc in hexane as an eluent to afford the title compound (0.5 g, 73.03%). LC-MS: 282 [M+H]⁺

### Step-4: 5-Bromo-2-(fluoro(4-fluorophenyl)methyl)pyridine

To a solution of (5-bromopyridin-2-yl)(4-fluorophenyl)methanol (0.5 g, 1.772 mmol) in DCM (5 mL) was added DAST (0.43 g, 2.65 mmol) at 0° C. and stirred at RT for 1 h. The reaction mixture was quenched by sodium bicarbonate solution, extracted with DCM, dried over anhydrous Na₂SO₄ and concentrated to obtain a residue. This was purified by combi flash column chromatography using 1% EtOAc in hexane as an eluent to afford the title compound (0.35 g, 69.52%). LC-MS: 286 [M+H]⁺

### Step-5: 6-(Fluoro(4-fluorophenyl)methyl)nicotinonitrile

To a solution of 5-bromo-2-(fluoro(4-fluorophenyl)methyl)pyridine (0.35 g, 1.23 mmol) in DMF (5 mL) was added zinc cyanide (0.029 g, 0.24 mmol). The reaction mixture was degassed for 15 mins with argon followed by addition of tetrakis(triphenylphosphine)palladium(0) (0.14 g, 0.12 mmol), and heated at 100° C. for 1 h in microwave. The reaction mixture was quenched with KMnO₄ solution and EtOAc was added thereto. The organic layer was separated, dried over anhydrous Na₂SO₄ and concentrated to obtain a residue. This was purified by combi flash column chromatography using 10% EtOAc in hexane as an eluent to afford the title compound (0.22 g, 77.57%). LC-MS: 231 [M+H]⁺

### Step-6: tert-Butyl ((6-(fluoro(4-fluorophenyl)methyl)pyridin-3-yl)methyl)carbamate

To a stirred solution of 6-(fluoro(4-fluorophenyl)methyl)nicotinonitrile (0.22 g, 0.96 mmol) in MeOH (5 mL) was added nickel(II) chloride hexahydrate (0.11 g, 0.47 mmol) followed by di-tert-butyl dicarbonate (0.42 g, 1.91 mmol). Then NaBH₄ (0.054 g, 1.43 mmol) was added in portions thereto at 0° C. and the reaction mixture was stirred at RT for 2 h. The reaction mass was concentrated, and the residue was diluted with EtOAc and filtered through celite pad. The filtrate was concentrated and purified by combi flash column chromatography using 8% EtOAc in hexane as an eluent to afford the title compound (0.14 g, 42.23%). LC-MS: 335 [M+H]⁺

### Step-7: (6-(Fluoro(4-fluorophenyl)methyl)pyridin-3-yl)methanamine hydrochloride

To a stirred solution of tert-butyl ((6-(fluoro(4-fluorophenyl)methyl)pyridin-3-yl)methyl)carbamate (0.14 g, 0.40 mmol) in 1,4-dioxane (1 mL) was added 4 N HCl in 1,4-dioxane (2 mL) at 0° C. and the reaction mixture was stirred at RT for 2 h. The reaction mass was concentrated, solid was washed with diethyl ether to get the title compound (0.11 g, 42.23%). LC-MS: 235 [M+H]⁺

### Step-8: N-((6-(Fluoro(4-fluorophenyl)methyl)pyridin-3-yl)methyl)-1-methyl-2-oxo-2,3-dihydro-1H-benzimidazole-5-carboxamide (Compound 16)

To a solution of 1-methyl-2-oxo-2,3-dihydro-1H-benzimidazole-5-carboxylic acid (0.09 g, 0.47 mmol) in DMF (2 mL) were added EDC·HCl (0.108 g, 0.56 mmol) and HOBt (0.076 g, 0.55 mmol), and stirred at RT for 10 min. Then (6-(fluoro(4-fluorophenyl)methyl)pyridin-3-yl)methanamine hydrochloride (0.11 g, 0.42 mmol) was added thereto followed by triethylamine (0.14 g, 1.4 mmol) and the mixture was stirred at RT for 2 h. The reaction mixture was poured into ice cold water, extracted with EtOAc, dried over anhydrous Na₂SO₄ and concentrated to obtain a residue. This was purified by washing with diethyl ether (3×25 mL) to afford the title compound (0.022 g, 11.51%). LC-MS: 409 [M+H]⁺; ¹H-NMR (400 MHz, DMSO-D₆) δ 11.08 (s, 1H), 8.98 (t, 1H), 8.50 (bs, 1H), 7.80 (d, 1H), 7.63-7.61 (d, 1H), 7.58-7.56 (d, 1H), 7.5 (s, 1H), 7.4 (t, 2H), 7.23-7.19 (t, 2H), 7.16-7.14 (d, 1H), 6.57 (s, 1H), 4.4 (s, 2H), 3.3 (s, 3H).

### Example-17

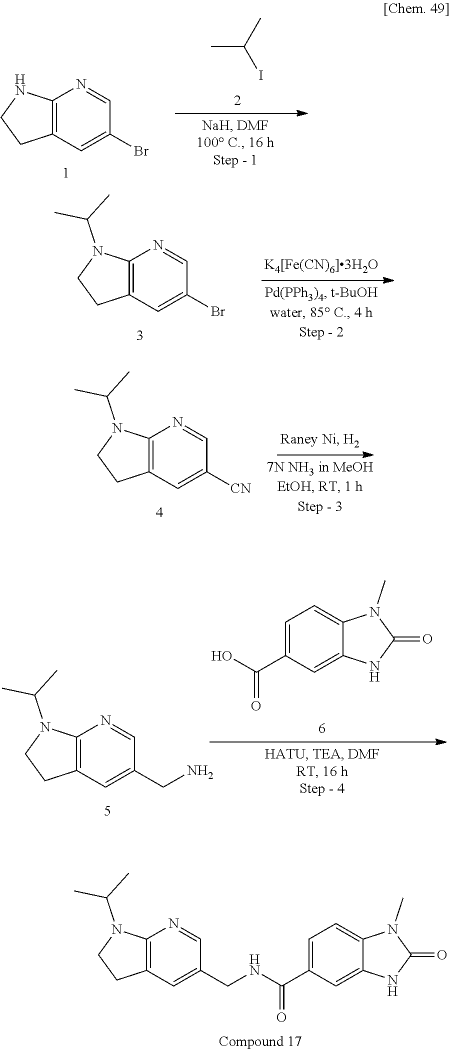

### Step-1: 5-Bromo-1-isopropyl-2,3-dihydro-1H-pyrrolo[2,3-b]pyridine

To a stirred solution of 5-bromo-2,3-dihydro-1H-pyrrolo[2,3-b]pyridine (4.0 g, 20.09 mmol) in DMF (40 mL) was added sodium hydride (0.69 g, 30.14 mmol) at 0° C. and stirred for 10 mins. Then 2-iodopropane (4.44 g, 26.12 mmol) was added thereto at 0° C. and the reaction mixture was stirred at 100° C. for 16 hr. The reaction mixture was quenched with ice cold water, extracted with EtOAc, dried over anhydrous Na₂SO₄ and concentrated to obtain a residue. This was purified by combi flash column chromatography using 20% EtOAc in hexane as an eluent to afford the title compound (2.8 g, 57.79%). LC-MS: 241 [M+H]⁺

### Step-2: 1-Isopropyl-2,3-dihydro-1H-pyrrolo[2,3-b]pyridine-5-carbonitrile

To a stirred solution of 5-bromo-1-isopropyl-2,3-dihydro-1H-pyrrolo[2,3-b]pyridine (1.1 g, 4.56 mmol) in t-BuOH (10 mL) and water (10 mL) were added K₄[Fe(CN)₆]·3H₂O (0.77 g, 1.82 mmol) and DBU (0.17 g, 1.14 mmol). The reaction mixture was degassed for 15 mins with argon followed by addition of tetrakis(triphenylphosphine)palladium(0) (0.26 g, 0.22 mmol), and the reaction mixture heated to 85° C. for 4 h. The reaction mixture was cooled to RT, diluted with 10% MeOH in DCM and filtered through celite. The filtrate was concentrated and purified by combi flash column chromatography using 20% EtOAc in hexane as an eluent to afford the title compound (0.5 g, 58.54%). LC-MS: 188 [M+H]⁺

### Step-3: (1-Isopropyl-2,3-dihydro-1H-pyrrolo[2,3-b]pyridin-5-yl)methanamine

To a stirred solution of 1-isopropyl-2,3-dihydro-1H-pyrrolo[2,3-b]pyridine-5-carbonitrile (0.3 g, 1.60 mmol) in EtOH (15 mL) was added Raney nickel (0.019 g, 0.32 mmol) followed by methanolic ammonia (1 mL) and stirred at RT, under hydrogen bladder pressure for 1 h. The reaction mass was filtered through celite pad and the filtrate concentrated to afford the title compound (0.3 g, 98%). LC-MS: 192.15 [M+H]⁺

### Step-4: N-((1-Isopropyl-2,3-dihydro-1H-pyrrolo[2,3-b]pyridin-5-yl)methyl)-1-methyl-2-oxo-2,3-dihydro-1H-benzimidazole-5-carboxamide (Compound 17)

To a solution of 1-methyl-2-oxo-2,3-dihydro-1H-benzimidazole-5-carboxylic acid (1.3 g, 6.76 mmol) in DMF (10 mL) was added HATU (3.087 g, 8.11 mmol) and stirred at RT for 10 min. Then (1-isopropyl-2,3-dihydro-1H-pyrrolo[2,3-b]pyridin-5-yl)methanamine (1.16 g, 6.09 mmol) was added thereto followed by triethylamine (3.42 g, 33.82 mmol) and stirred at RT for 16 h. The reaction mixture was added into ice cold water, extracted with EtOAc, dried over anhydrous Na₂SO₄ and concentrated to obtain a residue. This was purified by prep HPLC using method B to afford the pure title compound (0.28 g, 11.33%). LC-MS: 366 [M+H]⁺; ¹HNMR (400 MHz, DMSO-D₆) δ 11.01 (s, 1H), 8.88 (t, 1H), 7.61 (dd, 1H), 7.54 (bs, 1H), 7.4 (d, 1H), 7.1 (d, 1H), 4.2 (d, 2H), 4.1 (t, 1H), 3.77-3.73 (t, 2H), 3.2 (s, 3H), 3.1-3.06 (t, 2H), 2.83 (m, 1H), 1.2 (d, 6H)

### Example-18

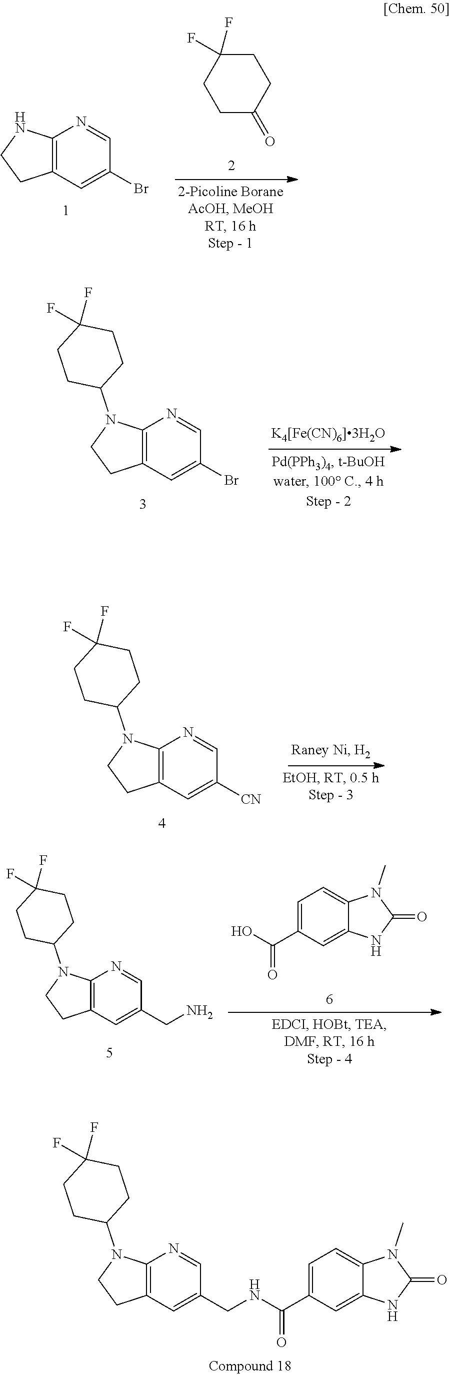

### Step-1: 5-Bromo-1-(4,4-difluorocyclohexyl)-2,3-dihydro-1H-pyrrolo[2,3-b]pyridine

To a stirred solution of 5-bromo-2,3-dihydro-1H-pyrrolo[2,3-b]pyridine (2.7 g, 13.56 mmol) in MeOH (60 mL) were added 4,4-difluorocyclohexan-1-one (2.73 g, 20.34 mmol), acetic acid (2.04 g, 33.9 mmol) and 2-picoline borane complex (3.525 g, 33.91 mmol) at 0° C., and stirred at RT for 16 h. The reaction mixture was poured into ice cold water, extracted with EtOAc, dried over anhydrous Na₂SO₄ and concentrated to obtain a residue. This was purified by combi flash column chromatography using 20% EtOAc in hexane as an eluent to afford the title compound (3.6 g, 83.68%). LC-MS: 319 [M+H]⁺

### Step-2: 1-(4,4-Difluorocyclohexyl)-2,3-dihydro-1H-pyrrolo[2,3-b]pyridine-5-carbonitrile

To a stirred solution of 5-bromo-1-(4,4-difluorocyclohexyl)-2,3-dihydro-1H-pyrrolo[2,3-b]pyridine (3.6 g, 11.35 mmol) in t-BuOH (50 mL) and water (50 mL) was added DBU (0.43 g, 2.83 mmol) followed by potassium ferricyanide (1.92 g, 4.54 mmol). The reaction mass was degassed for 15 min with argon. Then tetrakis(triphenylphosphine)palladium(0) (0.66 g, 0.56 mmol) was added thereto and heated at 100° C. for 4 h. The reaction mass was concentrated to remove t-BuOH and the residue diluted with water, extracted with EtOAc, dried over anhydrous Na₂SO₄ and concentrated. The crude residue was purified by combi flash column chromatography using 30% EtOAc in hexane as an eluent to afford the title compound (2.4 g, 80.32%). LC-MS: 264 [M+H]⁺

### Step-3: (1-(4,4-Difluorocyclohexyl)-2,3-dihydro-1H-pyrrolo[2,3-b]pyridin-5-yl)methanamine

To a stirred solution of 1-(4,4-difluorocyclohexyl)-2,3-dihydro-1H-pyrrolo[2,3-b]pyridine-5-carbonitrile (2.4 g, 9.11 mmol) in EtOH (40 mL) was added Raney nickel (2.4 g) followed by methanolic ammonia (2.4 mL) and stirred at RT for 30 mins under hydrogen bladder pressure. The reaction mass filtered through celite pad and the filtrate concentrated to afford the title compound (2.4 g, 98.49%). LC-MS: 268 [M+H]⁺

### Step-4: N-((1-(4,4-difluorocyclohexyl)-2,3-dihydro-1H-pyrrolo[2,3-b]pyridin-5-yl)methyl)-1-met hyl-2-oxo-2,3-dihydro-1H-benzimidazole-5-carboxamide (Compound 18)

To a solution of 1-methyl-2-oxo-2,3-dihydro-1H-benzimidazole-5-carboxylic acid (2.32 g, 12.12 mmol) in DMF (2 mL) were added EDC·HCl (1.72 g, 8.97 mmol) and HOBt (1.09 g, 8.07 mmol), and stirred at RT for 10 min. Then (1-(4,4-difluorocyclohexyl)-2,3-dihydro-1H-pyrrolo[2,3-b]pyridin-5-yl)methanamine (2.4 g, 8.97 mmol) was added thereto followed by triethylamine (4.54 g, 44.85 mmol) and the mixture was stirred at RT for 16 h. The reaction mixture was added into ice cold water, and the precipitate formed was collected by filtration and dried. The obtained crude compound was washed with diethyl ether (3×50 mL) to afford the title compound (1.2 g). LC-MS: 442 [M+H]⁺; ¹H-NMR (400 MHz, DMSO-D₆) δ 11.0 (bs, 1H), 8.7 (t, 1H), 7.7 (s, 1H), 7.6 (d, 1H), 7.5 (bs, 1H), 7.2 (bs, 1H), 7.1 (d, 1H), 4.2 (d, 2H), 4.0 (s, 1H), 3.4 (t 2H), 3.29 (s, 3H), 2.9-2.8 (t, 2H), 2.06 (s, 3H), 1.95 (s, 1H), 1.7-1.6 (t, 4H).

### Example-19

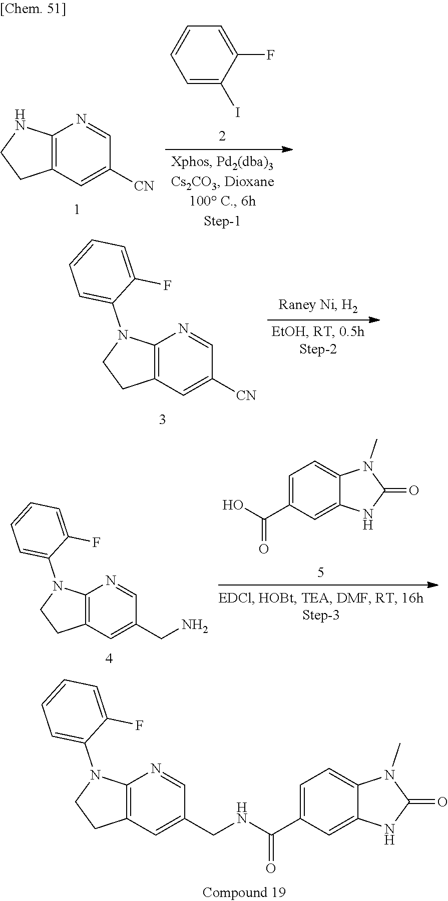 [SMILES: Fc1ccccc1I ; N#Cc1cnc2c(c1)CCN2 ; N#Cc1cnc2c(c1)CCN2c1ccccc1F ; NCc1cnc2c(c1)CCN2c1ccccc1F ; Cn1c(=O)[nH]c2cc(C(=O)O)ccc21 ; Cn1c(=O)[nH]c2cc(C(=O)NCc3cnc4c(c3)CCN4c3ccccc3F)ccc21]

### Step-1: 1-(2-Fluorophenyl)-2,3-dihydro-1H-pyrrolo[2,3-b]pyridine-5-carbonitrile

To a stirred solution of 2,3-dihydro-1H-pyrrolo[2,3-b]pyridine-5-carbonitrile (0.25 g, 1.72 mmol) in 1,4-dioxane (3 mL) was added 1-fluoro-2-iodobenzene (0.90 g, 5.17 mmol) followed by caesium carbonate (1.68 g, 50.52 mmol). The reaction mass was degassed for 15 min with argon. Xphos (0.082 g, 0.17 mmol) and Pd₂(dba)₃ (0.079 g, 0.08 mmol) were added thereto and heated to 100° C. for 6 h. The reaction mass was filtered through celite bed and concentrated to obtain a residue. This was purified by combi flash chromatography using 40% EtOAc in hexane as an eluent to afford the title compound (0.26 g, 61.89%). LC-MS: 240 [M+H]⁺

### Step-2: (1-(2-Fluorophenyl)-2,3-dihydro-1H-pyrrolo[2,3-b]pyridin-5-yl)methanamine

To a solution of 1-(2-fluorophenyl)-2,3-dihydro-1H-pyrrolo[2,3-b]pyridine-5-carbonitrile (0.13 g, 5.43 mmol) in EtOH (4 mL) was added Raney nickel (0.13 g) and stirred at RT for 30 min under hydrogen bladder pressure. The reaction mass was filtered through celite pad and the filtrate concentrated to afford the title compound (0.13 g, 100%). LC-MS: 244 [M+H]⁺

### Step-3: N-((1-(2-Fluorophenyl)-2,3-dihydro-1H-pyrrolo[2,3-b]pyridin-5-yl)methyl)-1-methyl-2-o xo-2,3-dihydro-1H-benzimidazole-5-carboxamide (Compound 19)

To a solution of 1-methyl-2-oxo-2,3-dihydro-1H-benzimidazole-5-carboxylic acid (0.15 g, 0.80 mmol) in DMF (2 mL) were added EDC·HCl (0.12 g, 0.64 mmol) and HOBt (0.098 g, 0.64 mmol), and stirred at RT for 10 min. Then (1-(2-fluorophenyl)-2,3-dihydro-1H-pyrrolo[2,3-b]pyridin-5-yl)methanamine (0.13 g, 0.53 mmol) was added thereto followed by triethylamine (0.22 g, 2.13 mmol) and the mixture was stirred at RT for 16 h. The reaction mixture was added into ice cold water, and the precipitate formed was collected by filtration and dried to obtain a residue. This was further purified by preparative HPLC using method A to afford the title compound (0.1 g, 44.86%). LC-MS: 418 [M+H]⁺; ¹H-NMR (400 MHz, CD₃OD) δ 7.89 (s, 1H), 7.68 (dd, 1H), 7.67-7.6 (t, 2H), 7.58-7.52 (m, 2H), 7.38-7.35 (m, 2H), 7.21-7.19 (d, 1H), 4.49 (s, 2H), 4.35 (t, 2H), 3.46 (t, 2H), 3.42 (s, 3H).

### Example-20

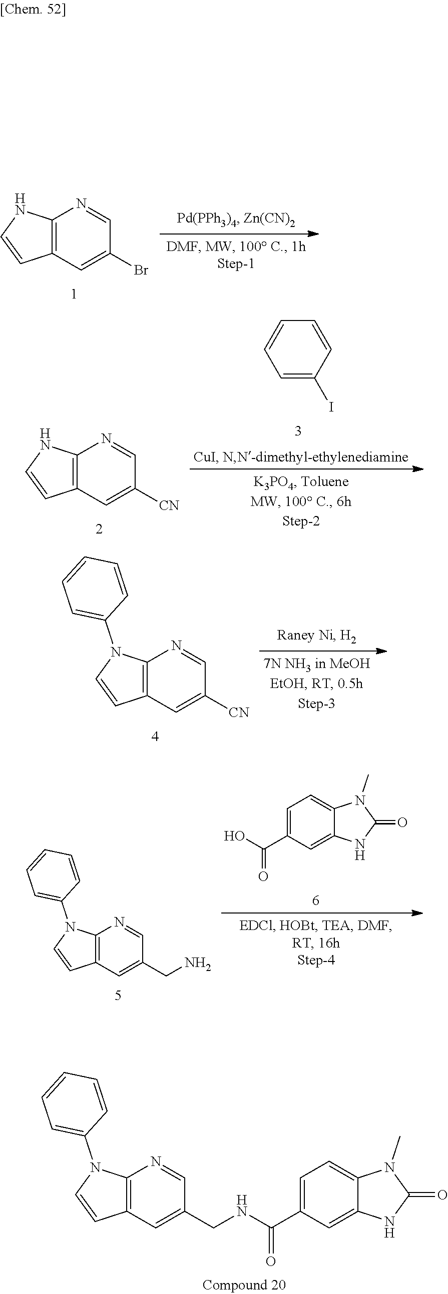

### Step-1: 1H-Pyrrolo[2,3-b]pyridine-5-carbonitrile

To a stirred solution of 5-bromo-1H-pyrrolo[2,3-b]pyridine (0.65 g, 3.30 mmol) in DMF (10 mL) was added zinc cyanide (0.47 g, 3.95 mmol). The reaction mixture was degassed for 15 mins with argon followed by addition of tetrakis(triphenylphosphine)palladium(0) (0.38 g, 0.33 mmol) and the reaction mixture was heated at 100° C. in microwave for 1 h. The reaction mixture was quenched with KMnO₄ solution and added ice cold water mixture, extracted with EtOAc, dried over anhydrous Na₂SO₄ and concentrated to obtain a residue. This was purified by combi flash column chromatography using 20% EtOAc in hexane as an eluent to afford the title compound (0.4 g, 84.7%). LC-MS: 144 [M+H]⁺

### Step-2: 1-Phenyl-1H-pyrrolo[2,3-b]pyridine-5-carbonitrile

To a stirred solution of 1H-pyrrolo[2,3-b]pyridine-5-carbonitrile (0.4 g, 2.79 mmol) in toluene (8 mL) were added iodobenzene (1.14 g, 5.59 mmol), copper iodide (0.053 g, 0.27 mmol), N,N′-dimethylethylenediamine (0.049 g, 0.55 mmol) and K₃PO₄ (0.38 g, 2.79 mmol), and degassed for 15 min with argon. Then the reaction mixture was heated to 100° C. for 6 h in microwave. The reaction mixture was diluted with water, extracted with EtOAc, dried over anhydrous Na₂SO₄ and concentrated to obtain a residue. This was purified by combi flash column chromatography using 20% EtOAc in hexane as an eluent to afford the title compound (0.38 g, 62.04%). LC-MS: 220 [M+H]⁺

### Step-3: (1-Phenyl-1H-pyrrolo[2,3-b]pyridin-5-yl)methanamine

To a stirred solution of 1-phenyl-1H-pyrrolo[2,3-b]pyridine-5-carbonitrile (0.4 g, 1.82 mmol) in EtOH (15 mL) was added Raney nickel (0.4 g) followed by methanolic ammonia (0.5 mL) and stirred at RT for 30 mins under hydrogen bladder pressure. The reaction mass was filtered through celite pad and concentrated to afford the title compound (0.4 g, 99%). LC-MS: 224 [M+H]⁺

### Step-4: 1-Methyl-2-oxo-N-((1-phenyl-1H-pyrrolo[2,3-b]pyridin-5-yl)methyl)-2,3-dihydro-1H-benzimidazole-5-carboxamide (Compound 20)

To a solution of 1-methyl-2-oxo-2,3-dihydro-1H-benzimidazole-5-carboxylic acid (0.22 g, 1.12 mmol) in DMF (4 mL) were added EDC·HCl (0.26 g, 1.34 mmol) and HOBt (0.18 g, 1.34 mmol), and stirred at RT for 10 min. Then (1-phenyl-1H-pyrrolo[2,3-b]pyridin-5-yl)methanamine (0.2 g, 0.90 mmol) was added thereto followed by triethylamine (0.34 g, 3.35 mmol) and the mixture was stirred at RT for 16 h. The reaction mixture was diluted with ice cold water, extracted with EtOAc, dried over anhydrous Na₂SO₄ and concentrated to obtain a residue. This was purified by combi flash column chromatography using 60% EtOAc in hexane as an eluent to afford the title compound (0.09 g, 20.24%). LC-MS: 398 [M+H]⁺; ¹H-NMR (400 MHz, DMSO-D₆) δ 11.0 (bs, 1H), 9.0-8.9 (t, 1H), 8.59 (s, 1H), 8.31 (s, 1H), 8.0 (d, 1H), 7.94-7.93 (d, 1H), 7.90-7.88 (d, 1H), 7.6 (d, 1H), 7.5 (t, 3H), 7.3 (t, 1H), 7.1 (d, 1H), 6.7 (d, 1H), 4.5 (s, 2H), 3.29 (s, 3H).

### Example-21

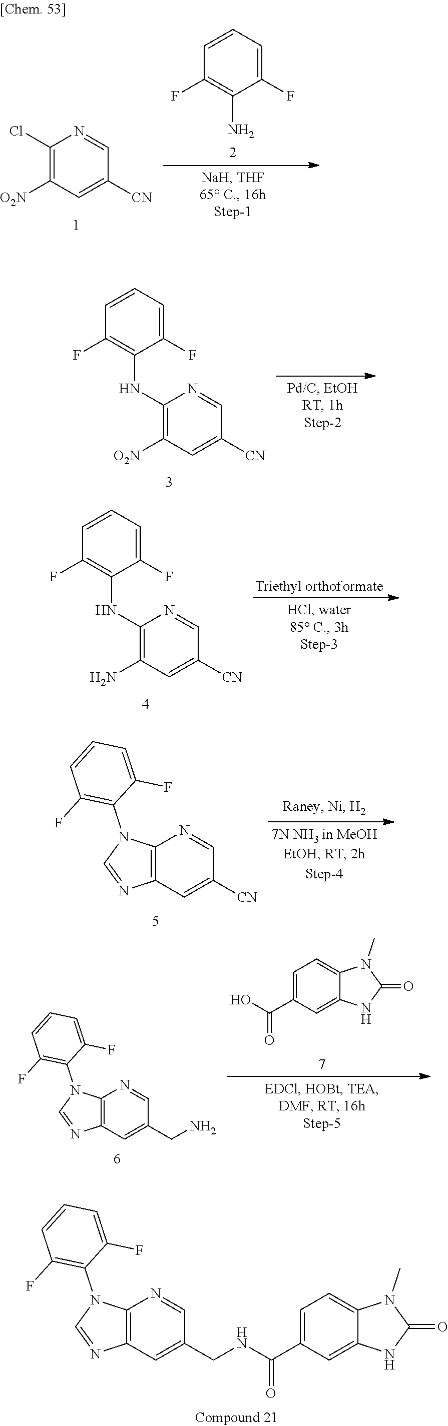

### Step-1: 6-((2,6-Difluorophenyl)amino)-5-nitronicotinonitrile

To a stirred solution of 6-chloro-5-nitronicotinonitrile (0.25 g, 1.36 mmol) in DMF (28 mL) was added NaH (60% dispersion in mineral oil) (0.082 g, 2.04 mmol) at 0° C. and stirred at RT for 1 h, further 2,6-difluoroaniline (0.22 g, 1.70 mmol) was added thereto and refluxed for 16 h. The reaction mixture was diluted with EtOAc, washed with water, dried over anhydrous Na₂SO₄ and concentrated. The obtained crude compound was purified by combi flash chromatography using 20% EtOAc in hexane as an eluent to afford the title compound (0.13 g, 34.56%). LC-MS: 275 [M−H]⁻

### Step-2: 5-Amino-6-((2,6-difluorophenyl)amino)nicotinonitrile

To a solution of 6-((2,6-difluorophenyl)amino)-5-nitronicotinonitrile (0.13 g, 0.45 mmol) in ethanol (4 mL) was added Pd/C (0.025 g) under nitrogen atmosphere and stirred under hydrogen bladder pressure for 1 h at RT. The reaction mass was filtered through celite, and concentrated to afford the title compound (0.1 g, 90%). LC-MS: 247 [M+H]⁺.

### Step-3: 3-(2,6-Difluorophenyl)-3H-imidazo[4,5-b]pyridine-6-carbonitrile

To a stirred solution of 5-amino-6-((2,6-difluorophenyl)amino)nicotinonitrile (0.1 g, 0.41 mmol) in triethylorthoformate (3 mL) was added HCl (0.028 g, 0.81 mmol) and refluxed at 85° C. for 3 h. The reaction mixture was concentrated and the residue purified by column chromatography using 40% EtOAc in hexane as an eluent to afford the title compound (0.09 g, 86.52%). LC-MS: 257 [M+H]⁺.

### Step-4: (3-(2,6-Difluorophenyl)-3H-imidazo[4,5-b]pyridin-6-yl)methanamine

To a solution of 3-(2,6-difluorophenyl)-3H-imidazo[4,5-b]pyridine-6-carbonitrile (0.080 g, 0.31 mmol) in EtOH (4 mL) was added Raney nickel (0.080 g) and stirred under hydrogen bladder pressure and for 2 h at RT. The reaction mass was filtered through celite, filtrate was concentrated to afford the title compound (0.080 g, 98%). LC-MS: 261 [M+H]⁺.

### Step-5: N-((3-(2,6-Difluorophenyl)-3H-imidazo[4,5-b]pyridin-6-yl)methyl)-1-methyl-2-oxo-2,3-dihydro-1H-benzimidazole-5-carboxamide trifluoroacetate (Compound 21)

To a solution of 1-methyl-2-oxo-2,3-dihydro-1H-benzimidazole-5-carboxylic acid (0.08 g, 0.42 mmol) in DMF (4 mL) were added EDC·HCl (0.084 g, 0.43 mmol) and HOBt (0.059 g, 0.43 mmol), and stirred at RT for 10 min. Then (3-(2,6-difluorophenyl)-3H-imidazo[4,5-b]pyridin-6-yl)methanamine (0.065 g, 0.25 mmol) followed by triethylamine (0.15 g, 1.45 mmol) was added thereto and the mixture was stirred at RT for 16 h. The reaction mixture was poured into ice cold water, and the precipitate formed was collected by filtration and dried. This was further purified by preparative HPLC using method A to afford the title compound as a TFA salt (0.017 g, 12.4%). LC-MS: 435 [M+H]⁺; ¹HNMR (400 MHz, CD₃OD) δ 8.6 (s, 1H), 8.45 (d, 1H), 8.2 (s, 1H), 7.7-7.6 (m, 2H), 7.6 (d, 1H), 7.3 (t, 2H), 7.15 (d, 1H), 5.0-4.7 (m, 2H), 3.5-3.2 (m, 3H).

### Example-22

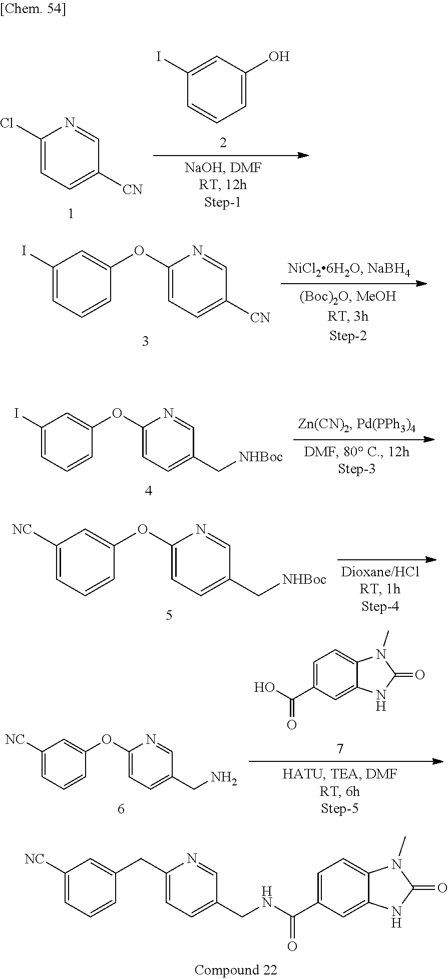

### Step-1: 6-(3-Iodophenoxy)nicotinonitrile

To a stirred solution of 3-iodophenol (2 g, 9.08 mmol) in DMF (15 mL) was added NaOH (0.55 g, 13.75 mmol) followed by 6-chloronicotinonitrile (1.25 g, 9.02 mmol) and stirred at RT for 12 h. The reaction mixture was poured into ice cold water and stirred for another 2 h. The solid separated was collected by filtration and dried to get the title compound (2.2 g, 75.7%). LC-MS: 323 [M+H]⁺

### Step-2: tert-Butyl ((6-(3-iodophenoxy)pyridin-3-yl)methyl)carbamate

To a solution of 6-(3-iodophenoxy)nicotinonitrile (2.2 g, 6.83 mmol) in MeOH (20 mL) was added NiCl₂·6H₂O (0.81 g, 3.42 mmol) followed by dropwise addition of di-tert-butyl dicarbonate (2.99 g, 13.70 mmol) at 0° C. and stirred for 5 min. Then NaBH₄ (0.39 g, 10.26 mmol) was added in portions by maintaining the same temperature. After stirring for 3 h at RT, the reaction mass was concentrated to remove solvent, diluted with EtOAc and filtered on celite. The filtrate was washed with water, dried over anhydrous Na₂SO₄ and concentrated to get a crude compound, which was purified by combi flash column chromatography using 10% EtOAc in hexane as an eluent to afford the title compound (1.3 g, 44.8%). LC-MS: 427 [M+H]⁺

### Step-3: tert-Butyl ((6-(3-cyanophenoxy)pyridin-3-yl)methyl)carbamate

To a solution of tert-butyl ((6-(3-iodophenoxy)pyridin-3-yl)methyl)carbamate (1.2 g, 2.81 mmol) in DMF (10 mL) was added zinc cyanide (0.07 g, 0.56 mmol) followed by tetrakis (triphenylphosphine)palladium(0) (0.32 g, 0.28 mmol) and stirred at 80° C. for 12 h. The reaction mixture was poured into ice cold water, extracted with EtOAc, washed with brine, dried over anhydrous Na₂SO₄ and concentrated to get a crude compound. This was purified by combi flash column chromatography using 23% EtOAc in hexane as an eluent to get the title compound (0.26 g, 28.5%). LC-MS: 326 [M+H]⁺

### Step-4: 3-((5-(Aminomethyl)pyridin-2-yl)oxy)benzonitrile hydrochloride

To a solution of tert-butyl ((6-(3-cyanophenoxy)pyridin-3-yl)methyl)carbamate (0.26 g, 0.79 mmol) in 1,4-dioxane (2 mL) was added 4 N HCl in 1,4-dioxane (2.85 mL) and stirred for 1 h at RT. The reaction mixture was then concentrated, added diethyl ether and the solid was collected by filtration to afford the crude title compound (0.25 g), which was taken further without purification. LC-MS: 226 [M+H]⁺

### Step-5: N-((6-(3-Cyanophenoxy)pyridin-3-yl)methyl)-1-methyl-2-oxo-2,3-dihydro-1H-benzimid azole-5-carboxamide trifluoroacetate (Compound 22)

To a solution of 1-methyl-2-oxo-2,3-dihydro-1H-benzimidazole-5-carboxylic acid (0.23 g, 1.19 mmol) in DMF (10 mL) were added HATU (0.55 g, 1.43 mmol) and triethylamine (0.61 g, 5.98 mmol), and stirred at RT for 10 min. Then 3-((5-(aminomethyl)pyridin-2-yl)oxy)benzonitrile hydrochloride (0.25 g, 0.95 mmol) was added and the mixture was stirred at RT for 6 h. The reaction mixture was added into ice cold water, and the precipitate formed was collected by filtration and dried. The crude residue was further purified by preparative HPLC using method A to afford the title compound as a TFA salt (0.03 g, 5%). LC-MS: 400 [M+H]⁺; ¹H-NMR (400 MHz, DMSO-D₆) δ 11.06 (s, 1H), 8.93 (t, 1H), 8.09 (s, 1H), 7.83-7.81 (dd, 1H), 7.65-7.64 (m, 2H), 7.60-7.57 (m, 2H), 7.47-7.46 (m, 2H), 7.13-7.11 (dd, 1H), 7.08-7.06 (dd, 1H), 4.40-4.39 (d, 2H) 3.27 (s, 3H)

### Example-23

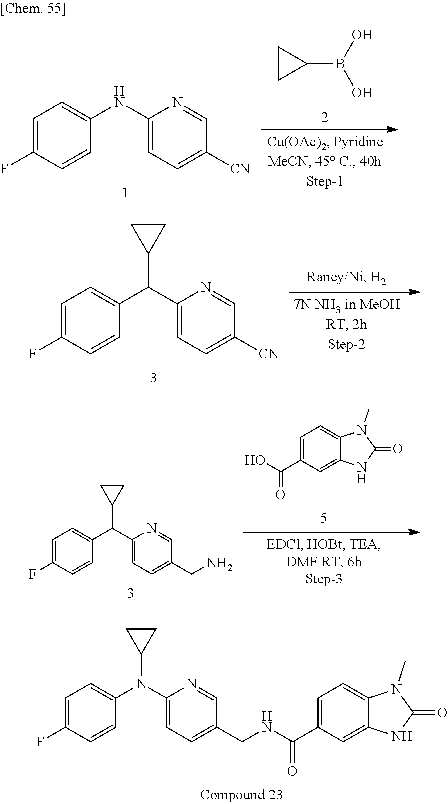 [SMILES: OB(O)C1CC1 ; N#Cc1ccc(Nc2ccc(F)cc2)nc1 ; N#Cc1ccc(C(c2ccc(F)cc2)C2CC2)nc1 ; NCc1ccc(C(c2ccc(F)cc2)C2CC2)nc1 ; Cn1c(=O)[nH]c2cc(C(=O)O)ccc21 ; Cn1c(=O)[nH]c2cc(C(=O)NCc3ccc(N(c4ccc(F)cc4)C4CC4)nc3)ccc21]

### Step-1: 6-(Cyclopropyl(4-fluorophenyl)amino)nicotinonitrile

To a solution of 6-((4-fluorophenyl)amino)nicotinonitrile (1 g, 4.68 mmol) in acetonitrile (10 mL) were added pyridine (1.29 g, 16.41 mmol), cyclopropyl boronic acid (0.80 g, 9.30 mmol) and copper(II) acetate (0.17 g, 0.93 mmol) then stirred for 40 h at 45° C. The reaction mixture was poured into water and extracted with EtOAc. The separated organic layer was washed with brine, dried over anhydrous Na₂SO₄ and concentrated to get a crude compound. The crude compound was purified by combi flash column chromatography using 20% EtOAc in hexane as an eluent to afford the title compound (0.45 g, 37.9%). LC-MS: 254 [M+H]⁺. ¹H-NMR (400 MHz, DMSO-D₆) δ 8.45 (d, 1H), 7.97-7.95 (dd, 1H), 7.27-7.25 (m, 4H), 7.02-7.00 (d, 1H), 3.13-3.10 (m, 1H), 0.94-0.92 (m, 2H), 0.53-0.51 (m, 2H).

### Step-2: 5-(Aminomethyl)-N-cyclopropyl-N-(4-fluorophenyl)pyridin-2-amine

A solution of 6-(cyclopropyl(4-fluorophenyl)amino)nicotinonitrile (0.22 g, 0.86 mmol) in MeOH (2 mL) was cooled to 0° C. To the solution was added 7 N ammonia in MeOH (0.3 mL) followed by the addition of Raney nickel (0.22 g) and stirred under hydrogen bladder pressure for 2 h at RT. The reaction mass was filtered through celite and the filtrate was concentrated to afford the crude title compound (0.22 g), which was taken further without purification. LC-MS: 258 [M+H]⁺.

### Step-3: N-((6-(Cyclopropyl(4-fluorophenyl)amino)pyridin-3-yl)methyl)-1-methyl-2-oxo-2,3-dihy dro-1H-benzimidazole-5-carboxamide (Compound 23)

To a solution of 1-methyl-2-oxo-2,3-dihydro-1H-benzimidazole-5-carboxylic acid (0.2 g, 1.04 mmol) in DMF (4 mL) were added EDC·HCl (0.23 g, 1.2 mmol) and HOBt (0.16 g, 1.2 mmol), and stirred at RT for 10 min. Then 5-(aminomethyl)-N-cyclopropyl-N-(4-fluorophenyl)pyridin-2-amine (0.21 g, 0.8 mmol) was added thereto followed by triethylamine (0.31 g, 3.12 mmol) and the mixture was stirred at RT for 6 h. The reaction mixture was poured into ice cold water, extracted with EtOAc, dried over anhydrous Na₂SO₄ and concentrated to get a crude compound. This was purified by combi flash column chromatography using 5% MeOH in DCM as an eluent to afford the title compound. (0.01 g, 2.3%) LC-MS: 432 [M+H]⁺; ¹H-NMR (400 MHz, DMSO-D₆) δ 11.06 (s, 1H), 8.84 (m, 1H), 8.02 (d, 1H), 7.62-7.6 (m, 2H), 7.49 (s, 1H), 7.23-7.14 (m, 4H), 6.98-6.96 (d, 1H), 4.33-4.31 (d, 2H), 3.32-3.30 (m, 3H), 2.98 (m, 2H), 0.89 (m, 2H), 0.47-0.46 (m, 2H).

### Example-24

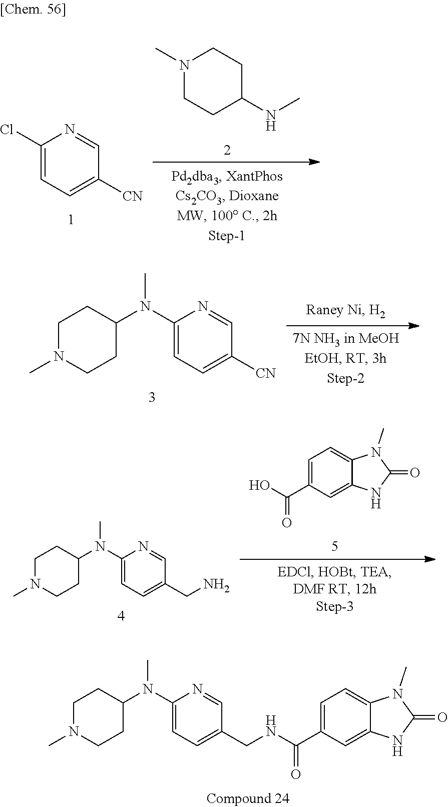

### Step-1: 6-(Methyl(1-methylpiperidin-4-yl)amino)nicotinonitrile

To a stirred solution of 6-chloronicotinonitrile (0.2 g, 1.44 mmol) in 1,4-dioxane (4 mL) was added N,1-dimethylpiperidin-4-amine (0.20 g, 1.59 mmol) followed by cesium carbonate (1.41 g, 4.33 mmol). The reaction mass was degassed for 15 min with argon. Xantphos (0.17 g, 0.28 mmol) and Pd₂(dba)₃ (0.07 g, 0.07 mmol) was added thereto, and the mixture was heated to 100° C. in microwave for 2 h. The reaction mass was filtered through celite and concentrated. This crude residue was dissolved in water and extracted using EtOAc. The separated organic layer was washed with brine, dried over anhydrous Na₂SO₄ and concentrated to get a crude compound. The crude compound was purified by combi flash chromatography using 10% MeOH in DCM as an eluent to afford the title compound (0.33 g, 99.4%). LC-MS: 231 [M+H]⁺

### Step-2: 5-(Aminomethyl)-N-methyl-N-(1-methylpiperidin-4-yl)pyridin-2-amine

A solution of 6-(methyl(1-methylpiperidin-4-yl)amino)nicotinonitrile (0.33 g, 1.43 mmol) in MeOH (4 mL) was cooled to 0° C., Raney nickel (0.33 g) was added thereto followed by 7 N ammonia in MeOH (0.4 mL) and stirred under hydrogen bladder pressure for 3 h at RT. The reaction mass was filtered through celite and the filtrate was concentrated to get a crude compound. The crude compound was purified by washing with diethyl ether to afford the title compound (0.3 g, 89.5%). LC-MS: 235 [M+H]⁺

### Step-3: 1-Methyl-N-((6-(methyl(1-methylpiperidin-4-yl)amino)pyridin-3-yl)methyl)-2-oxo-2,3-dihydro-1H-benzimidazole-5-carboxamide (Compound 24)

To a solution of 1-methyl-2-oxo-2,3-dihydro-1H-benzimidazole-5-carboxylic acid (0.18 g, 0.93 mmol) in DMF (4 mL) was added EDC·HCl (0.18 g, 0.93 mmol), HOBt (0.13 g, 0.96 mmol) and stirred at RT for 10 min. Then 5-(aminomethyl)-N-methyl-N-(1-methylpiperidin-4-yl)pyridin-2-amine (0.15 g, 0.64 mmol) was added thereto followed by triethylamine (0.24 g, 2.34 mmol), and the mixture was stirred at RT for 12 h. The reaction mixture was poured into ice cold water, extracted with EtOAc, dried over anhydrous Na₂SO₄ and concentrated. This was purified by prep HPLC using method B to afford the title compound (0.03 g, 8.98%). LC-MS: 409 [M+H]⁺; ¹H-NMR (400 MHz, DMSO-D₆) δ 7.95-7.85 (m, 2H), 7.59-7.55 (d, 1H), 7.48 (s, 1H), 7.34-7.28 (d, 2H), 7.16-7.14 (d, 1H), 4.35 (s, 2H), 4.32-4.28 (bs, 1H), 3.55-3.45 (d, 2H), 3.28 (s, 3H), 3.1-3.0 (t, 2H), 2.94 (s, 3H), 2.77 (s, 3H), 2.05-1.98 (m, 3H), 1.92-1.85 (d, 2H).

### Example-25

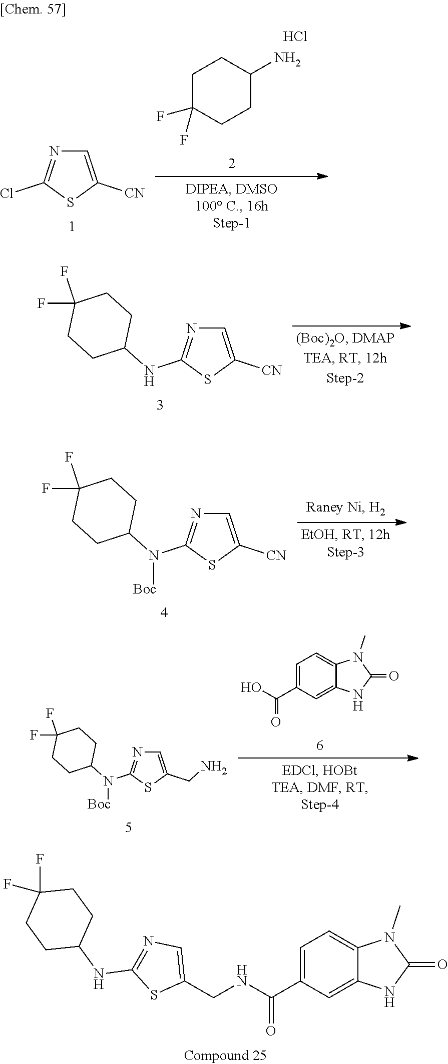

### Step-1: 2-((4,4-Difluorocyclohexyl)amino)thiazole-5-carbonitrile

To a stirred solution of 2-chlorothiazole-5-carbonitrile (0.50 g, 3.46 mmol) in DMSO (5 mL) was added DIPEA (1.34 g, 10.37 mmol) followed by 4,4-difluorocyclohexan-1-amine hydrochloride (0.59 g, 3.46 mmol) and stirred at 100° C. for 16 h. The reaction mixture was poured into ice cold water and extracted with EtOAc. The separated organic layer was washed with brine, dried over anhydrous Na₂ SO₄ and concentrated to get a crude compound. The crude compound was purified by combi flash column chromatography using 20% EtOAc in hexane as an eluent to afford the title compound (0.40 g, 47.56%). LC-MS: 242 [M−H]⁻.

### Step-2: tert-Butyl (5-cyanothiazol-2-yl)(4,4-difluorocyclohexyl)carbamate

To a solution of 2-((4,4-difluorocyclohexyl)amino)thiazole-5-carbonitrile (0.20 g, 0.82 mmol) in acetonitrile (5 mL) were added triethylamine (0.25 g, 2.47 mmol) and DMAP (0.01 g, 0.08 mmol) followed by dropwise addition of di-tert-butyl dicarbonate (0.21 g, 0.98 mmol) at 0° C. The reaction mixture was stirred for 12 h at RT, then poured into water and extracted with EtOAc. The separated organic layer was washed with brine, dried over anhydrous Na₂SO₄ and concentrated to get a crude compound. The crude compound was purified by combi flash column chromatography using 30% EtOAc in hexane as an eluent to afford the title compound (0.16 g, 56.93%). LC-MS: 344 [M+H]⁺.

### Step-3: tert-Butyl (5-(aminomethyl)thiazol-2-yl)(4,4-difluorocyclohexyl)carbamate

To a solution of tert-butyl (5-cyanothiazol-2-yl)(4,4-difluorocyclohexyl)carbamate (0.29 g, 0.84 mmol) in EtOH (5 mL) was added Raney nickel (0.29 g) and stirred under hydrogen bladder pressure for 12 h at RT. The reaction mass was filtered through celite and the filtrate was concentrated to afford the title compound (0.25 g, 85.61%). LC-MS: 349 [M+H]⁺.

### Step-4: tert-Butyl (4,4-difluorocyclohexyl)(5-((1-methyl-2-oxo-2,3-dihydro-1H-benzimidazole-5-carbox amido)methyl)thiazol-2-yl)carbamate

To a solution of 1-methyl-2-oxo-2,3-dihydro-1H-benzimidazole-5-carboxylic acid (0.21 g, 1.09 mmol) in DMF (5 mL) were added EDC·HCl (0.19 g, 0.99 mmol) and HOBt (0.13 g, 0.96 mmol), and stirred at RT for 10 min. Then tert-butyl (5-(aminomethyl)thiazol-2-yl)(4,4-difluorocyclohexyl)carbamate (0.25 g, 0.72 mmol) was added thereto followed by triethylamine (0.33 g, 3.24 mmol), and the mixture was stirred at RT for 16 h. The reaction mixture was poured into ice cold water, and the precipitate formed was collected by filtration and dried to afford the crude title compound (0.15 g, 39.94%). LC-MS: 522 [M+H]⁺.

### Step-5: N-((2-((4,4-Difluorocyclohexyl)amino)thiazol-5-yl)methyl)-1-methyl-2-oxo-2,3-dihydro-1H-benzimidazole-5-carboxamide (Compound 25)

To a solution of tert-butyl (4,4-difluorocyclohexyl)(5-((1-methyl-2-oxo-2,3-dihydro-1H-benzimidazole-5-carbox amido)methyl)thiazol-2-yl)carbamate (0.15 g, 0.29 mmol) in 1,4-dioxane (2 mL) was added 4N HCl in 1,4-dioxane (4 mL), and stirred for 2 h at RT. The reaction mass was concentrated to get a crude compound. This was purified by prep HPLC using method B to afford the title compound (0.03 g, 22.0%). LC-MS: 422 [M+H]⁺; ¹H-NMR (400 MHz, CD₃OD) δ 7.67-7.65 (dd, 1H), 7.60-7.59 (d, 1H), 7.26 (s, 1H), 7.21-7.20 (d, 1H), 4.55 (s, 2H), 3.70-3.67 (m, 1H), 3.44 (s, 3H), 2.16-2.10 (m, 4H), 2.01-1.92 (m, 2H), 1.77-1.72 (m, 2H).

### Example-26

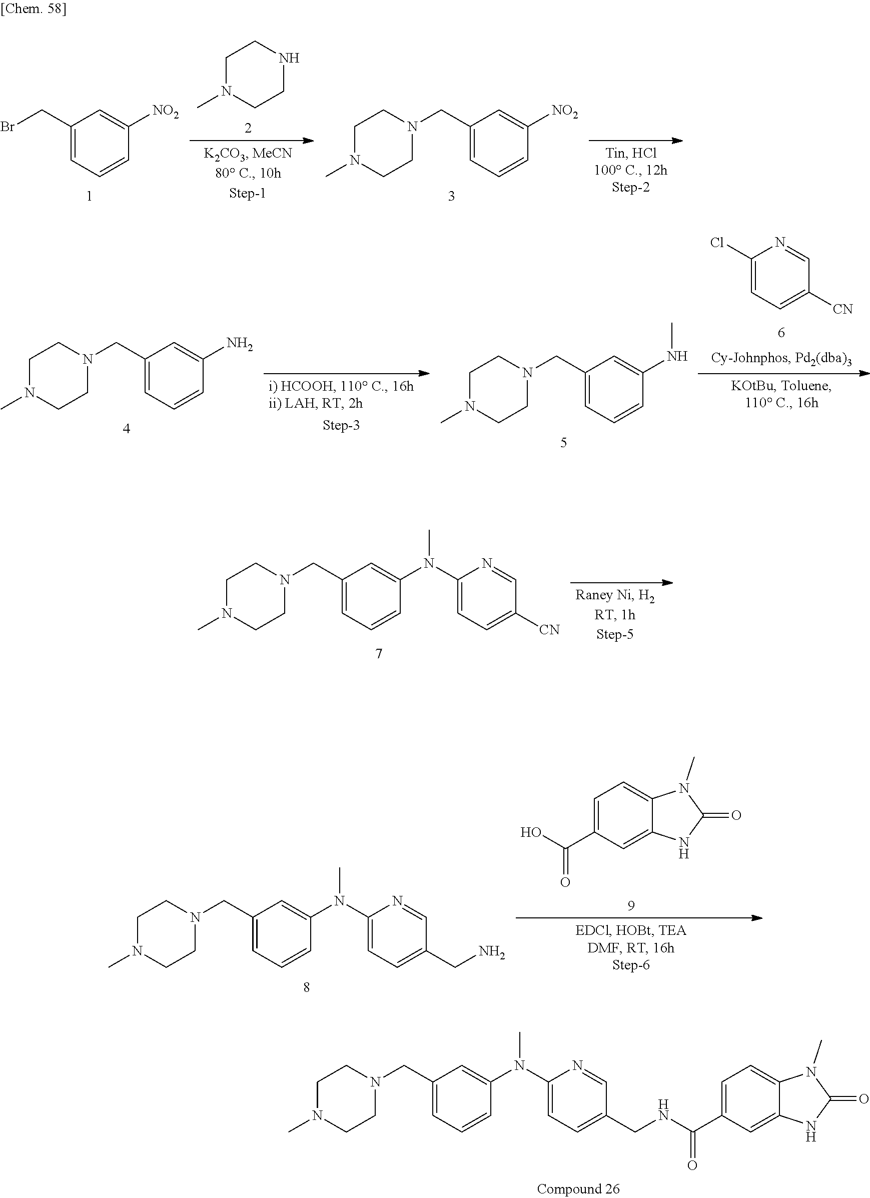

### Step-1: 1-Methyl-4-(3-nitrobenzyl)piperazine

To a stirred solution of 1-methylpiperazine (3.24 g, 32.40 mmol) in acetonitrile (80 mL) was added K₂CO₃ (8.96 g, 64.80 mmol) followed by 1-(bromomethyl)-3-nitrobenzene (7.0 g, 32.40 mmol) and stirred at 80° C. for 10 h. The reaction mixture was poured into ice cold water and extracted with EtOAc. The separated organic layer was washed with brine, dried over anhydrous Na₂SO₄ and concentrated to afford the title compound (5.6 g, 73.56%). LCMS: 236 [M+H]⁺

### Step-2: 3-((4-Methylpiperazin-1-yl)methyl)aniline

To a stirred solution of 1-methyl-4-(3-nitrobenzyl)piperazine (5.0 g, 21.25 mmol) in conc. HCl (15 mL) was added Tin powder (5.04 g, 42.50 mmol) at 0° C. and stirred at 100° C. for 12 h. The reaction mixture was poured into water, basified with sodium bicarbonate, and extracted with 15% MeOH in DCM. The extract was dried over anhydrous Na₂SO₄ and concentrated to afford the title compound (2.7 g, 61.9%). LCMS: 206 [M+H]⁺.

### Step-3: N-Methyl-3-((4-methylpiperazin-1-yl)methyl)aniline

A solution of 3-((4-methylpiperazin-1-yl)methyl)aniline (0.8 g, 3.89 mmol) in formic acid (0.2 g, 4.86 mmol) was stirred at 110° C. for 16 h. The reaction mixture was concentrated, the residue was diluted with THF (10 mL), and 2M solution of lithium aluminum hydride in THF (0.18 g, 4.74 mmol) was added thereto at 0° C. The reaction mixture was stirred at RT for 2 h, and then quenched with EtOAc. The reaction mass was filtered through celite and the filtrate was concentrated. The crude compound was purified by combi flash column chromatography using 10% MeOH in DCM as an eluent to afford the title compound (0.6 g, 70.22%). LCMS: 220 [M+H]⁺

### Step-4: 6-(Methyl(3-((4-methylpiperazin-1-yl)methyl)phenyl)amino)nicotinonitrile

To a solution of N-methyl-3-((4-methylpiperazin-1-yl)methyl)aniline (0.36 g, 1.64 mmol) in toluene (6 mL) was added 6-chloronicotinonitrile (0.6 g, 4.33 mmol) followed by KOtBu (0.37 g, 3.28 mmol). The reaction mixture was degassed with argon for 15 min. Pd₂(dba)₃ (0.08 g, 0.08 mmol) and 2-(dicyclohexylphosphino)biphenyl (0.03 g, 0.08 mmol) was added and stirred at 110° C. for 16 h. The reaction mixture was concentrated to afford a crude compound. This was further purified by preparative HPLC using method B to afford the title compound (0.21 g, 39.80%). LCMS: 322 [M+H]⁺.

### Step-5: 5-(Aminomethyl)-N-methyl-N-(3-((4-methylpiperazin-1-yl)methyl)phenyl)pyridin-2-a mine

To the stirred solution of 6-(methyl(3-((4-methylpiperazin-1-yl)methyl)phenyl)amino)nicotinonitrile (0.07 g, 0.218 mmol) in MeOH (5 mL) was added Raney nickel (0.07 g) followed by methanolic ammonia (1 mL) and stirred at RT under hydrogen bladder pressure for 1 h. The reaction mass was filtered through celite and the filtrate was concentrated to afford the title compound (0.07 g, 98.5%). LC-MS: 326 [M+H]⁺.

### Step-6: 1-Methyl-N-((6-(methyl(3-((4-methylpiperazin-1-yl)methyl)phenyl)amino)pyridin-3-yl)methyl)-2-oxo-2,3-dihydro-1H-benzimidazole-5-carboxamide trifluoroacetate (Compound 26)

To a solution of 1-methyl-2-oxo-2,3-dihydro-1H-benzimidazole-5-carboxylic acid (0.10 g, 0.54 mmol) in DMF (4 mL) were added EDC·HCl (0.07 g, 0.39 mmol) and HOBt (0.05 g, 0.35 mmol), and stirred at RT for 10 min. Then 5-(aminomethyl)-N-methyl-N-(3-((4-methylpiperazin-1-yl)methyl)phenyl)pyridin-2-a mine (0.13 g, 0.40 mmol) was added thereto followed by triethylamine (0.24 g, 2.39 mmol), and the mixture was stirred at RT for 16 h. The reaction mixture was concentrated to afford a crude compound. This was further purified by preparative HPLC using method A to afford the title compound as a TFA Salt (0.03 g, 10.2%). LC-MS: 500 [M+H]⁺; ¹H-NMR (400 MHz, CD₃OD) δ 8.03-8.00 (dd, 1H), 7.91 (d, 1H), 7.65-7.61 (m, 3H), 7.57-7.53 (m, 2H), 7.47-7.44 (d, 1H), 7.16-7.14 (d, 1H), 7.04-7.01 (d, 1H), 4.47 (s, 2H), 4.14 (s, 2H), 3.55 (s, 3H), 3.50 (bs, 4H), 3.39 (s, 3H), 3.30-3.28 (m, 4H), 2.91 (s, 3H).

### Example-27

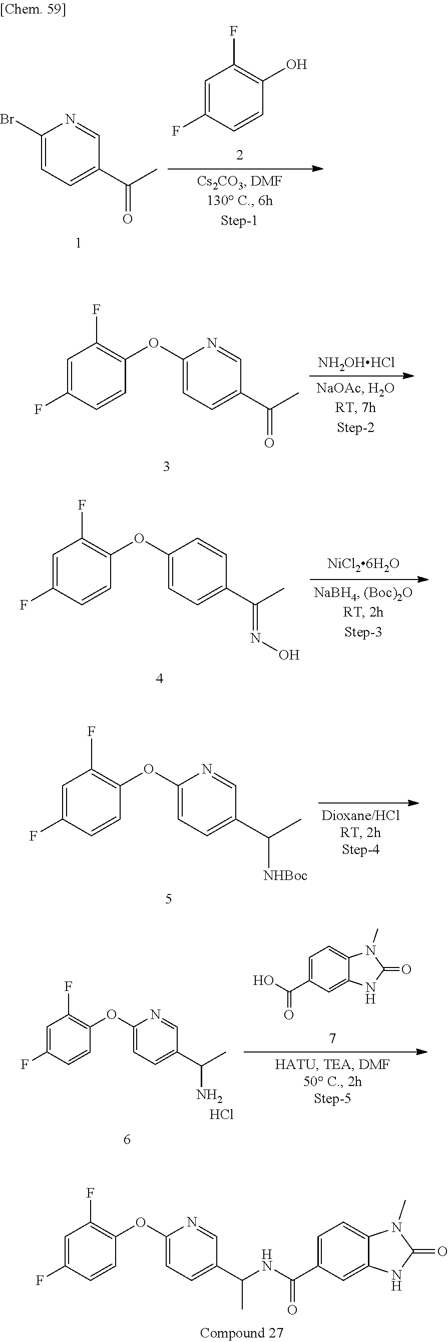

### Step-1: 1-(6-(2,4-Difluorophenoxy)pyridin-3-yl)ethan-1-one

To a stirred solution of 1-(6-bromopyridin-3-yl)ethan-1-one (0.5 g, 2.5 mmol) in DMF (10 mL) was added Cs₂CO₃ (1.63 g, 5.0 mmol) followed by 2,4-difluorophenol (0.32 g, 2.5 mmol) and stirred at 130° C. for 6 h. The reaction mixture was poured into ice cold water, and the solid separated was collected by filtration and dried to afford the title compound (0.62 g, 100%). LC-MS: 250 [M+H]⁺.

### Step-2: (E)-1-(6-(2,4-Difluorophenoxy)pyridin-3-yl)ethan-1-one oxime

To a stirred solution of 1-(6-(2,4-difluorophenoxy)pyridin-3-yl)ethan-1-one (0.8 g, 3.2 mmol) in water (10 mL) was added sodium acetate (0.79 g, 9.63 mmol) followed by hydroxylamine hydrochloride (0.45 g, 6.42 mmol) and stirred at RT for 7 h. The reaction mixture was concentrated to remove the solvent, water was added thereto and then extracted with ethyl acetate which was dried over anhydrous Na₂SO₄ and concentrated under reduced pressure to obtain a crude compound (0.55 g, 83.45%). LC-MS: 265 [M+H]⁺.

### Step-3: tert-Butyl (1-(6-(2,4-difluorophenoxy)pyridin-3-yl)ethyl)carbamate

A solution of (E)-1-(6-(2,4-difluorophenoxy)pyridin-3-yl)ethan-1-one oxime (0.55 g, 1.88 mmol) in methanol (10 mL) was cooled to 0° C., NiCl₂·6H₂O (0.25 g, 1.04 mmol) was added thereto followed by dropwise addition of di-tert-butyl dicarbonate (0.68 g, 3.12 mmol) and stirred for 5 min. Then NaBH₄ (0.12 g, 3.12 mmol) was added thereto in portions by maintaining same temperature. After stirring for 1 h at RT, the reaction mass concentrated to remove methanol. The residue was diluted with ethyl acetate, washed with water, dried over anhydrous Na₂SO₄ and concentrated to afford the title compound (0.5 g, 68.59%). LC-MS: 351 [M+H]⁺.

### Step-4: 1-(6-(2,4-Difluorophenoxy)pyridin-3-yl)ethan-1-amine hydrochloride

To a solution of tert-butyl (1-(6-(2,4-difluorophenoxy)pyridin-3-yl)ethyl)carbamate (0.25 g, 0.71 mmol) in 1,4-dioxane (1 mL) was added 4 M HCl in 1,4-dioxane (2 mL) and stirred for 2 h at RT. The reaction mixture was then concentrated, diethyl ether was added thereto, and the solid was collected by filtration to get title compound (0.16 g, 78.43%). LC-MS: 251.05 [M+H]⁺.

### Step-5: N-(1-(6-(2,4-Difluorophenoxy)pyridin-3-yl)ethyl)-1-methyl-2-oxo-2,3-dihydro-1H-benzi midazole-5-carboxamide (Compound 27)

To a solution of 2-oxo-2,3-dihydro-1H-benzimidazole-5-carboxylic acid (0.10 g, 0.52 mmol) in DMF (3 mL) was added HATU (0.122 g, 0.57 mmol) and stirred at RT for 10 min. Then 1-(6-(2,4-difluorophenoxy)pyridin-3-yl)ethan-1-amine hydrochloride (0.15 g, 0.52 mmol) followed by triethylamine (0.21 g, 2.08 mmol) was added thereto and the mixture was stirred at RT for 16 h. The reaction mixture was added into ice cold water, and the precipitate formed was collected by filtration and washed with water and dried. This was purified by combi flash column chromatography using 5% MeOH in DCM as an eluent to afford the title compound (0.02 g, 11.4%). LC-MS: 425 [M+H]⁺.

¹HNMR (400 MHz, DMSO-D₆) δ 11.07 (s, 1H), 8.73-8.71 (d, 1H), 8.09-8.08 (d, 1H), 7.91-7.88 (dd, 1H), 7.64-7.62 (d, 1H), 7.51 (d, 1H), 7.42-7.36 (m, 2H), 7.15-7.09 (m, 3H), 5.15 (t, 1H), 3.30 (s, 3H), 1.49-1.47 (d, 3H).

### Preparation-1

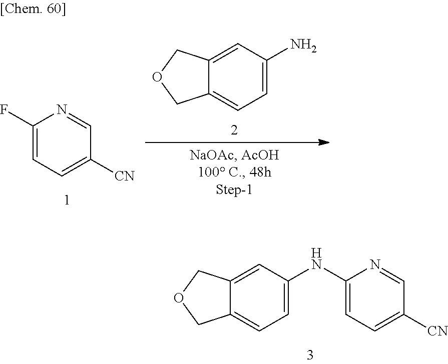 [SMILES: N#Cc1ccc(F)nc1 ; Nc1ccc2c(c1)COC2 ; N#Cc1ccc(Nc2ccc3c(c2)COC3)nc1]

### Step-1: 6-((1,3-Dihydroisobenzofuran-5-yl)amino)nicotinonitrile

To a stirred solution of 6-fluoronicotinonitrile (0.02 g, 0.18 mmol) in acetic acid (1 mL) was added 1,3-dihydroisobenzofuran-5-amine (0.02 g, 0.18 mmol) followed by sodium acetate (0.01 g, 0.18 mmol) and the mixture was stirred at 100° C. for 48 h. The reaction mass was concentrated and the residue was purified by column chromatography using 40% EtOAc in n-hexane as an eluent to get the title compound (0.02 g, 37%). LC-MS: 238 [M+H]⁺.

### Preparation-2

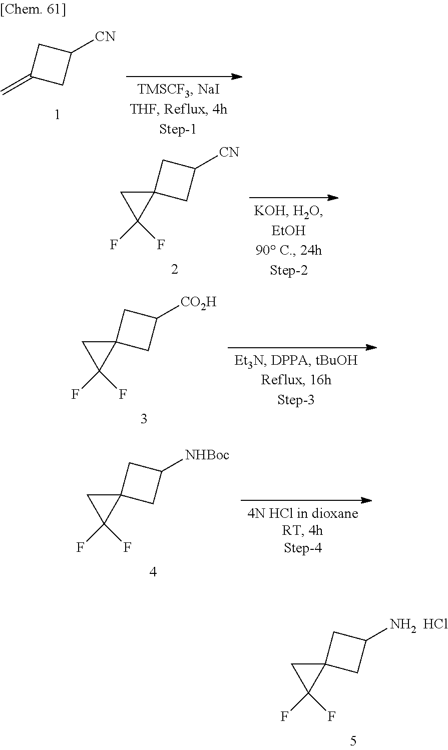

### Step-1: 1,1-Difluorospiro[2.3]hexane-5-carbonitrile

To a stirred solution of 3-methylenecyclobutane-1-carbonitrile (1.0 g, 10.73 mmol) in THF (10 mL) was added trimethyl(trifluoromethyl)silane (5.87 g, 37.5 mmol) followed by sodium iodide (0.8 g, 5.36 mmol) and the mixture was stirred at 70° C. for 4 h. The residue was diluted with EtOAc, washed with water, dried over anhydrous Na₂SO₄ and concentrated. The residue was purified by combi flash column chromatography using 20% EtOAc in n-hexane as an eluent to get the title compound (1.1 g, 74.3%)¹H-NMR (400 MHz, CDCl₃): δ 3.28-3.25 (m, 1H), 2.74-2.64 (m, 2H), 2.63-2.61 (m, 2H), 1.37-1.32 (m, 2H).

### Step-2: 1,1-Difluorospiro[2.3]hexane-5-carboxylic acid

To a stirred solution of 1,1-difluorospiro[2.3]hexane-5-carbonitrile (0.5 g, 3.49 mmol) in EtOH (5 mL) and water (2.5 mL) was added KOH (0.98 g, 17.46 mmol) and the mixture was stirred at 90° C. for 24 h. The reaction mixture was concentrated and the residue was acidified with 1 N HCl, extracted with DCM, dried over anhydrous Na₂SO₄ and concentrated to afford the title compound (0.5 g, 88.0%). ¹H-NMR (400 MHz, DMSO-d₆): δ 12.35 (bs, 1H), 4.04-4.03 (s, 1H), 3.32-3.16 (m, 4H), 2.31-1.99 (m, 2H).

### Step-3: tert-Butyl (1,1-difluorospiro[2.3]hexan-5-yl)carbamate

To a stirred solution of 1,1-difluorospiro[2.3]hexane-5-carboxylic acid (0.5 g, 3.08 mmol) in toluene (10 mL) was added triethylamine (0.68 g, 6.78 mmol) followed by DPPA (1.27 g, 4.62 mmol) and the mixture was stirred at RT for 10 min. Then t-BuOH (1 mL) was added and stirred at 80° C. for 16 h. The reaction mixture was poured into ice cold water, extracted with EtOAc, dried over anhydrous Na₂SO₄ and concentrated. The residue was purified by combi flash column chromatography using 20% EtOAc in n-hexane as an eluent to afford the title compound (0.5 g, 69.5%). 1H-NMR (400 MHz, CDCl₃): δ 7.03-7.01 (m, 1H), 4.37-4.33 (m, 1H), 2.66-2.60 (m, 2H), 2.29-2.28 (m, 2H), 1.47 (s, 9H), 1.25-1.20 (m, 2H).

### Step-4: 1,1-Difluorospiro[2.3]hexan-5-amine hydrochloride

To a solution of tert-butyl (1,1-difluorospiro[2.3]hexan-5-yl)carbamate (0.5 g, 2.14 mmol) in 1,4-dioxane (5 mL) was added 4 N HCl in 1,4-dioxane (5 mL) and the mixture was stirred at RT for 4 h. The reaction mixture was concentrated to get the title compound (0.3 g, 82%). LC-MS: 134 [M+H]⁺.

### Preparation-3

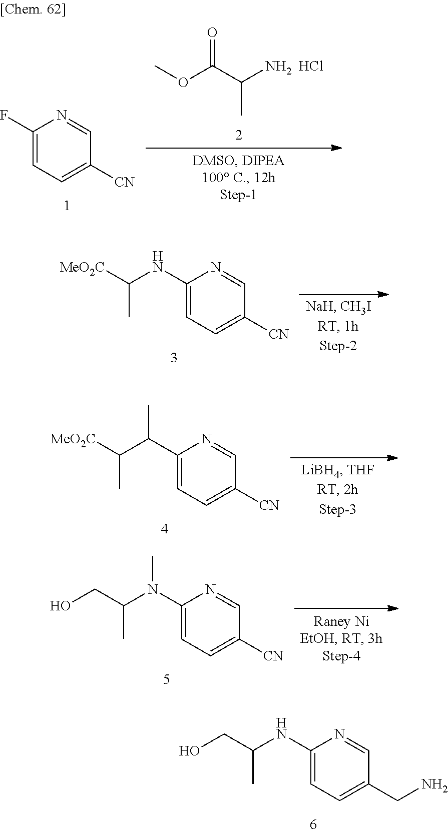

### Step-1: Methyl (5-cyanopyridin-2-yl)alaninate

To a stirred solution of 6-fluoronicotinonitrile (0.5 g, 4.09 mmol) in DMSO (10 mL) was added DIPEA (2.64 g, 20.47 mmol) followed by addition of methyl alaninate hydrochloride (0.57 g, 4.09 mmol) and the mixture was stirred at 100° C. for 12 h. The reaction mixture was concentrated to remove DIPEA, poured into ice cold water, and the solid separated was collected by filtration and dried to afford the title compound (0.58 g, 69%). LC-MS: 204 [M−H]⁻.

### Step-2: Methyl N-(5-cyanopyridin-2-yl)-N-methylalaninate

To a stirred solution of methyl (5-cyanopyridin-2-yl)alaninate (0.58 g, 2.82 mmol) in DMF (6 mL) was added NaH (60% dispersion in mineral oil) (0.17 g, 4.23 mmol) followed by dropwise addition of iodomethane (0.48 g, 3.39 mmol) at 0° C. and the mixture was stirred at RT for 1 h. The reaction mixture was poured into ice cold water, and the solid separated was collected by filtration and dried to afford the title compound (0.59 g, 95%). LC-MS: 220 [M+H]⁺.

### Step-3: 6-((1-Hydroxypropan-2-yl)(methyl)amino)nicotinonitrile

To a stirred solution of methyl N-(5-cyanopyridin-2-yl)-N-methylalaninate (0.09 g, 0.41 mmol) in THF (2 mL) was added LiBH₄ (0.03 g, 1.64 mmol) at 0° C. and the mixture was stirred at RT for 2 h. The reaction mixture was quenched with saturated sodium sulphate solution and extracted with EtOAc. The separated organic layer washed with brine, dried over anhydrous Na₂SO₄ and concentrated to get the title compound. (0.09 g, 114.6%). LC-MS: 192 [M+H]⁺.

### Step-4: 2-((5-(Aminomethyl)pyridin-2-yl)(methyl)amino)propan-1-ol

To a stirred solution of 6-((1-hydroxypropan-2-yl)(methyl)amino)nicotinonitrile (0.09 g, 0.47 mmol) in EtOH (10 mL) was added 7 M ammonia in MeOH (2 mL) followed Raney nickel (0.1 g) and stirred under hydrogen pressure for 3 h at RT. The reaction mass was filtered through Celite and the filtrate was concentrated to afford the title compound (0.09 g, 97%). LC-MS: 196 [M+H]⁺.

### Preparation-4

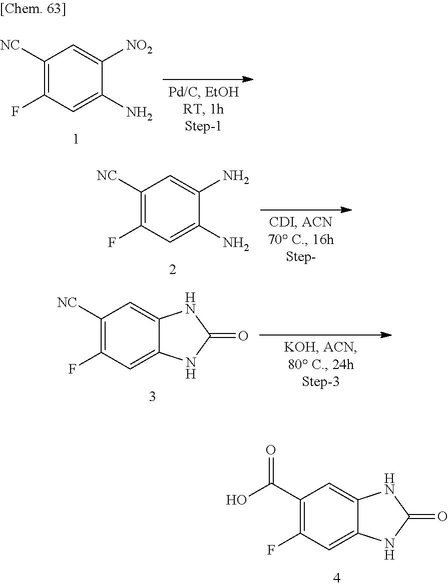 [SMILES: N#Cc1cc([N+](=O)[O-])c(N)cc1F ; N#Cc1cc(N)c(N)cc1F ; N#Cc1cc2[nH]c(=O)[nH]c2cc1F ; O=C(O)c1cc2[nH]c(=O)[nH]c2cc1F]

### Step-1: 4,5-Diamino-2-fluorobenzonitrile

To a stirred solution of 4-amino-2-fluoro-5-nitrobenzonitrile (0.80 g, 4.41 mmol) in EtOH (4 mL) was added Pd/C (0.4 g) and stirred under hydrogen bladder pressure for 1 h at RT. The reaction mass was filtered through Celite, and the filtrate was concentrated to afford the title compound (0.65 g 97%). LC-MS: 152 [M+H]⁺.

### Step-2: 6-Fluoro-2-oxo-2,3-dihydro-1H-benzimidazole-5-carbonitrile

To a stirred solution of 4,5-diamino-2-fluorobenzonitrile (0.65 g, 4.30 mmol) in THF (15 mL) was added CDI (1.05 g, 6.45 mmol) and the mixture was stirred at 70° C. for 16 h. The reaction mixture was poured into ice cold water, and the solid separated was collected by filtration and dried to afford the title compound (0.7 g, 92%). LC-MS: 178 [M+H]⁺.

### Step-3: 6-Fluoro-2-oxo-2,3-dihydro-1H-benzimidazole-5-carboxylic acid

To a stirred solution of 6-fluoro-2-oxo-2,3-dihydro-1H-benzimidazole-5-carbonitrile (065 g, 3.7 mmol) in EtOH (12 mL) was added KOH (3.7 g, 66.6 mmol) in water (12 mL) and the mixture was stirred at 80° C. for 24 h. The reaction mixture was concentrated to remove EtOH, diluted with water and acidified with 1 N HCl. The precipitate formed was collected by filtration and dried to get the title compound (0.55 g, 76%). LC-MS: 197 [M+H]⁺.

### Preparation-5

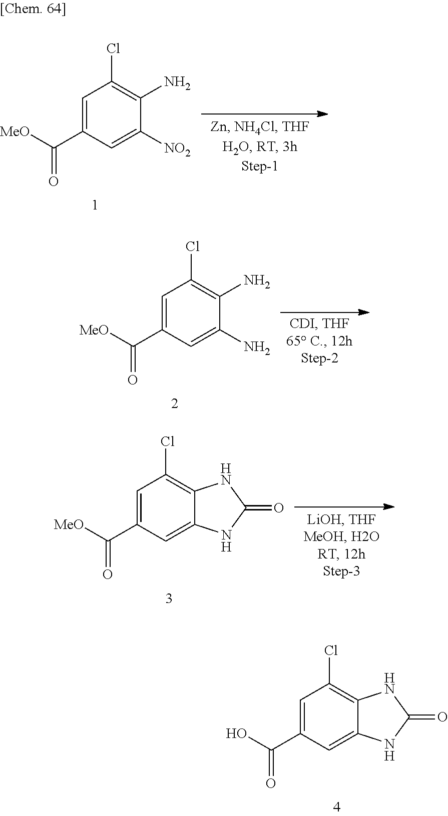 [SMILES: COC(=O)c1cc(Cl)c(N)c([N+](=O)[O-])c1 ; COC(=O)c1cc(N)c(N)c(Cl)c1 ; COC(=O)c1cc(Cl)c2[nH]c(=O)[nH]c2c1 ; O=C(O)c1cc(Cl)c2[nH]c(=O)[nH]c2c1]

### Step-1: Methyl 3,4-diamino-5-chlorobenzoate

To a stirred solution of methyl 4-amino-3-chloro-5-nitrobenzoate (0.75 g, 3.25 mmol) in THF (15 mL) and water (6 mL) was added ammonium chloride (1.04 g, 19.5 mmol) followed by zinc dust (0.64 g, 3.36 mmol) and the mixture was stirred at RT for 3 h. The reaction mass was diluted with EtOAc and filtered through Celite bed. The filtrate was washed with water, dried over anhydrous Na₂SO₄ and concentrated to afford the title compound (0.63 g, 96%). LC-MS: 201 [M+H]⁺.

### Step-2: Methyl 7-chloro-2-oxo-2,3-dihydro-1H-benzimidazole-5-carboxylate

To a stirred solution of methyl 3,4-diamino-5-chlorobenzoate (0.73 g, 3.64 mmol) in THF (15 mL) was added CDI (1.18 g, 7.28 mmol) and the mixture was stirred at 65° C. for 12 h. The reaction mixture was poured into ice cold water, and the solid separated was collected by filtration and dried to afford the title compound (0.7 g, 85%). LC-MS: 227 [M+H]⁺.

### Step-3: 7-Chloro-2-oxo-2,3-dihydro-1H-benzimidazole-5-carboxylic acid

To a stirred solution of methyl 7-chloro-2-oxo-2,3-dihydro-1H-benzimidazole-5-carboxylate (0.7 g, 3.08 mmol) in THF (6 mL) and MeOH (6 mL) was added LiOH (0.39 g, 9.24 mmol) in water (3 mL) and stirred at RT for 12 h. The reaction mixture was concentrated to remove THF and MeOH, diluted with water and acidified with 1 N HCl. The precipitate formed was collected by filtration and dried to get the title compound (0.65 g, 99%). LC-MS: 213 [M+H]⁺.

### Preparation-6

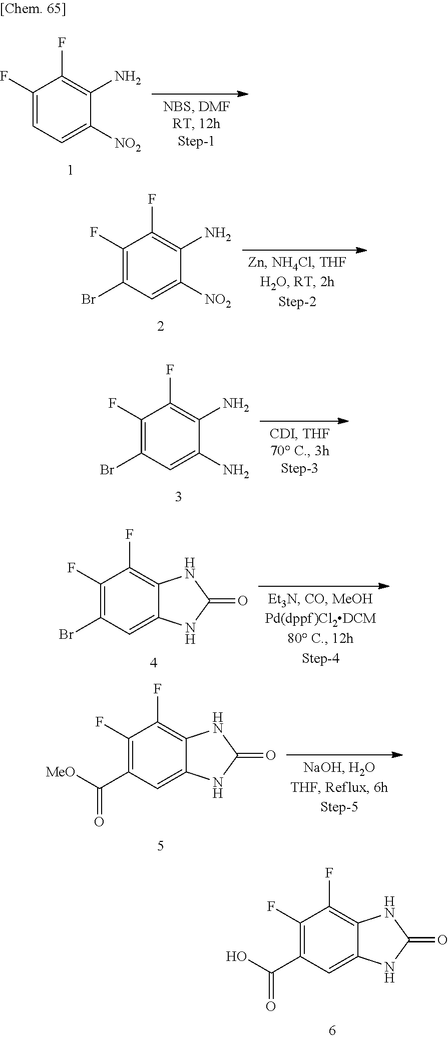 [SMILES: Nc1c([N+](=O)[O-])ccc(F)c1F ; Nc1c([N+](=O)[O-])cc(Br)c(F)c1F ; Nc1cc(Br)c(F)c(F)c1N ; O=c1[nH]c2cc(Br)c(F)c(F)c2[nH]1 ; COC(=O)c1cc2[nH]c(=O)[nH]c2c(F)c1F ; O=C(O)c1cc2[nH]c(=O)[nH]c2c(F)c1F]

### Step-1: 4-Bromo-2,3-difluoro-6-nitroaniline

To a stirred solution of 2,3-difluoro-6-nitroaniline (1.0 g, 5.74 mmol) in DMF (20 mL) was added N-bromosuccinimide (1.02 g, 5.74 mmol) at 0° C. and the mixture was stirred at RT for 12 h. After completion of the reaction, the reaction mixture was added ice cold water, and the solid precipitated was collected by filtration and dried to get the title compound (1.0 g, 68.82%). LC-MS: 253 [M+H]⁺.

### Step-2: 5-Bromo-3,4-difluorobenzene-1,2-diamine

To a stirred solution of 4-bromo-2,3-difluoro-6-nitroaniline (1.0 g, 3.95 mmol) in THF (30 mL) was added ammonium chloride (1.26 g, 23.71 mmol) in water (10 mL) followed by zinc dust (0.77 g, 11.85 mmol) and the mixture was stirred at RT for 2 h. The reaction mixture was filtered through Celite and the filtrate was extracted with EtOAc. The organic layer was dried over anhydrous Na₂SO₄ and concentrated to get the title compound (0.83 g, 94.1%). LC-MS: 221 [M−H]⁻.

### Step-3: 6-Bromo-4,5-difluoro-1,3-dihydro-2H-benzimidazol-2-one

To a stirred solution of 5-bromo-3,4-difluorobenzene-1,2-diamine (0.82 g, 3.67 mmol) in THF (20 mL) was added CDI (1.3 g, 8.06 mmol) and stirred at 70° C. for 3 h. The reaction mixture was concentrated to remove THF then poured into ice cold water, and the solid precipitated was collected by filtration and dried to afford the title compound (0.9 g, 98.30%). LC-MS: 247 [M−H]⁻.

### Step-4: Methyl 6,7-difluoro-2-oxo-2,3-dihydro-1H-benzimidazole-5-carboxylate

To a stirred solution of 6-bromo-4,5-difluoro-1,3-dihydro-2H-benzimidazol-2-one (0.5 g, 2.0 mmol) in MeOH (5 mL) was added triethylamine (1.46 g, 14.4 mmol). The reaction mass was degassed with argon for 15 min. Then [1,1′-bis(diphenylphosphino)ferrocene]palladium(II) dichloride (0.35 g, 0.48 mmol) was added thereto and stirred with 80 psi of carbon monoxide at 80° C. for 12 h. The reaction mixture was filtered through Celite and the filtrate was concentrated to get title compound (0.4 g, 87%). LC-MS: 227 [M−H]⁻.

### Step-5: 6,7-Difluoro-2-oxo-2,3-dihydro-1H-benzimidazole-5-carboxylic acid

To a stirred solution of methyl 6,7-difluoro-2-oxo-2,3-dihydro-1H-benzimidazole-5-carboxylate (0.35 g, 1.53 mmol) in THF (1 mL) was added NaOH (0.24 g, 6.13 mmol) in water (2 mL) and stirred at 65° C. for 6 h. The reaction mixture was concentrated to remove THF and acidified with 1 N HCl. The precipitate formed was collected by filtration and dried to get the title compound (0.28 g, 85.24%). LC-MS: 213 [M−H]⁻.

### Preparation-7

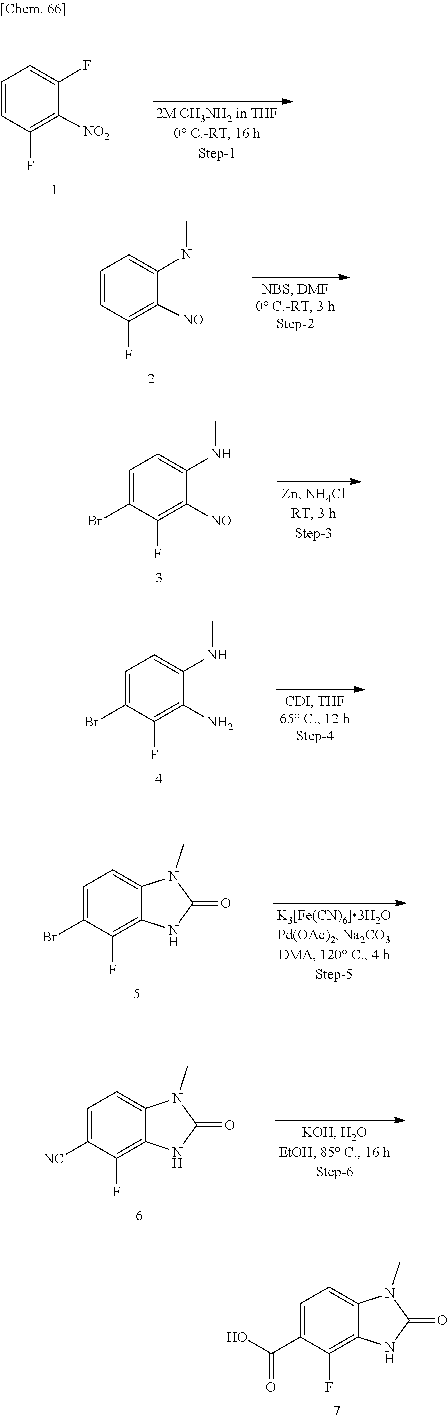

### Step-1: 3-Fluoro-N-methyl-2-nitroaniline

To a stirred solution of 1,3-difluoro-2-nitrobenzene (2.0 g, 12.57 mmol) in MeOH (40 mL) was added 2 M methylamine in THF (0.39 g, 12.57 mmol) at 0° C. and the mixture was stirred at RT for 16 h. The reaction mass was concentrated and diluted with ice cold water. The mixture was extracted with EtOAc, dried over anhydrous Na₂ SO₄ and concentrated to get title compound (1.5 g, 70.13%). LC-MS: 171 [M+H]⁺.

### Step-2: 4-Bromo-3-fluoro-N-methyl-2-nitroaniline

To a stirred solution of 3-fluoro-N-methyl-2-nitroaniline (1.5 g, 8.81 mmol) in DMF (20 mL) was added N-bromosuccinimide (1.56 g, 8.81 mmol) at 0° C. and the mixture was stirred at RT for 3 h. After completion of the reaction, the reaction mass was added ice cold water, extracted with EtOAc, dried over anhydrous Na₂SO₄ and concentrated. The residue was purified by column chromatography using 15% EtOAc in n-hexane as an eluent to get the title compound (1.0 g, 45.55%). LC-MS: 249 [M+H]⁺.

### Step-3: 4-Bromo-3-fluoro-N¹-methylbenzene-1,2-diamine

To a stirred solution of 4-bromo-3-fluoro-N-methyl-2-nitroaniline (1.0 g, 4.01 mmol) in THF (30 mL) was added ammonium chloride (1.29 g, 24.09 mmol) in water (5 mL) followed by zinc dust (0.78 g, 12.04 mmol) and the mixture was stirred at RT for 3 h. The reaction mixture was filtered through Celite and the filtrate extracted with EtOAc. The organic layer was dried over anhydrous Na₂SO₄ and concentrated to get the title compound (0.8 g, 90.95%). LC-MS: 219 [M+H]⁺.

### Step-4: 5-Bromo-4-fluoro-1-methyl-1,3-dihydro-2H-benzimidazol-2-one

To a stirred solution of 4-bromo-3-fluoro-N¹-methylbenzene-1,2-diamine (0.8 g, 3.65 mmol) in acetonitrile (10 mL) was added CDI (1.18 g, 7.3 mmol) and stirred at 65° C. for 12 h. The reaction mixture was concentrated and poured into ice cold water, and the solid precipitated was collected by filtration and dried to afford the title compound (0.8 g, 89.39%). LC-MS: 245 [M+H]⁺.

### Step-5: 4-Fluoro-1-methyl-2-oxo-2,3-dihydro-1H-benzimidazole-5-carbonitrile

To a stirred solution of 5-bromo-4-fluoro-1-methyl-1,3-dihydro-2H-benzimidazol-2-one (0.75 g, 3.06 mmol) in N,N-dimethylacetamide (40 ml) was added sodium carbonate (0.08 g, 0.81 mmol) followed by potassium ferrocyanide trihydrate (0.55 g, 1.3 mmol) and degassed for 15 min with argon. The reaction mixture was added palladium diacetate (0.07 g, 0.32 mmol) and stirred at 120° C. for 4 h. The reaction mass was filtered through Celite and the filtrate was diluted with water and extracted with EtOAc, dried over anhydrous Na₂ SO₄ and concentrated. The residue was purified by column chromatography using 70% EtOAc in n-hexane as an eluent to get the title compound (0.36 g, 57.68%). LC-MS: 190 [M−H]⁻.

### Step-6: 4-Fluoro-1-methyl-2-oxo-2,3-dihydro-1H-benzimidazole-5-carboxylic acid

To a stirred solution of 4-fluoro-1-methyl-2-oxo-2,3-dihydro-1H-benzimidazole-5-carbonitrile (0.33 g, 1.72 mmol) in EtOH (15 mL) was added KOH (0.29 g, 5.17 mmol) dissolved in water (3 mL) and stirred at 85° C. for 16 h. Then the reaction mixture was concentrated and acidified with 1 N HCl. The solid precipitated was collected by filtration and dried to get the title compound (0.13 g, 35.83%). LC-MS: 211 [M+H]⁺.

### Example-103

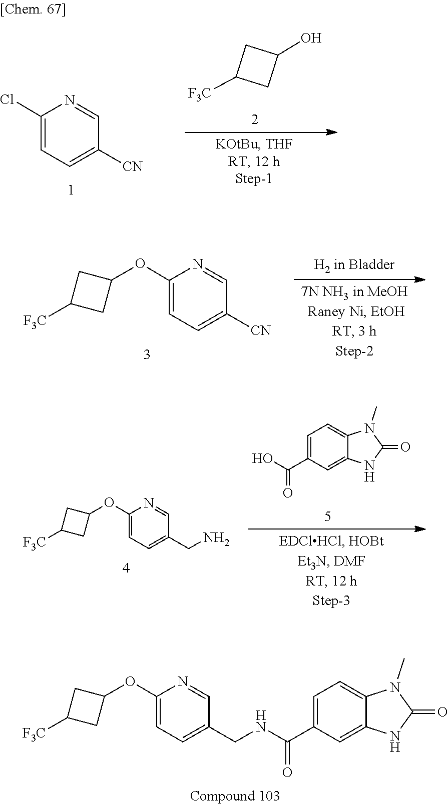 [SMILES: OC1CC(C(F)(F)F)C1 ; N#Cc1ccc(Cl)nc1 ; N#Cc1ccc(OC2CC(C(F)(F)F)C2)nc1 ; Cn1c(=O)[nH]c2cc(C(=O)O)ccc21 ; NCc1ccc(OC2CC(C(F)(F)F)C2)nc1 ; Cn1c(=O)[nH]c2cc(C(=O)NCc3ccc(OC4CC(C(F)(F)F)C4)nc3)ccc21]

### Step-1: 6-(3-(Trifluoromethyl)cyclobutyloxy)nicotinonitrile

To a stirred solution of 6-chloronicotinonitrile (0.18 g, 1.29 mmol) in THF (4 mL) was added 3-(trifluoromethyl)cyclobutan-1-ol (0.2 g, 1.42 mmol) followed by KOtBu (0.22 g, 1.95 mmol) and the mixture was stirred at RT for 12 h. The reaction mixture was quenched with ice cold water and extracted with EtOAc. The separated organic layer was dried over anhydrous Na₂SO₄ and concentrated to get the title compound (0.3 g, 95%). LC-MS: 243 [M+H]⁺.

### Step-2: (6-(3-(Trifluoromethyl)cyclobutyloxy)pyridin-3-yl)methanamine

To a stirred solution of 6-(3-(trifluoromethyl)cyclobutyloxy)nicotinonitrile (0.45 g, 1.85 mmol) in EtOH (30 mL) was added 7 M ammonia in MeOH (2 mL) followed by Raney nickel (0.5 g) and the mixture was stirred at RT for 3 h under hydrogen bladder pressure. The reaction mass was filtered through Celite and the filtrate was concentrated to afford the title compound (0.42 g). LC-MS: 230 [M-NH2]⁺.

### Step-3: 1-Methyl-2-oxo-N-((6-(3-(trifluoromethyl)cyclobutoxy)pyridin-3-yl)methyl)-2,3-dihyd ro-1H-benzimidazole-5-carboxamide (Compound 103)

A solution of 1-methyl-2-oxo-2,3-dihydro-1H-benzimidazole-5-carboxylic acid (0.28 g, 1.46 mmol) in DMF (6 mL) was added EDC·HCl (0.24 g, 1.29 mmol) followed by HOBt (0.08 g, 0.6 mmol) and the mixture was stirred at RT for 10 mins. To the reaction mixture was added triethylamine (0.98 g, 9.7 mmol) followed by (6-(3-(trifluoromethyl)cyclobutyloxy)pyridin-3-yl)methanamine (0.4 g, 1.62 mmol) and stirred at RT for 12 h. The reaction mass was quenched with ice cold water and extracted with EtOAc. The separated organic layer was dried over anhydrous Na₂SO₄ and concentrated. The residue was purified by preparative HPLC using method C to get the title compound (0.08 g, 11%).

LC-MS: 421 [M+H]⁺; ¹H-NMR (400 MHz, DMSO-d₆): δ 11.06 (s, 1H), 8.89-8.86 (t, 1H), 8.05 (s, 1H), 7.65-7.57 (dd, 2H), 7.46 (s, 1H), 7.12-7.10 (d, 1H), 6.76-6.74 (d, 1H), 5.11-5.04 (m, 1H), 4.35-4.33 (d, 2H), 3.29-3.09 (t, 3H), 2.99-2.88 (m, 1H), 2.66-2.61 (m, 2H), 2.09-2.02 (m, 2H).

### Example-104

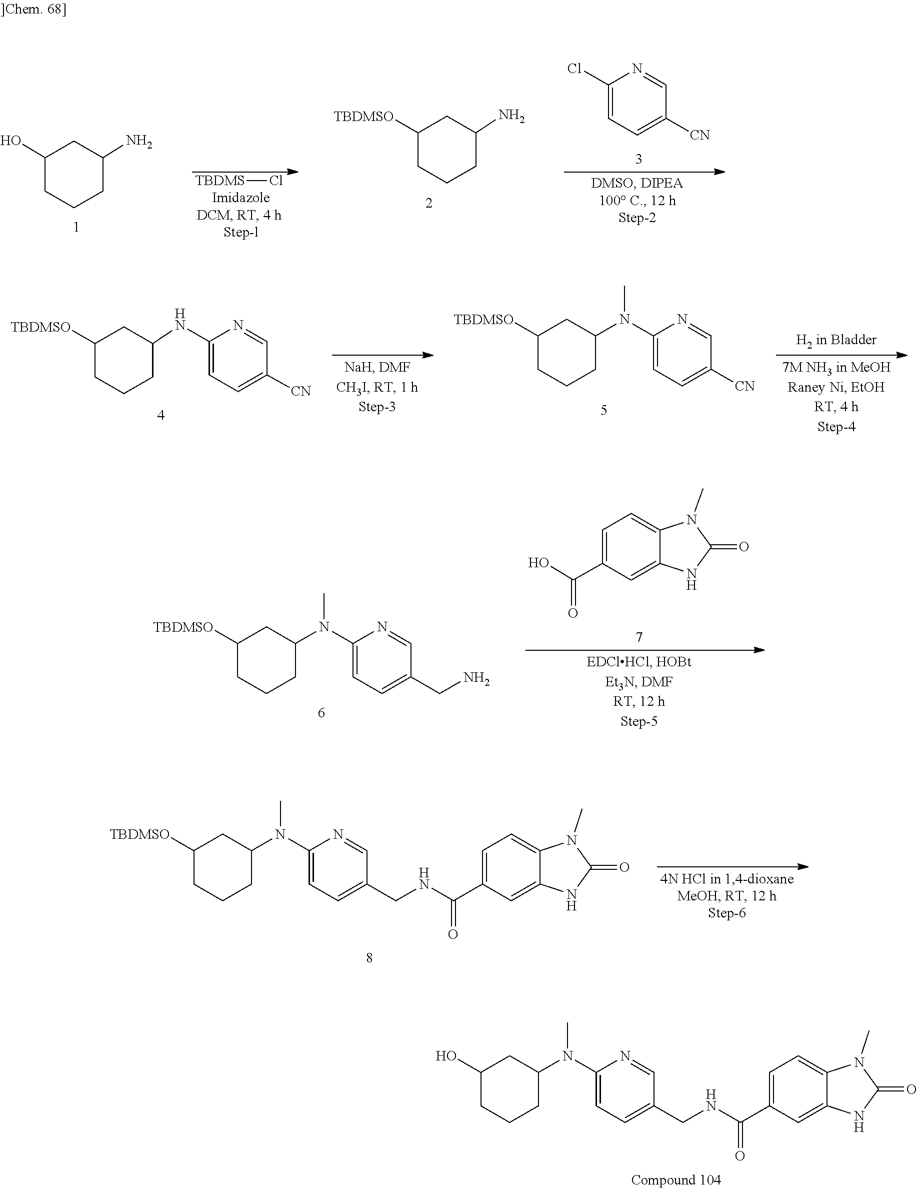

### Step-1: 3-((tert-Butyldimethylsilyl)oxy)cyclohexan-1-amine

To a stirred solution 3-aminocyclohexan-1-ol (1.3 g, 11.3 mmol) in DCM (20 mL) was added imidazole (1.77 g, 26.04 mmol) followed by tert-butyldimethylsilyl chloride (1.96 g, 13.02 mmol) at 0° C. and the mixture was stirred at RT for 4 h. The reaction mixture was quenched with ice cold water and extracted with DCM. The separated organic layer was dried over anhydrous Na₂SO₄ and concentrated to get the title compound (2.5 g, 96%). LC-MS: 230 [M+H]⁺.

### Step-2: 6-((3-((tert-Butyldimethylsilyl)oxy)cyclohexyl)amino)nicotinonitrile

To a stirred solution of 6-chloronicotinonitrile (1.5 g, 10.8 mmol) in DMSO (20 mL) was added 3-((tert-butyldimethylsilyl)oxy)cyclohexan-1-amine (2.48 g, 10.8 mmol) followed by DIPEA (5.58 g, 43.3 mmol) and the mixture was stirred at 100° C. for 12 h. The reaction mixture was poured into ice cold water and extracted with EtOAc. The separated organic layer was dried over anhydrous Na₂SO₄ and concentrated. The residue was purified by column chromatography using 10% EtOAc in n-hexane as an eluent to get the title compound (1.0 g, 27.86%). LC-MS: 332 [M+H]⁺.

### Step-3: 6-((3-((tert-Butyldimethylsilyl)oxy)cyclohexyl)(methyl)amino)nicotinonitrile

To a stirred solution of 6-((3-((tert-butyldimethylsilyl)oxy)cyclohexyl)amino)nicotinonitrile (1.0 g, 3.01 mmol) in DMF (10 mL) was added NaH (60% dispersion in mineral oil) (0.18 g, 4.52 mmol) followed by dropwise addition of iodomethane (0.51 g, 3.61 mmol) at 0° C. and the mixture was stirred at RT for 1 h. The reaction mixture was poured into ice cold water and extracted with EtOAc. The separated organic layer was dried over anhydrous Na₂SO₄ and concentrated. The residue was purified by column chromatography using 10% EtOAc in n-hexane as an eluent to get the title compound (0.8 g, 76.7%). LC-MS: 346 [M+H]⁺.

### Step-4: 5-(Aminomethyl)-N-(3-((tert-butyldimethylsilyl)oxy)cyclohexyl)-N-methylpyridin-2-a mine

To a solution of 6-((3-((tert-butyldimethylsilyl)oxy)cyclohexyl)(methyl)amino)nicotinonitrile (0.8 g, 2.43 mmol) in EtOH (10 mL) was added 7 M ammonia in MeOH (3 mL) followed by Raney nickel (0.8 g) and the mixture was stirred under hydrogen bladder pressure for 4 h at RT. The reaction mass was filtered through Celite and the filtrate was concentrated to afford the title compound (0.8 g, 98%). LC-MS: 350 [M+H]⁺.

### Step-5: N-((6-((3-((tert-Butyldimethylsilyl)oxy)cyclohexyl)(methyl)amino)pyridin-3-yl)methyl)-1-methyl-2-oxo-2,3-dihydro-1H-benzimidazole-5-carboxamide

To a solution of 1-methyl-2-oxo-2,3-dihydro-1H-benzimidazole-5-carboxylic acid (0.39 g, 2.05 mmol) in DMF (10 mL) were added EDC·HCl (0.39 g, 2.05 mmol) and HOBt (0.14 g, 1.03 mmol) and the mixture was stirred at RT for 10 min. To the mixture was added 5-(aminomethyl)-N-(3-((tert-butyldimethylsilyl)oxy)cyclohexyl)-N-methylpyridin-2-a mine (0.8 g, 2.29 mmol) followed by triethylamine (1.15 g, 11.44 mmol) and the mixture was stirred at RT for 12 h. The reaction mixture was poured into ice cold water, and the solid precipitated was collected by filtration and dried to get a crude compound, which was purified by column chromatography using 90% EtOAc in n-hexane as an eluent to afford the title compound (0.23 g, 19.2%). LC-MS: 524 [M+H]⁺

### Step-6: N-((6-((3-Hydroxycyclohexyl)(methyl)amino)pyridin-3-yl)methyl)-1-methyl-2-oxo-2,3-d ihydro-1H-benzimidazole-5-carboxamide (Compound 104)

To a solution of N-((6-((3-((tert-butyldimethylsilyl)oxy)cyclohexyl)(methyl)amino)pyridin-3-yl)methyl)-1-methyl-2-oxo-2,3-dihydro-1H-benzimidazole-5-carboxamide (0.23 g, 0.44 mmol) in MeOH (4 mL) was added 4 N HCl in 1,4-dioxane (2 ml) and the mixture was stirred at RT for 12 h. The reaction mixture was concentrated to remove solvents. The residue was dissolved with water and the pH was adjusted to neutral. The solid precipitated was collected by filtration and dried to get the title compound (0.09 g, 50.0%).

LC-MS: 410 [M+H]⁺; ¹H-NMR (400 MHz, DMSO-d₆): δ 11.3-11.2 (s, 1H), 9.04-9.01 (t, 1H), 7.97-7.95 (d, 1H), 7.90 (s, 1H), 7.64-7.61 (dd, 1H), 7.50 (s, 1H), 7.34-7.32 (s, 1H), 7.17-7.15 (s, 1H), 4.38-4.36 (dd, 2H), 4.09 (bs, 1H), 3.59-3.51 (bs, 2H), 3.31 (s, 3H), 3.01 (s, 3H), 1.88-1.80 (m, 2H), 1.73-1.70 (d, 1H), 1.60-1.58 (d, 1H), 1.51-1.37 (m, 3H), 1.10-1.06 (q, 1H).

### Example-105

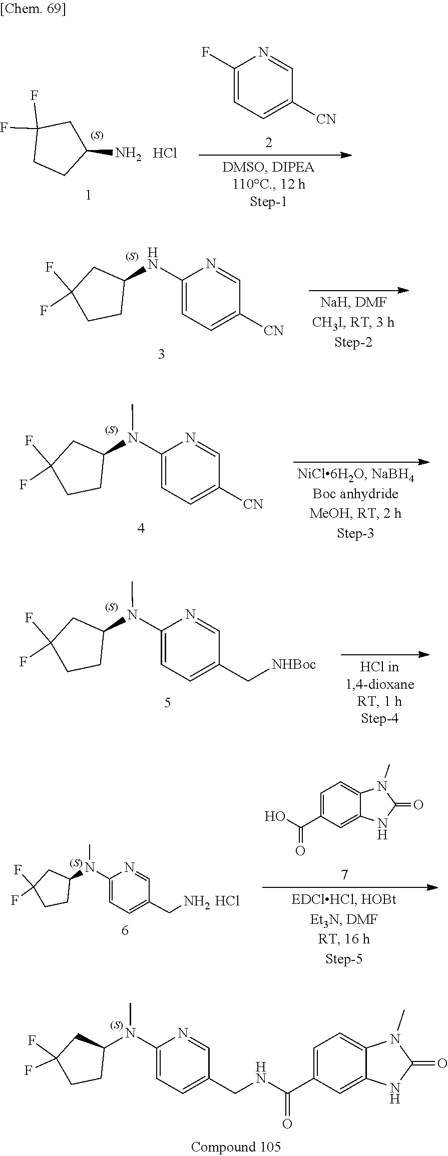

### Step-1: (S)-6-((3,3-Difluorocyclopentyl)amino)nicotinonitrile

To a stirred solution of 6-fluoronicotinonitrile (0.1 g, 0.82 mmol) in DMSO (5 mL) was added DIPEA (0.27 g, 1.6 mmol) followed by (S)-3,3-difluorocyclopentan-1-amine hydrochloride (0.13 g, 0.82 mmol) and the mixture was stirred at 110° C. for 12 h. The reaction mixture was concentrated to remove DIPEA then poured into ice cold water. The solid separated was collected by filtration and dried to afford the title compound (0.18 g, 99%). LC-MS: 224 [M+H]⁺

### Step-2: (S)-6-((3,3-Difluorocyclopentyl)(methyl)amino)nicotinonitrile

To a stirred solution of (S)-6-((3,3-difluorocyclopentyl)amino)nicotinonitrile (0.2 g, 0.89 mmol) in DMF (8 mL) was added NaH (60% dispersion in mineral oil) (0.05 g, 1.34 mmol) followed by dropwise addition of iodomethane (0.15 g, 1.07 mmol) at 0° C. and the mixture was stirred at RT for 3 h. The reaction mixture was poured into ice cold water, and the solid separated was collected by filtration and dried to afford the title compound (0.2 g, 95%). LC-MS: 238 [M+H]⁺

### Step-3: tert-Butyl (S)-((6-((3,3-difluorocyclopentyl)(methyl)amino)pyridin-3-yl)methyl)carbamate

To a solution of (S)-6-((3,3-difluorocyclopentyl)(methyl)amino)nicotinonitrile (0.2 g, 0.84 mmol) in MeOH (40 mL) was added NiCl₂·6H₂O (0.03 g, 1.27 mmol) followed by dropwise addition of di-tert-butyl dicarbonate (0.27 g, 1.26 mmol) at 0° C. and the mixture was stirred for 5 min. To the reaction mixture was added NaBH₄ (0.22 g, 5.82 mmol) in portion by maintaining the same temperature. After stirring for 2 h at RT, the reaction mass was concentrated to remove solvent, diluted with EtOAc and filtered on Celite bed. The filtrate was washed with water, dried over anhydrous Na₂SO 4 and concentrated. The residue was purified by combi flash column chromatography using 20% EtOAc in n-hexane as an eluent to afford the title compound (0.17 g, 59%). LC-MS: 342 [M+H]⁺

### Step-4: (S)-5-(Aminomethyl)-N-(3,3-difluorocyclopentyl)-N-methylpyridin-2-amine hydrochloride

To a stirred solution of tert-butyl (S)-((6-((3,3-difluorocyclopentyl)(methyl)amino)pyridin-3-yl)methyl)carbamate (0.15 g, 0.44 mmol) in 1,4-dioxane (5 mL) was added 4 N HCl in 1,4-dioxane (5 mL) and the mixture was stirred at RT for 1 h. The reaction mixture was concentrated to get the title compound (0.1 g, 95%). LC-MS: 242 [M+H]⁺

### Step-5: (S)—N-((6-((3,3-Difluorocyclopentyl)(methyl)amino)pyridin-3-yl)methyl)-1-methyl-2-o xo-2,3-dihydro-1H-benzimidazole-5-carboxamide (Compound 105)

To a stirred solution of 1-methyl-2-oxo-2,3-dihydro-1H-benzimidazole-5-carboxylic acid (0.09 g, 0.46 mmol) in DMF (4 mL) was added EDC·HCl (0.09 g, 0.46 mmol) followed by HOBt (0.03 g, 0.22 mmol) and the mixture was stirred at RT for 10 min. Then (S)-5-(aminomethyl)-N-(3,3-difluorocyclopentyl)-N-methylpyridin-2-amine hydrochloride (0.11 g, 0.46 mmol) was added thereto followed by triethylamine (0.28 g, 2.7 mmol) and the mixture was stirred at RT for 16 h. The reaction mixture was poured into ice cold water, extracted with EtOAc, dried over anhydrous Na₂SO₄ and concentrated. The residue was purified by preparative HPLC using method C to afford the title compound (0.03 g, 18.0%). LC-MS: 416 [M+H]⁺; ¹H-NMR (400 MHz, CD₃OD): δ 8.10 (d, 1H), 7.66-7.64 (dd, 1H), 7.62-7.58 (dd, 2H), 7.18-7.16 (d, 1H), 6.71-6.69 (d, 1H), 5.27-5.23 (m, 1H), 4.45 (s, 2H), 3.43 (s, 3H), 2.91 (s, 3H), 2.36-1.96 (m, 6H).

### Example-106

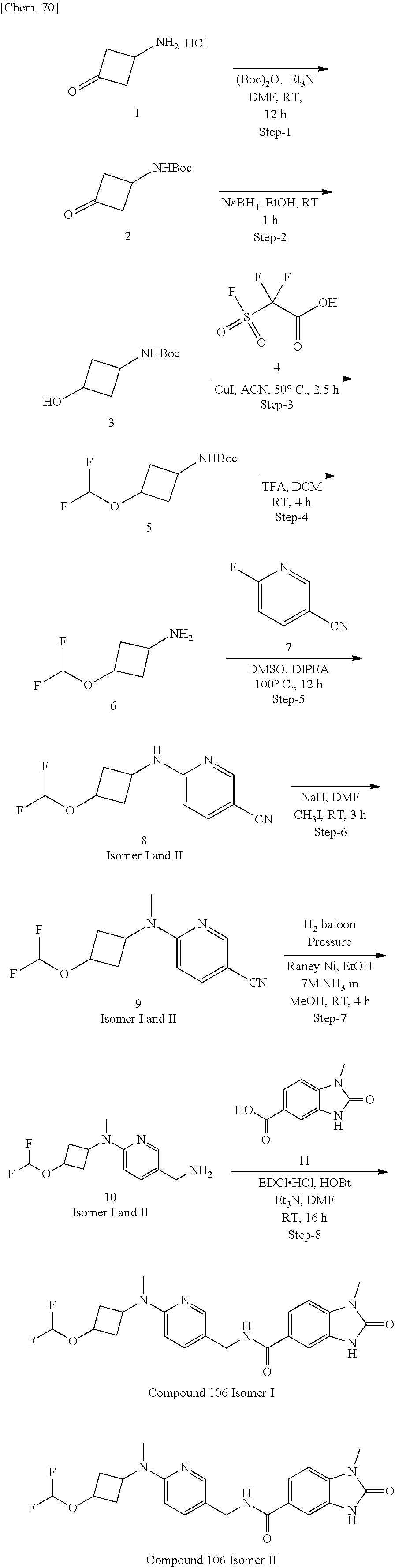

### Step-1: tert-Butyl (3-oxocyclobutyl)carbamate

To a stirred solution of 3-aminocyclobutan-1-one hydrochloride (1.5 g, 12.33 mmol) in DMF (20 mL) was added triethylamine (1.88 g, 18.5 mmol) followed by di-tert-butyl dicarbonate (3.2 g, 14.80 mmol) at 0° C. and the mixture was stirred at RT for 12 h. The reaction mixture was concentrated to remove solvent. The residue was purified by combi flash column chromatography using 5% MeOH in DCM as an eluent to afford the title compound (1.7 g, 74.3%). ¹H-NMR (400 MHz, DMSO-d₆): δ 7.43-7.40 (bs, 1H), 4.13-4.11 (m, 1H), 3.32-3.22 (m, 2H), 3.02-3.00 (m, 2H), 1.40 (d, 9H).

### Step-2: tert-Butyl (3-hydroxycyclobutyl)carbamate

To a stirred solution of tert-butyl (3-oxocyclobutyl)carbamate (0.7 g, 3.77 mmol) in EtOH (20 mL) was added NaBH₄ (0.07 g, 1.78 mmol) at 0° C. and the mixture was stirred at RT for 1 h. The reaction mass was concentrated to remove EtOH. The residue was diluted with EtOAc, washed with water, dried over anhydrous Na₂SO₄ and concentrated to afford the title compound (0.6 g, 59%). ¹H-NMR (400 MHz, DMSO-d₆): δ 7.03-7.01 (d, 1H), 4.98-4.96 (d, 1H), 3.74-3.70 (m, 1H), 3.39-3.32 (m, 1H), 2.43-2.37 (m, 2H), 2.08-2.00 (m, 2H), 1.37-1.36 (d, 9H).

### Step-3: tert-Butyl (3-(difluoromethoxy)cyclobutyl)carbamate

To a stirred solution of tert-butyl (3-hydroxycyclobutyl)carbamate (0.6 g, 3.2 mmol) in acetonitrile (20 mL) was added copper iodide (0.12 g, 0.64 mmol) followed by 2,2-difluoro-2-(fluorosulfonyl)acetic acid (0.68 g, 3.84 mmol) and the mixture was stirred at 50° C. for 2.5 h. The reaction mass was concentrated to remove solvent and diluted with EtOAc, washed with water, dried over anhydrous Na₂SO₄ and concentrated to afford the title compound (0.45 g, 59%). ¹H-NMR (400 MHz, DMSO-d₆): δ 7.40 (m, 1H), 6.78-6.40 (m, 1H), 3.98-3.84 (m, 1H), 2.23-2.17 (m, 2H), 1.99-1.98 (m, 1H), 1.52-1.48 (m, 2H), 1.38-1.36 (d, 9H).

### Step-4: 3-(Difluoromethoxy)cyclobutan-1-amine

To a solution of tert-butyl (3-(difluoromethoxy)cyclobutyl)carbamate (0.45 g, 1.26 mmol) in DCM (15 mL) was added TFA (1.5 mL) and the mixture was stirred at RT for 4 h. The reaction mixture was concentrated to get the title compound (0.24 g, 92%). LC-MS: 138 [M+H]⁺

### Step-5: 6-((3-(Difluoromethoxy)cyclobutyl)amino)nicotinonitrile

To a stirred solution of 6-fluoronicotinonitrile (0.26 g, 2.18 mmol) in DMSO (8 mL) was added DIPEA (1.7 g, 13.1 mmol) followed by 3-(difluoromethoxy)cyclobutan-1-amine (0.3 g, 2.18 mmol) and the mixture was stirred at 100° C. for 12 h. The reaction mixture was concentrated to remove DIPEA, the residue was diluted with EtOAc, washed with water, dried over anhydrous Na₂SO₄ and concentrated. The residue was purified by combi flash column chromatography using 30% EtOAc in n-hexane as an eluent to afford both cis- and trans-isomers of the title compound, Isomer I (0.07 g, 13%) and Isomer II (0.05 g, 10%). LC-MS: 240 [M+H]⁺

### Isomer I

### Step-6: 6-((3-(Difluoromethoxy)cyclobutyl)(methyl)amino)nicotinonitrile

To a stirred solution of 6-((3-(difluoromethoxy)cyclobutyl)amino)nicotinonitrile (Isomer I of the Step-5 above: 0.06 g, 0.25 mmol) in DMF (5 mL) was added NaH (60% dispersion in mineral oil) (0.01 g, 0.37 mmol) followed by dropwise addition of iodomethane (0.04 g, 0.28 mmol) at 0° C. and the mixture of stirred at RT for 3 h. The reaction mixture was quenched with water, extracted with EtOAc, dried over anhydrous Na₂SO₄ and concentrated to afford the title compound (0.06 g, 95%). LC-MS: 254 [M+H]⁺.

### Step-7: 5-(Aminomethyl)-N-(3-(difluoromethoxy)cyclobutyl)-N-methylpyridin-2-amine

To a stirred solution of 6-((3-(difluoromethoxy)cyclobutyl)(methyl)amino)nicotinonitrile (0.06 g, 0.23 mmol) in EtOH (5 mL) was added Raney nickel (0.07 g) followed by 7 M ammonia in MeOH (2 mL) and the mixture was stirred at RT for 4 h under hydrogen bladder pressure. The reaction mass was filtered through Celite and the filtrate was concentrated to afford the title compound (0.045 g, 73.2%). LC-MS: 258 [M+H]⁺.

### Step-8: N-((6-((3-(Difluoromethoxy)cyclobutyl)(methyl)amino)pyridin-3-yl)methyl)-1-methyl-2-oxo-2,3-dihydro-1H-benzimidazole-5-carboxamide (Compound 106 Isomer I)

To a stirred solution of 1-methyl-2-oxo-2,3-dihydro-1H-benzimidazole-5-carboxylic acid (0.03 g, 0.18 mmol) in DMF (4 mL) was added EDC·HCl (0.03 g, 0.18 mmol) followed by HOBt (0.01 g, 0.09 mmol) and the mixture was stirred at RT for 10 min. To the reaction mixture was added 5-(aminomethyl)-N-(3-(difluoromethoxy)cyclobutyl)-N-methylpyridin-2-amine (0.04 g, 0.18 mmol) followed by triethylamine (0.07 g, 0.73 mmol) and the mixture was stirred at RT for 16 h. The reaction mixture was poured into ice cold water, extracted with EtOAc, dried over anhydrous Na₂SO₄ and concentrated. The residue was purified by preparative HPLC method C to afford the title compound (0.01 g, 12.7%). LC-MS: 432 [M+H]⁺; ¹H-NMR (400 MHz, CDCl₃): δ 8.18-8.17 (d, 1H), 8.08 (bs, 1H), 7.56-7.45 (m, 3H), 6.99-6.97 (d, 1H), 6.54-6.51 (d, 1H), 6.39-6.02 (m, 2H), 4.6-4.54 (m, 3H), 4.53-4.41 (m, 1H), 3.45 (s, 3H), 3.05 (s, 3H), 2.78-2.73 (m, 2H), 2.37-2.30 (m, 2H).

### Isomer II

### Step-6A: 6-((3-(Difluoromethoxy)cyclobutyl)(methyl)amino)nicotinonitrile

To a stirred solution of 6-((3-(difluoromethoxy)cyclobutyl)amino)nicotinonitrile (Isomer II of the Step-5 above: 0.04 g, 0.18 mmol) in DMF (5 mL) was added NaH (60% dispersion in mineral oil) (0.01 g, 0.28 mmol) followed by dropwise addition of iodomethane (0.03 g, 0.22 mmol) at 0° C. and the mixture was stirred at RT for 3 h. The residue was diluted with EtOAc, washed with water, dried over anhydrous Na₂SO₄ and concentrated to afford the title compound (0.04 g, 84%). LC-MS: 254 [M+H]⁺.

### Step-7A: 5-(Aminomethyl)-N-(3-(difluoromethoxy)cyclobutyl)-N-methylpyridin-2-amine

To a stirred solution of 6-((3-(difluoromethoxy)cyclobutyl)(methyl)amino)nicotinonitrile (0.04 g, 0.23 mmol) in EtOH (5 mL) was added Raney nickel (0.04 g) followed by 7 M ammonia in MeOH (2 mL) and the mixture was stirred at RT for 4 h under hydrogen bladder pressure. The reaction mass was filtered through Celite and the filtrate was concentrated to afford the title compound (0.02 g, 73.2%). LC-MS: 258 [M+H]⁺.

### Step-8A: N-((6-((3-(Difluoromethoxy)cyclobutyl)(methyl)amino)pyridin-3-yl)methyl)-1-methyl-2-oxo-2,3-dihydro-1H-benzimidazole-5-carboxamide (Compound 106 Isomer II)

To a stirred solution of 1-methyl-2-oxo-2,3-dihydro-1H-benzimidazole-5-carboxylic acid (0.02 g, 0.1 mmol) in DMF (4 mL) was added EDC·HCl (0.02 g, 0.1 mmol) followed by HOBt (0.01 g, 0.05 mmol) and stirred at RT for 10 min. Then 5-(aminomethyl)-N-(3-(difluoromethoxy)cyclobutyl)-N-methylpyridin-2-amine (0.02 g, 0.1 mmol) was added thereto followed by triethylamine (0.04 g, 0.43 mmol) and the mixture was stirred at RT for 16 h. The reaction mixture was poured into ice cold water, extracted with EtOAc, dried over anhydrous Na₂SO₄ and concentrated to get crude compound. The crude was further purified by preparative HPLC method C to afford the title compound (0.003 g, 6.7%). LC-MS: 432 [M+H]⁺; ¹H-NMR (400 MHz, CDCl₃): δ 8.58 (s, 1H), 8.20-8.19 (s, 1H), 7.56-7.51 (m, 3H), 6.99-6.97 (d, 1H), 6.52-6.50 (d, 1H), 6.28-6.05 (m, 2H), 5.05-5.01 (m, 1H), 4.76-4.75 (m, 1H), 4.54-4.53 (d, 2H), 3.45 (s, 3H), 3.03-3.01 (s, 3H), 2.58-2.55 (m, 4H).

### Example-107

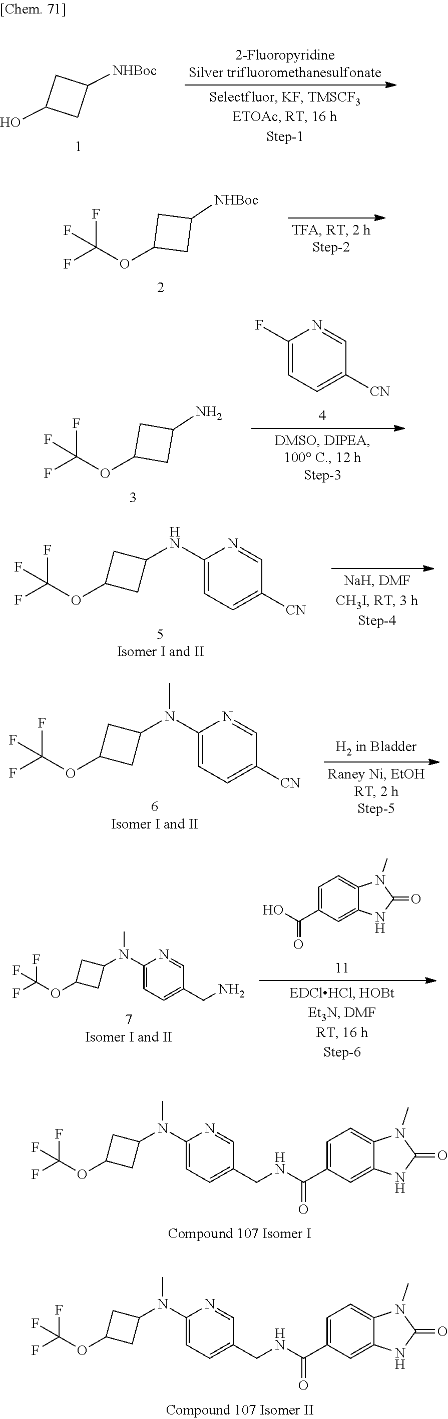

### Step-1: tert-Butyl (3-(trifluoromethoxy)cyclobutyl)carbamate

To a stirred solution of tert-butyl (3-hydroxycyclobutyl)carbamate (0.9 g, 4.8 mmol) in EtOAc (50 mL) were added silver trifluoromethanesulfonate (4.94 g, 19.22 mmol), KF (1.11 g, 19.22 mmol), 1-(chloromethyl)-4-fluoro-1,4-diazoniabicyclo[2.2.2]octane bis(tetrafluoroborate) (2.55 g, 7.21 mmol), and 2-fluoropyridine (1.86 g, 19.22 mmol) followed by trimethyl(trifluoromethyl)silane (1.87 g, 12.01 mmol) and the mixture was stirred at RT for 16 h. The reaction mass was filtered through Celite and the filtrate was washed with water, dried over anhydrous Na₂SO₄ and concentrated. The residue was purified by combi flash column chromatography using 10% EtOAc in n-hexane as an eluent to get the title compound (0.65 g, 53%). ¹H-NMR (400 MHz, DMSO-d₆): δ 7.23-7.21 (d, 1H), 4.53-4.45 (m, 1H), 3.65-3.61 (m, 1H), 2.67-2.50 (m, 1H), 2.45-2.43 (m, 1H), 2.34-2.23 (m, 1H), 2.15-2.07 (m, 1H), 1.40-1.37 (d, 9H).

### Step-2: 3-(Trifluoromethoxy)cyclobutan-1-amine

To a solution of tert-butyl (3-(trifluoromethoxy)cyclobutyl)carbamate (0.62 g, 2.44 mmol) in DCM (25 mL) was added TFA (3 mL) and the mixture was stirred at RT for 2 h. The reaction mixture was concentrated to get the title compound (0.3 g, 80%). ¹H-NMR (400 MHz, DMSO-d₆): δ 8.17 (s, 2H), 4.67-4.63 (m, 1H), 3.38-3.37 (m, 1H), 2.58-2.57 (m, 2H), 2.38-2.30 (m, 2H).

### Step-3: 6-((3-(Trifluoromethoxy)cyclobutyl)amino)nicotinonitrile

To a stirred solution of 6-fluoronicotinonitrile (0.5 g, 4.09 mmol) in DMSO (20 mL) was added DIPEA (3.17 g, 24.57 mmol) followed by 3-(trifluoromethoxy)cyclobutan-1-amine (0.63 g, 4.09 mmol) and the mixture was stirred at 100° C. for 12 h. The reaction mixture was concentrated to remove DIPEA, and the residue was diluted with EtOAc, washed with water, dried over anhydrous Na₂ SO₄ and concentrated. The residue was purified by combi flash column chromatography using 30% EtOAc in n-hexane as an eluent to afford both cis- and trans-isomers of the title compound, Isomer I (0.3 g, 29%) and Isomer II (0.15 g, 14%). LC-MS: 258 [M+H]⁺.

### Isomer I

### Step-4: 6-(Methyl(3-(trifluoromethoxy)cyclobutyl)amino)nicotinonitrile

To a stirred solution of 6-((3-(trifluoromethoxy)cyclobutyl)amino)nicotinonitrile (Isomer I of the Step-3 above: 0.3 g, 1.16 mmol) in DMF (10 mL) was added NaH (60% dispersion in mineral oil) (0.14 g, 3.49 mmol) followed by dropwise addition of iodomethane (0.33 g, 2.33 mmol) at 0° C. and the mixture was stirred at RT for 3 h. The reaction mixture was quenched with ice cold water, extracted with EtOAc, dried over anhydrous Na₂SO₄ and concentrated. The residue was purified by combi flash column chromatography using 10% EtOAc in n-hexane as an eluent to get the title compound (0.25 g, 79%). LC-MS: 272 [M+H]⁺.

### Step-5: 5-(Aminomethyl)-N-methyl-N-(3-(trifluoromethoxy)cyclobutyl)pyridin-2-amine

To a stirred solution of 6-(methyl(3-(trifluoromethoxy)cyclobutyl)amino)nicotinonitrile (0.25 g, 0.92 mmol) in EtOH (10 mL) was added Raney nickel (0.25 g) and the mixture was stirred at RT for 2 h under hydrogen bladder pressure. The reaction mass was filtered through Celite, and the filtrate was concentrated to afford the title compound (0.25 g, 98%). LC-MS: 276 [M+H]⁺.

### Step-6: 1-Methyl-N-((6-(methyl(3-(trifluoromethoxy)cyclobutyl)amino)pyridin-3-yl)methyl)-2-oxo-2,3-dihydro-1H-benzimidazole-5-carboxamide (Compound 107 Isomer I)

To a stirred solution of 1-methyl-2-oxo-2,3-dihydro-1H-benzimidazole-5-carboxylic acid (0.14 g, 0.72 mmol) in DMF (8 mL) was added EDC·HCl (0.16 g, 0.81 mmol) followed by HOBt (0.05 g, 0.4 mmol) and the mixture was stirred at RT for 10 min. To the reaction mixture was added 5-(aminomethyl)-N-methyl-N-(3-(trifluoromethoxy)cyclobutyl)pyridin-2-amine (0.25 g, 0.9 mmol) followed by triethylamine (0.45 g, 4.54 mmol) and the mixture was stirred at RT for 16 h. The reaction mixture was poured into ice cold water, and the solid thrown out was collected by filtration and dried. This was purified by Preparative TLC using 5% MeOH in DCM as an eluent to get the title compound (0.03 g, 7%). LC-MS: 450 [M+H]⁺; ¹H-NMR (400 MHz, CD₃OD): δ 8.16-8.11 (d, 1H), 7.66-7.58 (m, 3H), 7.18-7.16 (d, 1H), 6.68-6.67 (d, 1H), 4.59-4.57 (m, 1H), 4.46 (s, 2H), 4.41-4.36 (m, 1H), 3.43 (s, 3H), 3.00 (s, 3H), 2.83-2.77 (m, 2H), 2.42-2.37 (m, 2H).

### Isomer II

### Step-4A: 6-(Methyl(3-(trifluoromethoxy)cyclobutyl)amino)nicotinonitrile

To a stirred solution of 6-((3-(trifluoromethoxy)cyclobutyl)amino)nicotinonitrile (Isomer II of the Step-3 above: 0.15 g, 0.58 mmol) in DMF (5 mL) was added NaH (60% dispersion in mineral oil) (0.07 g, 1.74 mmol) followed by dropwise addition of iodomethane (0.16 g, 1.16 mmol) at 0° C. and the mixture was stirred at RT for 3 h. The reaction mixture was quenched with ice cold water and extracted with EtOAc, dried over anhydrous Na₂SO₄ and concentrated. The residue was purified by combi flash column chromatography using 10% EtOAc in n-hexane as an eluent to get the title compound (0.11 g, 69%). LC-MS: 272 [M+H]⁺;

### Step-5A: 5-(Aminomethyl)-N-methyl-N-(3-(trifluoromethoxy)cyclobutyl)pyridin-2-amine

To a stirred solution of 6-(methyl(3-(trifluoromethoxy)cyclobutyl)amino)nicotinonitrile (0.11 g, 0.40 mmol) in EtOH (5 mL) was added Raney nickel (0.12 g) and stirred at RT for 2 h under hydrogen bladder pressure. The reaction mass was filtered through Celite and the filtrate was concentrated to afford the title compound (0.08 g, 71%). LC-MS: 276 [M+H]⁺.

### Step-6A: 1-Methyl-N-((6-(methyl(3-(trifluoromethoxy)cyclobutyl)amino)pyridin-3-yl)methyl)-2-oxo-2,3-dihydro-1H-benzimidazole-5-carboxamide (Compound 107 Isomer II)

To a stirred solution of 1-methyl-2-oxo-2,3-dihydro-1H-benzimidazole-5-carboxylic acid (0.04 g, 0.72 mmol) in DMF (3 mL) was added EDC·HCl (0.05 g, 0.26 mmol) followed by HOBt (0.02 g, 0.13 mmol) and the mixture was stirred at RT for 10 min. To the reaction mixture was added 5-(aminomethyl)-N-methyl-N-(3-(trifluoromethoxy)cyclobutyl)pyridin-2-amine (0.08 g, 0.9 mmol) followed by triethylamine (0.15 g, 1.45 mmol) and the mixture was stirred at RT for 16 h. The reaction mixture was poured into ice cold water to get solid which was collected by filtration and dried. This was purified by Preparative TLC using 5% MeOH in DCM as an eluent to get the title compound (0.03 g, 23%). LC-MS: 450 [M+H]⁺; ¹H-NMR (400 MHz, CDCl₃): δ 8.64 (d, 1H), 8.20-8.19 (d, 1H), 7.57-7.52 (m, 3H), 6.99-6.97 (d, 1H), 6.53-6.51 (d, 1H), 6.29 (t, 1H), 5.08-5.04 (m, 1H), 4.84-4.81 (m, 1H), 4.55-4.54 (d, 2H), 3.45 (s, 3H), 3.01 (s, 3H), 2.64-2.59 (m, 4H).

### Example-108

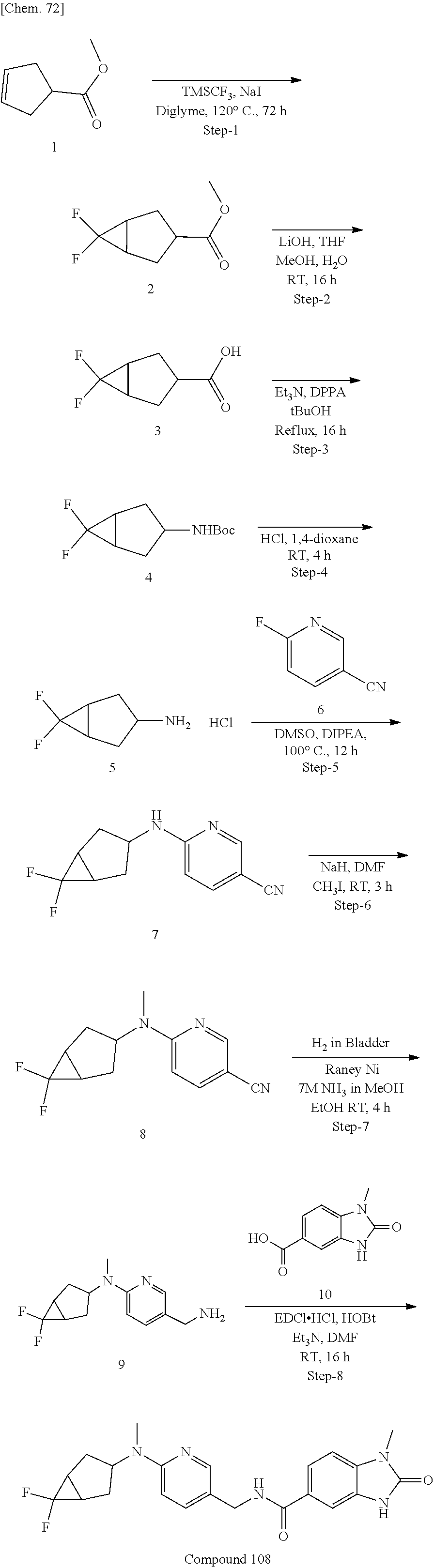

### Step-1: Methyl 6,6-difluorobicyclo[3.1.0]hexane-3-carboxylate

To a stirred solution of methyl cyclopent-3-ene-1-carboxylate (3.0 g, 23.7 mmol) in diglyme (15 mL) was added trimethyl(trifluoromethyl)silane (9.28 g, 59.4 mmol) followed by sodium iodide (1.78 g, 11.88 mmol) at 120° C. and the mixture was stirred for 72 h. The reaction mass was diluted with DCM, washed with water, dried over anhydrous Na₂SO₄ and concentrated to afford the title compound (2.8 g, 66.8%). 1H-NMR (400 MHz, CD₃OD): δ 3.23-3.10 (m, 1H), 2.90-2.80 (m, 1H), 2.66 (d, 3H), 2.33-2.24 (m, 4H), 2.05-2.01 (dd, 1H).

### Step-2: 6,6-Difluorobicyclo[3.1.0]hexane-3-carboxylic acid

To a stirred solution of methyl 6,6-difluorobicyclo[3.1.0]hexane-3-carboxylate (2.5 g, 14.15 mmol) in THF (15 mL) and MeOH (10 mL) was added LiOH (0.6 g, 14.2 mmol) in water (5 mL) and the mixture was stirred at RT for 16 h. The reaction mixture was concentrated to remove THF and MeOH, acidified with 1 N HCl and extracted with DCM. The combined organic layer was dried over anhydrous Na₂SO₄ and concentrated to afford the title compound (2.0 g, 87.0%). ¹H-NMR (400 MHz, CDCl₃): δ 3.21-3.16 (m, 1H), 2.91-2.90 (m, 1H), 2.38-2.27 (m, 4H), 2.07-2.03 (dd, 2H).

### Step-3: tert-Butyl (6,6-difluorobicyclo[3.1.0]hexan-3-yl)carbamate

To a stirred solution of 6,6-difluorobicyclo[3.1.0]hexane-3-carboxylic acid (1.5 g, 9.25 mmol) in toluene (20 mL) was added triethylamine (1.8 g, 18.5 mmol) followed by DPPA (3.8 g, 13.8 mmol) and the mixture was stirred at RT for 10 min. The reaction mixture was added t-BuOH (9.0 g, 120.25 mmol) and stirred at 80° C. for 16 h. The reaction mixture was poured into ice cold water, extracted with EtOAc, dried over anhydrous Na₂SO₄ and concentrated. The residue was purified by combi flash column chromatography using 20% EtOAc in n-hexane as an eluent to afford the title compound (1.2 g, 55.6%). ¹H-NMR (400 MHz, DMSO-d₆): δ 5.70-5.60 (d, 1H), 4.25-4.15 (m, 1H), 2.25-2.15 (m, 4H), 1.90-1.75 (m, 2H), 1.38-1.37 (d, 9H).

### Step-4: 6,6-Difluorobicyclo[3.1.0]hexan-3-amine hydrochloride

To a stirred solution of tert-butyl (6,6-difluorobicyclo[3.1.0]hexan-3-yl)carbamate (1.2 g, 5.14 mmol) in 1,4-dioxane (5 mL) was added 4 N HCl in 1,4-dioxane (10 mL) and the mixture was stirred at RT for 4 h. The reaction mixture was concentrated to get the title compound (0.3 g, 34%). LC-MS: 134 [M+H]⁺

### Step-5: 6-((6,6-Difluorobicyclo[3.1.0]hexan-3-yl)amino)nicotinonitrile

To a stirred solution of 6-fluoronicotinonitrile (0.2 g, 1.63 mmol) in DMSO (6 mL) was added DIPEA (1.26 g, 9.8 mmol) followed by 6,6-difluorobicyclo[3.1.0]hexan-3-amine hydrochloride (0.27 g, 1.63 mmol) and the mixture was stirred at 100° C. for 12 h. The reaction mixture was concentrated to remove DIPEA and the residue was diluted with EtOAc, washed with water, dried over anhydrous Na₂SO₄ and concentrated. The residue was purified by combi flash column chromatography using 30% EtOAc in n-hexane as an eluent to afford the title compound (0.04 g, 84%). LC-MS: 236 [M+H]⁺.

### Step-6: 6-((6,6-Difluorobicyclo[3.1.0]hexan-3-yl)(methyl)amino)nicotinonitrile

To a stirred solution of 6-((6,6-difluorobicyclo[3.1.0]hexan-3-yl)amino)nicotinonitrile (0.2 g, 0.85 mmol) in DMF (5 mL) was added NaH (60% dispersion in mineral oil) (0.05 g, 1.27 mmol) followed by dropwise addition of iodomethane (0.15 g, 1.02 mmol) at 0° C. and the mixture was stirred at RT for 3 h. The residue was diluted with EtOAc, washed with water, dried over anhydrous Na₂SO₄ and concentrated to afford the title compound (0.2 g, 94%). LC-MS: 250 [M+H]⁺.

### Step-7: 5-(Aminomethyl)-N-(6,6-difluorobicyclo[3.1.0]hexan-3-yl)-N-methylpyridin-2-amine

To a stirred solution of 6-((6,6-difluorobicyclo[3.1.0]hexan-3-yl)(methyl)amino)nicotinonitrile (0.19 g, 0.76 mmol) in EtOH (10 mL) was added Raney nickel (0.2 g) followed by 7 M ammonia in MeOH (5 mL) and the mixture was stirred at RT for 4 h under hydrogen bladder pressure. The reaction mass was filtered through Celite and the filtrate was concentrated to afford the title compound (0.19 g, 98.2%). LC-MS: 254 [M+H]⁺.

### Step-8: N-((6-((6,6-Difluorobicyclo[3.1.0]hexan-3-yl)(methyl)amino)pyridin-3-yl)methyl)-1-met hyl-2-oxo-2,3-dihydro-1H-benzimidazole-5-carboxamide (Compound 108)

To a stirred solution of 1-methyl-2-oxo-2,3-dihydro-1H-benzimidazole-5-carboxylic acid (0.15 g, 0.78 mmol) in DMF (4 mL) was added EDC·HCl (0.15 g, 0.78 mmol) followed by HOBt (0.06 g, 0.39 mmol) and the mixture was stirred at RT for 10 min. To the reaction mixture was added 5-(aminomethyl)-N-(6,6-difluorobicyclo[3.1.0]hexan-3-yl)-N-methylpyridin-2-amine (0.2 g, 0.78 mmol) followed by triethylamine (0.31 g, 3.12 mmol) and the mixture was stirred at RT for 16 h. The reaction mixture was poured into ice cold water, extracted with EtOAc, dried over anhydrous Na₂SO₄ and concentrated. The residue was purified by preparative HPLC using method C to afford the title compound (0.06 g, 18.0%). LC-MS: 428 [M+H]⁺; ¹H-NMR (400 MHz, CD₃OD): δ 8.09-8.02 (dd, 1H), 7.85 (d, 1H), 7.65-7.62 (dd, 1H), 7.57-7.50 (dd, 1H), 7.31-7.29 (dd, 1H), 7.18-7.16 (dd, 1H), 4.60-4.55 (m, 1H), 4.47 (s, 2H), 3.41 (s, 3H), 3.11 (s, 3H), 2.39-2.30 (m, 4H), 2.25-2.1 (m, 2H).

### Example-109

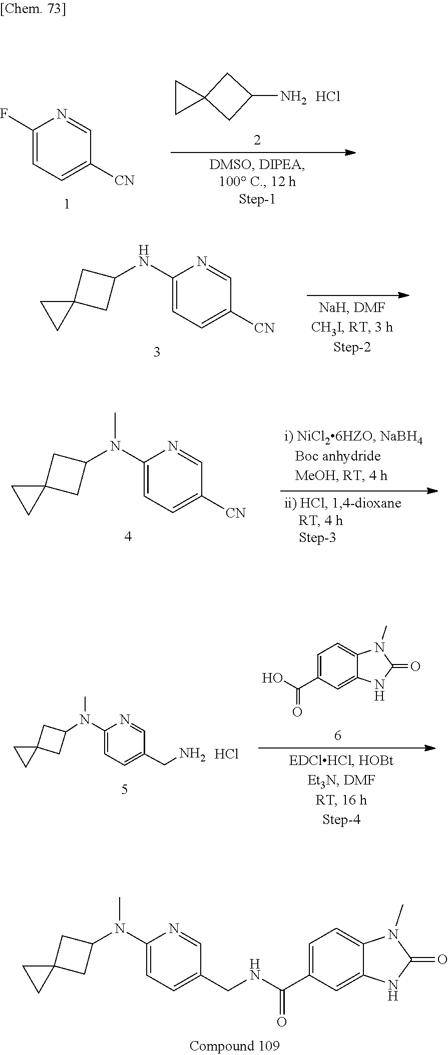

### Step-1: 6-(Spiro[2.3]hexan-5-ylamino)nicotinonitrile

To a stirred solution of 6-fluoronicotinonitrile (0.13 g, 1.06 mmol) in DMSO (5 mL) was added DIPEA (0.28 g, 2.12 mmol) followed by spiro[2.3]hexan-5-amine hydrochloride (0.14 g, 1.06 mmol) and the mixture was stirred at 100° C. for 12 h. The reaction mixture was concentrated to remove DIPEA, poured into ice cold water, and the solid separated was collected by filtration and dried to afford the title compound (0.20 g, 94%). LC-MS: 200 [M+H]⁺.

### Step-2: 6-(Methyl(spiro[2.3]hexan-5-yl)amino)nicotinonitrile

To a stirred solution of 6-(spiro[2.3]hexan-5-ylamino)nicotinonitrile (0.20 g, 1.00 mmol) in DMF (8 mL) was added NaH (60% dispersion in mineral oil) (0.06 g, 1.50 mmol) followed by dropwise addition of iodomethane (0.17 g, 1.50 mmol) at 0° C. and the mixture was stirred at RT for 3 h. The reaction mixture poured into ice cold water, and the solid separated was collected by filtration and dried to afford the title compound (0.16 g, 74%). LC-MS: 214 [M+H]⁺.

### Step-3: 5-(Aminomethyl)-N-methyl-N-(spiro[2.3]hexan-5-yl)pyridin-2-amine hydrochloride

To a stirred solution of 6-(methyl(spiro[2.3]hexan-5-yl)amino)nicotinonitrile (0.13 g, 0.63 mmol) in MeOH (20 mL) was added NiCl₂·6H₂O (0.08 g, 0.09 mmol) followed by dropwise addition of di-tert-butyl dicarbonate (0.21 g, 0.96 mmol) at 0° C. and the mixture was stirred for 5 min. To the reaction mixture was added NaBH₄ (0.17 g, 4.43 mmol) in portion by maintaining the same temperature. After stirring at RT for 4 h, the reaction mass was concentrated to remove solvent. The residue was diluted with EtOAc, washed with water, dried over anhydrous Na₂SO₄ and concentrated. The residue was purified by combi flash column chromatography using 20% EtOAc in n-hexane as an eluent. This was taken with 1,4-dioxane (2 mL) and 4 M HCl in 1,4-dioxane (2 mL) was added then stirred at RT for 4 h. The reaction mixture was concentrated to get the title compound (0.12 g, 90.6%). LCMS: 218 [M+H]⁺.

### Step-4: 1-Methyl-N-((6-(methyl(spiro[2.3]hexan-5-yl)amino)pyridin-3-yl)methyl)-2-oxo-2,3-d ihydro-1H-benzimidazole-5-carboxamide formate (Compound 109)

To a stirred solution of 1-methyl-2-oxo-2,3-dihydro-1H-benzimidazole-5-carboxylic acid (0.10 g, 0.52 mmol) in DMF (4 mL) was added EDC·HCl (0.10 g, 0.52 mmol) followed by HOBt (0.03 g, 0.26 mmol) and the mixture was stirred at RT for 10 min. To the reaction mixture was added 5-(aminomethyl)-N-methyl-N-(spiro[2.3]hexan-5-yl)pyridin-2-amine hydrochloride (0.11 g, 0.52 mmol) followed by triethylamine (0.31 g, 3.12 mmol) and the mixture was stirred at RT for 16 h. The reaction mixture was poured into ice cold water, extracted with EtOAc, dried over anhydrous Na₂SO₄ and concentrated. The residue was purified using preparative HPLC method C to get the title compound as a formic acid salt (0.02 g, 10.0%). LC-MS: 392 [M+H]⁺; ¹H-NMR (400 MHz, DMSO-d₆): δ 11.08 (s, 1H), 8.82-8.79 (t, 1H), 8.47 (s, 1H), 8.07-8.06 (d, 1H), 7.63-7.60 (dd, 1H), 7.50-7.40 (dd, 2H), 7.15-7.13 (d, 1H), 6.62-6.60 (d, 1H), 5.01 (m, 1H), 4.31-4.29 (d, 2H), 3.33-3.30 (s, 3H), 2.97 (s, 3H), 2.42-2.33 (m, 2H), 2.18-2.15 (m, 2H), 0.52-0.49 (m, 2H), 0.44-0.41 (m, 2H).

### Example-110

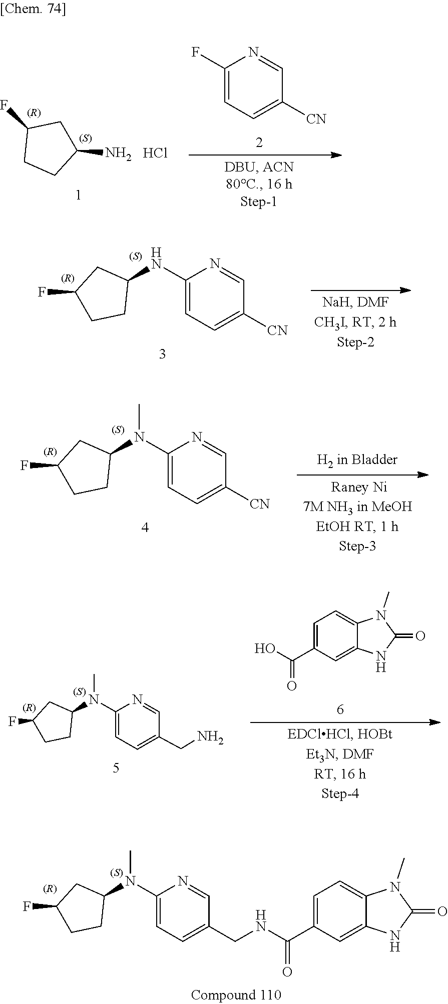

### Step-1: 6-(((1S,3R)-3-Fluorocyclopentyl)amino)nicotinonitrile

To a stirred solution of 6-fluoronicotinonitrile (0.15 g, 1.22 mmol) in acetonitrile (6 mL) was added DBU (0.15 g, 0.98 mmol) followed by (1S,3R)-3-fluorocyclopentan-1-amine hydrochloride (0.17 g, 1.22 mmol) and the mixture was stirred at 80° C. for 16 h. The reaction mixture was concentrated to get the title compound (0.25 g, 99%). LC-MS: 206 [M+H]⁺;

### Step-2: 6-(((1S,3R)-3-Fluorocyclopentyl)(methyl)amino)nicotinonitrile

To a stirred solution of 6-(((1S,3R)-3-fluorocyclopentyl)amino)nicotinonitrile (0.25 g, 1.21 mmol) in DMF (6 mL) was added NaH (60% dispersion in mineral oil) (0.07 g, 1.82 mmol) followed by dropwise addition of iodomethane (0.20 g, 1.46 mmol) at 0° C. and the mixture was stirred at RT for 2 h. The reaction mixture was poured into ice cold water and extracted with EtOAc. The separated organic layer was washed with brine, dried over anhydrous Na₂SO₄ and concentrated. The residue was purified by combi flash column chromatography using 30% EtOAc in n-hexane as an eluent to afford the title compound (0.18 g, 67%). LC-MS: 220 [M+H]⁺

### Step-3: 5-(Aminomethyl)-N-((1S,3R)-3-fluorocyclopentyl)-N-methylpyridin-2-amine

To a stirred solution of 6-(((1S,3R)-3-fluorocyclopentyl)(methyl)amino)nicotinonitrile (0.1 g, 0.45 mmol) in EtOH (10 mL) was added 7 M ammonia in MeOH (4 mL) followed by the addition of Raney nickel (0.03 g) and stirred under hydrogen pressure for 1 h at RT. The reaction mass was filtered through Celite and the filtrate was concentrated to afford the title compound (0.1 g, 98%). LC-MS: 224 [M+H]⁺.

### Step-4: N-((6-(((1S,3R)-3-Fluorocyclopentyl)(methyl)amino)pyridin-3-yl)methyl)-1-methyl-2-ox o-2,3-dihydro-1H-benzimidazole-5-carboxamide (Compound 110)

To a stirred solution of 1-methyl-2-oxo-2,3-dihydro-1H-benzimidazole-5-carboxylic acid (0.09 g, 0.46 mmol) in DMF (5 mL) was added EDC·HCl (0.09 g, 0.46 mmol) followed by HOBt (0.03 g, 0.23 mmol) and the mixture was stirred at RT for 10 min. To the reaction mixture was added 5-(aminomethyl)-N-((1S,3R)-3-fluorocyclopentyl)-N-methylpyridin-2-amine (0.10 g, 0.46 mmol) followed by triethylamine (0.19 g, 1.87 mmol) and the mixture was stirred at RT for 16 h. The reaction mixture was poured into ice cold water, and the precipitate formed was collected by filtration and dried. The crude product was purified using preparative HPLC method C to get the title compound (0.04 g, 22.4%). LC-MS: 398 [M+H]⁺; ¹H-NMR (400 MHz, CD₃OD): δ 8.07-8.06 (d, 1H), 7.67-7.64 (dd, 1H), 7.61-7.58 (dd, 2H), 7.18-7.16 (d, 1H), 6.73-6.71 (d, 1H), 5.20-5.19 (m, 1H), 5.08-5.06 (m, 1H), 4.45 (s, 2H), 3.43 (s, 3H), 2.93 (s, 3H), 2.37-2.03 (m, 1H), 1.94-1.88 (m, 1H), 1.82-1.70 (m, 4H).

### Example-111

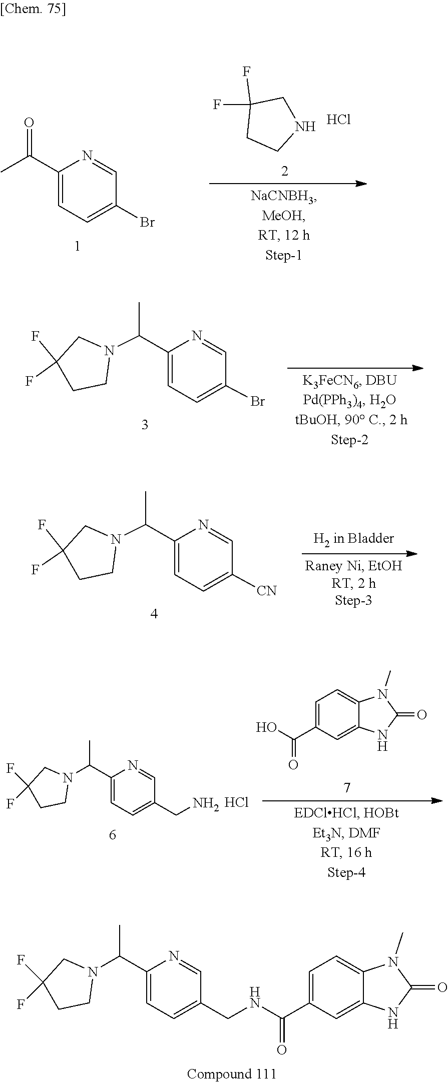

### Step-1: 5-Bromo-2-(1-(3,3-difluoropyrrolidin-1-yl)ethyl)pyridine

To a stirred solution of 1-(5-bromopyridin-2-yl)ethan-1-one (0.5 g, 2.5 mmol) in MeOH (10 mL) was added 3,3-difluoropyrrolidine hydrochloride (0.46 g, 3.25 mmol) followed by sodium cyanoborohydride (0.47 g, 7.5 mmol) and the mixture was stirred at RT for 12 h. The reaction mixture was poured into ice cold water, extracted with EtOAc, dried over Na₂SO₄ and concentrated. The residue was purified by combi flash column chromatography using 10% EtOAc in n-hexane as an eluent to afford the title compound (0.48 g, 66%). LC-MS: 291 [M+H]⁺.

### Step-2: 6-(1-(3,3-Difluoropyrrolidin-1-yl)ethyl)nicotinonitrile

To a stirred solution of 5-bromo-2-(1-(3,3-difluoropyrrolidin-1-yl)ethyl)pyridine (0.25 g, 0.85 mmol) in t-BuOH and water was added DBU (0.03 g, 0.21 mmol) followed by potassium ferrocyanide trihydrate (0.14 g, 0.34 mmol). The reaction mass was degassed with argon for 15 min, then tetrakis(triphenylphosphine)palladium (0.05 g, 0.04 mmol) was added thereto and stirred at 90° C. for 12 h. The reaction mass was concentrated and the crude compound was purified by combi flash column chromatography using 10% EtOAc in n-hexane as an eluent to afford the title compound (0.13 g, 63.79%). LC-MS: 238 [M+H]⁺.

### Step-3: (6-(1-(3,3-Difluoropyrrolidin-1-yl)ethyl)pyridin-3-yl)methanamine

To a stirred solution of 6-(1-(3,3-difluoropyrrolidin-1-yl)ethyl)nicotinonitrile (0.11 g, 0.46 mmol) in EtOH (5 mL) was added Raney nickel (0.14 g) and stirred under hydrogen bladder pressure for 2 h at RT. The reaction mixture was filtered through Celite and the filtrate concentrated to get the title compound (0.12 g). LC-MS: 242 [M+H]⁺.

### Step-4: N-((6-(1-(3,3-Difluoropyrrolidin-1-yl)ethyl)pyridin-3-yl)methyl)-1-methyl-2-oxo-2,3-dih ydro-1H-benzimidazole-5-carboxamide hydrochloride (Compound 111)

To a stirred solution of 1-methyl-2-oxo-2,3-dihydro-1H-benzimidazole-5-carboxylic acid (0.76 g, 0.39 mmol) in DMF (3 mL) was added triethylamine (0.3 g, 2.98 mmol), EDC·HCl (0.08 g, 0.44 mmol), HOBt (0.03 g, 0.22 mmol) followed by (6-(1-(3,3-difluoropyrrolidin-1-yl)ethyl)pyridin-3-yl)methanamine (0.12 g, 0.89 mmol) and the mixture was stirred at RT for 12 hours. The reaction mass was quenched with water, extracted with EtOAc, dried over Na₂SO₄, and concentrated. The residue was purified by preparative HPLC using method D to get the title compound as a HCl salt (0.02 g, 41.91%). LC-MS: 416 [M+H]⁺; ¹H-NMR (400 MHz, CD₃OD): δ 8.75-8.74 (d, 1H), 8.0-7.98 (dd, 1H), 7.72-7.69 (dd, 1H), 7.63 (d, 1H), 7.57-7.55 (d, 1H), 7.21-7.19 (d, 1H), 4.72-4.70 (q, 1H), 4.65 (s, 2H), 3.87-3.84 (m, 2H), 3.70 (m, 1H), 3.60 (m, 1H), 3.43 (s, 3H), 2.66 (m, 2H), 1.69-1.68 (d, 3H).

### Example-112

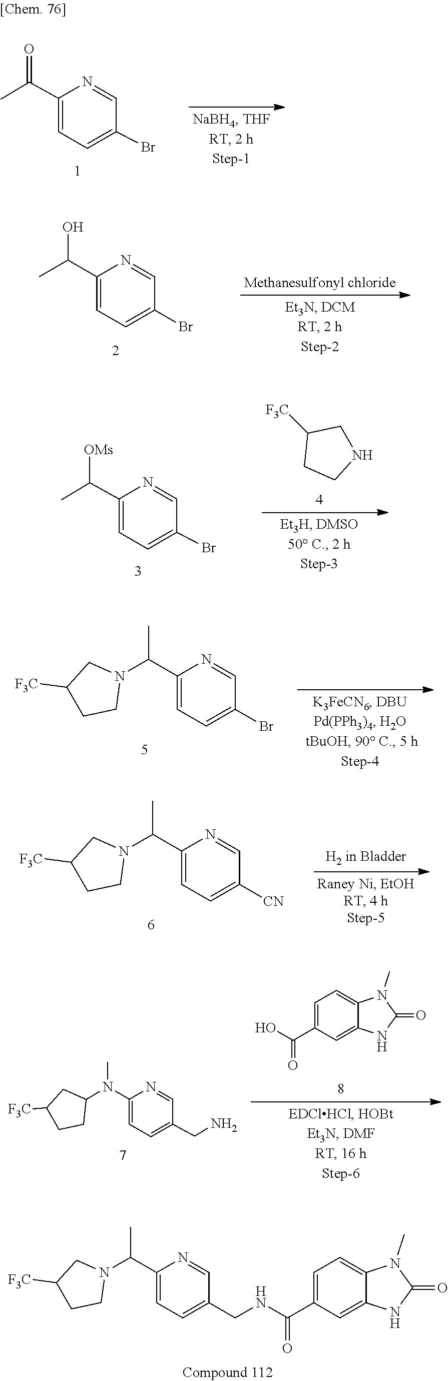

### Step-1: 1-(5-Bromopyridin-2-yl)ethan-1-ol

To a stirred solution of 1-(5-bromopyridin-2-yl)ethan-1-one (0.5 g, 2.5 mmol) in THF (5 mL) was added NaBH4 (0.19 g, 5.0 mmol) at 0° C. and the mixture was stirred at RT for 2 h. The reaction mass was concentrated to remove THF, quenched with ice cold water and extracted with EtOAc. The separated organic layer was washed with brine, dried over anhydrous Na₂SO₄ and concentrated to get the title compound (0.4 g 79%). LC-MS: 202 [M+H]⁺.

### Step-2: 1-(5-Bromopyridin-2-yl)ethyl methanesulfonate

To a stirred solution of 1-(5-bromopyridin-2-yl)ethan-1-ol (0.4 g, 1.98 mmol) in DCM (10 mL) was added triethylamine (0.4 g, 3.96 mmol) followed by methane-sulfonyl chloride (0.27 g, 2.37 mmol) and the mixture was stirred at RT for 2 h. The reaction mixture was diluted with DCM, washed with water, dried over anhydrous Na₂ SO₄ and concentrated. The residue was purified by combi flash column chromatography using 20% EtOAc in n-hexane as an eluent to get the title compound (0.48 g, 86%). LC-MS: 280 [M+H]⁺

### Step-3: 5-Bromo-2-(1-(3-(trifluoromethyl)pyrrolidin-1-yl)ethyl)pyridine

To a stirred solution of 1-(5-bromopyridin-2-yl)ethyl methanesulfonate (0.3 g, 1.07 mmol) in DMSO (5 mL) was added triethylamine (0.32 g, 3.21 mmol) followed by 3-(trifluoromethyl)pyrrolidine hydrochloride (0.23 g, 1.28 mmol) and the mixture was stirred at 50° C. for 2 h. The reaction mixture concentrated and the residue was purified by combi flash column chromatography using 20% EtOAc in n-hexane as an eluent to get the title compound (0.08 g, 23%). LC-MS: 323 [M+H]⁺.

### Step-4: 6-(1-(3-(Trifluoromethyl)pyrrolidin-1-yl)ethyl)nicotinonitrile

To a stirred solution of 5-Bromo-2-(1-(3-(trifluoromethyl)pyrrolidin-1-yl)ethyl)pyridine (0.08 g, 0.25 mmol) in t-BuOH (2 mL) and water (2 mL) was added DBU (0.01 g, 0.07 mmol) followed by potassium ferrocyanide trihydrate (0.05 g, 0.12 mmol). The reaction mixture was degassed for 15 mins with argon and tetrakis(triphenylphosphine)palladium (0.03 g, 0.02 mmol) was added thereto and the mixture was stirred at 90° C. for 5 h. The reaction mixture was cooled to RT, diluted with 10% MeOH in DCM and filtered through Celite. The filtrate was concentrated and purified by combi flash column chromatography using 20% EtOAc in n-hexane as an eluent to afford the title compound (0.06 g, 97%). LC-MS: 270 [M+H]⁺.

### Step-5: (6-(1-(3-(Trifluoromethyl)pyrrolidin-1-yl)ethyl)pyridin-3-yl)methanamine

To a stirred solution of 6-(1-(3-(trifluoromethyl)pyrrolidin-1-yl)ethyl)nicotinonitrile (0.06 g, 0.24 mmol) in EtOH (5 mL) was added Raney nickel (0.1 g) followed by 7 M ammonia in MeOH (2 mL) and the mixture was stirred at RT for 4 h under hydrogen bladder pressure. The reaction mass was filtered through Celite and the filtrate was concentrated to afford the title compound (0.06 g, 98%). LC-MS: 274 [M+H]⁺.

### Step-6: 1-Methyl-2-oxo-N-((6-(1-(3-(trifluoromethyl)pyrrolidin-1-yl)ethyl)pyridin-3-yl)methyl)-2,3-dihydro-1H-benzimidazole-5-carboxamide (Compound 112)

To a stirred solution of 1-methyl-2-oxo-2,3-dihydro-1H-benzimidazole-5-carboxylic acid (0.04 g, 0.20 mmol) in DMF (4 mL) was added EDC·HCl (0.04 g, 0.23 mmol) followed by HOBt (0.02 g, 0.12 mmol) and the mixture was stirred at RT for 10 min. To the reaction mixture was added (6-(1-(3-(trifluoromethyl)pyrrolidin-1-yl)ethyl)pyridin-3-yl)methanamine (0.07 g, 0.25 mmol) followed by triethylamine (0.13 g, 1.3 mmol) and the mixture was stirred at RT for 16 h. The reaction mixture was poured into ice cold water, extracted with EtOAc, dried over anhydrous Na₂SO₄ and concentrated. The residue was further purified by using preparative HPLC method C to get the title compound (0.02 g, 17%). LC-MS: 448 [M+H]⁺; ¹H-NMR (400 MHz, CD₃OD): δ 8.5 (s, 1H), 7.86-7.84 (dd, 1H), 7.7-7.68 (dd, 1H), 7.61 (d, 1H), 7.52-7.50 (dd, 1H), 7.2-7.18 (d, 1H), 4.62 (s, 2H), 3.55-3.53 (m, 1H), 3.44 (s, 3H), 3.0-2.5 (m, 5H), 2.15-1.85 (m, 2H), 1.44-1.42 (d, 3H).

### Example-113

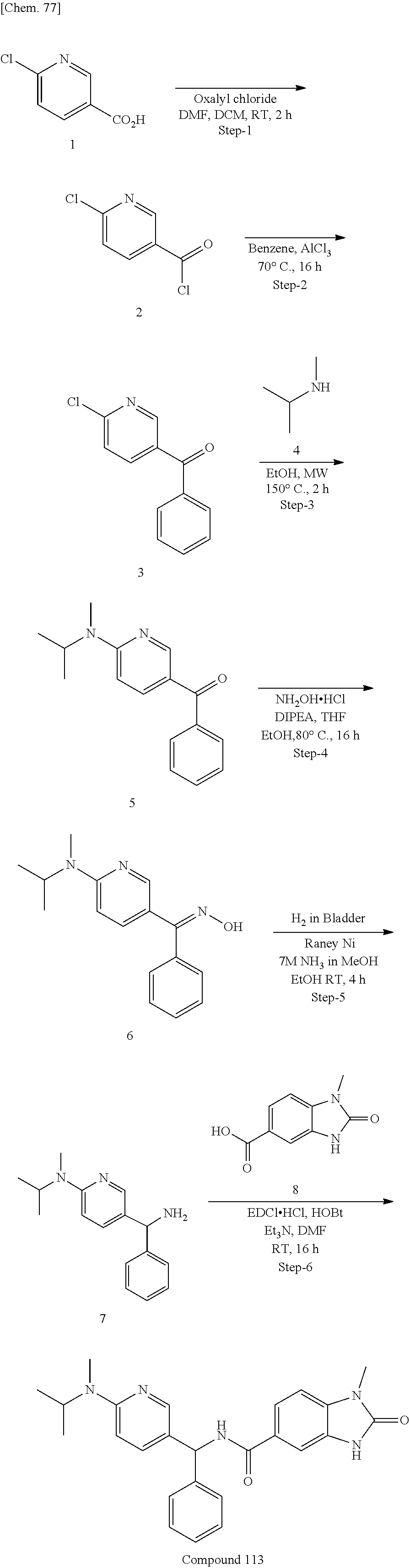

### Step-1: 6-Chloronicotinoyl chloride

To a stirred solution of 6-chloronicotinic acid (0.5 g, 3.17 mmol) in DCM (5 mL) was added oxalyl chloride (0.8 g, 6.3 mmol) at 0° C. To the mixture was added catalytic amount of DMF and stirred at RT for 2 h. The reaction mixture was concentrated under argon atmosphere to afford the title compound, which was further taken into next step without purification.

### Step-2: 6-Chloropyridin-3-yl(phenyl)methanone

To a stirred solution of 6-chloronicotinoyl chloride (0.5 g, 3.17 mmol) in benzene (15 mL) was added aluminum chloride (0.8 g, 6.3 mmol) at 0° C. and stirred at 70° C. for 16 h. The reaction mixture was poured into water, extracted with EtOAc, dried over anhydrous Na₂SO₄ and concentrated. This was purified by combi flash column chromatography using 20% EtOAc in n-hexane as an eluent to afford the title compound (0.5 g, 80.6%). LC-MS: 218 [M+H]⁺.

### Step-3: (6-(Isopropyl(methyl)amino)pyridin-3-yl)(phenyl)methanone

To a stirred solution of 6-chloropyridin-3-yl(phenyl)methanone (0.3 g, 1.37 mmol) in EtOH (5 mL) was added N-methylpropan-2-amine (0.5 g 6.8 mmol) and stirred at 150° C. for 2 h under microwave irradiation. The reaction mixture was poured into water, extracted with EtOAc, dried over anhydrous Na₂SO₄ and concentrated to afford the title compound (0.45 g, 128.2%). LC-MS: 255 [M+H]⁺.

### Step-4: N—[(E)-(6-(isopropylamino)pyridin-3-yl)(phenyl)methylidene]hydroxylamine

To a stirred solution of (6-(isopropyl(methyl)amino)pyridin-3-yl)(phenyl)methanone (0.32 g, 1.25 mmol) in EtOH (3 mL) and THF (3 mL) was added hydroxylamine hydrochloride (0.28 g, 4.0 mmol) followed by DIPEA (1.03 g, 8.0 mmol) and the mixture was stirred at 80° C. for 16 h. The reaction mass was concentrated to remove EtOH and THF. The residue was diluted with EtOAc, washed with water, dried over anhydrous Na₂SO₄, and concentrated to afford the title compound (0.38 g, 112%). LC-MS: 270 [M+H]⁺

### Step-5: 5-(Amino(phenyl)methyl)-N-isopropyl-N-methylpyridin-2-amine

To a stirred solution of N—[(E)-(6-(isopropylamino)pyridin-3-yl)(phenyl)methylidene]hydroxylamine (0.3 g, 1.1 mmol) in EtOH (10 mL) was added Raney nickel (0.32 g) followed by 7 M ammonia in MeOH (5 mL) and stirred at RT for 4 h under hydrogen bladder pressure. The reaction mass was filtered through Celite, and the filtrate was concentrated to afford the title compound (0.2 g, 70.2%). LC-MS: 256 [M+H]⁺.

### Step-6: N-((6-(Isopropyl(methyl)amino)pyridin-3-yl)(phenyl)methyl)-1-methyl-2-oxo-2,3-dihydr o-1H-benzimidazole-5-carboxamide (Compound 113)

To a stirred solution of 1-methyl-2-oxo-2,3-dihydro-1H-benzimidazole-5-carboxylic acid (0.15 g, 0.78 mmol) in DMF (4 mL) was added EDC·HCl (0.15 g, 0.78 mmol) followed by HOBt (0.06 g, 0.46 mmol) and the mixture was stirred at RT for 10 min. To the reaction mixture was added 5-(amino(phenyl)methyl)-N-isopropyl-N-methylpyridin-2-amine (0.2 g, 0.78 mmol) followed by triethylamine (0.47 g, 4.68 mmol) and the mixture was stirred at RT for 16 h. The reaction mixture was poured into ice cold water, extracted with EtOAc, dried over anhydrous Na₂SO₄ and concentrated. The residue was purified by preparative HPLC using method C to afford the title compound (0.1 g, 29.7%). LC-MS: 430 [M+H]⁺; ¹H-NMR (400 MHz, CD₃OD): δ 7.93-7.91 (d, 1H), 7.74-7.68 (bs, 2H), 7.64 (bs, 1H), 7.41-7.28 (m, 6H), 7.12-7.10 (d, 1H), 6.37 (bs, 1H), 4.43-4.34 (m, 1H), 3.34 (s, 3H), 3.05 (s, 3H), 1.29-1.27 (d, 6H).

### Example-114

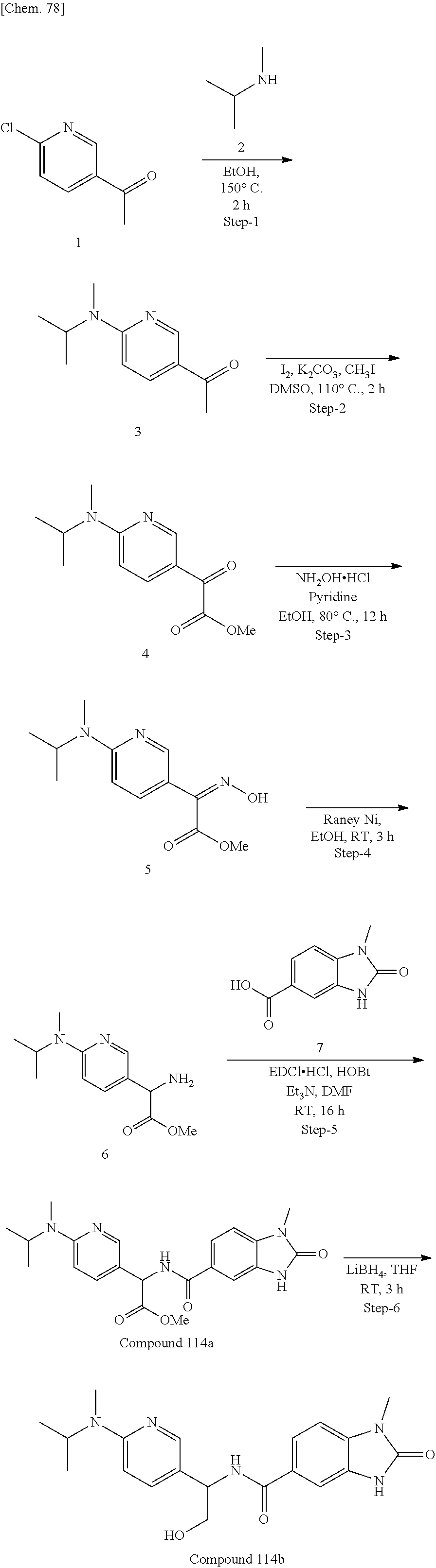

### Step-1: 1-(6-(Isopropyl(methyl)amino)pyridin-3-yl)ethan-1-one

To a stirred solution of 1-(6-chloropyridin-3-yl)ethan-1-one (2.0 g, 12.85 mmol) in EtOH (5 mL) was added N-methylpropan-2-amine (2.82 g, 38.56 mmol) and stirred at 150° C. for 2 h under microwave irradiation. The reaction mixture was concentrated and the residue was purified by combi flash column chromatography using 20% EtOAc in n-hexane as an eluent to get title compound (2.0 g, 81%). LC-MS: 193 [M+H]⁺.

### Step-2: Methyl 2-(6-(isopropyl(methyl)amino)pyridin-3-yl)-2-oxoacetate

To a stirred solution of 1-(6-(isopropyl(methyl)amino)pyridin-3-yl)ethan-1-one (0.5 g, 2.60 mmol) in DMSO (10 mL) was added iodine (1.98 g, 7.8 mmol) followed by addition of K₂CO₃ (2.15 g, 15.6 mmol) and the mixture was stirred at 110° C. for 2 h. The reaction mixture was cooled to RT, iodomethane (0.44 g, 3.12 mmol) was added thereto and the mixture was stirred at 35° C. for 1 h. The reaction mixture was quenched with saturated sodium sulfite solution and extracted with toluene. The separated organic layer was washed with brine, dried over anhydrous Na₂SO₄ and concentrated. The residue was purified by Preparative TLC using 20% EtOAc in n-hexane as an eluent to get the title compound (0.2 g, 32%). LC-MS: 237 [M+H]⁺.

### Step-3: Methyl (Z)-2-(hydroxyimino)-2-(6-(isopropyl(methyl)amino)pyridin-3-yl)acetate

To a stirred solution of methyl 2-(6-(isopropyl(methyl)amino)pyridin-3-yl)-2-oxoacetate (0.2 g, 0.84 mmol) in EtOH (5 mL) was added hydroxylamine hydrochloride (0.12 g, 1.69 mmol) followed by pyridine (0.13 g, 1.69 mmol) and the mixture was stirred at 80° C. for 12 h. The reaction mixture was concentrated to get the title compound (0.22 g). LC-MS: 252 [M+H]⁺.

### Step-4: Methyl 2-amino-2-(6-(isopropyl(methyl)amino)pyridin-3-yl)acetate

To a stirred solution of methyl (Z)-2-(hydroxyimino)-2-(6-(isopropyl(methyl)amino)pyridin-3-yl)acetate (0.22 g) in EtOH (5 mL) was added Raney nickel (0.25 g) and the mixture was stirred under hydrogen pressure for 3 h at RT. The reaction mass was filtered through Celite and the filtrate was concentrated to afford the title compound (0.2 g). LC-MS: 238 [M+H]⁺.

### Step-5: Methyl 2-(6-(isopropyl(methyl)amino)pyridin-3-yl)-2-(1-methyl-2-oxo-2,3-dihydro-1H-benzi midazole-5-carboxamido)acetate (Compound 114a)

To a stirred solution of 1-methyl-2-oxo-2,3-dihydro-1H-benzimidazole-5-carboxylic acid (0.13 g, 0.67 mmol) in DMF (5 mL) was added EDC·HCl (0.14 g, 0.75 mmol) followed by HOBt (0.05 g, 0.37 mmol) and the mixture was stirred at RT for 10 min. To the reaction mixture was added methyl 2-amino-2-(6-(isopropyl(methyl)amino)pyridin-3-yl)acetate (0.2 g, 0.84 mmol) followed by triethylamine (0.42 g, 4.21 mmol) and the mixture was stirred at RT for 16 h. The reaction mixture was poured into ice cold water, extracted with EtOAc, dried over anhydrous Na₂SO₄ and concentrated (0.15 g). The residue (0.1 g) was purified using preparative HPLC method C to get the title compound (0.03 g). LC-MS: 412 [M+H]⁺; ¹H-NMR (400 MHz, CD₃OD): δ 8.07-8.02 (dd, 2H), 7.72-7.70 (d, 1H), 7.61-7.60 (d, 1H), 7.33-7.31 (d, 1H), 7.22-7.20 (d, 1H), 5.75 (s, 1H), 4.47 (m, 1H), 3.82 (s, 3H), 3.44 (s, 3H), 3.08 (s, 3H), 1.34-1.33 (s, 6H).

### Step-6: N-(2-hydroxy-1-(6-(Isopropyl(methyl)amino)pyridin-3-yl)ethyl)-1-methyl-2-oxo-2,3-dih ydro-1H-benzimidazole-5-carboxamide (Compound 114b)

To a stirred solution of methyl 2-(6-(isopropyl(methyl)amino)pyridin-3-yl)-2-(1-methyl-2-oxo-2,3-dihydro-1H-benzi midazole-5-carboxamido)acetate (0.04 g, 0.41 mmol) in THF (2 mL) was added LiBH₄ (0.004 g, 0.19 mmol) at 0° C. and the mixture was stirred at RT for 3 h. The reaction mixture was quenched with saturated sodium sulfite solution and extracted with EtOAc. The separated organic layer was washed, dried over anhydrous Na₂SO₄ and concentrated. The residue was purified by Preparative TLC using 5% MeOH in DCM as an eluent to get the title compound (0.02 g, 53%). LC-MS: 384 [M+H]⁺; ¹H-NMR (400 MHz, CD₃OD): δ 8.11-8.10 (d, 1H), 7.72-7.69 (dd, 1H), 7.62-7.59 (dd, 2H), 7.19-7.18 (d, 1H), 6.69-6.66 (d, 1H), 5.12-5.09 (m, 1H), 4.73-4.70 (m, 1H), 3.89-3.85 (m, 2H), 3.44 (s, 3H), 2.85 (s, 3H), 1.20-1.18 (d, 6H).

### Example-115

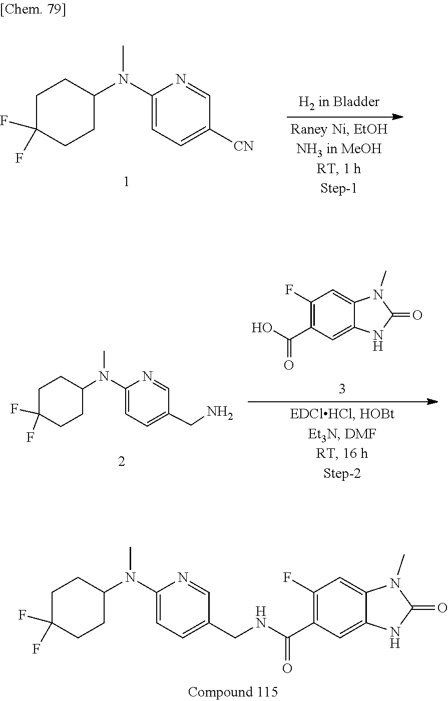 [SMILES: CN(c1ccc(C#N)cn1)C1CCC(F)(F)CC1 ; CN(c1ccc(CN)cn1)C1CCC(F)(F)CC1 ; Cn1c(=O)[nH]c2cc(C(=O)O)c(F)cc21 ; CN(c1ccc(CNC(=O)c2cc3[nH]c(=O)n(C)c3cc2F)cn1)C1CCC(F)(F)CC1]

### Step-1: 5-(Aminomethyl)-N-(4,4-difluorocyclohexyl)-N-methylpyridin-2-amine

To a stirred solution of 6-((4,4-difluorocyclohexyl)(methyl)amino)nicotinonitrile (0.1 g, 0.39 mmol) in EtOH (5 mL) was added Raney nickel (0.1 g) followed by 7 M ammonia in MeOH (2 mL) and stirred at RT for 1 h under hydrogen bladder pressure. The reaction mass was filtered through Celite and the filtrate was concentrated to afford the title compound (0.09 g, 89%). LC-MS: 256 [M+H]⁺.

### Step-2: N-((6-((4,4-Difluorocyclohexyl)(methyl)amino)pyridin-3-yl)methyl)-6-fluoro-1-methyl-2-oxo-2,3-dihydro-1H-benzimidazole-5-carboxamide (Compound 115)

To a stirred solution of 6-fluoro-1-methyl-2-oxo-2,3-dihydro-1H-benzimidazole-5-carboxylic acid (0.16 g, 0.78 mmol) in DMF (4 mL) was added EDC·HCl (0.15 g, 0.78 mmol) followed by HOBt (0.05 g, 0.39 mmol) and the mixture was stirred at RT for 10 min. To the reaction mixture was added 5-(aminomethyl)-N-(4,4-difluorocyclohexyl)-N-methylpyridin-2-amine (0.2 g, 0.78 mmol) followed by triethylamine (0.24 g, 2.34 mmol) and the mixture was stirred at RT for 16 h. The reaction mixture was poured into ice cold water, extracted with EtOAc, dried over anhydrous Na₂SO₄ and concentrated. The residue was purified by preparative HPLC method C, to afford the title compound (0.03 g, 10%). LC-MS: 448 [M+H]⁺; ¹H-NMR (400 MHz, DMSO-d₆): δ 11.03 (s, 1H), 8.55-8.45 (m, 1H), 8.06 (s, 1H), 7.52-7.5 (dd, 1H), 7.20-7.14 (m, 2H), 6.67-6.65 (d, 1H), 4.64-4.58 (m, 1H), 4.31-4.29 (d, 2H), 3.26 (s, 3H), 2.81 (s, 3H), 2.1-2.07 (m, 4H), 1.8-1.6 (m, 4H).

### Example-116

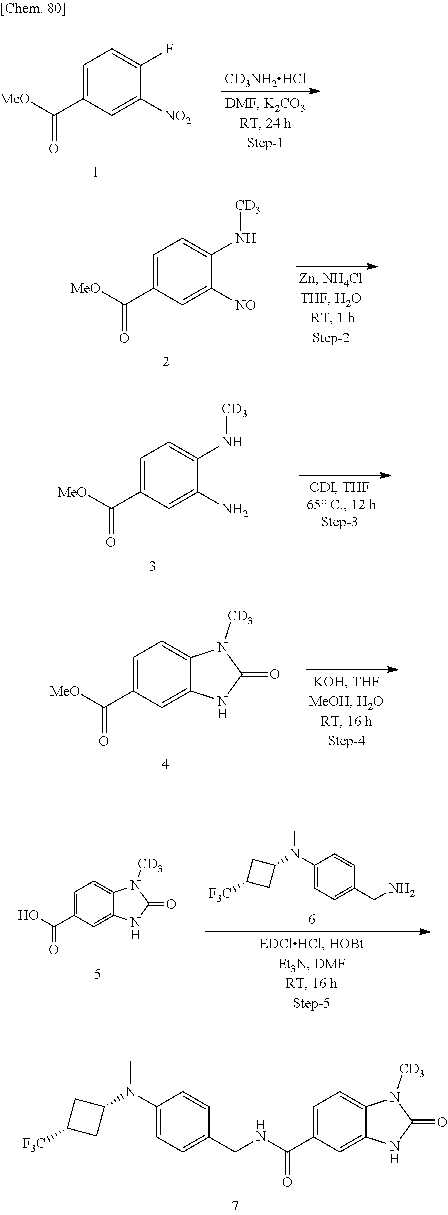

### Step-1: Methyl 4-((trideuteromethyl)amino)-3-nitrobenzoate

To a stirred solution of methyl 4-fluoro-3-nitrobenzoate (0.5 g, 2.51 mmol) in DMF (8 mL) was added K₂CO₃ (0.69 g, 5.02 mmol) followed by methan-d₃-amine hydrochloride (0.18 g, 2.51 mmol) at 10° C. and the mixture was stirred at RT for 24 h. The reaction mixture was poured into ice cold water, and the solid separated was collected by filtration and dried to afford the title compound (0.5 g, 93%). LC-MS: 214 [M+H]⁺.

### Step-2: Methyl 3-amino-4-((trideuteromethyl)amino)benzoate

To a stirred solution of methyl 4-((trideuteromethyl)amino)-3-nitrobenzoate (0.09 g, 0.42 mmol) in THF (2 mL) and water (1 mL) was added ammonium chloride (0.23 g, 4.2 mmol) followed by zinc dust (0.22 g, 3.36 mmol) and the mixture was stirred at RT for 1 h. The reaction mass was diluted with EtOAc and filtered through Celite bed. The filtrate was washed with water, dried over anhydrous Na₂SO₄ and concentrated to afford the title compound (0.07 g, 90%). LC-MS: 184 [M+H]⁺.

### Step-3: Methyl 1-(trideuteromethyl)-2-oxo-2,3-dihydro-1H-benzimidazole-5-carboxylate

To a stirred solution of methyl 3-amino-4-((trideuteromethyl)amino)benzoate (0.07 g, 0.42 mmol) in THF (2 mL) was added CDI (0.13 g, 0.84 mmol) and the mixture was stirred at 65° C. for 12 h. The reaction mixture was poured into ice cold water, and the solid separated was collected by filtration and dried to afford the title compound (0.05 g, 57%). LC-MS: 210 [M+H]⁺

### Step-4: 1-(Trideuteromethyl)-2-oxo-2,3-dihydro-1H-benzimidazole-5-carboxylic acid

To a stirred solution of methyl 1-(trideuteromethyl)-2-oxo-2,3-dihydro-1H-benzimidazole-5-carboxylate (0.05 g, 0.24 mmol) in THF (2 mL) and MeOH (1 mL) was added KOH (0.02 g, 0.36 mmol) in water (1 mL) and stirred at RT for 16 h. The reaction mixture was concentrated to remove THF and MeOH, diluted with water and acidified with 1 N HCl. The precipitate formed was collected by filtration and dried to get the title compound (0.04 g, 85%). LC-MS: 196 [M+H]⁺

### Step-5: 1-(Trideuteromethyl)-N-((6-(methyl(cis-3-(trifluoromethyl)cyclobutyl)amino)pyridin-3-yl)methyl)-2-oxo-2,3-dihydro-1H-benzimidazole-5-carboxamide (Compound 116)

To a solution of 1-(trideuteromethyl)-2-oxo-2,3-dihydro-1H-benzimidazole-5-carboxylic acid (0.04 g, 0.2 mmol) in DMF (2 mL) was added EDC·HCl (0.04 g, 0.2 mmol), HOBt (0.01 g, 0.07 mmol) and the mixture was stirred at RT for 10 minutes. To the reaction mixture was added 5-(aminomethyl)-N-methyl-N-(cis-3-(trifluoromethyl)cyclobutyl)pyridin-2-amine (0.06 g, 0.23 mmol) followed by triethylamine (0.12 g, 1.2 mmol) and the mixture was stirred at RT for 16 h. The reaction mixture was poured into ice cold water, and the precipitate formed was collected by filtration and dried. This was sonicated and stirred with 10% DCM in n-hexane (10 mL) for 30 mins, then collected by filtration and dried to afford the title compound (0.04 g, 36%). LC-MS: 437 [M+H]⁺; ¹H-NMR (400 MHz, CD3OD): δ 8.11 (d, 1H), 7.66-7.58 (m, 3H), 7.18-7.16 (d, 1H), 6.7-6.68 (d, 1H), 4.8-4.6 (m, 1H), 4.45 (s, 2H), 3.0 (s, 3H), 2.82-2.80 (m, 1H), 2.5-2.46 (m, 2H), 2.34-2.30 (m, 2H).

### Prep HPLC Methods:

Method A: Column-LUNA C18 (250 mm×19 mm), 5.0μ; Eluent-A 0.05% TFA in water, B-ACN, gradient-10% B at 0 min, 20% B at 2 min, 50% at 10 min, 70% at 15 min), Flow: 18 mL/min
Method B: Column—Waters, Xselect, c18 (250 mm×21.2 mm), 5.0μ; Eluent-A 0.05% TFA in water, B-ACN, gradient-10% B at 0 min, 30% B at 2 min, 70% at 8 min), Flow: 13 mL/min
Method C: Column-LUNA C18 (250 mm×19 mm), 5.0μ; Eluent-A 0.1% FA in water, B-ACN, gradient-10% B at 0 min, 20% B at 2 min, 50% at 10 min, 70% at 15 min), Flow: 18 mL/min
Method D: Column-LUNA C18 (250 mm×19 mm), 5.0μ; Eluent-A 0.1% HCl in water, B-ACN, gradient-10% B at 0 min, 20% B at 2 min, 50% at 10 min, 70% at 15 min), Flow: 18 mL/min

### Examples-28 to 102 and 117 to 147

Compounds of Examples (Ex.)-28 to 102 and 117 to 147 shown in Tables 2 to 97 were prepared according to a similar manner to that of the referred Example and step. For example, the term “E4-S4” in the column “Ref.” of the table shows that the compound was prepared according to a similar manner to that of referred “E”xample-“4”-“S”tep-“4” above. Likewise, the term “P5-S1” in the column “Ref.” of the table shows that the compound was prepared according to a similar manner to that of referred “P”reparation-“5”-“S”tep-“1” above. It is understood that compounds can be obtained as a free form or a salt form due to isolation condition.

The compound having a chemical structural formula with “HCl” in the vicinity thereof represents that the compound was isolated as hydrochloride.

The compound having a chemical structural formula marked with “*” represents that the compound is an isomer having the configuration of the represented structure.

The obtained Compounds 82 and 130 were separated to obtain two optical isomers of the compounds as Compound 82 Peak 1 and Compound 82 Peak 2; and Compound 130 Peak 1 and Compound 130 Peak 2; respectively.

(Preparative HPLC method: Column—Chiral pak-IG (250×21.2 mm) 5μ; Eluent: A: n-Hexane, B: ETOH; Isocratic: A:B (50:50); Flow: 15 mL/min).

TABLE 2

| Ex. | Ref. | Structure | LC-MS, NMR |
|---|---|---|---|
| 28 | E4-S4 | 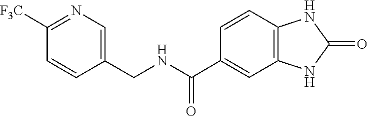 [SMILES: O=C(NCc1ccc(C(F)(F)F)nc1)c1ccc2[nH]c(=O)[nH]c2c1] | LC-MS: 337.0 [M + H]⁺; ¹H-NMR (400 MHz, DMSO-D₆) δ 10.59 (d, 2H), 9.07-9.04 (t, 1H), 8.73 (s, 1H), 7.99-7.97 (d, 1H), 7.88-7.86 (d, 1H), 7.574-7.57 (d, 1H), 7.47 (s, 1H), 6.99-6.97 (d, 1H), 4.57-4.56 (d, 2H). |
| | | (Compound 28) | |
| 29 | E4-S4 | 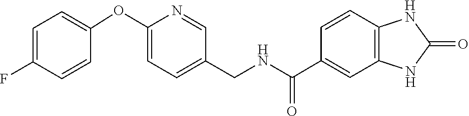 [SMILES: O=C(NCc1ccc(Oc2ccc(F)cc2)nc1)c1ccc2[nH]c(=O)[nH]c2c1] | LC-MS: 378.95 [M+H]⁺; ¹H-NMR (400 MHz, DMSO-D₆) δ 10.88-10.85 (d, 2H), 8.92-8.91 (t, 1H), 8.08 (s, 1H), 7.78-7.77 (d, 1H), 7.52 (d, 1H), 7.45 (s, 1H), 7.25-7.22 (m, 2H), 7.16-7.13 (m, 2H), 7.01- 6.94 (m, 2H), 4.40-4.39 (d, 2H). |
| | | (Compound 29) | |

TABLE 3

| Ex. | Ref. | Structure | LC-MS, NMR |
|---|---|---|---|
| 30 | E4-S1 | 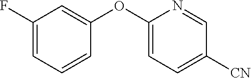 [SMILES: N#Cc1ccc(Oc2cccc(F)c2)nc1] | LC-MS: 215 [M + H]⁺ |
| | E10-S3 | 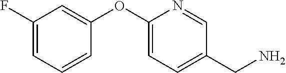 [SMILES: NCc1ccc(Oc2cccc(F)c2)nc1] | LC-MS: 219 [M + H]⁺ |
| | E4-S4 | 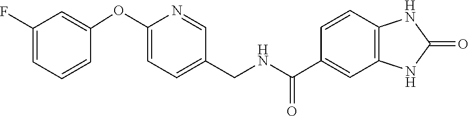 [SMILES: O=C(NCc1ccc(Oc2cccc(F)c2)nc1)c1ccc2[nH]c(=O)[nH]c2c1] | LC-MS: 378.95 [M + H]⁻; ¹H-NMR (400 MHz, DMSO-D₆) δ 10.90-10.86 (d, 2H), 8.95-8.92 (t, 1H), 8.13 (s, 1H), 7.818-7.812 (d, 1H), 7.553-7.532 (d, 1H), 7.46-7.42 (m, 2H), 7.06-7.04 (m, 3H), 7.01- 6.94 (m, 2H), 4.43-4.41 (d, 2H). |
| | | (Compound 30) | |
| 31 | E4-S1 | 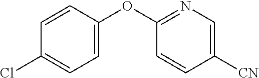 [SMILES: N#Cc1ccc(Oc2ccc(Cl)cc2)nc1] | LC-MS: 231 [M + H]⁺ |
| | E10-S3 | 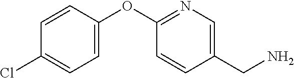 [SMILES: NCc1ccc(Oc2ccc(Cl)cc2)nc1] | LC-MS: 235 [M + H]⁺ |
| | E4-S4 | 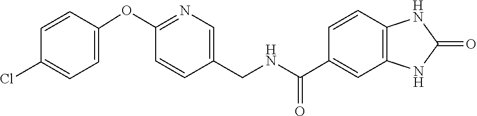 [SMILES: O=C(NCc1ccc(Oc2ccc(Cl)cc2)nc1)c1ccc2[nH]c(=O)[nH]c2c1] | LC-MS: 394.95 [M + H]⁻; ¹H-NMR (400 MHz, DMSO-D₆) δ 10.88-10.85 (d, 2H), 8.915 (t, 1H), 8.01 (s, 1H), 7.82-7.79 (d, 1H), 7.525- 7.522 (d, 1H), 7.45-7.43 (d, 3H), 7.14- 7.12 (d, 2H), 7.04-7.02 (d, 1H), 6.97- 6.94 (d, 1H), 4.41-4.40 (d, 2H). |
| | | (Compound 31) | |

TABLE 4

| Ex. | Ref. | Structure | LC-MS, NMR |
|---|---|---|---|
| 32 | E4-S4 | 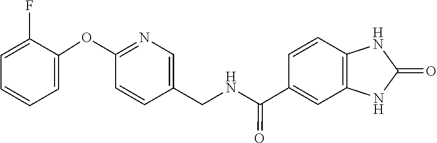 [SMILES: O=C(NCc1ccc(Oc2ccccc2F)nc1)c1ccc2[nH]c(=O)[nH]c2c1] | LC-MS: 378.95 [M + H]⁺; ¹H-NMR (400 MHz, DMSO-D₆) δ 10.85 (d, 2H), 8.89 (t, 1H), 8.03 (s, 1H), 7.82-7.80 (d, 1H), 7.54-7.52 (d, 1H), 7.45 (s, 1H), 7.36-7.21 (m, 4H), 7.09-7.07 (d, 1H), 6.96-6.94 (d, 1H), 4.41-4.39 (d, 2H). |
| | | (Compound 32) | |
| 33 | E4-S4 | 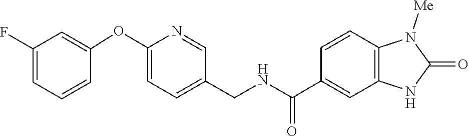 [SMILES: Cn1c(=O)[nH]c2cc(C(=O)NCc3ccc(Oc4cccc(F)c4)nc3)ccc21] | LC-MS: 393.0 [M + H]⁺; ¹H-NMR (400 MHz, DMSO-D₆) δ 11.09 (s, 1H), 8.98-8.95 (t, 1H), 8.13-8.12 (s, 1H), 7.84-7.81 (d, 1H), 7.62-7.61 (d, 1H), 7.51 (s, 1H), 7.42 (m, 1H), 7.16-7.14 (d, 1H), 7.06-7.01 (m, 3H), 6.96-6.93 (d, 1H), 4.43-4.42 (d, 2H), 3.30 (s, 3H). |
| | | (Compound 33) | |

TABLE 5

| Ex. | Ref. | Structure | LC-MS, NMR |
|---|---|---|---|
| 34 | E4-S4 | 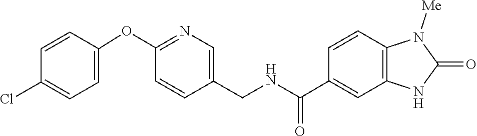 [SMILES: Cn1c(=O)[nH]c2cc(C(=O)NCc3ccc(Oc4ccc(Cl)cc4)nc3)ccc21] | LC-MS: 409.0 [M + H]⁺; ¹H-NMR (400 MHz, DMSO-D₆) δ 11.05 (s, 1H), 9.92 (t, 1H), 8.09 (s, 1H), 7.81-7.79 (d, 1H), 7.62-7.60 (d, 1H), 7.49 (s, 1H), 7.44-7.42 (d, 2H), 7.14- 7.11 (m, 3H), 7.03-7.01 (d, 1H), 4.41- 4.40 (d, 2H), 3.37 (s, 3H). |
| | | (Compound 34) | |

TABLE 6

| Ex. | Ref. | Structure | LC-MS, NMR |
|---|---|---|---|
| 35 | E2-S1 | 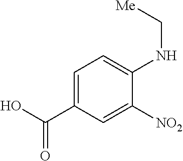 [SMILES: CCNc1ccc(C(=O)O)cc1[N+](=O)[O-]] | LC-MS: 209 [M − H]− |
| | E2-S2 | 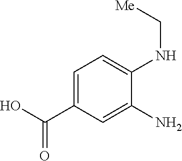 [SMILES: CCNc1ccc(C(=O)O)cc1N] | LC-MS: 181 [M + H]⁺ |
| | E2-S3 | 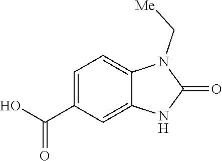 [SMILES: CCn1c(=O)[nH]c2cc(C(=O)O)ccc21] | LC-MS: 207 [M + H]⁺ |
| | E2-S4 | 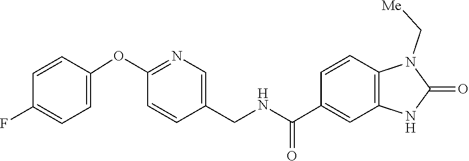 [SMILES: CCn1c(=O)[nH]c2cc(C(=O)NCc3ccc(Oc4ccc(F)cc4)nc3)ccc21] | LC-MS: 407.2 [M + H]⁺; ¹H-NMR (400 MHz, DMSO-D₆) δ 11.08 (s, 1H), 8.95 (t, 1H), 8.095-8.090 (d, 1H), 7.81-7.78 (dd, 1H), 7.63-7.61 (dd, 1H), 7.51 (s, 1H), 7.25-7.13 (m, 5H), 7.012-6.99 (d, 1H), 4.42- 4.41 (d, 2H), 3.87-3.82 (q, 2H), 1.21-1.18 (t, 3H). |
| | | (Compound 35) | |

TABLE 7

| Ex. | Ref. | Structure | LC-MS, NMR |
|---|---|---|---|
| 36 | E4-S1 | 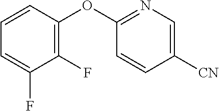 [SMILES: N#Cc1ccc(Oc2cccc(F)c2F)nc1] | LC-MS: 233 [M + H]⁺ |
| | E4-S2 | 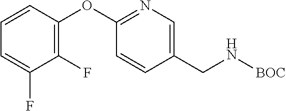 [SMILES: CC(C)(C)OC(=O)NCc1ccc(Oc2cccc(F)c2F)nc1] | LC-MS: 337 [M + H]⁺ |
| | E4-S3 | 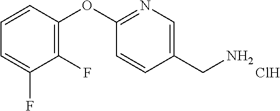 [SMILES: NCc1ccc(Oc2cccc(F)c2F)nc1 ; Cl] | LC-MS: 237 [M + H]⁺ |
| | E4-S4 | 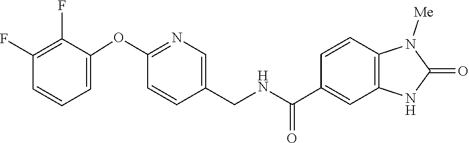 [SMILES: Cn1c(=O)[nH]c2cc(C(=O)NCc3ccc(Oc4cccc(F)c4F)nc3)ccc21] | LC-MS: 410.95 [M + H]⁺; ¹H-NMR (400 MHz, DMSO-D₆) δ 11.09 (s, 1H), 8.955 (t, 1H), 8.069- 8.064 (d, 1H), 7.864-7.837 (dd, 1H), 7.637-7.613 (dd, 1H), 7.507 (s, 1H), 7.4-7.3 (m, 1H), 7.3-7.2 (m, 1H), 7.174-7.142 (d, 3H), 4.429-4.414 (d, 2H), 3.301 (s, 3H). |
| | | (Compound 36) | |

TABLE 8

| Ex. | Ref. | Structure | LC-MS, NMR |
|---|---|---|---|
| 37 | E4-S1 | 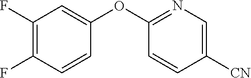 [SMILES: N#Cc1ccc(Oc2ccc(F)c(F)c2)nc1] | LC-MS: 233 [M + H]⁺ |
| | E4-S2 | 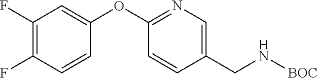 [SMILES: CC(C)(C)OC(=O)NCc1ccc(Oc2ccc(F)c(F)c2)nc1] | ¹H-NMR (400 MHz, CDCl₃) δ 8.07-8.07 (d, 1H), 7.6-7.7 (d, 1H), 7.17 (q, 1H), 6.99-7.00 (m, 1H), 6.92-6.89 (d, 1H), 6.90-6.88 |
| | | | (m, 1H), 4.85 (bs, 1H), |
| | | | 4.28-4.27 (d, 2H), 1.50 (s, |
| | | | 9H). |
| | E4-S3 | 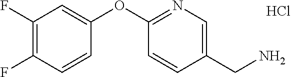 [SMILES: NCc1ccc(Oc2ccc(F)c(F)c2)nc1 ; Cl] | LC-MS: 237 [M + H]⁺ |
| | E4-S4 | 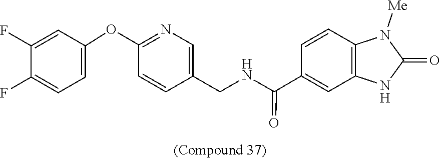 [SMILES: Cn1c(=O)[nH]c2cc(C(=O)NCc3ccc(Oc4ccc(F)c(F)c4)nc3)ccc21] | LC-MS: 411.2 [M + H]⁺; ¹H-NMR (400 MHz, CD₃OD) δ 8.15 (s, 1H), 7.87 (d, 1H), 7.65 (d, 1H), 7.577 (s, 1H), 7.3-7.25 (m, 1H), 7.18 (d, 1H), 7.1 (m, 1H), 7.0 (d, 1H), 6.9 (m, 1H), 4.544 (s, 2H), 3.416 (s, 3H). |

TABLE 9

| Ex. | Ref. | Structure | LC-MS, NMR |
|---|---|---|---|
| 38 | E4-S1 | 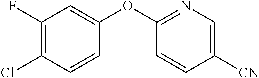 [SMILES: N#Cc1ccc(Oc2ccc(Cl)c(F)c2)nc1] | LC-MS: 249 [M + H]⁺ |
| | E4-S2 | 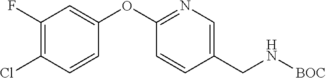 [SMILES: CC(C)(C)OC(=O)NCc1ccc(Oc2ccc(Cl)c(F)c2)nc1] | LC-MS: 353 [M + H]⁺ |
| | E4-S3 | 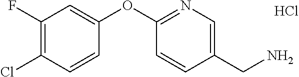 [SMILES: NCc1ccc(Oc2ccc(Cl)c(F)c2)nc1 ; Cl] | ¹H-NMR (400 MHz, DMSO-D₆) δ 8.47 (bs, 2H), 8.26 (d, 1H), 8.07-8.04 (d, 1H), 7.66-7.62 (m, 1H), 7.36-7.32 (dd, 1H), 7.19- 7.17 (d, 1H), 7.05-7.03 (d, 1H), 4.03-4.02 (d, 2H). |
| | E4-S4 | 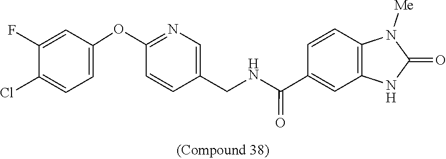 [SMILES: Cn1c(=O)[nH]c2cc(C(=O)NCc3ccc(Oc4ccc(Cl)c(F)c4)nc3)ccc21] | LC-MS: 427.3 [M + H]⁺; ¹H-NMR (400 MHz, DMSO-D₆) δ 11.096 (s, 1H), 8.967 (t, 1H), 8.128 (s, 1H), 7.856-7.829 (dd, 1H), 7.636-7.585 (m, 2H), 7.506 (s, 1H), 7.332-7.306 (dd, 1H), 7.165-7.144 (d, 1H), 7.095-7.073 (d, 1H), 7.027- 7.024 (d, 1H), 4.438-4.423 (d, 2H), 3.302 (s, 3H). |

TABLE 10

| Ex. | Ref. | Structure | LC-MS, NMR |
|---|---|---|---|
| 39 | E4-S1 | 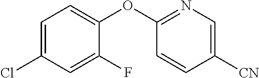 [SMILES: N#Cc1ccc(Oc2ccc(Cl)cc2F)nc1] | LC-MS: 249 [M + H]⁺ |
| | E4-S2 | 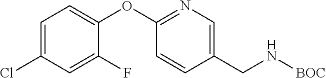 [SMILES: CC(C)(C)OC(=O)NCc1ccc(Oc2ccc(Cl)cc2F)nc1] | LC-MS: 353 [M + H]⁺ |
| | E4-S3 | 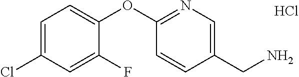 [SMILES: NCc1ccc(Oc2ccc(Cl)cc2F)nc1 ; Cl] | ¹H-NMR (400 MHz, DMSO-D₆) δ 8.47 (bs, 2H), 8.26-8.25 (d, 1H), 8.06-8.04 (d, 1H), 7.66-7.62 (m, 1H), 7.35-7.32 (dd, 1H), 7.19- 7.16 (d, 1H), 7.04-7.02 (d, 1H), 4.03-4.01 (d, 2H). |
| | E4-S4 | 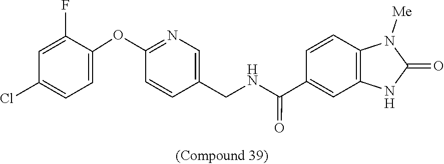 [SMILES: Cn1c(=O)[nH]c2cc(C(=O)NCc3ccc(Oc4ccc(Cl)cc4F)nc3)ccc21] | LC-MS: 427.1 [M + H]⁺; ¹H-NMR (400 MHz, DMSO-D₆) δ 11.088 (s, 1H), 8.947 (t, 1H), 8.044 (s, 1H), 7.845-7.824 (dd, 1H), 7.630-7.590 (m, 2H), 7.501 (s, 1H), 7.357-7.338 (m, 2H), 7.159-7.144 (t, 2H), 4.420-4.405 (d, 2H), 3.299 (s, 3H). |

TABLE 11

| Ex. | Ref. | Structure | LC-MS, NMR |
|---|---|---|---|
| 40 | E4-S1 | 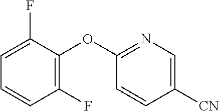 [SMILES: N#Cc1ccc(Oc2c(F)cccc2F)nc1] | ¹H-NMR (400 MHz, DMSO-D₆) δ 8.66-8.56 (d, 1H), 8.40-8.33 (dd, 1H), 7.51-7.49 (d, 1H), 7.43-7.37 (q, 1H), 7.39-7.28 (t, 2H). |
| | E4-S2 | 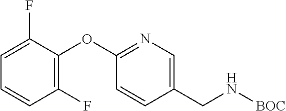 [SMILES: CC(C)(C)OC(=O)NCc1ccc(Oc2c(F)cccc2F)nc1] | LC-MS: 337 [M + H]⁺ |
| | E4-S3 | 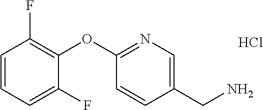 [SMILES: NCc1ccc(Oc2c(F)cccc2F)nc1 ; Cl] | LC-MS: 237 [M + H]⁺ |
| | E4-S4 | 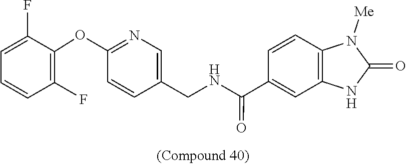 [SMILES: Cn1c(=O)[nH]c2cc(C(=O)NCc3ccc(Oc4c(F)cccc4F)nc3)ccc21] | LC-MS: 411.2 [M + H]⁺; ¹H-NMR (400 MHz, DMSO-D₆) δ 11.075 (s, 1H), 8.934 (t, 1H), 8.028- 8.023 (d, 1H), 7.862-7.835 (dd, 1H), 7.626-7.601 (dd, 1H), 7.496-7.493 (d, 1H), 7.309 (m, 1H), 7.266-7.246 (m, 2H), 7.203-7.182 (d, 1H), 7.148-7.128 (d, 1H), 4.415-4.401 (d, 2H), 3.290 (s, 3H). |

TABLE 12

| Ex. | Ref. | Structure | LC-MS, NMR |
|---|---|---|---|
| 41 | E4-S1 | 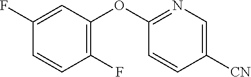 [SMILES: N#Cc1ccc(Oc2cc(F)ccc2F)nc1] | LC-MS: 233 [M + H]⁺ |
| | E4-S2 | 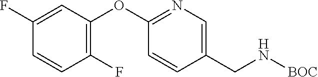 [SMILES: CC(C)(C)OC(=O)NCc1ccc(Oc2cc(F)ccc2F)nc1] | LC-MS: 337 [M + H]⁺ |
| | E4-S3 | 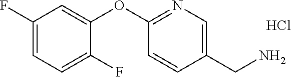 [SMILES: NCc1ccc(Oc2cc(F)ccc2F)nc1 ; Cl] | LC-MS: 237 [M + H]⁺ |
| | E4-S4 | 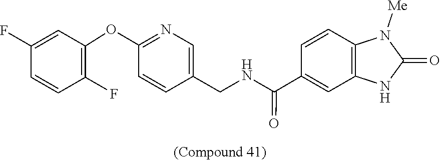 [SMILES: Cn1c(=O)[nH]c2cc(C(=O)NCc3ccc(Oc4cc(F)ccc4F)nc3)ccc21] | LC-MS: 411.0 [M + H]⁺; ¹H-NMR (400 MHz, DMSO-D₆) δ 11.05 (s, 1H), 8.94 (t, 1H), 8.05 (d, 1H), 7.84-7.81 (dd, 1H), 7.62- 7.60 (d, 1H), 7.49 (s, 1H), 7.43-7.37 (m, 1H), 7.32- 7.27 (m, 1H), 7.16-7.10 (m, 3H), 4.41-4.40 (d, 2H), 3.290 (s, 3H). |

TABLE 13

| Ex. | Ref. | Structure | LC-MS, NMR |
|---|---|---|---|
| 42 | E4-S1 | 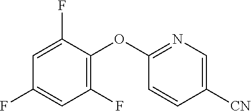 [SMILES: N#Cc1ccc(Oc2c(F)cc(F)cc2F)nc1] | LC-MS: 251 [M + H]⁺ |
| | E4-S2 | 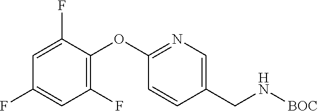 [SMILES: CC(C)(C)OC(=O)NCc1ccc(Oc2c(F)cc(F)cc2F)nc1] | LC-MS: 355 [M + H]⁺ |
| | E4-S3 | 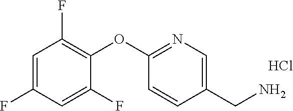 [SMILES: NCc1ccc(Oc2c(F)cc(F)cc2F)nc1 ; Cl] | LC-MS: 255 [M + H]⁺ |
| | E4-S4 | 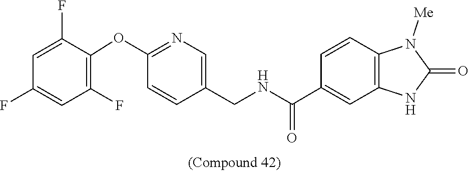 [SMILES: Cn1c(=O)[nH]c2cc(C(=O)NCc3ccc(Oc4c(F)cc(F)cc4F)nc3)ccc21] | LC-MS:429.20 [M + H]⁺; ¹H-NMR (400 MHz, DMSO-D₆) δ 11.05 (s, 1H), 8.92-8.89 (t, 1H), 8.01-8.00 (d, 1H), 7.84-7.81 (dd, 1H), 7.60-7.57 (dd, 1H), 7.47- 7.46 (d, 1H), 7.38-7.34 (m, 2H), 7.18-7.16 (d, 1H), 7.12-7.10 (d, 1H), 4.39-4.37 (d, 2H), 3.266 (s, 3H). |

TABLE 14

| Ex. | Ref. | Structure | LC-MS, NMR |
|---|---|---|---|
| 43 | E4-S1 | 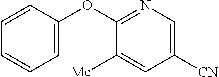 [SMILES: Cc1cc(C#N)cnc1Oc1ccccc1] | LC-MS: 211 [M + H]⁺ |
| | E4-S2 | 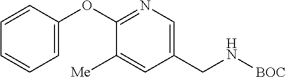 [SMILES: Cc1cc(CNC(=O)OC(C)(C)C)cnc1Oc1ccccc1] | LC-MS: 315 [M + H]⁺ |
| | E4-S3 | 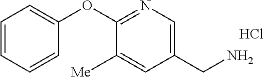 [SMILES: Cc1cc(CN)cnc1Oc1ccccc1 ; Cl] | LC-MS: 215 [M + H]⁺ |
| | E4-S4 | 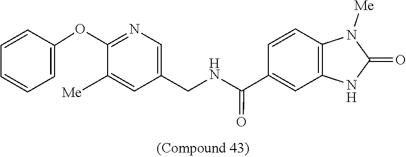 [SMILES: Cc1cc(CNC(=O)c2ccc3c(c2)[nH]c(=O)n3C)cnc1Oc1ccccc1] | LC-MS:389.20 [M + H]⁺; ¹H- NMR (400 MHz, DMSO- D₆) δ 11.15 (s, 1H), 8.97- 8.94 (t, 1H), 7.935-7.931 (d, 1H), 7.70-7.65 (m, 2H), 7.555-7.552 (d, 1H), 7.44- 7.40 (m, 2H), 7.22-7.18 (m, 2H), 7.10-7.08 (m, 2H), 4.439-4.424 (d, 2H), 3.34 (s, 3H), 2.315 (s, 3H). |

TABLE 15

| Ex | Ref. | Structure | LC-MS, NMR |
|---|---|---|---|
| 44 | E4-S1 | 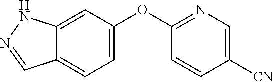 [SMILES: N#Cc1ccc(Oc2ccc3cn[nH]c3c2)nc1] | LC-MS: 237 [M + H]⁺ |
| | E4-S2 | 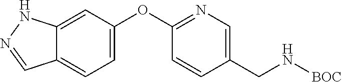 [SMILES: CC(C)(C)OC(=O)NCc1ccc(Oc2ccc3cn[nH]c3c2)nc1] | LC-MS: 341 [M + H]⁺ |
| | E4-S3 | 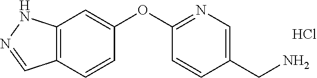 [SMILES: NCc1ccc(Oc2ccc3cn[nH]c3c2)nc1 ; Cl] | LC-MS: 241 [M + H]⁺ |
| | E4-S4 | 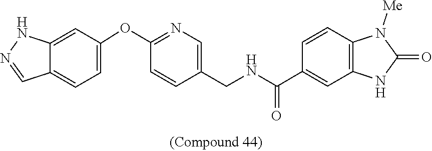 [SMILES: Cn1c(=O)[nH]c2cc(C(=O)NCc3ccc(Oc4ccc5cn[nH]c5c4)nc3)ccc21] | LC-MS:415.20 [M + H]⁺; ¹H-NMR (400 MHz, DMSO-D₆) δ 11.015 (s, 1H), 9.95 (s, 1H), 9.06 (t, 1H), 8.52 (m, 1H), 8.264 (s, 1H), 8.156 (s, 1H), 7.99- 7.93 (m, 1H), 7.714-7.68 (m, 2H), 7.58 (s, 1H), 7.22- 720 (d, 1H), 6.85-6.83 (m, 1H), 4.57-4.56 (d, 2H), 3.34 (s, 3H). |

TABLE 16

| Ex. | Ref. | Structure | LC-MS, NMR |
|---|---|---|---|
| 45 | E4-S1 | 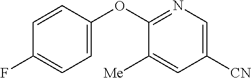 [SMILES: Cc1cc(C#N)cnc1Oc1ccc(F)cc1] | LC-MS: 229 [M + H]⁺ |
| | E4-S2 | 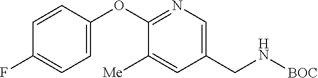 [SMILES: Cc1cc(CNC(=O)OC(C)(C)C)cnc1Oc1ccc(F)cc1] | LC-MS: 333 [M + H]⁺ |
| | E4-S3 | 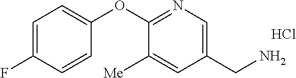 [SMILES: Cc1cc(CN)cnc1Oc1ccc(F)cc1 ; Cl] | LC-MS: 233 [M + H]⁺ |
| | E4-S4 | 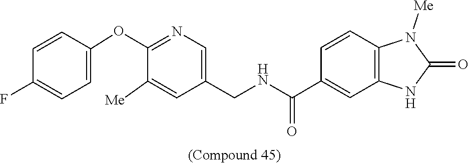 [SMILES: Cc1cc(CNC(=O)c2ccc3c(c2)[nH]c(=O)n3C)cnc1Oc1ccc(F)cc1] | LC-MS: 407.1 [M + H]⁺; ¹H-NMR (400 MHz, DMSO-D₆) δ 11.12 (s, 1H), 8.962-8.936 (t, 1H), 7.922- 7.917 (d, 1H), 7.698-7.656 (m, 2H), 7.552-7.549 (d, 1H), 7.276-7.202 (m, 2H), 7.181-7.137 (m, 3H), 4.432- 4.418 (d, 2H), 3.34 (s, 3H), 2.324 (s, 3H). |

TABLE 17

| Ex. | Ref. | Structure | LC-MS, NMR |
|---|---|---|---|
| 46 | E4-S1 | 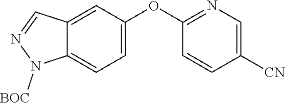 [SMILES: CC(C)(C)OC(=O)n1ncc2cc(Oc3ccc(C#N)cn3)ccc21] | LC-MS: 337 [M + H]⁺ |
| | E10-S3 | 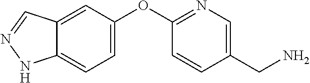 [SMILES: NCc1ccc(Oc2ccc3[nH]ncc3c2)nc1] | |
| | E4-S4 | 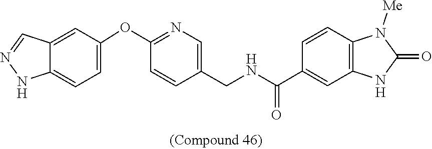 [SMILES: Cn1c(=O)[nH]c2cc(C(=O)NCc3ccc(Oc4ccc5[nH]ncc5c4)nc3)ccc21] | LC-MS: 415.1 [M + H]⁺; ¹H-NMR (400 MHz, DMSO-D₆) δ 13.2 (bs, 1H), 11.15 (s, 1H), 9.003-8.975 (t, 1H), 8.108-8.068 (m, 2H), 7.835-7.809 (dd, 1H), 7.674-7.653 (d, 1H), 7.604- 7.582 (d, 1H), 7.546 (s, 1H), 7.500-7.495 (d, 1H), 7.198- 7.137 (m, 2H), 7.016-6.994 |
| | | | (d, 1H), 4.452-4.439 (d, |
| | | | 2H), 3.34 (s, 3H). |

TABLE 18

| Ex. | Ref. | Structure | LC-MS, NMR |
|---|---|---|---|
| 47 | E4-S1 | 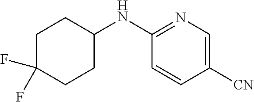 [SMILES: N#Cc1ccc(NC2CCC(F)(F)CC2)nc1] | LC-MS: 239 [M + H]⁺ |
| | E10-S3 | 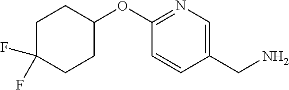 [SMILES: NCc1ccc(OC2CCC(F)(F)CC2)nc1] | LC-MS: 243 [M + H]⁺ |
| | E4-S4 | 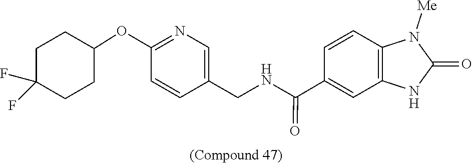 [SMILES: Cn1c(=O)[nH]c2cc(C(=O)NCc3ccc(OC4CCC(F)(F)CC4)nc3)ccc21] | LC-MS: 417.2 [M + H]⁺; ¹H- NMR (400 MHz, DMSO- D₆) δ 11.05 (s, 1H), 8.9 (t, 1H), 8.09 (s, 1H), 7.62-7.60 (m, 2H), 7.49 (s, 1H), 7.153-7.133 (d, 1H), 6.788- 6.768 (d, 1H), 5.17 (bs, 1H), 4.382-4.368 (d, 1H), 4.100- 4.087 (q, 1H), 3.298 (s, 3H), 3.169-3.156 (d, 3H), 2.080- 1.933 (m, 2H), 1.812 (m, 2H). |

TABLE 19

| Ex. | Ref. | Structure | LC-MS, NMR |
|---|---|---|---|
| 48 | E4-S1 | 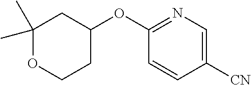 [SMILES: CC1(C)CC(Oc2ccc(C#N)cn2)CCO1] | LC-MS: 233 [M + H]⁺ |
| | E4-S2 | 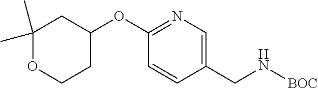 [SMILES: CC(C)(C)OC(=O)NCc1ccc(OC2CCOC(C)(C)C2)nc1] | LC-MS: 337 [M + H]⁺ |
| | E4-S3 | 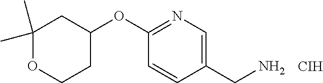 | ¹H-NMR (400 MHz, DMSO-D₆) δ 8.41 (bs, 2H), 8.24-8.24 (d, 1H), 7.87-7.84 (dd, 1H), 6.84-6.82 (d, 1H), 5.30 (m, 1H), 3.97-3.95 (d, 2H), 3.72 (m, 1H), 1.97- |
| | | | 1.96 (m, 2H), 1.44-1.41 (m, |
| | | | 2H), 1.21-1.18 (d, 6H). |
| | E4-S4 | 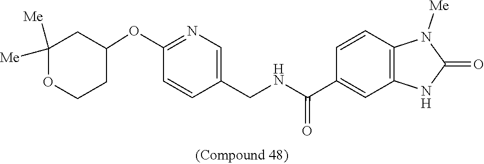 [SMILES: Cn1c(=O)[nH]c2cc(C(=O)NCc3ccc(OC4CCOC(C)(C)C4)nc3)ccc21] | LC-MS: 411.2 [M + H]⁺; ¹H-NMR (400 MHz, CD₃OD) δ 8.097-8.091 (d, 1H), 7.681-7.653 (dd, 1H), 7.634-7.610 (dd, 1H), 7.551-7.548 (d, 1H), 7.150- 7.130 (d, 1H), 6.714-6.692 (d, 1H), 5.283 (m, 1H), 4.470 (s, 2H), 3.845-3.720 (m, 2H), 3.391 (s, 3H), 2.029-1.93 (m, 2H), 1.611- |
| | | | 1.507 (m, 2H), 1.263-1.240 |
| | | | (d, 6H). |

TABLE 20

| Ex. | Ref. | Structure | LC-MS, NMR |
|---|---|---|---|
| 49 | E4-S1 | 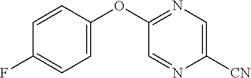 [SMILES: N#Cc1cnc(Oc2ccc(F)cc2)cn1] | ¹H-NMR (400 MHz, DMSO-D₆) δ 8.80 (d, 1H), 8.75 (d, 1H), 7.34-7.32 (m, 4H). |
| | E4-S2 | 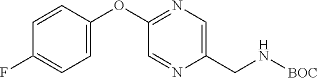 [SMILES: CC(C)(C)OC(=O)NCc1cnc(Oc2ccc(F)cc2)cn1] | LC-MS: 320 [M + H]⁺ |
| | E4-S3 | 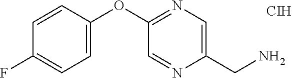 | LC-MS: 220 [M + H]⁺ |
| | E4-S4 | 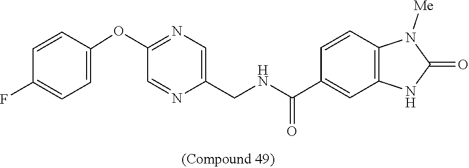 [SMILES: Cn1c(=O)[nH]c2cc(C(=O)NCc3cnc(Oc4ccc(F)cc4)cn3)ccc21] | LC-MS: 394.2 [M + H]⁺; ¹H-NMR (400 MHz, CD₃OD) δ 8.372 (s, 1H), 8.152 (s, 1H), 7.693-7.668 (dd, 1H), 7.598 (d, 1H), 7.189-7.126 (m, 5H), 4.665 (s, 2H), 3.422 (s, 3H). |
| 50 | E6-S3 | 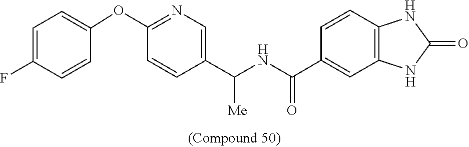 [SMILES: CC(NC(=O)c1ccc2[nH]c(=O)[nH]c2c1)c1ccc(Oc2ccc(F)cc2)nc1] | LC-MS: 393.0 [M + H]⁺; ¹H-NMR (400 MHz, DMSO-D₆) δ 10.9 (d, 2H) 8.682-8.662 (d, 1H), 8.115 (d, 1H), 7.863-7.784 (dd, 1H), 7.538-7.517 (d, 1H), 7.444 (s, 1H), 7.237-7.120 (m, 4H), 6.999-6.931 (m, 2H), 5.155-5.119 (t, 1H), 1.473-1.456 (d, 3H ). |

TABLE 21

| Ex. | Ref. | Structure | LC-MS, NMR |
|---|---|---|---|
| 51 | E27-S1 | 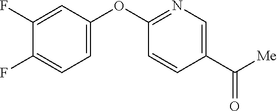 [SMILES: CC(=O)c1ccc(Oc2ccc(F)c(F)c2)nc1] | LC-MS: 250 [M + H]⁺ |
| | E27-S2 | 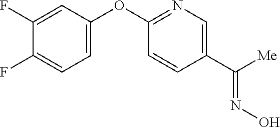 [SMILES: C/C(=N\O)c1ccc(Oc2ccc(F)c(F)c2)nc1] | LC-MS: 265 [M + H]⁺ |
| | E27-S3 | 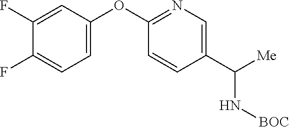 [SMILES: CC(NC(=O)OC(C)(C)C)c1ccc(Oc2ccc(F)c(F)c2)nc1] | LC-MS: 351 [M + H]⁺ |
| | E27-S4 | 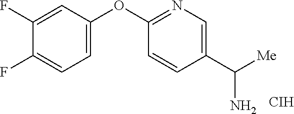 | LC-MS: 251 [M + H]⁺ |
| | E27-S5 | 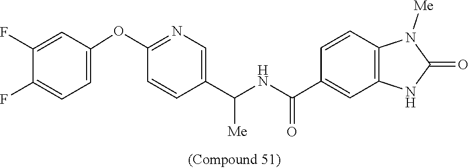 [SMILES: CC(NC(=O)c1ccc2c(c1)[nH]c(=O)n2C)c1ccc(Oc2ccc(F)c(F)c2)nc1] | LC-MS: 425.2 [M + H]⁺; ¹H-NMR (400 MHz, DMSO-D₆) δ 11.076 (s, 1H) 8.742-8.723 (d, 1H), 8.156- 8.151 (d, 1H), 7.908-7.881 (dd, 1H), 7.644-7.620 (dd, 1H), 7.509-7.427 (m, 2H), 7.376-7.323 (m, 1H), 7.158- 7.137 (d, 1H), 7.059-6.978 (m, 2H), 5.182-5.146 (t, 1H), 3.302 (s, 3H), 1.493- 1.475 (d, 3H). |

TABLE 22

| Ex. | Ref. | Structure | LC-MS, NMR |
|---|---|---|---|
| 52 | E27-S1 | 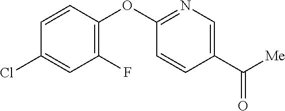 [SMILES: CC(=O)c1ccc(Oc2ccc(Cl)cc2F)nc1] | LC-MS: 266 [M + H]⁺ |
| | E27-S2 | 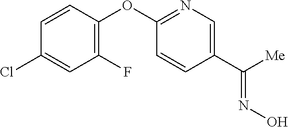 [SMILES: C/C(=N\O)c1ccc(Oc2ccc(Cl)cc2F)nc1] | LC-MS: 281 [M + H]⁺ |
| | E27-S3 | 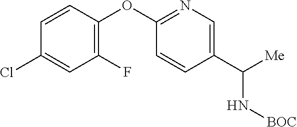 [SMILES: CC(NC(=O)OC(C)(C)C)c1ccc(Oc2ccc(Cl)cc2F)nc1] | LC-MS: 367 [M + H]⁺ |
| | E27-S4 | 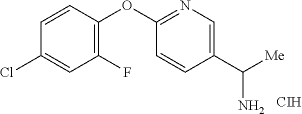 | LC-MS: 267 [M + H]⁺ |
| | E27-S5 | 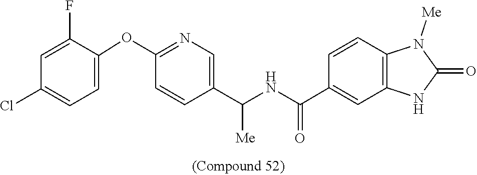 [SMILES: CC(NC(=O)c1ccc2c(c1)[nH]c(=O)n2C)c1ccc(Oc2ccc(Cl)cc2F)nc1] | LC-MS: 440.9 [M + H]⁺; ¹H-NMR (400 MHz, DMSO-D₆) δ 11.073 (s, 1H) 8.738-8.719 (d, 1H), 8.090 (s, 1H), 7.920-7.893 (dd, 1H), 7.639-7.586 (m, 2H), 7.505 (s, 1H), 7.357-7.330 (m, 2H), 7.156-7.116 (t, 2H), 5.155 (t, 1H), 3.303 (s, 3H), 1.488-1.471 (d, 3H). |

TABLE 23

| Ex. | Ref. | Structure | LC-MS, NMR |
|---|---|---|---|
| 53 | E27-S1 | 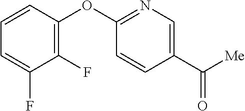 [SMILES: CC(=O)c1ccc(Oc2cccc(F)c2F)nc1] | LC-MS: 250 [M + H]⁺ |
| | E27-S2 | 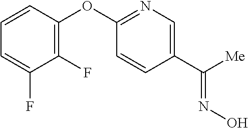 [SMILES: C/C(=N\O)c1ccc(Oc2cccc(F)c2F)nc1] | LC-MS: 265 [M + H]⁺ |
| | E27-S3 | 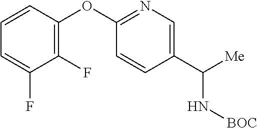 [SMILES: CC(NC(=O)OC(C)(C)C)c1ccc(Oc2cccc(F)c2F)nc1] | LC-MS: 351 [M + H]⁺ |
| | E27-S4 | 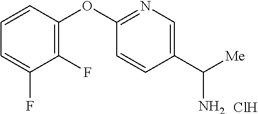 [SMILES: CC(N)c1ccc(Oc2cccc(F)c2F)nc1 ; Cl] | LC-MS: 251 [M + H]⁺ |
| | E27-S5 | 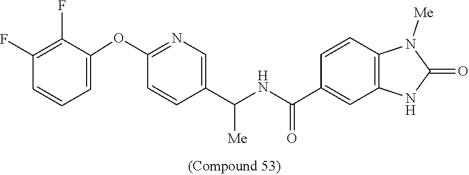 [SMILES: CC(NC(=O)c1ccc2c(c1)[nH]c(=O)n2C)c1ccc(Oc2cccc(F)c2F)nc1] | LC-MS: 425.1 [M + H]⁺; ¹H-NMR (400 MHz, CD₃OD) δ 8.135-8.129 (d, 1H), 7.925-7.897 (dd, 1H), 7.670-7.650 (dd, 2H), 7.563 (s, 1H), 7.217-7.157 (m, 2H), 7.069-7.020 (d, 2H), 5.276-5.225 (q, 1H), 3.418 (s, 3H), 1.607-1.590 (d, 3H). |

TABLE 24

| Ex. | Ref. | Structure | LC-MS, NMR |
|---|---|---|---|
| 54 | E7-S2 | 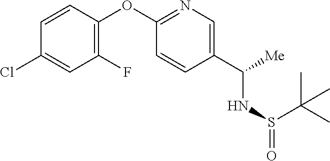 [SMILES: C[C@H](N[S@](=O)C(C)(C)C)c1ccc(Oc2ccc(Cl)cc2F)nc1] | LC-MS: 371 [M + H]⁺ |
| | E7-S3 | 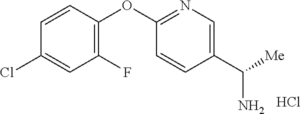 [SMILES: C[C@H](N)c1ccc(Oc2ccc(Cl)cc2F)nc1 ; Cl] | LC-MS: 267 [M + H]⁺ |
| | E7-S4 | 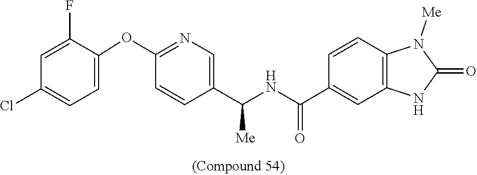 [SMILES: C[C@H](NC(=O)c1ccc2c(c1)[nH]c(=O)n2C)c1ccc(Oc2ccc(Cl)cc2F)nc1] | LC-MS: 441.0 [M + H]⁺; ¹H-NMR (400 MHz, DMSO-D₆) δ 11.12 (s, 1H) 8.752-8.800 (d, 1H), 8.135- 8130 (d, 1H), 7.900-7.970 (dd, 1H), 7.658 (m, 2H), 7.547 (s, 1H), 7.374-7.368 (m, 2H), 7.194-7.154 (t, 2H), 5.680-5.730 (t, 1H), 3.367-3.342 (d, 3H), 1.528- 1.510 (d, 3H). |

TABLE 25

| Ex. | Ref. | Structure | LC-MS, NMR |
|---|---|---|---|
| 55 | E7-S2 | 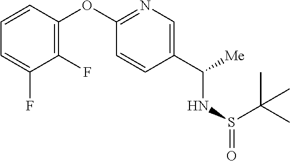 [SMILES: C[C@H](N[S@](=O)C(C)(C)C)c1ccc(Oc2cccc(F)c2F)nc1] | LC-MS: 355 [M + H]⁺ |
| | E7-S3 | 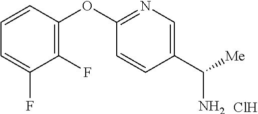 [SMILES: C[C@H](N)c1ccc(Oc2cccc(F)c2F)nc1 ; Cl] | LC-MS: 251 [M + H]⁺ |
| | E7-S4 | 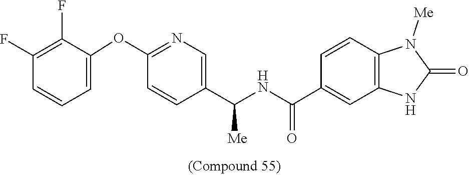 [SMILES: C[C@H](NC(=O)c1ccc2c(c1)[nH]c(=O)n2C)c1ccc(Oc2cccc(F)c2F)nc1] | LC-MS: 425.1 [M + H]⁺. ¹H-NMR (400 MHz, DMSO-D₆) δ 11.1 (s, 1H), 8.786-8.766 (d, 1H), 8.158- 8.153 (d, 1H), 7.977-7.950 (dd, 1H), 7.686-7.665 (d, 1H), 7.552 (s, 1H), 7.411- 7.263 (q, 2H), 7.206-7.176 (m, 3H), 5.240-5.169 (m, 1H), 3.343 (s, 3H), 1.535- 1.518 (d, 3H). |

TABLE 26

| Ex. | Ref. | Structure | LC-MS, NMR |
|---|---|---|---|
| 56 | E8-S2 | 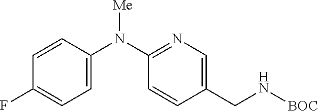 [SMILES: CN(c1ccc(F)cc1)c1ccc(CNC(=O)OC(C)(C)C)cn1] | LC-MS: 332 [M + H]⁺ |
| | E8-S3 | 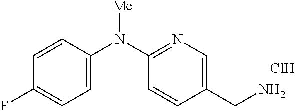 [SMILES: CN(c1ccc(F)cc1)c1ccc(CN)cn1 ; Cl] | LC-MS: 232 [M + H]⁺ |
| | E9-S4 | 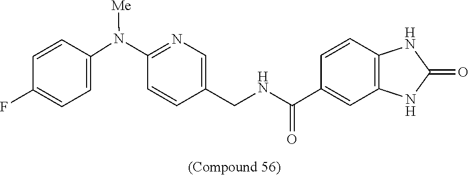 [SMILES: CN(c1ccc(F)cc1)c1ccc(CNC(=O)c2ccc3[nH]c(=O)[nH]c3c2)cn1] | LC-MS: 392.1 [M + H]⁺. ¹H-NMR (400 MHz, DMSO-D₆) δ 10.890-10.857 (s, 2H), 8.880 (t, 1H), 8.016 (s, 1H), 7.730-7.650 (m, 1H), 7.524-7.502 (d, 1H), 7.431-7.326 (m, 4H), 6.965- 6.945 (d, 1H), 6.732-6.650 (m, 1H), 4.343-4.329 (m, 3H), 3.393 (s, 3H), |
| 57 | E8-S4 | 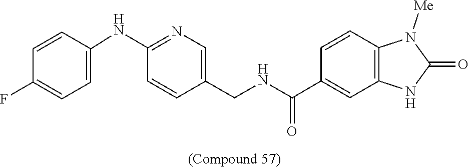 [SMILES: Cn1c(=O)[nH]c2cc(C(=O)NCc3ccc(Nc4ccc(F)cc4)nc3)ccc21] | LC-MS: 392.0 [M + H]⁺. ¹H-NMR (400 MHz, CD₃OD) δ 8.062-8.033 (dd, 1H), 7.844 (s, 1H), 7.664- 7.640 (dd, 1H), 7.578-7.574 (d, 1H), 7.438-7.404 (m, 2H), 7.290-7.247 (m, 2H), 7.192-7.130 (m, 2H), 4.483 (s, 2H), 3.420 (s, 3H). |

TABLE 27

| Ex. | Ref. | Structure | LC-MS, NMR |
|---|---|---|---|
| 58 | E9-S2 | 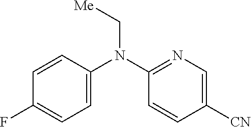 [SMILES: CCN(c1ccc(F)cc1)c1ccc(C#N)cn1] | LC-MS: 242 [M + H]⁺ |
| | E9-S3 | 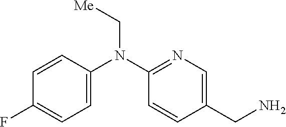 [SMILES: CCN(c1ccc(F)cc1)c1ccc(CN)cn1] | LC-MS: 246 [M + H]⁺ |
| | E9-S4 | 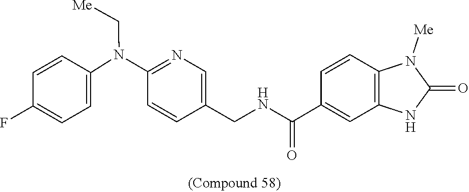 [SMILES: CCN(c1ccc(F)cc1)c1ccc(CNC(=O)c2ccc3c(c2)[nH]c(=O)n3C)cn1] | LC-MS: 420.3 [M + H]⁺. ¹H-NMR (400 MHz, DMSO-D₆) δ 11.15 (s, 1H), 8.944 (t, 1H), 8.054 (bs, 1H), 7.657-7.638 (d, 2H), 7.529 (bs, 1H), 7.388-7.368 (m, 4H), 7.194-7.174 (d, 1H), 6.586 (m, 1H), 4.374- 4.362 (d, 2H), 3.936-3.918 (q, 2H), 3.339 (s, 3H), 1.174-1.141 (t, 3H). |

TABLE 28

| Ex. | Ref. | Structure | LC-MS, NMR |
|---|---|---|---|
| 59 | E9-S1 | 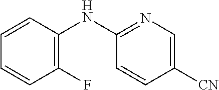 [SMILES: N#Cc1ccc(Nc2ccccc2F)nc1] | LC-MS: 214 [M + H]⁺ |
| | E9-S2 | 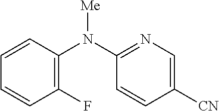 [SMILES: CN(c1ccc(C#N)cn1)c1ccccc1F] | LC-MS: 228 [M + H]⁺ |
| | E10-S3 | 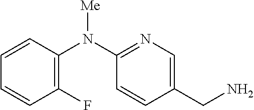 [SMILES: CN(c1ccc(CN)cn1)c1ccccc1F] | LC-MS: 232 [M + H]⁺ |
| | E9-S4 | 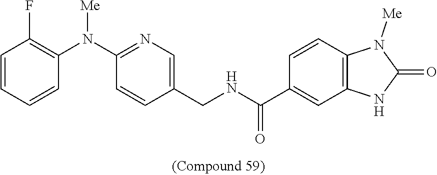 [SMILES: CN(c1ccc(CNC(=O)c2ccc3c(c2)[nH]c(=O)n3C)cn1)c1ccccc1F] | LC-MS: 406.2 [M + H]⁺. ¹H-NMR (400 MHz, DMSO-D₆) δ 11.06 (s, 1H), 8.912 (t, 1H), 8.023 (bs, 1H), 7.673 (m, 1H), 7.585- 7.565 (d, 1H), 7.481-7.277 (m, 5H), 7.115-7.095 (d, 1H), 6.579 (m, 1H), 4.328- 4.315 (d, 2H), 3.543-3.456 (m, 3H), 3.258 (s, 3H). |

TABLE 29

| Ex. | Ref. | Structure | LC-MS, NMR |
|---|---|---|---|
| 60 | E10-S1 | 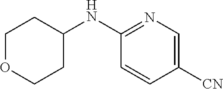 [SMILES: N#Cc1ccc(NC2CCOCC2)nc1] | LC-MS: 204 [M + H]⁺ |
| | E10-S2 | 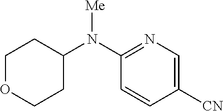 [SMILES: CN(c1ccc(C#N)cn1)C1CCOCC1] | LC-MS: 218 [M + H]⁺ |
| | E10-S3 | 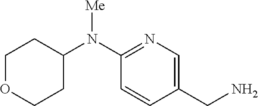 [SMILES: CN(c1ccc(CN)cn1)C1CCOCC1] | LC-MS: 222 [M + H]⁺ |
| | E10-S4 | 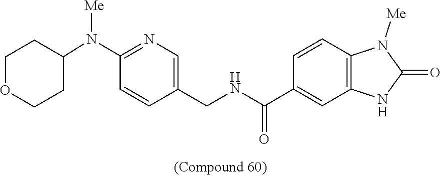 [SMILES: CN(c1ccc(CNC(=O)c2ccc3c(c2)[nH]c(=O)n3C)cn1)C1CCOCC1] | LC-MS: 396.2 [M + H]⁺. ¹H-NMR (400 MHz, DMSO-D₆) δ 11.152 (s, 1H), 8.979 (t, 1H), 8.004 (s, 1H), 7.833 (m, 1H), 7.661-7.641 (d, 1H), 7.530 (s, 1H), 7.201-7.180 (d, 2H), 4.478 (m, 1H), 4.390-4.377 (d, 2H), 3.988-3.951 (m, 2H), 3.563- 3.393 (m, 2H), 3.33 (s, 3H), 3.108 (s, 3H), 1.886-1.807 (m, 2H), 1.620-1.593 (m, 2H). |

TABLE 30

| Ex. | Ref. | Structure | LC-MS, NMR |
|---|---|---|---|
| 61 | E9-S1 | 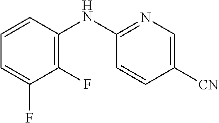 [SMILES: N#Cc1ccc(Nc2cccc(F)c2F)nc1] | LC-MS: 232 [M + H]⁺ |
| | E9-S2 | 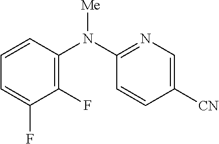 [SMILES: CN(c1ccc(C#N)cn1)c1cccc(F)c1F] | LC-MS: 246 [M + H]⁺ |
| | E8-S2 | 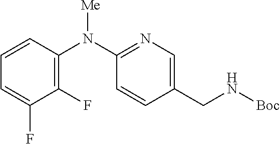 [SMILES: CN(c1ccc(CNC(=O)OC(C)(C)C)cn1)c1cccc(F)c1F] | LC-MS: 350 [M + H]⁺ |
| | E8-S3 | 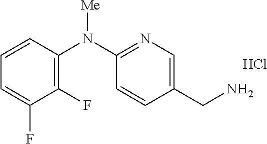 [SMILES: CN(c1ccc(CN)cn1)c1cccc(F)c1F ; Cl] | LC-MS: 250 [M + H]⁺ |
| | E9-S4 | 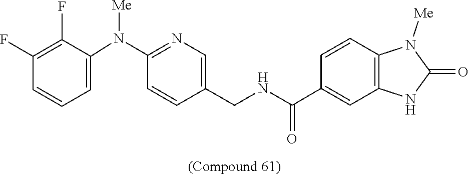 [SMILES: CN(c1ccc(CNC(=O)c2ccc3c(c2)[nH]c(=O)n3C)cn1)c1cccc(F)c1F] | LC-MS: 424.3 [M + H]⁺. ¹H-NMR (400 MHz, DMSO-D₆) δ 11.140 (s, 1H), 8.978 (bs, 1H), 8.116 (s, 1H), 7.723-7.708 (d, 1H), 7.656- 7.640 (d, 1H), 7.531 (s, 1H), 7.438 (bs, 1H), 7.332 (s, 2H), 7.180-7.165 (d, 1H), 6.746-6.730 (d, 1H), 4.391 (s, 2H), 3.441 (s, 3H), 3.326 (s, 3H). |

TABLE 31

| Ex. | Ref. | Structure | LC-MS, NMR |
|---|---|---|---|
| 62 | E8-S1 | 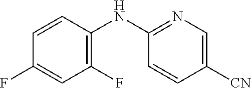 [SMILES: N#Cc1ccc(Nc2ccc(F)cc2F)nc1] | LC-MS: 232 [M + H]⁺ |
| | E9-S2 | 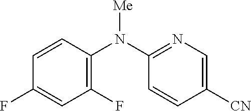 [SMILES: CN(c1ccc(C#N)cn1)c1ccc(F)cc1F] | LC-MS: 246 [M + H]⁺ |
| | E8-S2 | 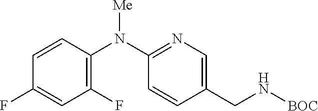 [SMILES: CN(c1ccc(CNC(=O)OC(C)(C)C)cn1)c1ccc(F)cc1F] | LC-MS: 350 [M + H]⁺ |
| | E8-S3 | 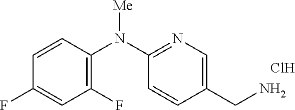 [SMILES: CN(c1ccc(CN)cn1)c1ccc(F)cc1F ; Cl] | LC-MS: 250 [M + H]⁺ |
| | E8-S4 | 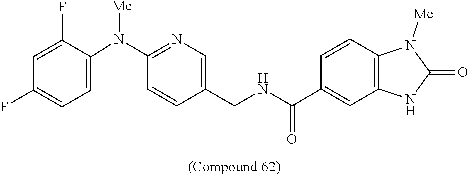 [SMILES: CN(c1ccc(CNC(=O)c2ccc3c(c2)[nH]c(=O)n3C)cn1)c1ccc(F)cc1F] | LC-MS: 424.2 [M + H]⁺. ¹H-NMR (400 MHz, DMSO-D₆) δ 11.121 (s, 1H), 8.923-8.895 (t, 1H), 8.105 (s, 1H), 7.658-7.6001 (m, 2H), 7.567-7.507 (m, 2H), 7.480-7.424 (m, 1H), 7.243-7.171 (m, 2H), 6.559-6.537 (d, 1H), 4.380-4.366 (d, 2H), 3.366-3.337 (d, 6H). |

TABLE 32

| Ex. | Ref. | Structure | LC-MS, NMR |
|---|---|---|---|
| 63 | E9-S1 | 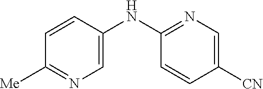 [SMILES: Cc1ccc(Nc2ccc(C#N)cn2)cn1] | LC-MS: 211 [M + H]⁺ |
| | E9-S2 | 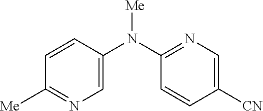 [SMILES: Cc1ccc(N(C)c2ccc(C#N)cn2)cn1] | LC-MS: 225 [M + H]⁺ |
| | E9-S3 | 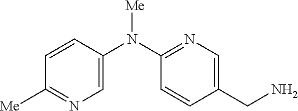 [SMILES: Cc1ccc(N(C)c2ccc(CN)cn2)cn1] | LC-MS: 229 [M + H]⁺ |
| | E9-S4 | 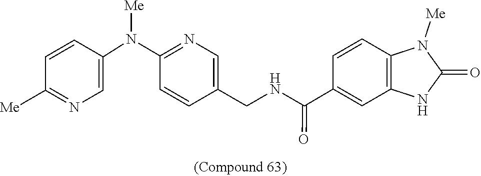 [SMILES: Cc1ccc(N(C)c2ccc(CNC(=O)c3ccc4c(c3)[nH]c(=O)n4C)cn2)cn1] | LC-MS: 403.3 [M + H]⁺. ¹H-NMR (400 MHz, CD₃OD) δ 8.585-8.579 (d, 1H), 8.177 (s, 1H), 8.147- 8.124 (dd, 1H), 7.865-7.837 (dd, 1H), 7.672-7.643 (m, 2H), 7.580 (s, 1H), 7.184-7.163 (d, 1H), 7.100-7.078 (d, 1H), 4.520 (s, 2H), 3.523 (s, 3H), 3.417 (s, 3H), 2.669 (s, 3H). |

TABLE 33

| Ex. | Ref. | Structure | LC-MS, NMR |
|---|---|---|---|
| 64 | E9-S4 | 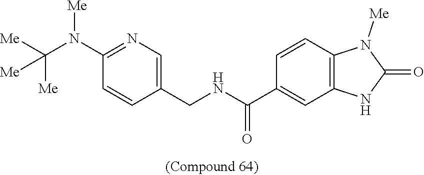 [SMILES: CN(c1ccc(CNC(=O)c2ccc3c(c2)[nH]c(=O)n3C)cn1)C(C)(C)C] | LC-MS: 368.2 [M + H]⁺. ¹H-NMR (400 MHz, DMSO-D₆) δ 11.06 (s, 1H), 8.88-8.80 (t, 1H), 8.08 (t, 1H), 7.65-7.60 (dd, 1H), 7.52 (d, 1H), 7.48-7.43 (dd, 1H), 7.16- 7.12 (d, 1H), 6.75-6.70 (d, 1H), 4.330-4.315 (d, 2H), 3.304 (s, 3H), 2.885 (s, 3H), 1.387 (s, 9H). |

TABLE 34

| Ex. | Ref. | Structure | LC-MS, NMR |
|---|---|---|---|
| 65 | E10-S1 | 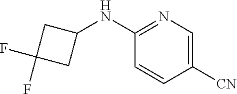 [SMILES: N#Cc1ccc(NC2CC(F)(F)C2)nc1] | LC-MS: 210 [M + H]⁺ |
| | E10-S2 | 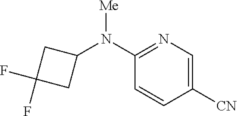 [SMILES: CN(c1ccc(C#N)cn1)C1CC(F)(F)C1] | LC-MS: 224 [M + H]⁺ |
| | E10-S3 | 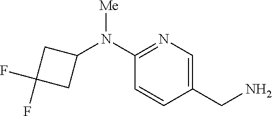 [SMILES: CN(c1ccc(CN)cn1)C1CC(F)(F)C1] | LC-MS: 228 [M + H]⁺ |
| | E10-S4 | 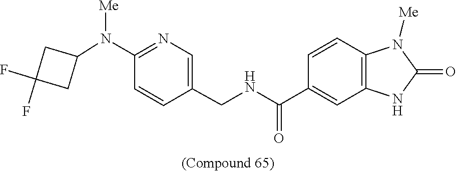 [SMILES: CN(c1ccc(CNC(=O)c2ccc3c(c2)[nH]c(=O)n3C)cn1)C1CC(F)(F)C1] | LC-MS: 402.0 [M + H]⁺. ¹H-NMR (400 MHz, DMSO-D₆) δ 11.049 (s, 1H), 8.823-8.794 (t, 1H), 8.052-8.047 (d, 1H), 7.588-7.567 (dd, 1H), 7.502-7.461 (m, 2H), 7.118-7.083 (d, 1H), 6.672-6.651 (d, 1H), 4.645-4.597 (m, 1H), 4.285-4.271 (d, 2H), 3.265 (s, 3H), 2.871-2.753 (m, 5H), 2.732-2.637 (m, 2H). |

TABLE 35

| Ex. | Ref. | Structure | LC-MS, NMR |
|---|---|---|---|
| 66 | E10-S1 | 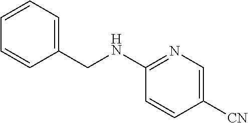 [SMILES: N#Cc1ccc(NCc2ccccc2)nc1] | LC-MS: 210 [M + H]⁺ |
| | E10-S2 | 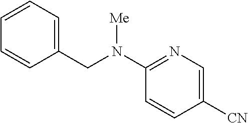 [SMILES: CN(Cc1ccccc1)c1ccc(C#N)cn1] | LC-MS: 224 [M + H]⁺ |
| | E10-S3 | 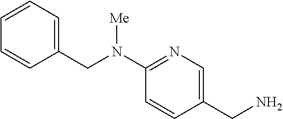 [SMILES: CN(Cc1ccccc1)c1ccc(CN)cn1] | LC-MS: 228 [M + H]⁺ |
| | E10-S4 | 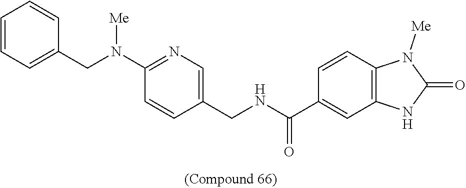 [SMILES: CN(Cc1ccccc1)c1ccc(CNC(=O)c2ccc3c(c2)[nH]c(=O)n3C)cn1] | LC-MS: 402.2 [M + H]⁺. ¹H-NMR (400 MHz, DMSO-D₆) δ 11.06 (s, 1H), 8.85-8.75 (t, 1H), 8.049 (s, 1H), 7.65-7.60 (d, 1H), 7.501 (m, 2H), 7.292-7.273 (m, 2H), 7.190-7.148 (m, 4H), 6.75-6.70 (d, 1H), 4.768 (s, 2H), 4.313-4.299 (d, 2H), 3.301 (s, 3H), 3.003 (s, 3H). |

TABLE 36

| Ex. | Ref. | Structure | LC-MS, NMR |
|---|---|---|---|
| 67 | E10-S1 | 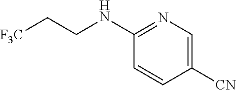 [SMILES: N#Cc1ccc(NCCC(F)(F)F)nc1] | LC-MS: 216 [M + H]⁺ |
| | E10-S2 | 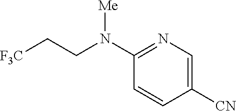 [SMILES: CN(CCC(F)(F)F)c1ccc(C#N)cn1] | LC-MS: 230 [M + H]⁺ |
| | E10-S3 | 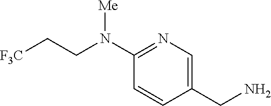 [SMILES: CN(CCC(F)(F)F)c1ccc(CN)cn1] | LC-MS: 234 [M + H]⁺ |
| | E10-S4 | 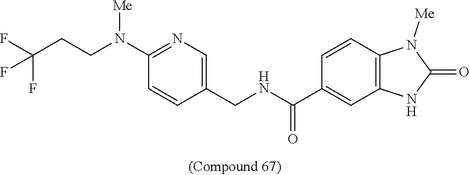 [SMILES: CN(CCC(F)(F)F)c1ccc(CNC(=O)c2ccc3c(c2)[nH]c(=O)n3C)cn1] | LC-MS: 408.3 [M + H]⁺. ¹H-NMR (400 MHz, DMSO-D₆) δ 11.073 (s, 1H), 8.836-8.808 (t, 1H), 8.073-8.067 (d, 1H), 7.631-7.606 (dd, 1H), 7.526-7.501 (m, 2H), 7.154-7.133 (d, 1H), 6.619-6.597 (d, 1H), 4.318-4.303 (d, 2H), 3.794- 3.759 (t, 2H), 3.303 (s, 3H), 2.968 (s, 3H), 2.509 (m, 2H). |

TABLE 37

| Ex. | Ref. | Structure | LC-MS, NMR |
|---|---|---|---|
| 68 | E10-S1 | 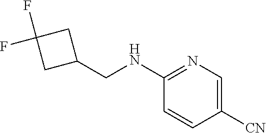 [SMILES: N#Cc1ccc(NCC2CC(F)(F)C2)nc1] | LC-MS: 224 [M + H]⁺ |
| | E10-S2 | 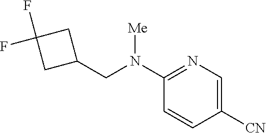 [SMILES: CN(CC1CC(F)(F)C1)c1ccc(C#N)cn1] | LC-MS: 238 [M + H]⁺ |
| | E10-S3 | 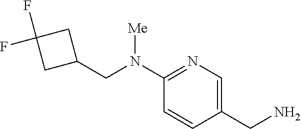 [SMILES: CN(CC1CC(F)(F)C1)c1ccc(CN)cn1] | LC-MS: 242 [M + H]⁺ |
| | E10-S4 | 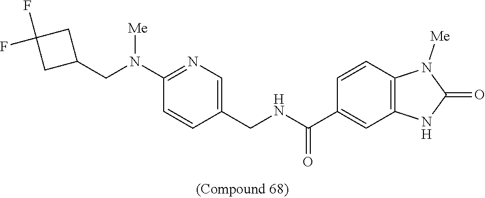 [SMILES: CN(CC1CC(F)(F)C1)c1ccc(CNC(=O)c2ccc3c(c2)[nH]c(=O)n3C)cn1] | LC-MS: 416.3 [M + H]⁺. ¹H-NMR (400 MHz, CD₃OD) δ 7.976 (s, 1H), 7.732-7.714 (d, 1H), 7.629- 7.605 (dd, 1H), 7.547 (s, 1H), 7.145- 7.124 (d, 1H), 6.877-6.855 (d, 1H), 4.426 (s, 2H), 3.708-3.691 (d, 2H), 3.387 (s, 3H), 3.101 (s, 3H) 2.658- 2.552 (m, 2H), 2.495 (m, 1H), 2.407- 2.284 (m, 2H). |

TABLE 38

| Ex. | Ref. | Structure | LC-MS, NMR |
|---|---|---|---|
| 69 | E10-S1 | 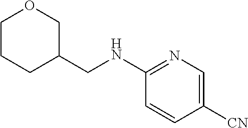 [SMILES: N#Cc1ccc(NCC2CCCOC2)nc1] | LC-MS: 218 [M + H]⁺ |
| | E10-S2 | 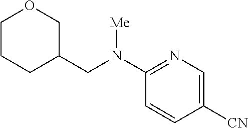 [SMILES: CN(CC1CCCOC1)c1ccc(C#N)cn1] | LC-MS: 232 [M + H]⁺ |
| | E10-S3 | 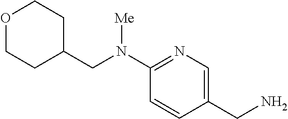 [SMILES: CN(CC1CCOCC1)c1ccc(CN)cn1] | LC-MS: 236 [M + H]⁺ |
| | E10-S4 | 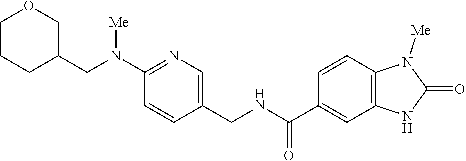 [SMILES: CN(CC1CCCOC1)c1ccc(CNC(=O)c2ccc3c(c2)[nH]c(=O)n3C)cn1] | LC-MS: 410.0 [M + H]⁺. ¹H-NMR (400 MHz, CD₃OD) δ 8.031-8.026 (d, 1H), 7.625-7.601 (dd, 1H), 7.545-7.520 (dd, 2H), 7.14- 7.119 (d, 1H), 6.613-6.591 (d, 1H), 4.405 (s, 2H), 3.783-3.726 (m, 2H), 3.5-3.4 (m, 4H), 3.35 (m, 1H) 3.110 (m, 1H), 3.005-2.840 (m, 3H), |
| | | (Compound 69) | 2.188-2.014 (m, 1H) 1.868- |
| | | | 1.756 (m, 1H), 1.659-1.623 |
| | | | (m, 1H), 1.576-1.490 (m, 1H), |
| | | | 1.378-1.240 (m, 2H). |

TABLE 39

| Ex. | Ref. | Structure | LC-MS, NMR |
|---|---|---|---|
| 70 | E10-S1 | 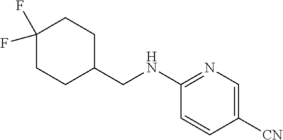 [SMILES: N#Cc1ccc(NCC2CCC(F)(F)CC2)nc1] | LC-MS: 252 [M + H]⁺ |
| | E10-S2 | 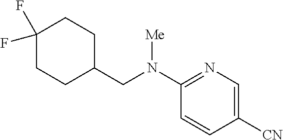 [SMILES: CN(CC1CCC(F)(F)CC1)c1ccc(C#N)cn1] | LC-MS: 266 [M + H]⁺ |
| | E10-S3 | 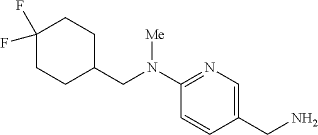 [SMILES: CN(CC1CCC(F)(F)CC1)c1ccc(CN)cn1] | LC-MS: 270 [M + H]⁺ |
| | E10-S4 | 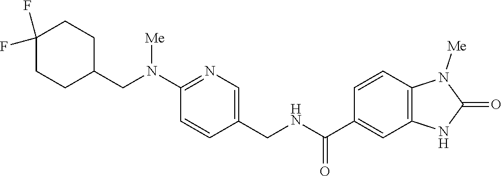 [SMILES: CN(CC1CCC(F)(F)CC1)c1ccc(CNC(=O)c2ccc3c(c2)[nH]c(=O)n3C)cn1] | LC-MS: 444.3 [M + H]⁺. ¹H-NMR (400 MHz, DMSO-D₆) δ 11.060 (s, 1H), 8.805-8.776 (t, 1H), 8.009-8.004 (d, 1H), 7.616-7.592 (dd, 1H), 7.491- 7.487 (d, 1H), 7.459-7.431 (dd, 1H), 7.138-7.117 (d, 1H), 6.571- 6.549 (d, 1H), 4.283-4.268 (d, 2H), 3.409-3.390 (d, 2H), 3.289 (s, 3H), 2.964 (s, 3H), 2.075-1.955 (m, 2H), 1.841-1.762 (m, |
| | | (Compound 70) | 2H), 1.711-1.642 (m, 2H), |
| | | | 1.249-1.146 (m, 3H). |

TABLE 40

| Ex. | Ref. | Structure | LC-MS, NMR |
|---|---|---|---|
| 71 | E10-S1 | 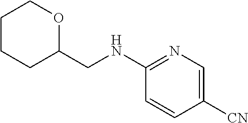 [SMILES: N#Cc1ccc(NCC2CCCCO2)nc1] | LC-MS: 218 [M + H]⁺ |
| | E10-S2 | 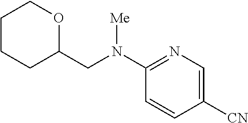 [SMILES: CN(CC1CCCCO1)c1ccc(C#N)cn1] | LC-MS: 232 [M + H]⁺ |
| | E10-S3 | 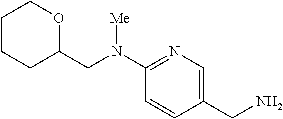 [SMILES: CN(CC1CCCCO1)c1ccc(CN)cn1] | LC-MS: 236 [M + H]⁺ |
| | E10-S4 | 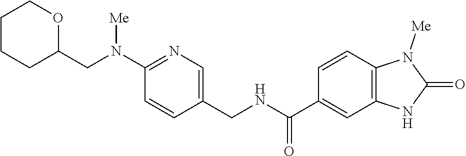 [SMILES: CN(CC1CCCCO1)c1ccc(CNC(=O)c2ccc3c(c2)[nH]c(=O)n3C)cn1] | LC-MS: 410.1 [M + H]⁺. ¹H-NMR (400 MHz, CD₃OD) δ 8.015 (s, 1H), 7.626- 7.606 (d, 1H), 7.545-7.514 (m, 2H), 7.146-7.125 (d, 1H), 6.627-6.605 (d, 1H), 4.405 (s, 2H), 3.905-3.879 (d, 1H), 3.612- 3.550 (m, 2H), 3.432-3.336 (m, 5H), 3.042 (s, 3H), 1.822 (m, 1H), 1.621-1.589 (m, 1H), |
| | | | 1.543-1.40 (m, 2H), 1.35 (m, |
| | | | 1H), 1.268-1.233 (m, 1H). |
| | | (Compound 71) | |

TABLE 41

| Ex. | Ref. | Structure | LC-MS, NMR |
|---|---|---|---|
| 72 | E10-S1 | 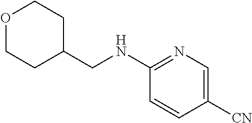 [SMILES: N#Cc1ccc(NCC2CCOCC2)nc1] | LC-MS: 218 [M + H^{]+} |
| | E10-S2 | 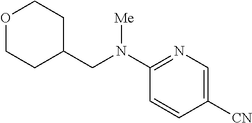 [SMILES: CN(CC1CCOCC1)c1ccc(C#N)cn1] | LC-MS: 232 [M + H]⁺ |
| | E10-S3 | 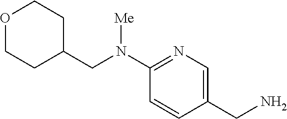 [SMILES: CN(CC1CCOCC1)c1ccc(CN)cn1] | LC-MS: 236 [M + H]⁺ |
| | E10-S4 | 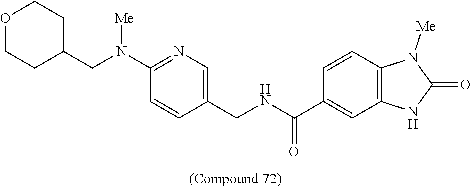 [SMILES: CN(CC1CCOCC1)c1ccc(CNC(=O)c2ccc3c(c2)[nH]c(=O)n3C)cn1] | LC-MS: 410.3 [M + H]⁺. ¹H-NMR (400 MHz, CD₃OD) δ 8.026-8.022 (d,1H), 7.626-7.602 (dd, 1H), 7.546-7.521 (dd, 2H), 7.142-7.122 (d, 1H), 6.620-6.597 (d, 1H), 4.403 (s, 2H), 3.916-3.879 (dd, 2H), 3.408 (m, 1H), 3.389 (s, 3H), 3.365-3.334 (m, 2H), 3.032 (s, 3H), 2.020-1.982 (m, 1H), 1.565-1.534 (d, 2H), 1.358- 1.267 (m, 3H). |

TABLE 42

| Ex | Ref. | Structure | LC-MS, NMR |
|---|---|---|---|
| 73 | E10-S1 | 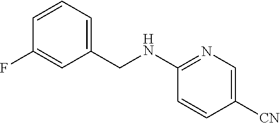 [SMILES: N#Cc1ccc(NCc2cccc(F)c2)nc1] | LC-MS: 228 [M + H]⁺ |
| | E10-S2 | 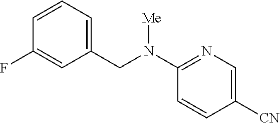 [SMILES: CN(Cc1cccc(F)c1)c1ccc(C#N)cn1] | LC-MS: 242 [M + H]⁺ |
| | E10-S3 | 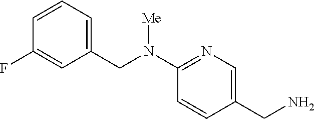 [SMILES: CN(Cc1cccc(F)c1)c1ccc(CN)cn1] | LC-MS: 246 [M + H]⁺ |
| | E10-S4 | 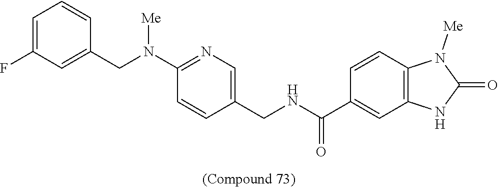 [SMILES: CN(Cc1cccc(F)c1)c1ccc(CNC(=O)c2ccc3c(c2)[nH]c(=O)n3C)cn1] | LC-MS: 420.1 [M + H]⁺. ¹H-NMR (300 MHz, DMSO-D₆) δ 11.040 (s, 1H), 8.834-8.798 (t, 1H), 8.049 (s, 1H), 7.628-7.600 (d, 1H), 7.500-7.483 (m, 2H), 7.346-7.298 (q, 1H), 7.151-7.123 (d, 1H), 7.060-6.959 (m, 3H), 6.638- 6.609 (d, 1H), 4.785 (s, 2H), 4.316-4.298 (d, 2H), 3.297 (s, 3H), 3.012 (s, 3H). |

TABLE 43

| Ex | Ref. | Structure | LC-MS, NMR |
|---|---|---|---|
| 74 | E10-S1 | 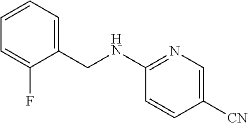 [SMILES: N#Cc1ccc(NCc2ccccc2F)nc1] | LC-MS: 228 [M + H]⁺ |
| | E10-S2 | 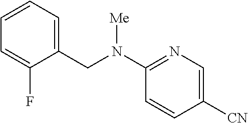 [SMILES: CN(Cc1ccccc1F)c1ccc(C#N)cn1] | LC-MS: 242 [M + H]⁺ |
| | E10-S3 | 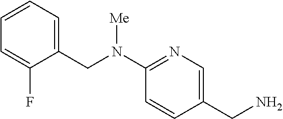 [SMILES: CN(Cc1ccccc1F)c1ccc(CN)cn1] | LC-MS: 246 [M + H]⁺ |
| | E10-S4 | 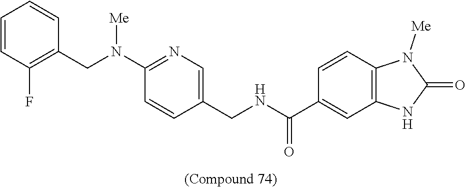 [SMILES: CN(Cc1ccccc1F)c1ccc(CNC(=O)c2ccc3c(c2)[nH]c(=O)n3C)cn1] | LC-MS: 420.2 [M + H]⁺. ¹H-NMR (400 MHz, DMSO-D₆) δ 11.030 (s, 1H), 8.791-8.762 (t, 1H), 8.011-8.005 (d, 1H), 7.592- 7.568 (dd, 1H), 7.478-7.450 (m, 2H), 7.256-7.213 (m, 1H), 7.169-7.053 (m, 4H), 6.624-6.602 (d, 1H), 4.780 (s, 2H), 4.279-4.265 (d, 2H), 3.265 (s, 3H), 2.999 (s, 3H). |

TABLE 44

| Ex. | Ref. | Structure | LC-MS, NMR |
|---|---|---|---|
| 75 | E10-S1 | 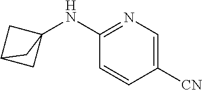 [SMILES: N#Cc1ccc(NC23CC(C2)C3)nc1] | LC-MS: 186 [M + H]⁺ |
| | E10-S2 | 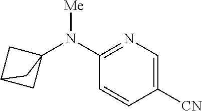 [SMILES: CN(c1ccc(C#N)cn1)C12CC(C1)C2] | LC-MS: 200 [M + H]⁺ |
| | E10-S3 | 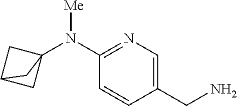 [SMILES: CN(c1ccc(CN)cn1)C12CC(C1)C2] | LC-MS: 204 [M + H]⁺ |
| | E10-S4 | 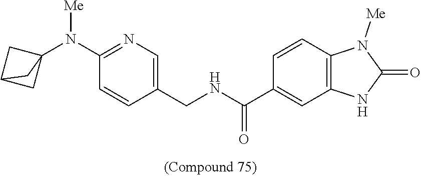 [SMILES: CN(c1ccc(CNC(=O)c2ccc3c(c2)[nH]c(=O)n3C)cn1)C12CC(C1)C2] | LC-MS: 378.2 [M + H]⁺. ¹H-NMR (400 MHz, CD₃OD) δ 8.095-8.066 (dd, 1H), 7.966-7.962 (d, 1H), 7.688-7.664 (dd, 1H), 7.601-7.598 (d, 1H), 7.577- 7.554 (d, 1H), 7.206-7.186 (d, 1H), 4.516 (s, 2H), 3.436 (s, 3H), 3.198 (s, 3H), 2.642 (s, 1H), 2.380 (s, 6H). |

TABLE 45

| Ex. | Ref. | Structure | LC-MS, NMR |
|---|---|---|---|
| 76 | E10-S1 | 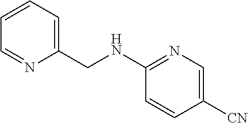 [SMILES: N#Cc1ccc(NCc2ccccn2)nc1] | LC-MS: 211 [M + H]⁺ |
| | E10-S2 | 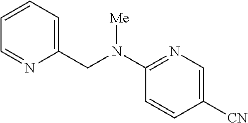 [SMILES: CN(Cc1ccccn1)c1ccc(C#N)cn1] | LC-MS: 225 [M + H]⁺ |
| | E10-S3 | 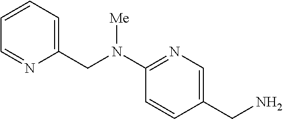 [SMILES: CN(Cc1ccccn1)c1ccc(CN)cn1] | LC-MS: 229 [M + H]⁺ |
| | E10-S4 | 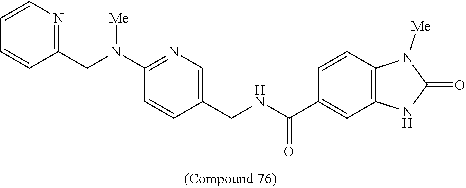 [SMILES: CN(Cc1ccccn1)c1ccc(CNC(=O)c2ccc3c(c2)[nH]c(=O)n3C)cn1] | LC-MS: 403.3 [M + H]⁺. ¹H-NMR (400 MHz, DMSO-D₆) δ 11.079 (s, 1H), 8.502 (d, 1H), 8.011- 8.005 (d, 1H), 7.7-7.65 (m, 1H), 7.6 (dd, 1H), 7.5-7.45 (m, 2H), 7.25 (m, 1H), 7.15- 7.08 (dd, 2H), 6.65 (d, 1H), 4.83 (s, 2H),4.295 (s, 2H), 3.292 (s, 3H), 3.1 (s, 3H). |

TABLE 46

| Ex. | Ref. | Structure | LC-MS, NMR |
|---|---|---|---|
| 77 | E10-S1 | 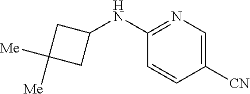 [SMILES: CC1(C)CC(Nc2ccc(C#N)cn2)C1] | LC-MS: 202 [M + H]⁺ |
| | E10-S2 | 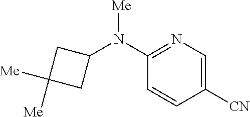 [SMILES: CN(c1ccc(C#N)cn1)C1CC(C)(C)C1] | LC-MS: 216 [M + H]⁺ |
| | E10-S3 | 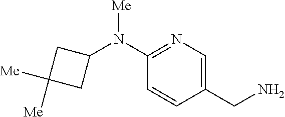 [SMILES: CN(c1ccc(CN)cn1)C1CC(C)(C)C1] | LC-MS: 220 [M + H]⁺ |
| | E10-S4 | 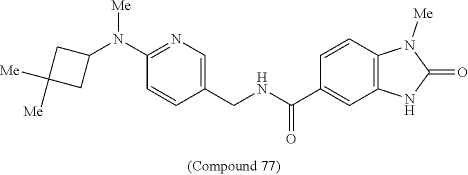 [SMILES: CN(c1ccc(CNC(=O)c2ccc3c(c2)[nH]c(=O)n3C)cn1)C1CC(C)(C)C1] | LC-MS: 394.3 [M + H]⁺. ¹H-NMR (400 MHz, CD₃OD) δ 8.054-8.025 (dd, 1H), 7.901-7.896 (d, 1H), 7.681-7.656 (dd, 1H), 7.6 (d, 1H), 7.267-7.191 (m, 2H), 4.49 (s, 3H), 3.43 (s, 3H), 3.183 (s, 3H), 2.329-2.279 (m, 2H), 2.118-2.088 (m, 2H), 1.290 (s, 3H), 1.203 (s, 3H). |

TABLE 47

| Ex. | Ref. | Structure | LC-MS, NMR |
|---|---|---|---|
| 78 | E10-S1 | 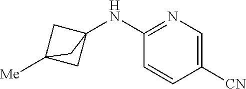 [SMILES: CC12CC(Nc3ccc(C#N)cn3)(C1)C2] | LC-MS: 200 [M + H]⁺ |
| | E10-S2 | 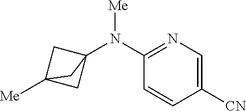 [SMILES: CN(c1ccc(C#N)cn1)C12CC(C)(C1)C2] | LC-MS: 214 [M + H]⁺ |
| | E10-S3 | 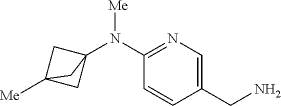 [SMILES: CN(c1ccc(CN)cn1)C12CC(C)(C1)C2] | LC-MS: 218 [M + H]⁺ |
| | E10-S4 | 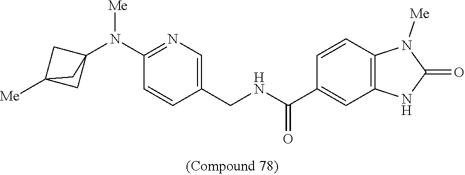 [SMILES: CN(c1ccc(CNC(=O)c2ccc3c(c2)[nH]c(=O)n3C)cn1)C12CC(C)(C1)C2] | LC-MS: 392.2 [M + H]⁺. ¹H-NMR (400 MHz, CD₃OD) δ 8.088-8.059 (dd, 1H), 7.948-7.941 (d, 1H), 7.686-7.661 (dd, 1H), 7.598-7.595 (d, 1H), 7.562- 7.538 (d, 1H), 7.211-7.190 (d, 1H), 4.511 (s, 2H), 3.438 (s, 3H), 3.193 (s, 3H), 2.233 (s, 6H), 1.345 (s, 3H). |

TABLE 48

| Ex. | Ref. | Structure | LC-MS, NMR |
|---|---|---|---|
| 79 | E10-S1 | 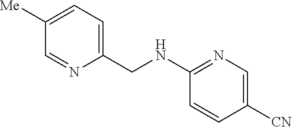 [SMILES: Cc1ccc(CNc2ccc(C#N)cn2)nc1] | LC-MS: 225 [M + H]⁺ |
| | E10-S2 | 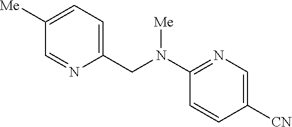 [SMILES: Cc1ccc(CN(C)c2ccc(C#N)cn2)nc1] | LC-MS: 239 [M + H]⁺ |
| | E10-S3 | 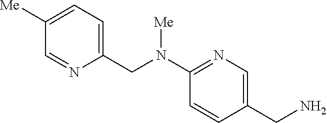 [SMILES: Cc1ccc(CN(C)c2ccc(CN)cn2)nc1] | LC-MS: 243 [M + H]⁺ |
| | E10-S4 | 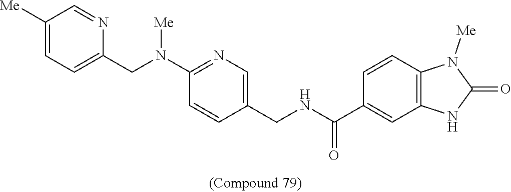 [SMILES: Cc1ccc(CN(C)c2ccc(CNC(=O)c3ccc4c(c3)[nH]c(=O)n4C)cn2)nc1] | LC-MS: 417.3 [M + H]^{+.} ¹H-NMR (400 MHz, CD₃OD) δ 8.462 (bs, 1H), 8.012-7.993 (m, 2H), 7.944- 7.925 (m, 1H), 7.676-7.651 (dd, 1H), 7.591-7.587 (d, 1H), 7.541-7.522 (m, 1H), 7.245-7.175 (m, 2H), 5.027 (s, 2H), 4.501 (s, 2H), 3.430 (s, 3H), 3.33 (s, 3H), 2.420 (s, 3H). |

TABLE 49

| Ex. | Ref. | Structure | LC-MS, NMR |
|---|---|---|---|
| 80 | E10-S1 | 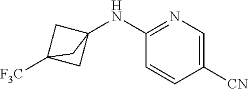 [SMILES: N#Cc1ccc(NC23CC(C(F)(F)F)(C2)C3)nc1] | LC-MS: 254 [M + H]⁺ |
| | E10-S2 | 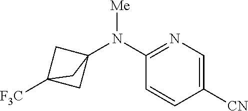 [SMILES: CN(c1ccc(C#N)cn1)C12CC(C(F)(F)F)(C1)C2] | LC-MS: 268 [M + H]⁺ |
| | E10-S3 | 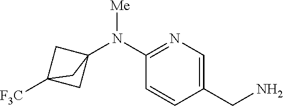 [SMILES: CN(c1ccc(CN)cn1)C12CC(C(F)(F)F)(C1)C2] | LC-MS: 272 [M + H]⁺ |
| | E10-S4 | 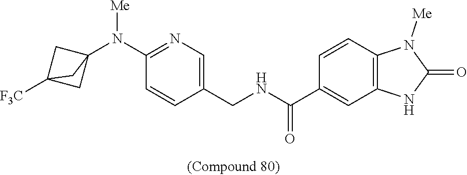 [SMILES: CN(c1ccc(CNC(=O)c2ccc3c(c2)[nH]c(=O)n3C)cn1)C12CC(C(F)(F)F)(C1)C2] | LC-MS: 446.1 [M + H]⁺. ¹H-NMR (400 MHz, DMSO-D₆) δ 7.974 (bs, 1H), 7.736-7.708 (dd, 1H), 7.562-7.537 (m, 1H), 7.456 (d, 1H), 7.131-7.110 (d, 1H), 7.081-7.058 (d, 1H), 4.309 (s, 2H), 3.255 (s, 3H), 2.948 (s, 3H), 2.414 (s, 6H). |

TABLE 50

| Ex. | Ref. | Structure | LC-MS, NMR |
|---|---|---|---|
| 81 | E10-S1 | 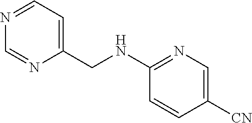 [SMILES: N#Cc1ccc(NCc2ccncn2)nc1] | LC-MS: 212 [M + H]⁺ |
| | E10-S2 | 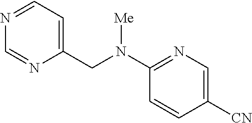 [SMILES: CN(Cc1ccncn1)c1ccc(C#N)cn1] | LC-MS: 226 [M + H]⁺ |
| | E10-S3 | 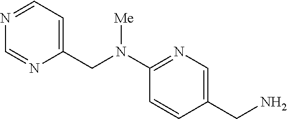 [SMILES: CN(Cc1ccncn1)c1ccc(CN)cn1] | LC-MS: 230 [M + H] ⁺ |
| | E10-S4 | 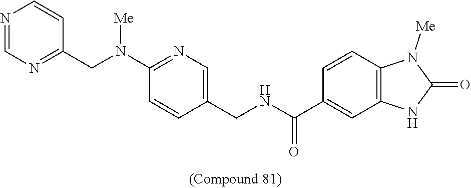 [SMILES: CN(Cc1ccncn1)c1ccc(CNC(=O)c2ccc3c(c2)[nH]c(=O)n3C)cn1] | LC-MS: 404.1 [M + H]⁺. ¹H-NMR (400 MHz, CD₃OD) δ 9.044-9.042 (d, 1H), 8.753-8.740 (d, 1H), 8.045-8.016 (dd, 1H), 7.920 (bs, 1H), 7.639-7.614 (dd, 1H), 7.554-7.549 (d, 1H), 7.527-7.516 (d, 1H), 7.278-7.254 (d, 1H), 7.169-7.148 (d, 1H), 5.03 (s, 2H), 4.473 (s, 2H), 3.397 (s, 3H), 3.296 (s, 3H). |

TABLE 51

| Ex. | Ref. | Structure | LC-MS, NMR |
|---|---|---|---|
| 82 | E11-S1 | 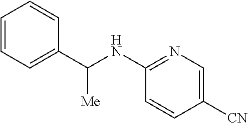 [SMILES: CC(Nc1ccc(C#N)cn1)c1ccccc1] | LC-MS: 224 [M + H]⁺ |
| | E11-S2 | 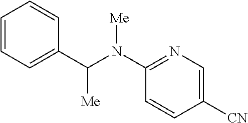 [SMILES: CC(c1ccccc1)N(C)c1ccc(C#N)cn1] | LC-MS: 238 [M + H]⁺ |
| | E11-S3 | 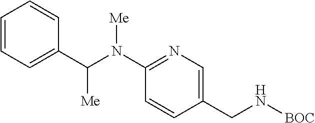 [SMILES: CC(c1ccccc1)N(C)c1ccc(CNC(=O)OC(C)(C)C)cn1] | LC-MS: 342 [M + H]⁺ |
| | E11-S4 | 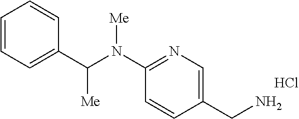 [SMILES: CC(c1ccccc1)N(C)c1ccc(CN)cn1 ; Cl] | LC-MS: 242 [M + H]⁺ |
| | E11-S5 | 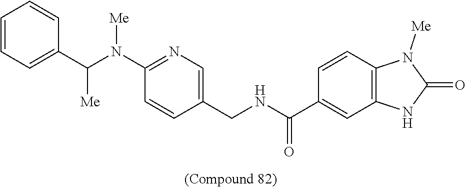 [SMILES: CC(c1ccccc1)N(C)c1ccc(CNC(=O)c2ccc3c(c2)[nH]c(=O)n3C)cn1] | LC-MS: 416.3 [M + H]⁺. ¹H-NMR (400 MHz, CD₃OD) δ 8.073-8.044 (dd, 1H), 7.909-7.905 (d, 1H), 7.658-7.633 (dd, 1H), 7.571-7.568 (d, 1H), 7.457-7.433 (d, 1H), 7.387-7.368 (m, 2H), 7.344-7.319 (m, 3H), 7.170-7.149 (d, 1H), 5.531-5.515 (q, 1H), 4.479 (s, 2H), 3.395 (s, 3H), 2.948 (s, 3H), 1.707-1.689 (d, 3H). |

TABLE 52

| Ex. | Ref. | Structure | LC-MS, NMR |
|---|---|---|---|
| 82 | | 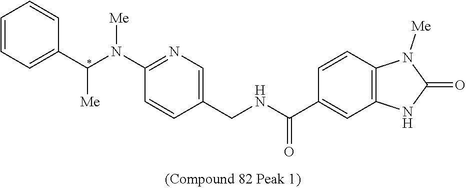 [SMILES: CC(c1ccccc1)N(C)c1ccc(CNC(=O)c2ccc3c(c2)[nH]c(=O)n3C)cn1] | LC-MS: 416.2 [M + H]^{+.} ¹H-NMR (400 MHz, CD₃OD) δ 8.084-8.079 (d, 1H), 7.639-7.558 (m, 3H), 7.287-7.250 (m, 4H), 7.218- 7.202 (m, 1H), 7.153-7.132 (d, 1H), 6.692-6.670 (d, 1H), 5.9 (q, 1H), 4.434 (s, 2H), 3.396 (s, 3H), 2.694 (s, 3H), 1.548-1.531 (d, 3H). |
| | | 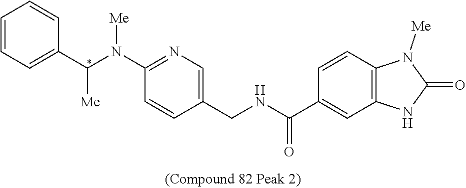 [SMILES: CC(c1ccccc1)N(C)c1ccc(CNC(=O)c2ccc3c(c2)[nH]c(=O)n3C)cn1] | LC-MS: 416.3 [M + H]⁺. ¹H-NMR (400 MHz, CD₃OD) δ 8.008-8.082 (d, 1H), 7.639-7.592 (m, 1H), 7.587-7.554 (m, 2H), 7.305- 7.249 (m, 4H), 7.216-7.199 (m, 1H), 7.153-7.132 (d, 1H), 6.678-6.656 (d, 1H), 5.9 (q, 1H), 4.43 (s, 2H), 3.396 (s, 3H), 2.690 (s, 3H), 1.546-1.528 (d, 3H). |

TABLE 53

| Ex. | Ref. | Structure | LC-MS, NMR |
|---|---|---|---|
| 83 | E11-S1 | 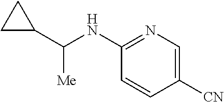 [SMILES: CC(Nc1ccc(C#N)cn1)C1CC1] | LC-MS: 188 [M + H]⁺ |
| | E11-S2 | 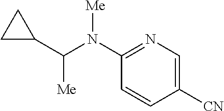 [SMILES: CC(C1CC1)N(C)c1ccc(C#N)cn1] | LC-MS: 202 [M + H]⁺ |
| | E11-S3 | 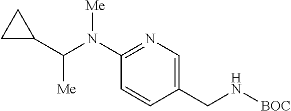 [SMILES: CC(C1CC1)N(C)c1ccc(CNC(=O)OC(C)(C)C)cn1] | LC-MS: 306 [M + H]⁺ |
| | E11-S4 | 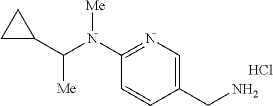 [SMILES: CC(C1CC1)N(C)c1ccc(CN)cn1 ; Cl] | LC-MS: 206 [M + H]⁺ |
| | E11-S5 | 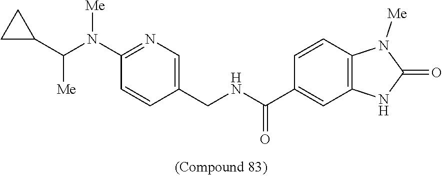 [SMILES: CC(C1CC1)N(C)c1ccc(CNC(=O)c2ccc3c(c2)[nH]c(=O)n3C)cn1] | LC-MS: 380.0 [M + H]⁺. ¹H-NMR (400 MHz, CD₃OD) δ 7.984-7.954 (dd, 1H), 7.821-7.817 (d, 1H), 7.640-7.615 (dd, 1H), 7.554-7.550 (d, 1H), 7.288- 7.264 (d, 1H), 7.162-7.141 (d, 1H), 4.441 (s, 2H), 3.509-3.470 (m, 1H), 3.391 (s, 3H), 3.174 (s, 3H), 1.356-1.339 (d, 3H), 1.186- |
| | | | 1.165 (m, 1H), 0.725-0.700 |
| | | | (m, 1H), 0.571-0.539 (m, |
| | | | 1H), 0.476-0.439 (m, 1H), |
| | | | 0.234-0.197 (m, 1H). |

TABLE 54

| Ex. | Ref. | Structure | LC-MS, NMR |
|---|---|---|---|
| 84 | E11-S1 | 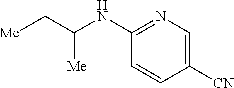 [SMILES: CCC(C)Nc1ccc(C#N)cn1] | LC-MS: 176 [M + H]⁺ |
| | E11-S2 | 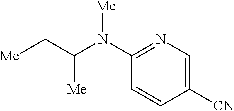 [SMILES: CCC(C)N(C)c1ccc(C#N)cn1] | LC-MS: 190 [M + H]⁺ |
| | E11-S3 | 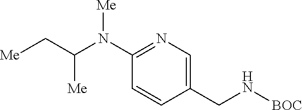 [SMILES: CCC(C)N(C)c1ccc(CNC(=O)OC(C)(C)C)cn1] | LC-MS: 294 [M + H]⁺ |
| | E11-S4 | 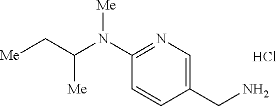 [SMILES: CCC(C)N(C)c1ccc(CN)cn1 ; Cl] | LC-MS: 194 [M + H]⁺ |
| | E11-S5 | 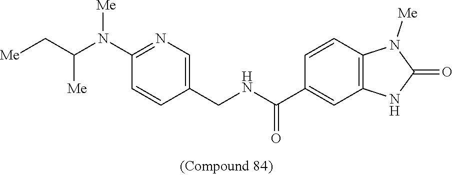 [SMILES: CCC(C)N(C)c1ccc(CNC(=O)c2ccc3c(c2)[nH]c(=O)n3C)cn1] | LC-MS: 368.2 [M + H]⁺. ¹H-NMR (400 MHz, CD₃OD) δ 9.054-9.025 (t, 1H), 8.051-8.022 (dd, 1H), 7.875-7.871 (d, 1H), 7.684- 7.659 (dd, 1H), 7.598-7.595 (d, 1H), 7.419-7.395 (d, 1H), 7.213-7.193 (d, 1H), 4.501-4.489 (d, 2H), 4.229- 4.176 (m, 1H), 3.440 (s, 3H), 3.06 (s, 3H), 1.786- |
| | | | 1.687 (m, 2H), 1.323-1.307 |
| | | | (d, 3H), 0.937-0.900 (t, 3H). |

TABLE 55

| Ex | Ref. | Structure | LC-MS, NMR |
|---|---|---|---|
| 85 | E14-S1 | 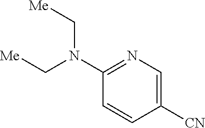 [SMILES: CCN(CC)c1ccc(C#N)cn1] | LC-MS: 176 [M + H]⁺ |
| | E14-S2 | 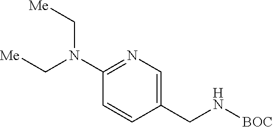 [SMILES: CCN(CC)c1ccc(CNC(=O)OC(C)(C)C)cn1] | LC-MS: 280 [M + H]⁺ |
| | E14-S3 | 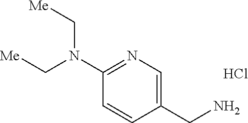 [SMILES: CCN(CC)c1ccc(CN)cn1 ; Cl] | LC-MS: 180 [M + H]⁺ |
| | E14-S4 | 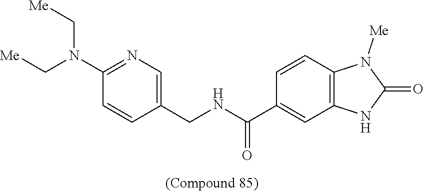 [SMILES: CCN(CC)c1ccc(CNC(=O)c2ccc3c(c2)[nH]c(=O)n3C)cn1] | LC-MS: 354.2 [M + H]⁺. ¹H-NMR (400 MHz, CD₃OD) δ 8.012-7.982 (dd, 1H), 7.821-7.817 (d, 1H), 7.644-7.618 (dd, 1H), 7.558-7.554 (d, 1H), 7.241- 7.217 (d, 1H), 7.171-7.151 (d, 1H), 4.445 (s, 2H), 3.648-3.594 (q, 4H), 3.398 (s, 3H), 1.278-1.243 (t, 6H). |

TABLE 56

| Ex. | Ref. | Structure | LC-MS, NMR |
|---|---|---|---|
| 86 | E11-S1 | 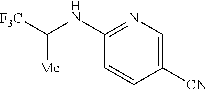 [SMILES: CC(Nc1ccc(C#N)cn1)C(F)(F)F] | LC-MS: 216 [M + H]⁺ |
| | E11-S2 | 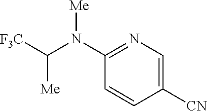 [SMILES: CC(N(C)c1ccc(C#N)cn1)C(F)(F)F] | LC-MS: 230 [M + H]⁺ |
| | E11-S3 | 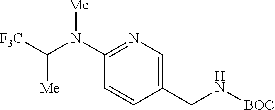 [SMILES: CC(N(C)c1ccc(CNC(=O)OC(C)(C)C)cn1)C(F)(F)F] | LC-MS: 334 [M + H]⁺ |
| | E11-S4 | 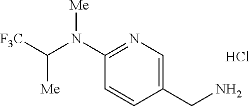 [SMILES: CC(N(C)c1ccc(CN)cn1)C(F)(F)F ; Cl] | LC-MS: 234 [M + H]⁺ |
| | E11-S5 | 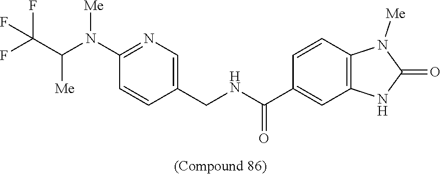 [SMILES: CC(N(C)c1ccc(CNC(=O)c2ccc3c(c2)[nH]c(=O)n3C)cn1)C(F)(F)F] | LC-MS: 408.2 [M + H]⁺. ¹H-NMR (400 MHz, CD₃OD) δ 8.114-8.109 (d, 1H), 7.630-7.600 (m, 2H), 7.549-7.546 (d, 1H), 7.147- 7.126 (d, 1H), 6.710-6.688 (d, 1H), 5.760 (m, 1H), 4.440 (s, 2H), 3.391 (s, 3H), 2.914 (s, 3H), 1.391-1.372 (d, 3H). |

TABLE 57

| Ex. | Ref. | Structure | LC-MS, NMR |
|---|---|---|---|
| 87 | E12-S1 | 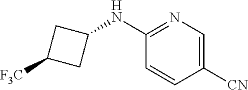 [SMILES: N#Cc1ccc(N[C@H]2C[C@H](C(F)(F)F)C2)nc1] | LC-MS: 242 [M + H]⁺ |
| | E12-S2 | 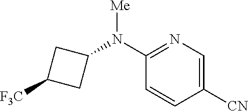 [SMILES: CN(c1ccc(C#N)cn1)[C@H]1C[C@H](C(F)(F)F)C1] | LC-MS: 256 [M + H]⁺ |
| | E12-S3 | 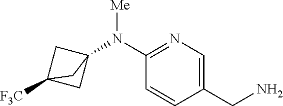 [SMILES: CN(c1ccc(CN)cn1)[C@]12C[C@@](C(F)(F)F)(C1)C2] | LC-MS: 260 [M + H]⁺ |
| | E12-S4 | 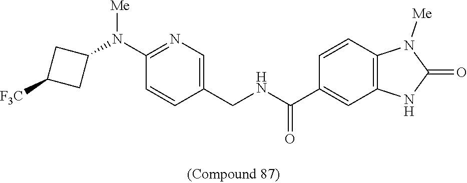 [SMILES: Cn1c(=O)[nH]c2cc(C(=O)NCc3ccc(N(C)[C@H]4C[C@H](C(F)(F)F)C4)nc3)ccc21] | LC-MS: 434.1 [M + H]⁺. ¹H-NMR (400 MHz, DMSO-D6) δ 11.068 (bs, 1H), 8.813-8.800 (t, 1H), 8.063 (bs, 1H), 7.617-7.596 (d, 1H), 7.507-7.490 (m, 2H), 7.146-7.125 (d, 1H), 6.608-6.586 (d, 1H), 5.144- 5.102 (m, 1H), 4.304-4.290 (d, 2H), 3.298 (s, 3H), 3.050-2.996 (m, 1H), 2.928 |
| | | | (s, 3H), 2.584 (m, 2H), |
| | | | 2.360 (m, 2H). |

TABLE 58

| Ex. | Ref. | Structure | LC-MS, NMR |
|---|---|---|---|
| 88 | E13-S3 | 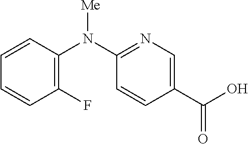 [SMILES: CN(c1ccc(C(=O)O)cn1)c1ccccc1F] | LC-MS: 246 [M + H]⁺ |
| | E13-S4 | 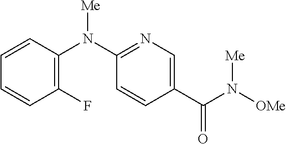 [SMILES: CON(C)C(=O)c1ccc(N(C)c2ccccc2F)nc1] | LC-MS: 290 [M + H]⁺ |
| | E13-S5 | 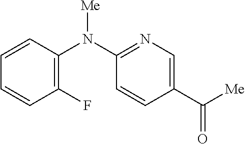 [SMILES: CC(=O)c1ccc(N(C)c2ccccc2F)nc1] | LC-MS: 245 [M + H]⁺ |
| | E13-S6 | 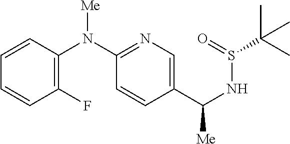 [SMILES: C[C@H](N[S@](=O)C(C)(C)C)c1ccc(N(C)c2ccccc2F)nc1] | LC-MS: 350 [M + H]⁺ |
| | E13-S7 | 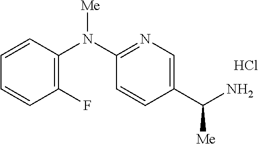 [SMILES: C[C@H](N)c1ccc(N(C)c2ccccc2F)nc1 ; Cl] | LC-MS: 246 [M + H]⁺ |
| | E13-S8 | 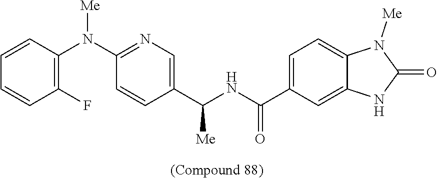 [SMILES: C[C@H](NC(=O)c1ccc2c(c1)[nH]c(=O)n2C)c1ccc(N(C)c2ccccc2F)nc1] | LC-MS: 420.2 [M + H]⁺. ¹H-NMR (400 MHz, CD₃OD) δ 8.01-7.961 (m, 2H), 7.641-7.621 (d, 1H), 7.531-7.45 (m, 3H), 7.38- 7.339 (m, 2H), 7.157-7.136 (d, 1H), 6.923-6.900 (d, 1H), 5.159-5.141 (q, 1H), 3.503 (s, 3H), 3.391 (s, 3H), 1.583-1.565 (d, 3H). |

TABLE 59

| Ex. | Ref. | Structure | LC-MS, NMR |
|---|---|---|---|
| 89 | E10-S1 | 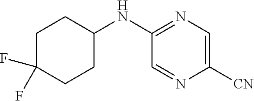 [SMILES: N#Cc1cnc(NC2CCC(F)(F)CC2)cn1] | LC-MS: 239 [M + H]⁺ |
| | E10-S2 | 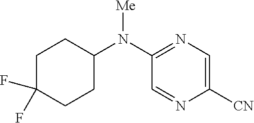 [SMILES: CN(c1cnc(C#N)cn1)C1CCC(F)(F)CC1] | LC-MS: 253 [M + H]⁺ |
| | E10-S3 | 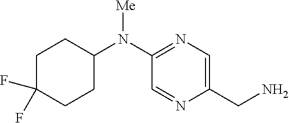 [SMILES: CN(c1cnc(CN)cn1)C1CCC(F)(F)CC1] | LC-MS: 257 [M + H]⁺ |
| | E10-S4 | 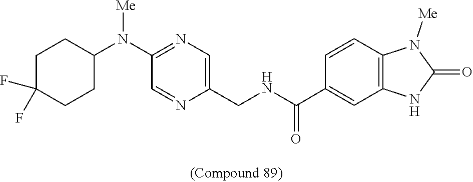 | LC-MS: 431.0 [M + H]⁺. ¹H-NMR (400 MHz, DMSO-D₆) δ 11.036 (bs, 1H), 8.860-8.831 (t, 1H), 8.094 (s, 1H), 8.025 (s, 1H), 7.611-7.589 (d, 1H), 7.482 (s, 1H), 7.118-7.098 (d, 1H), 4.478 (m, 1H), 4.388-4.374 (d, 2H), 3.271 (s, 3H), 2.845 (s, 3H), 2.054-1.956 (m, 4H), 1.765-1.723 (m, 2H), 1.648-1.618 (m, 2H). |

TABLE 60

| Ex. | Ref. | Structure | LC-MS, NMR |
|---|---|---|---|
| 90 | E10-S1 | 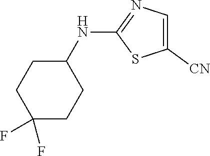 [SMILES: N#Cc1cnc(NC2CCC(F)(F)CC2)s1] | LC-MS: 244 [M + H]⁺ |
| | E10-S2 | 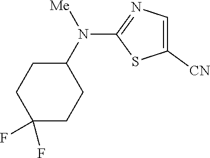 [SMILES: CN(c1ncc(C#N)s1)C1CCC(F)(F)CC1] | LC-MS: 258 [M + H]⁺ |
| | E10-S3 | 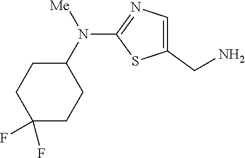 [SMILES: CN(c1ncc(CN)s1)C1CCC(F)(F)CC1] | LC-MS: 262 [M + H]⁺ |
| | E10-S4 | 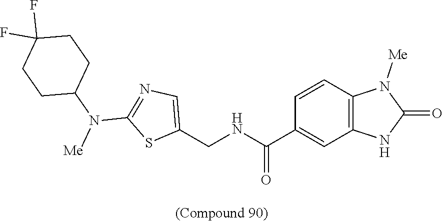 [SMILES: CN(c1ncc(CNC(=O)c2ccc3c(c2)[nH]c(=O)n3C)s1)C1CCC(F)(F)CC1] | LC-MS: 435.9 [M + H]⁺. ¹H-NMR (400 MHz, DMSO-D₆) δ 7.587-7.565 (dd, 1H), 7.494-7.490 (d, 1H), 7.159-7.138 (d, 1H), 6.987 (s, 1H), 4.411 (s, 2H), 3.996 (m, 1H), 3.290 (s, 3H), 2.804 (s, 3H), 2.073- 1.897 (m, 4H), 1.756-1.694 (m, 4H). |

TABLE 61

| Ex. | Ref. | Structure | LC-MS, NMR |
|---|---|---|---|
| 91 | E10-S1 | 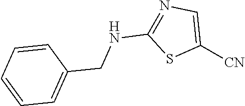 [SMILES: N#Cc1cnc(NCc2ccccc2)s1] | LC-MS: 216 [M + H]⁺ |
| | E10-S2 | 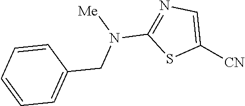 [SMILES: CN(Cc1ccccc1)c1ncc(C#N)s1] | LC-MS: 230 [M + H]⁺ |
| | E10-S3 | 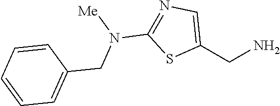 [SMILES: CN(Cc1ccccc1)c1ncc(CN)s1] | LC-MS: 234 [M + H]⁺ |
| | E10-S4 | 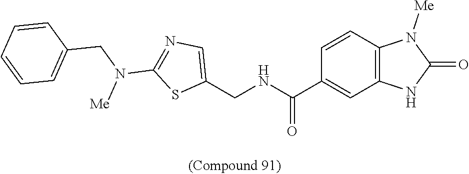 [SMILES: CN(Cc1ccccc1)c1ncc(CNC(=O)c2ccc3c(c2)[nH]c(=O)n3C)s1] | LC-MS: 408.2 [M + H]⁺. ¹H-NMR (400 MHz, CD₃OD) δ 7.672-7.647 (dd, 1H), 7.594-7.591 (d, 1H), 7.447-7.334 (m, 6H), 7.205- 7.185 (d, 1H), 4.815 (s, 2H), 4.570 (s, 2H), 3.434 (s, 3H), 3.282 (s, 3H). |

TABLE 62

| Ex. | Ref. | Structure | LC-MS, NMR |
|---|---|---|---|
| 92 | E10-S1 | 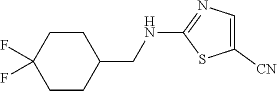 [SMILES: N#Cc1cnc(NCC2CCC(F)(F)CC2)s1] | LC-MS: 258 [M + H]⁺ |
| | E10-S2 | 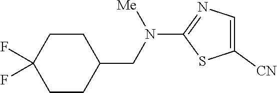 [SMILES: CN(CC1CCC(F)(F)CC1)c1ncc(C#N)s1] | LC-MS: 272 [M + H]⁺ |
| | E8-S2 | 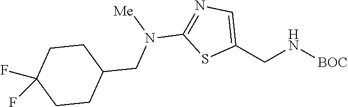 [SMILES: CN(CC1CCC(F)(F)CC1)c1ncc(CNC(=O)OC(C)(C)C)s1] | LC-MS: 376 [M + H]⁺ |
| | E8-S3 | 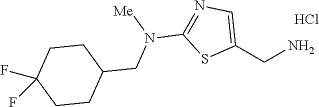 [SMILES: CN(CC1CCC(F)(F)CC1)c1ncc(CN)s1 ; Cl] | LC-MS: 276 [M + H]⁺ |
| | E10-S4 | 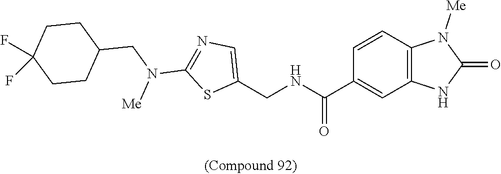 [SMILES: CN(CC1CCC(F)(F)CC1)c1ncc(CNC(=O)c2ccc3c(c2)[nH]c(=O)n3C)s1] | LC-MS: 450.3 [M + H]⁺. ¹H-NMR (400 MHz, CD₃OD) δ 7.677-7.673 (dd, 1H), 7.602-7.599 (d, 1H), 7.269 (s, 1H), 7.211-7.190 (d, 1H), 4.563 (s, 2H), 3.481-3.462 (d, 2H), 3.439 (s, 3H), 3.246 (s, 3H), 2.086-2.001 (m, 3H), 1.820- 1.742 (m, 4H), 1.384-1.319 (m, 2H). |

TABLE 63

| Ex. | Ref. | Structure | LC-MS, NMR |
|---|---|---|---|
| 93 | E10-S1 | 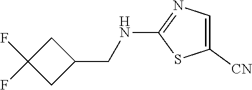 [SMILES: N#Cc1cnc(NCC2CC(F)(F)C2)s1] | LC-MS: 230 [M + H]⁺ |
| | E10-S2 | 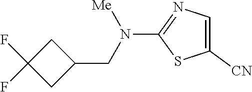 [SMILES: CN(CC1CC(F)(F)C1)c1ncc(C#N)s1] | LC-MS: 244 [M + H]⁺ |
| | E8-S2 | 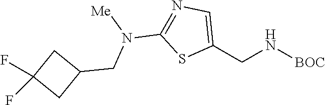 [SMILES: CN(CC1CC(F)(F)C1)c1ncc(CNC(=O)OC(C)(C)C)s1] | LC-MS: 348 [M + H]⁺ |
| | E8-S3 | 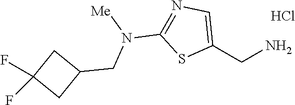 [SMILES: CN(CC1CC(F)(F)C1)c1ncc(CN)s1 ; Cl] | LC-MS: 248 [M + H]⁺ |
| | E10-S4 | 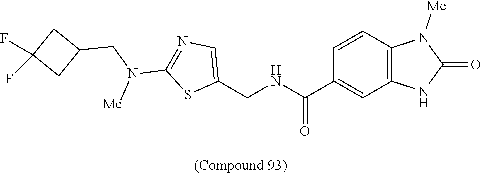 [SMILES: CN(CC1CC(F)(F)C1)c1ncc(CNC(=O)c2ccc3c(c2)[nH]c(=O)n3C)s1] | LC-MS: 422.1 [M + H]⁺. ¹H-NMR (400 MHz, CD₃OD) δ 7.673-7.648 (dd, 1H), 7.598-7.594 (d, 1H), 7.293 (s, 1H), 7.212-7.191 (d, 1H), 4.568 (s, 2H), 3.718-3.700 (d, 2H), 3.440 (s, 3H), 3.243 (s, 3H), 2.773-2.705 (m, 2H), 2.695- 2.626 (m, 1H), 2.493-2.287 (m, 2H). |

TABLE 64

| Ex. | Ref. | Structure | LC-MS, NMR |
|---|---|---|---|
| 94 | E11-S1 | 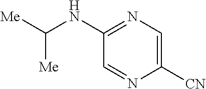 [SMILES: CC(C)Nc1cnc(C#N)cn1] | LC-MS: 163 [M + H]⁺ |
| | E11-S2 | 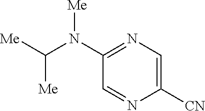 [SMILES: CC(C)N(C)c1cnc(C#N)cn1] | LC-MS: 177 [M + H]⁺ |
| | E10-S3 | 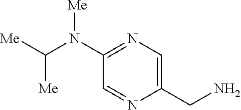 [SMILES: CC(C)N(C)c1cnc(CN)cn1] | LC-MS: 181 [M + H]⁺ |
| | E11-S5 | 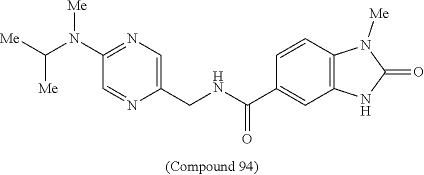 [SMILES: CC(C)N(C)c1cnc(CNC(=O)c2ccc3c(c2)[nH]c(=O)n3C)cn1] | LC-MS: 355.0 [M + H]⁺. ¹H-NMR (400 MHz, CD₃OD) δ 8.231 (s, 1H), 8.055 (s, 1H), 7.659-7.638 (d, 1H), 7.567 (s, 1H), 7.164-7.123 (d, 1H), 4.736- 4.720 (m, 1H), 4.540 (s, 2H), 3.398 (s, 3H), 2.859 (s, 3H), 1.235-1.219 (d, 6H). |

TABLE 65

| Ex. | Ref. | Structure | LC-MS, NMR |
|---|---|---|---|
| 95 | E12-S1 | 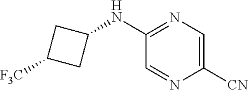 [SMILES: N#Cc1cnc(N[C@H]2C[C@@H](C(F)(F)F)C2)cn1] | LC-MS: 241 [M − H]⁻ |
| | E12-S2 | 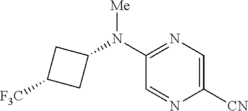 [SMILES: CN(c1cnc(C#N)cn1)[C@H]1C[C@@H](C(F)(F)F)C1] | LC-MS: 257 [M + H]⁺ |
| | E12-S3 | 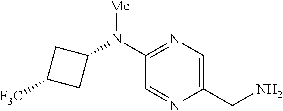 [SMILES: CN(c1cnc(CN)cn1)[C@H]1C[C@@H](C(F)(F)F)C1] | LC-MS: 261 [M + H]⁺ |
| | E12-S4 | 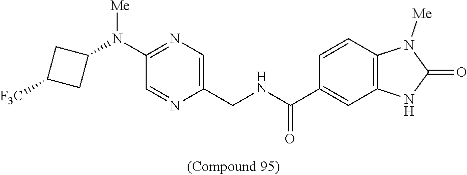 [SMILES: Cn1c(=O)[nH]c2cc(C(=O)NCc3cnc(N(C)[C@H]4C[C@@H](C(F)(F)F)C4)cn3)ccc21] | LC-MS: 435.2 [M + H]⁺. ¹H-NMR (400 MHz, DMSO-D₆) δ 11.084 (s, 1H), 8.908 (t, 1H), 8.102- 8.067 (dd, 2H), 7.643-7.618 (dd, 1H), 7.513-7.510 (d, 1H), 7.155-7.134 (d, 1H), 4.839 (m, 1H), 4.424-4.410 (d, 2H), 3.301 (s, 3H), 2.96- 2.929 (m, 4H), 2.441-2.379 (m, 2H), 2.372-2.235 (m, |
| | | | 2H). |

TABLE 66

| Ex. | Ref. | Structure | LC-MS, NMR |
|---|---|---|---|
| 96 | E10-S1 | 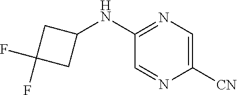 [SMILES: N#Cc1cnc(NC2CC(F)(F)C2)cn1] | LC-MS: 211 [M + H]⁺ |
| | E10-S2 | 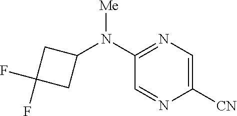 [SMILES: CN(c1cnc(C#N)cn1)C1CC(F)(F)C1] | LC-MS: 225 [M + H]⁺ |
| | E8-S2 | 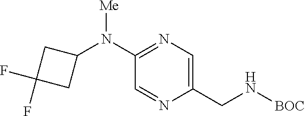 [SMILES: CN(c1cnc(CNC(=O)OC(C)(C)C)cn1)C1CC(F)(F)C1] | LC-MS: 329 [M + H]⁺ |
| | E8-S2 | 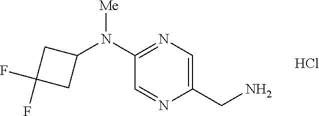 [SMILES: CN(c1cnc(CN)cn1)C1CC(F)(F)C1 ; Cl] | LC-MS: 229 [M + H]⁺ |
| | E10-S4 | 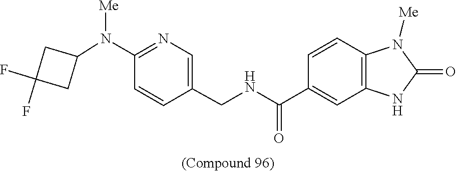 [SMILES: CN(c1ccc(CNC(=O)c2ccc3c(c2)[nH]c(=O)n3C)cn1)C1CC(F)(F)C1] | LC-MS: 403.1 [M + H]⁺. ¹H-NMR (400 MHz, CD₃OD) δ 8.145-8.141 (d, 1H), 8.053-8.049 (d, 1H), 7.660-7.635 (dd, 1H), 7.569-7.566 (d, 1H), 7.160- 7.139 (d, 1H), 4.640-4.610 (m, 1H), 4.542 (s, 2H), 3.398 (s, 3H), 3.040 (s, 3H), 2.932-2.826 (m, 2H), 2.805- 2.725 (m, 2H). |

TABLE 67

| Ex | Ref. | Structure | LC-MS, NMR |
|---|---|---|---|
| 97 | E14-S1 | 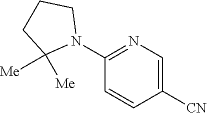 [SMILES: CC1(C)CCCN1c1ccc(C#N)cn1] | LC-MS: 202 [M + H]⁺ |
| | E10-S3 | 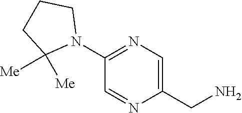 [SMILES: CC1(C)CCCN1c1cnc(CN)cn1] | LC-MS: 206 [M + H]⁺ |
| | E14-S4 | 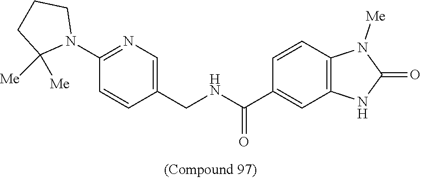 [SMILES: Cn1c(=O)[nH]c2cc(C(=O)NCc3ccc(N4CCCC4(C)C)nc3)ccc21] | LC-MS: 380.3 [M + H]⁺. ¹H-NMR (400 MHz, DMSO-D₆) δ 11.074 (s, 1H), 8.788 (t, 1H), 8.018- 8.013 (d, 1H), 7.635-7.610 (dd, 1H), 7.511-7.507 (d, 1H), 7.431-7.403 (dd, 1H), 7.152-7.132 (d, 1H), 6.396- 6.375 (d, 1H), 4.289-4.275 (d, 2H), 3.334-3.305 (m, 2H), 3.30 (s, 3H), 1.869- 1.844 (m, 4H), 1.463(s, 6H). |

TABLE 68

| Ex. | Ref. | Structure | LC-MS, NMR |
|---|---|---|---|
| 98 | E14-S1 | 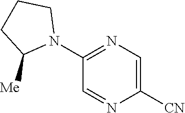 [SMILES: C[C@H]1CCCN1c1cnc(C#N)cn1] | LC-MS: 189 [M + H]⁺ |
| | E10-S3 | 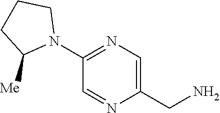 [SMILES: C[C@H]1CCCN1c1cnc(CN)cn1] | LC-MS: 193 [M + H]⁺ |
| | E14-S4 | 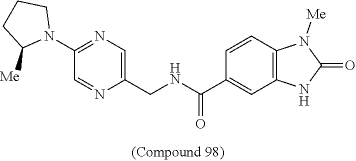 [SMILES: C[C@H]1CCCN1c1cnc(CNC(=O)c2ccc3c(c2)[nH]c(=O)n3C)cn1] | LC-MS: 367.1 [M + H]⁺. ¹H-NMR (400 MHz, CD₃OD) δ 8.156 (s, 1H), 7.966 (s, 1H), 7.657-7.631 (dd, 1H), 7.565- 7.560 (d, 1H), 7.160-7.139 (d, 1H), 4.532 (s, 2H), 4.277- 4.201 (m, 1H), 3.660-3.614 (m, 1H), 3.445-3.396 (m, 5H), 2.203-2.041 (m, 2H), 1.835- 1.798 (m, 1H), 1.235-1.201 (d, 3H). |

TABLE 69

| Ex. | Ref. | Structure | LC-MS, NMR |
|---|---|---|---|
| 99 | E14-S1 | 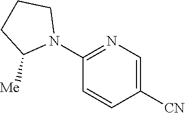 [SMILES: C[C@@H]1CCCN1c1ccc(C#N)cn1] | LC-MS: 188 [M + H]⁺ |
| | E14-S2 | 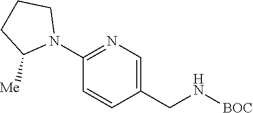 [SMILES: C[C@@H]1CCCN1c1ccc(CNC(=O)OC(C)(C)C)cn1] | LC-MS: 292 [M + H]⁺ |
| | E14-S3 | 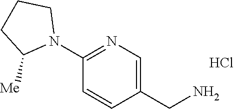 [SMILES: C[C@@H]1CCCN1c1ccc(CN)cn1 ; Cl] | LC-MS: 192 [M + H]⁺ |
| | E14-S4 | 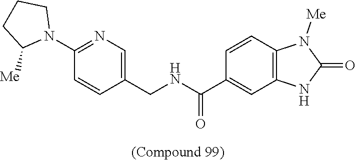 [SMILES: C[C@@H]1CCCN1c1ccc(CNC(=O)c2ccc3c(c2)[nH]c(=O)n3C)cn1] | LC-MS: 366.0 [M + H]⁺. ¹H-NMR (400 MHz, CD₃OD) δ 9.0 (t, 1H), 7.995-7.966 (dd, 1H), 7.818-7.815 (d, 1H), 7.639-7.615 (dd, 1H), 7.555- 7.550 (d, 1H), 7.171-7.150 (d, 1H), 7.127-7.104 (d, 1H), 4.443 (s, 2H), 4.259-4.228 (m, 1H), 3.696-3.650 (m, 1H), 3.468-3.443 (m, 1H), 3.398 (s, |
| | | | 3H), 2.256-2.124 (m, 3H), |
| | | | 1.902-1.876 (m, 1H), 1.255- |
| | | | 1.239 (d, 3H). |

TABLE 70

| Ex. | Ref. | Structure | LC-MS, NMR |
|---|---|---|---|
| 100 | E20-S2 | 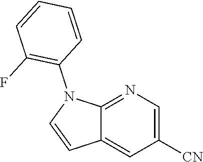 [SMILES: N#Cc1cnc2c(ccn2-c2ccccc2F)c1] | LC-MS: 238 [M + H]⁺ |
| | E20-S3 | 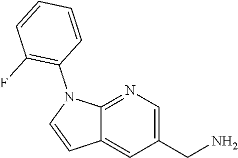 [SMILES: NCc1cnc2c(ccn2-c2ccccc2F)c1] | LC-MS: 242 [M + H]⁺ |
| | E20-S4 | 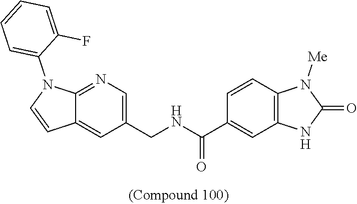 [SMILES: Cn1c(=O)[nH]c2cc(C(=O)NCc3cnc4c(ccn4-c4ccccc4F)c3)ccc21] | LC-MS: 416.2 [M + H]⁺. ¹H-NMR (400 MHz, DMSO- D₆) δ 11.077 (s, 1H), 8.995 (t, 1H), 8.256 (s, 1H) 8.016 (s, 1H), 7.712-7.634 (m, 3H), 7.526-7.470 (m, 3H), 7.382 (m, 1H), 7.157-7.138 (d, 1H), 6.717-6.709 (d, 1H), 4.576- 4.562 (d, 2H), 3.298 (s, 3H). |

TABLE 71

| Ex. | Ref. | Structure | LC-MS, NMR |
|---|---|---|---|
| 101 | E21-S1 | 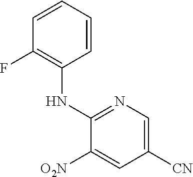 [SMILES: N#Cc1cnc(Nc2ccccc2F)c([N+](=O)[O-])c1] | LC-MS: 257 [M − H]⁻ |
| | E21-S2 | 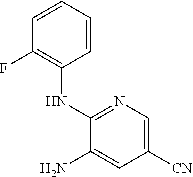 [SMILES: N#Cc1cnc(Nc2ccccc2F)c(N)c1] | LC-MS: 229 [M + H]⁺ |
| | E21-S3 | 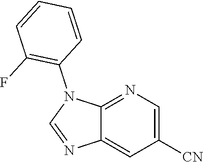 [SMILES: N#Cc1cnc2c(c1)ncn2-c1ccccc1F] | LC-MS: 239 [M + H]⁺ |
| | E21-S4 | 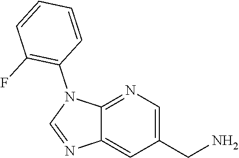 [SMILES: NCc1cnc2c(c1)ncn2-c1ccccc1F] | LC-MS: 243 [M + H]⁺ |
| | E21-S5 | 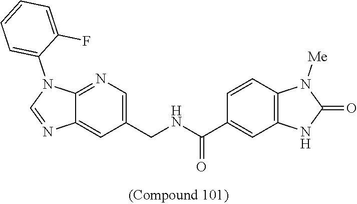 [SMILES: Cn1c(=O)[nH]c2cc(C(=O)NCc3cnc4c(c3)ncn4-c3ccccc3F)ccc21] | LC-MS: 417.2 [M + H]⁺. ¹H-NMR (400 MHz, CD₃OD) δ 9.102-9.074 (t, 1H), 8.568 (s, 1H), 8.452 (s, 1H), 8.190 (s, 1H), 7.757-7.715 (m, 1H), 7.612-7.647 (dd, 1H), 7.603- 7.550 (m, 2H), 7.451-7.395 (m, 2H), 7.162-7.141 (d, 1H), 4.741-4.732 (d, 2H), 3.395 (s, 3H). |

TABLE 72

| Ex. | Ref. | Structure | LC-MS, NMR |
|---|---|---|---|
| 102 | E20-S1 | 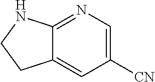 [SMILES: N#Cc1cnc2c(c1)CCN2] | LC-MS: 146 [M + H]⁺ |
| | E20-S2 | 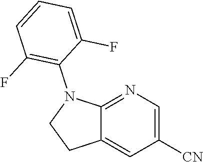 [SMILES: N#Cc1cnc2c(c1)CCN2c1c(F)cccc1F] | LC-MS: 258 [M + H]⁺ |
| | E8-S2 | 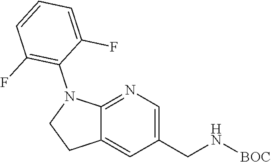 [SMILES: CC(C)(C)OC(=O)NCc1cnc2c(c1)CCN2c1c(F)cccc1F] | LC-MS: 362 [M + H]⁺ |
| | E8-S3 | 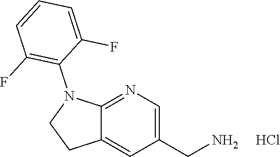 [SMILES: NCc1cnc2c(c1)CCN2c1c(F)cccc1F ; Cl] | LC-MS: 262 [M + H]⁺ |
| | E20-S4 | 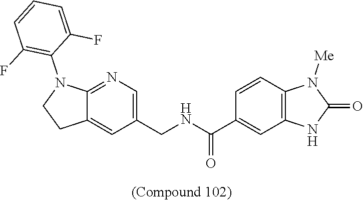 [SMILES: Cn1c(=O)[nH]c2cc(C(=O)NCc3cnc4c(c3)CCN4c3c(F)cccc3F)ccc21] | LC-MS: 436.1 [M + H]⁺. ¹H-NMR (400 MHz, CD₃OD) δ 7.731-7.727 (d, 1H), 7.647- 7.626 (m, 2H), 7.566-7.562 (d, 1H), 7.511-7.437 (m, 1H), 7.206-7.143 (m, 3H), 4.448 (s, 2H), 4.159-4.116 (t, 2H), 3.397 (s, 3H), 3.377-3.334 (t, 2H). |

TABLE 73

| Ex. | Ref. | Structure | LC-MS, NMR |
|---|---|---|---|
| 117 | E103-S1 | 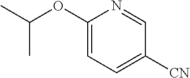 | LC-MS: 163 [M + H]⁺ |
| | E103-S2 | 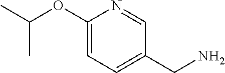 | LC-MS: 167 [M + H]⁺ |
| | E103-S3 | 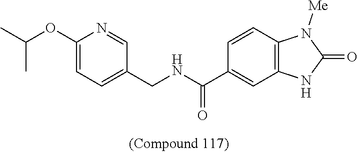 [SMILES: CC(C)Oc1ccc(CNC(=O)c2ccc3c(c2)[nH]c(=O)n3C)cn1] | LC-MS: 341 [M + H]⁺ ¹H-NMR (400 MHz, CD₃OD): δ 8.19-8.18 (s, 1H), 8.06-8.03 (dd, 1H), 7.64-7.62 (dd, 1H), 7.56-7.55 (s, 1H), 7.16-7.12 (t, 2H), 5.15-5.12 (m, 1H), 4.52 (s, 2H), 3.39(s, 3H), 1.38-1.36 (d, 6H). |

TABLE 74

| Ex. | Ref. | Structure | LC-MS, NMR |
|---|---|---|---|
| 118 | E103-S1 | 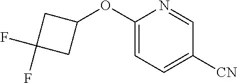 [SMILES: N#Cc1ccc(OC2CC(F)(F)C2)nc1] | LC-MS: 211 [M + H]⁺ |
| | E103-S2 | 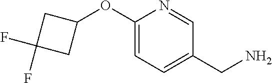 [SMILES: NCc1ccc(OC2CC(F)(F)C2)nc1] | LC-MS: 215 [M + H]⁺ |
| | E10-S4 | 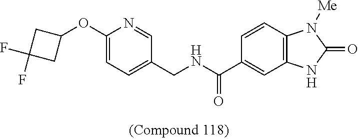 [SMILES: Cn1c(=O)[nH]c2cc(C(=O)NCc3ccc(OC4CC(F)(F)C4)nc3)ccc21] | LC-MS: 389 [M + H]⁺ ¹H-NMR (400 MHz, CD₃OD): δ 8.34-8.32 (m, 2H), 7.66-7.63 (dd, 1H), 7.57-7.56 (s, 1H), 7.34-7.32 (d, 1H), 7.17-7.14 (d, 1H), 5.20-5.15 (bs, 1H), 4.57 (s, 2H), 3.39 (s, 3H), 3.22-3.19 (m, 2H), 2.93-2.81 (m, 2H). |

TABLE 75

| Ex. | Ref. | Structure | LC-MS, NMR |
|---|---|---|---|
| 119 | E10-S2 | 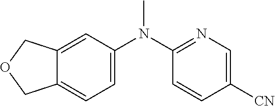 [SMILES: CN(c1ccc2c(c1)COC2)c1ccc(C#N)cn1] | LC-MS: 252 [M + H]⁺ |
| | E10-S3 | 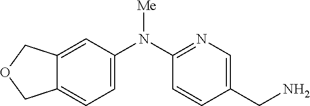 [SMILES: CN(c1ccc2c(c1)COC2)c1ccc(CN)cn1] | LC-MS: 256 [M + H]⁺ |
| | E10-S4 | 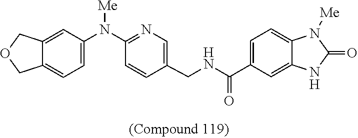 [SMILES: CN(c1ccc2c(c1)COC2)c1ccc(CNC(=O)c2ccc3c(c2)[nH]c(=O)n3C)cn1] | LC-MS: 430 [M + H]⁺ ¹H-NMR (400 MHz, CD₃OD): δ 8.67-8.51 (bs, 1H), 8.14-8.13 (d, 1H), 7.66-7.64 (dd, 1H), 7.58 (d, 1H), 7.50-7.47 (dd, 1H), 7.37-7.33 (dd, 1H), 7.19- 7.15 (m, 3H), 6.56-6.54 (dd, 1H), 5.09-5.08 (s, 4H), 4.46 (s, 2H), 3.43-3.42 (s, 6H). |

TABLE 76

| Ex. | Ref. | Structure | LC-MS, NMR |
|---|---|---|---|
| 120 | E10-S1 | 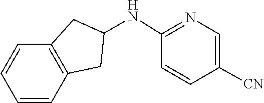 [SMILES: N#Cc1ccc(NC2Cc3ccccc3C2)nc1] | LC-MS: 236 [M + H]⁺ |
| | E10-S2 | 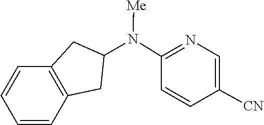 [SMILES: CN(c1ccc(C#N)cn1)C1Cc2ccccc2C1] | LC-MS: 250 [M + H]⁺ |
| | E10-S3 | 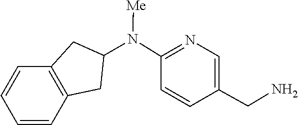 [SMILES: CN(c1ccc(CN)cn1)C1Cc2ccccc2C1] | LC-MS: 237 [M − NH2]⁺ |
| | E10-S4 | 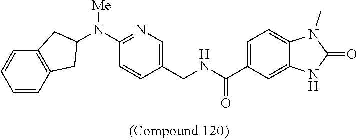 [SMILES: CN(c1ccc(CNC(=O)c2ccc3c(c2)[nH]c(=O)n3C)cn1)C1Cc2ccccc2C1] | LC-MS: 428 [M + H]⁺ ¹H-NMR (400 MHz, CD₃OD): δ 8.06-8.03 (dd, 1H), 7.87 (s, 1H), 7.64-7.62 (dd, 1H), 7.56 (s, 1H), 7.44-7.42 (d, 1H), 7.26-7.24 (m, 2H), 7.19-7.15 (m, 3H), 5.09-5.05 (m, 1H), 4.47-4.43 (s, 2H), 3.45-3.40 (s, 3H), 3.37-3.35 (d, 2H), |
| | | | 3.22-3.19 (d, 1H), 3.16-3.14 |
| | | | (d, 1H), 3.04 (s, 3H) |

TABLE 77

| Ex. | Ref. | Structure | LC-MS, NMR |
|---|---|---|---|
| 121 | E10-S1 | 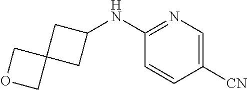 [SMILES: N#Cc1ccc(NC2CC3(COC3)C2)nc1] | LC-MS: 216 [M + H]⁺ |
| | E10-S2 | 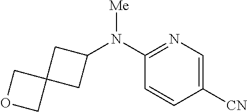 [SMILES: CN(c1ccc(C#N)cn1)C1CC2(COC2)C1] | LC-MS: 230 [M + H]⁺ |
| | E10-S3 | 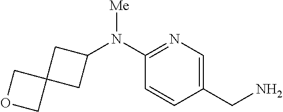 [SMILES: CN(c1ccc(CN)cn1)C1CC2(COC2)C1] | LC-MS: 234 [M + H]⁺ |
| | E10-S4 | 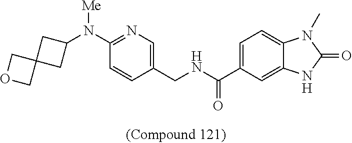 [SMILES: CN(c1ccc(CNC(=O)c2ccc3c(c2)[nH]c(=O)n3C)cn1)C1CC2(COC2)C1] | LC-MS: 408 [M + H]⁺ ¹H-NMR (400 MHz, CD₃OD): δ 8.05-8.04 (d, 1H), 7.62-7.60 (dd, 1H), 7.56-7.53 (m, 2H), 7.14-7.12 (d, 1H), 6.62-6.60 (d, 1H), 4.78 (s, 2H), 4.62 (s, 2H), 4.42-4.40 (m, 3H), 3.39 (s, 3H), 2.90 (s, 3H), 2.60- 2.56 (m, 2H), 2.36-2.30 (m, 2H). |

TABLE 78

| Ex. | Ref. | Structure | LC-MS, NMR |
|---|---|---|---|
| 122 | E105-S1 | 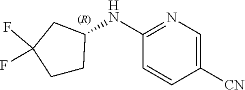 | LC-MS: 224 [M + H]⁺ |
| | E105-S2 | 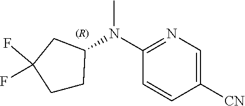 | LC-MS: 238 [M + H]⁺ |
| | E105-S3 | 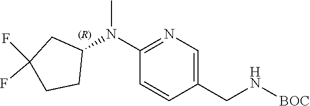 | LC-MS: 342 [M + H]⁺ |
| | E105-S4 | 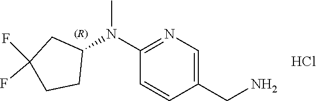 | LC-MS: 242 [M + H]⁺ |
| | E105-S5 | 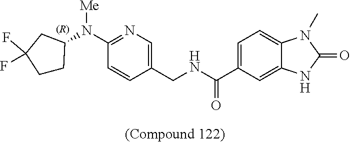 | LC-MS: 416 [M + H]⁺ ¹H-NMR (400 MHz, CD₃OD): δ 8.11-8.10 (d, 1H), 7.66-7.61 (dd, 1H), 7.60-7.58 (m, 2H), 7.18-7.16 (d, 1H), 6.70-6.65 (d, 1H), 5.27-5.23 (m, 1H), 4.45 (s, 2H), 3.43 (s, 3H), 2.91 (s, 3H), 2.36-1.96 (m, 6H). |

TABLE 79

| Ex. | Ref. | Structure | LC-MS, NMR |
|---|---|---|---|
| 123 | E10-S1 | 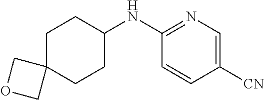 [SMILES: N#Cc1ccc(NC2CCC3(CC2)COC3)nc1] | LC-MS: 244 [M + H]⁺ |
| | E10-S2 | 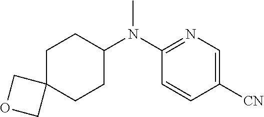 [SMILES: CN(c1ccc(C#N)cn1)C1CCC2(CC1)COC2] | LC-MS: 258 [M + H]⁺ |
| | E10-S3 | 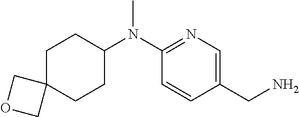 [SMILES: CN(c1ccc(CN)cn1)C1CCC2(CC1)COC2] | LC-MS: 262 [M + H]⁺ |
| | E10-S4 | 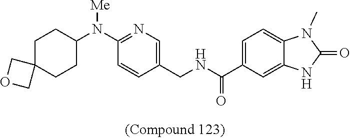 [SMILES: CN(c1ccc(CNC(=O)c2ccc3c(c2)[nH]c(=O)n3C)cn1)C1CCC2(CC1)COC2] | LC-MS: 436 [M + H]⁺ ¹H-NMR (400 MHz, CD₃OD): δ 8.39 (bs, 1H), 8.06-8.05 (d, 1H), 7.66-7.64 (dd, 1H), 7.60- 7.58 (m, 2H), 7.18-7.16 (d, 1H), 6.68-6.66 (d, 1H), 4.54 (s, 2H), 4.44 (s, 2H), 4.38 (s, 2H), 4.26 (m, 1H), 3.43 (s, 3H), 2.83 (s, 3H), 2.66-2.24 (d, 2H), 1.66-1.58 (m, 6H). |

TABLE 80

| Ex. | Ref. | Structure | LC-MS, NMR |
|---|---|---|---|
| 124 | E10-S1 | 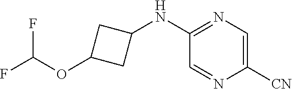 [SMILES: N#Cc1cnc(NC2CC(OC(F)F)C2)cn1] | LC-MS: 239 [M − H]⁻ |
| | E10-S2 | 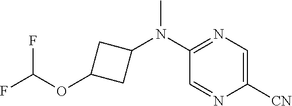 [SMILES: CN(c1cnc(C#N)cn1)C1CC(OC(F)F)C1] | LC-MS: 255 [M + H]⁺ |
| | E10-S3 | 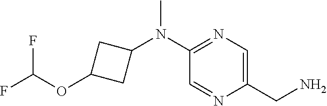 | LC-MS: 242 [M − NH2]⁻ |
| | E10-S4 | 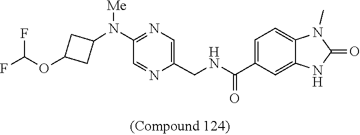 [SMILES: CN(c1cnc(CNC(=O)c2ccc3c(c2)[nH]c(=O)n3C)cn1)C1CC(OC(F)F)C1] | LC-MS: 433 [M + H]⁺ ¹H-NMR (400 MHz, CD₃OD): δ 8.16 (d, 1H), 8.07-8.06 (d, 1H), 7.70-7.67 (dd, 1H), 7.61- 7.60 (d, 1H), 7.20-7.18 (d, 1H), 6.56-6.18 (m, 1H), 4.58 (s, 2H), 4.47-4.43 (m, 2H), 3.44 (s, 3H), 3.09 (s, 3H), 2.79-2.76 (m, 2H), 2.36-2.32 (m, 2H). |

TABLE 81

| Ex. | Ref. | Structure | LC-MS, NMR |
|---|---|---|---|
| 125 | E10-S1 | 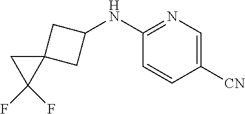 [SMILES: N#Cc1ccc(NC2CC3(C2)CC3(F)F)nc1] | LC-MS: 236 [M + H]⁺ |
| | E10-S2 | 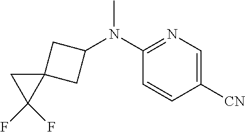 [SMILES: CN(c1ccc(C#N)cn1)C1CC2(C1)CC2(F)F] | LC-MS: 250 [M + H]⁺ |
| | E10-S3 | 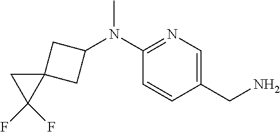 [SMILES: CN(c1ccc(CN)cn1)C1CC2(C1)CC2(F)F] | LC-MS: 254 [M + H]⁺ |
| | E10-S4 | 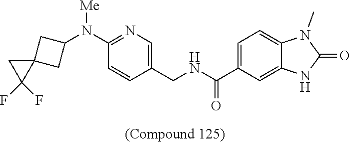 [SMILES: CN(c1ccc(CNC(=O)c2ccc3c(c2)[nH]c(=O)n3C)cn1)C1CC2(C1)CC2(F)F] | LC-MS: 428 [M + H]⁺ ¹H-NMR (400 MHz, CD₃OD): δ 8.11-8.08 (dd, 1H), 7.94 (s, 1H), 7.69-7.66 (dd, 1H), 7.60 (s, 1H), 7.36-7.34 (d, 1H), 7.21-7.19 (d, 1H), 4.74-4.72 (m, 1H), 4.51 (s, 2H), 3.43 (s, 3H), 3.28 (s, 3H), 2.73-2.63 (m, 4H), 1.50-1.38 (m, 2H). |

TABLE 82

| Ex. | Ref. | Structure | LC-MS, NMR |
|---|---|---|---|
| 126 | E10-S1 | 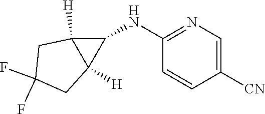 [SMILES: N#Cc1ccc(N[C@H]2[C@@H]3CC(F)(F)C[C@@H]32)nc1] | LC-MS: 263 [M + H]⁺ |
| | E10-S2 | 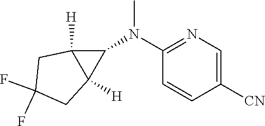 [SMILES: CN(c1ccc(C#N)cn1)[C@H]1[C@@H]2CC(F)(F)C[C@@H]21] | LC-MS: 250 [M + H]⁺ |
| | E10-S3 | 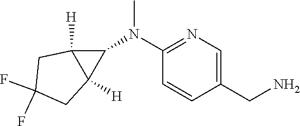 [SMILES: CN(c1ccc(CN)cn1)[C@H]1[C@@H]2CC(F)(F)C[C@@H]21] | LC-MS: 254 [M + H]⁺ |
| | E10-S4 | 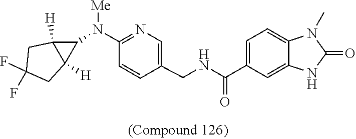 | LC-MS: 428 [M + H]⁺ ¹H-NMR (400 MHz, CD₃OD): δ 8.13-8.12 (d, 1H), 7.67-7.63 (m, 2H), 7.59-7.58 (d, 1H), 7.18-7.16 (d, 1H), 6.87-6.84 (dd, 1H), 4.47 (s, 2H), 3.43 (s, 3H), 3.09 (s, 3H), 2.55- 2.30 (m, 5H), 1.72-1.71 (m, 2H). |

TABLE 83

| Ex. | Ref. | Structure | LC-MS, NMR |
|---|---|---|---|
| 127 | E10-S1 | 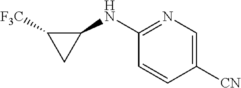 [SMILES: N#Cc1ccc(N[C@H]2C[C@@H]2C(F)(F)F)nc1] | LC-MS: 228 [M + H]⁺ |
| | E10-S2 | 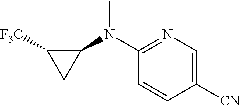 [SMILES: CN(c1ccc(C#N)cn1)[C@H]1C[C@@H]1C(F)(F)F] | LC-MS: 242 [M + H]⁺ |
| | E10-S3 | 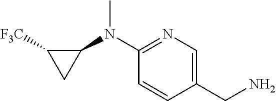 [SMILES: CN(c1ccc(CN)cn1)[C@H]1C[C@@H]1C(F)(F)F] | LC-MS: 246 [M + H]⁺ |
| | E10-S4 | 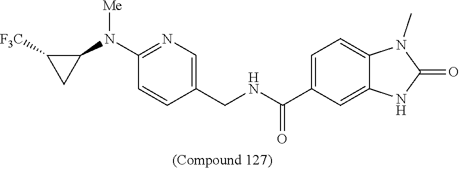 [SMILES: CN(c1ccc(CNC(=O)c2ccc3c(c2)[nH]c(=O)n3C)cn1)[C@H]1C[C@@H]1C(F)(F)F] | LC-MS: 420 [M + H]⁺ ¹H-NMR (400 MHz, CD₃OD) δ 8.17-8.16 (d, 1H), 7.67-7.65 (m, 2H), 7.59-7.58 (d, 1H), 7.19-7.17 (d, 1H), 7.0-6.98 (d, 1H), 4.48 (s, 2H), 3.43 (s, 3H), 3.12 (s, 3H), 2.97-2.93 (m, 1H), 2.06-2.0 (m, 1H), 1.45-1.40 (m, 1H), 1.32-1.31 (m, 1H). |
| 128 | E11-S5 | 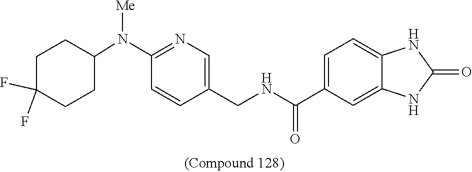 [SMILES: CN(c1ccc(CNC(=O)c2ccc3[nH]c(=O)[nH]c3c2)cn1)C1CCC(F)(F)CC1] | LC-MS: 416 [M + H]⁺ ¹H-NMR (400 MHz, CD₃OD): δ 8.98-8.96 (t, 1H), 8.03-8.0 (dd, 1H), 7.88-7.87 (d, 1H), 7.57-7.53 (m, 2H), 7.38-7.34 (d, 1H), 7.08- 7.06(d, 1H), 4.46 (s, 2H), 4.16-4.15 (m, 1H), 3.06 (s, 3H), 2.20-1.84 (m, 8H). |

TABLE 84

| Ex. | Ref. | Structure | LC-MS, NMR |
|---|---|---|---|
| 129 | E11-S5 | 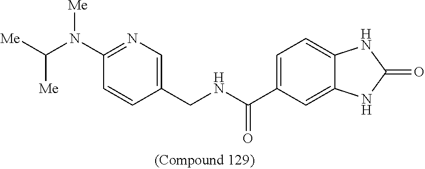 [SMILES: CC(C)N(C)c1ccc(CNC(=O)c2ccc3[nH]c(=O)[nH]c3c2)cn1] | LC-MS: 340 [M + H]⁺ ¹H-NMR (400 MHz, CD₃OD): δ 8.04-8.01 (dd, 1H), 7.88-7.87 (d, 1H), 7.62- 7.57 (m, 2H), 7.35-7.33 (d, 1H), 7.12-7.10 (d, 1H), 4.49 (s, 2H), 4.49-4.39 (m, 1H), 3.07 (s, 3H), 1.34-1.33 (d, 6H). |
| 130 | E11-S5 | 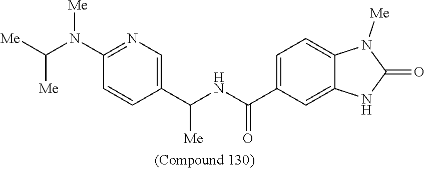 [SMILES: CC(NC(=O)c1ccc2c(c1)[nH]c(=O)n2C)c1ccc(N(C)C(C)C)nc1] | LC-MS: 368 [M + H]⁺ ¹H-NMR (400 MHz, CD₃OD): δ 8.06-8.03 (dd, 1H), 7.81-7.80 (d, 1H), 7.65- 7.63 (dd, 1H), 7.54-7.53 (d, 1H), 7.35-7.32 (d, 1H), 7.16- 7.14 (d, 1H), 5.13-5.11 (m, 1H), 4.39-4.36 (m, 1H), 3.40 (s, 3H), 3.04 (s, 3H), 1.59- 1.57 (d, 3H), 1.31-1.28 (m, 6H). |

TABLE 85

| Ex. | Ref. | Structure | LC-MS, NMR |
|---|---|---|---|
| 130 | | 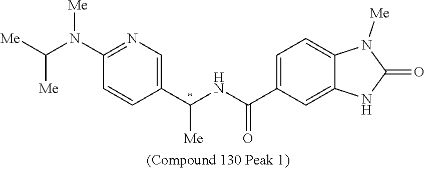 [SMILES: CC(NC(=O)c1ccc2c(c1)[nH]c(=O)n2C)c1ccc(N(C)C(C)C)nc1] | LC-MS: 368 [M + H]⁺ ¹H-NMR (400 MHz, CD₃OD): δ 8.03-8.02 (d, 1H), 7.64-7.61 (m, 2H), 7.53-7.52 (d, 1H), 7.14-7.12 (d, 1H), 6.73-6.71 (d, 1H), 5.14-5.12 (m, 1H), 4.64-4.60 (m, 1H), 3.39 (s, 3H), 2.84 (s, 3H), 1.55-1.53 (d, 3H), 1.17-1.16 (d, 6H). |
| | | 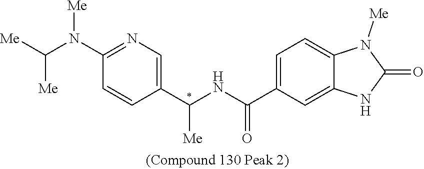 [SMILES: CC(NC(=O)c1ccc2c(c1)[nH]c(=O)n2C)c1ccc(N(C)C(C)C)nc1] | LC-MS: 368 [M + H]⁺ ¹H-NMR (400 MHz, CD₃OD): δ 8.03-8.02 (d, 1H), 7.64-7.61 (m, 2H), 7.53-7.52 (d, 1H), 7.13-7.11 (d, 1H), 6.74-6.71 (d, 1H), 5.14-5.12 (m, 1H), 4.64-4.60 (m, 1H), 3.38 (s, 3H), 2.84 (s, 3H), 1.55-1.53 (d, 3H), 1.17-1.16 (d, 6H). |

TABLE 86

| Ex. | Ref. | Structure | LC-MS, NMR |
|---|---|---|---|
| 131 | E11-S5 | 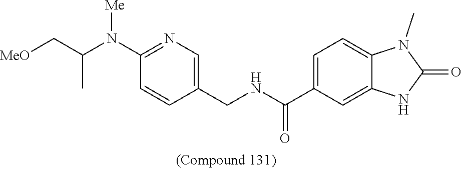 [SMILES: COCC(C)N(C)c1ccc(CNC(=O)c2ccc3c(c2)[nH]c(=O)n3C)cn1] | LC-MS: 384 [M + H]⁺ ¹H-NMR (400 MHz, CD₃OD): δ 8.03-8.02 (d, 1H), 7.63-7.60 (dd, 1H), 7.55-7.52 (m, 2H), 7.14-7.12 (d, 1H), 6.65-6.63 (d, 1H), 4.81-4.80 (m, 2H), 4.40 (s, 2H), 3.54-3.50 (m, 1H), 3.39 (s, 3H), 3.27 (s, 3H), 2.83 (s, 3H), 1.13-1.11 (d, 3H). |

TABLE 87

| Ex. | Ref. | Structure | LC-MS, NMR |
|---|---|---|---|
| 132 | E11-S1 | 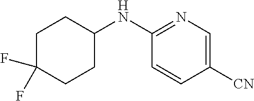 [SMILES: N#Cc1ccc(NC2CCC(F)(F)CC2)nc1] | LC-MS: 238 [M + H]⁺ |
| | E11-S3 | 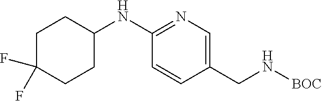 [SMILES: CC(C)(C)OC(=O)NCc1ccc(NC2CCC(F)(F)CC2)nc1] | LC-MS: 342 [M + H]⁺ |
| | E11-S4 | 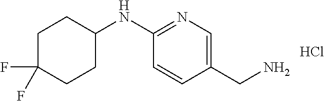 [SMILES: NCc1ccc(NC2CCC(F)(F)CC2)nc1 ; Cl] | LC-MS: 242 [M + H]⁺ |
| | E11-S5 | 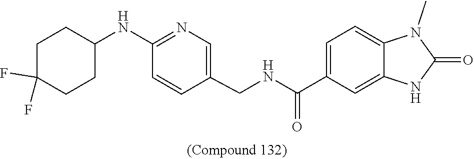 [SMILES: Cn1c(=O)[nH]c2cc(C(=O)NCc3ccc(NC4CCC(F)(F)CC4)nc3)ccc21] | LC-MS: 416 [M + H]⁺ ¹H-NMR (400 MHz, CD₃OD): δ 7.94-7.91 (dd, 1H), 7.79 (s, 1H), 7.65-7.62 (dd, 1H), 7.56-7.55 (d, 1H), 7.16-7.14 (d, 1H), 7.04-7.02 (d, 1H), 4.43 (s, 2H), 3.76-3.71 (m, 1H), 3.39 (s, 3H), 2.14-1.89 (m, 6H), 1.72-1.64 (m, 2H). |

TABLE 88

| Ex. | Ref. | Structure | LC-MS, NMR |
|---|---|---|---|
| 133 | E109- S3 | 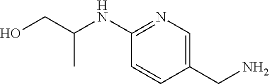 | LC-MS: 196 [M + H]⁺ |
| | E109- S4 | 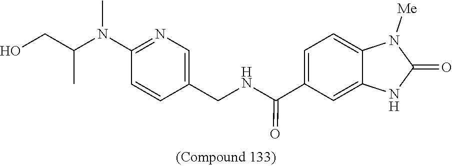 [SMILES: CC(CO)N(C)c1ccc(CNC(=O)c2ccc3c(c2)[nH]c(=O)n3C)cn1] | LC-MS: 370 [M + H]⁺ ¹H-NMR (400 MHz, CD₃OD): δ 8.05-8.0 (dd, 1H), 7.9-7.88 (d, 1H), 7.65- 7.62 (dd, 1H), 7.6-7.58 (d, 1H), 7.4-7.36 (d, 1H), 7.22- 7.20 (d, 1H), 4.5 (s, 2H), 4.44-4.40 (m, 1H), 3.74-3.68 (m, 2H), 3.45 (s, 3H), 3.1 (s, 3H), 1.78-1.76 (d, 3H). |

TABLE 89

| Ex. | Ref. | Structure | LC-MS, NMR |
|---|---|---|---|
| 134 | E110- S1 | 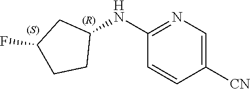 | LC-MS: 206 [M + H]⁺ |
| | E110- S2 | 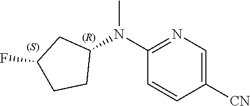 | LC-MS: 220 [M + H]⁺ |
| | E110- S3 | 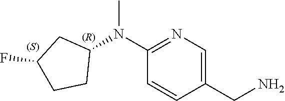 | LC-MS: 224 [M + H]⁺ |
| | E110- S4 | 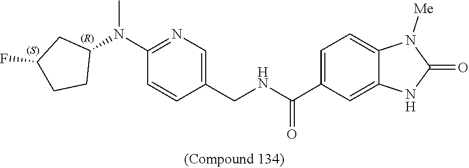 | LC-MS: 398 [M + H]⁺ ¹H-NMR (400 MHz, DMSO-d₆); δ 10.6 (bs, 1H), 8.72-8.68 (t, 1H), 8.03-8.02 (d, 1H), 7.62-7.58 (d, 1H), 7.5-7.48 (m, 2H), 7.14-7.12 (d, 1H), 6.62-6.59 (d, 1H), 5.25-5.05 (m, 3H), 4.29-4.27 (d, 2H), 3.29 (s, 3H), 2.80 (s, 3H), 2.32-2.18 (m, 1H), 2.0- 1.6 (m, 4H). |
| 135 | E3-S6 | 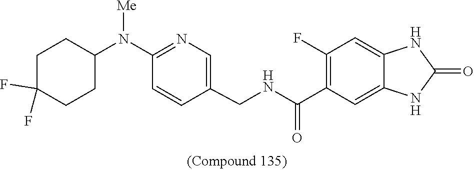 [SMILES: CN(c1ccc(CNC(=O)c2cc3[nH]c(=O)[nH]c3cc2F)cn1)C1CCC(F)(F)CC1] | LC-MS: 434 [M + H]⁺ ¹H-NMR (400 MHz, CD₃OD): δ 8.07-8.04 (dd, 1H), 7.89 (s, 1H), 7.43-7.38 (m, 2H), 6.89-6.87 (d, 1H), 4.48 (s, 2H), 4.15-4.12 (m, 1H), 3.07 (s, 3H), 2.17-1.85 (m, 8H). |

TABLE 90

| Ex. | Ref. | Structure | LC-MS, NMR |
|---|---|---|---|
| 136 | E3-S6 | 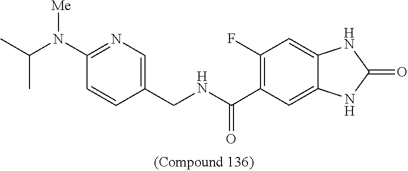 [SMILES: CC(C)N(C)c1ccc(CNC(=O)c2cc3[nH]c(=O)[nH]c3cc2F)cn1] | LC-MS: 358 [M + H]⁺ ¹H-NMR (400 MHz, CD₃OD): δ 8.03-8.00 (dd, 1H), 7.84-7.83 (d, 1H), 7.40- 7.32 (dd, 2H), 6.90-6.88 (d, 1H), 4.47 (s, 2H), 4.41-4.34 (m, 1H), 3.05 (s, 3H), 1.31- 1.30 (d, 6H). |
| 137 | E3-S6 | 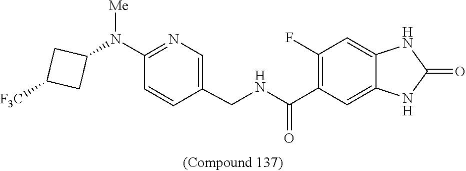 [SMILES: CN(c1ccc(CNC(=O)c2cc3[nH]c(=O)[nH]c3cc2F)cn1)[C@H]1C[C@@H](C(F)(F)F)C1] | LC-MS: 438 [M + H]⁺ ¹H-NMR (400 MHz, CD₃OD): δ 8.08-8.07 (d, 1H), 7.59-7.56 (dd, 1H), 7.37-7.35 (d, 1H), 6.87-6.84 (d, 1H), 6.67-6.65 (d, 1H), 4.78-4.71 (m, 1H), 4.23 (s, 2H), 2.96 (s, 3H), 2.83-2.48 (m, 1H), 2.47-2.43 (m, 2H), 2.33-2.25 (m, 2H). |

TABLE 91

| Ex. | Ref. | Structure | LC-MS, NMR |
|---|---|---|---|
| 138 | E105- S1 | 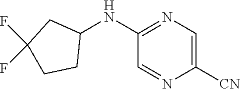 [SMILES: N#Cc1cnc(NC2CCC(F)(F)C2)cn1] | LC-MS: 225 [M + H]⁺ |
| | E105- S2 | 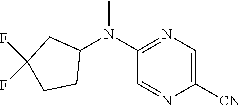 [SMILES: CN(c1cnc(C#N)cn1)C1CCC(F)(F)C1] | LC-MS: 237 [M − H]⁻ |
| | E105- S3 | 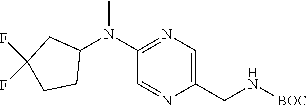 [SMILES: CN(c1cnc(CNC(=O)OC(C)(C)C)cn1)C1CCC(F)(F)C1] | LC-MS: 341 [M − H]⁻ |
| | E105- S4 | 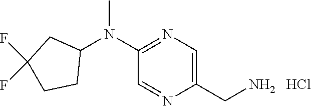 | LC-MS: 227 [M − NH2]⁻ |
| | E3-S6 | 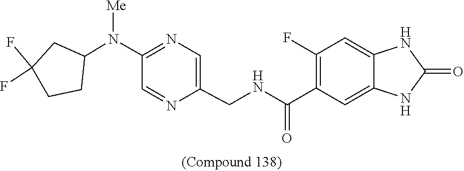 [SMILES: CN(c1cnc(CNC(=O)c2cc3[nH]c(=O)[nH]c3cc2F)cn1)C1CCC(F)(F)C1] | LC-MS: 421 [M + H]⁺ ¹H-NMR (400 MHz, CD₃OD): δ 8.17-8.16 (d, 1H), 8.08-8.07 (d, 1H), 7.47- 7.45 (d, 1H), 6.93-6.90 (d, 1H), 5.31-5.27 (m, 1H), 4.59 (s, 2H), 3.01 (s, 3H), 2.29- 2.23 (m, 1H), 2.19-2.08 (m, 2H), 2.07-2.00 (m, 3H). |

TABLE 92

| Ex. | Ref. | Structure | LC-MS, NMR |
|---|---|---|---|
| 139 | E3-S6 | 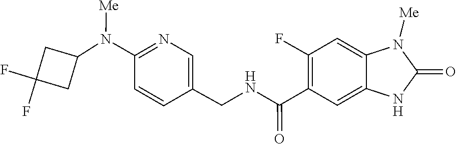 [SMILES: CN(c1ccc(CNC(=O)c2cc3[nH]c(=O)n(C)c3cc2F)cn1)C1CC(F)(F)C1] (Compound 139) | LC-MS: 420 [M + H]⁺ ¹H-NMR (400 MHz, DMSO-d₆): δ 11.10 (bs, 1H), 8.54-8.53 (q, 1H), 8.10-8.09 (d, 1H), 7.56-7.53 (dd, 1H), 7.20-7.13 (q, 2H), 6.72-6.70 (d, 1H), 4.69-4.65 (m, 1H), 4.33-4.31 (d, 2H), 3.29 (s, 3H), 2.92 (s, 3H), 2.90-2.87 (m, 2H), 2.80-2.73 (m, 2H). |
| 140 | E3-S6 | 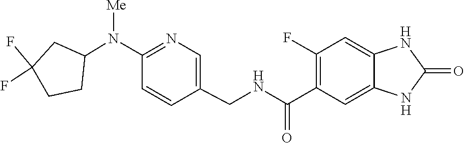 [SMILES: CN(c1ccc(CNC(=O)c2cc3[nH]c(=O)[nH]c3cc2F)cn1)C1CCC(F)(F)C1] | LC-MS: 420 [M + H]⁺ ¹H-NMR (400 MHz, CD₃OD): δ 8.11-8.10 (d, 1H), 7.64-7.61 (dd, 1H), 7.41-7.40 (d, 1H), 6.91-6.88 (d, 1H), 6.74-6.72 (d, 1H), 5.26-5.22 (m, 1H), 4.47 (s, 2H), 2.93 (s, 3H), 2.38 (m, 1H), 2.26-2.23 (m, 2H), 2.22-2.05 (m, 3H). |
| | | (Compound 140) | |

TABLE 93

| Ex. | Ref. | Structure | LC-MS, NMR |
|---|---|---|---|
| 141 | E3-S6 | 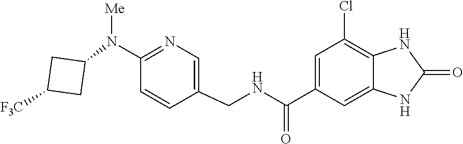 [SMILES: CN(c1ccc(CNC(=O)c2cc(Cl)c3[nH]c(=O)[nH]c3c2)cn1)[C@H]1C[C@@H](C(F)(F)F)C1] | LC-MS: 454 [M + H]⁺ ¹H-NMR (400 MHz, CD₃OD): δ 8.11-8.10 (d, 1H), 7.61-7.59 (m, 2H), 7.49-7.48 (d, 1H), 6.71-6.69 (d, 1H), 4.81-4.76 (m, 1H), 4.44 (s, 2H), 3.00 (s, 3H), 2.85-2.76 (m, 1H), 2.52-2.45 (m, 2H), 2.37-2.29 (m, 2H). |
| | | (Compound 141) | |

TABLE 94

| Ex. | Ref. | Structure | LC-MS, NMR |
|---|---|---|---|
| 142 | P5-S1 | 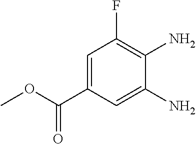 | LC-MS: 185 [M + H]⁺ |
| | P5-S2 | 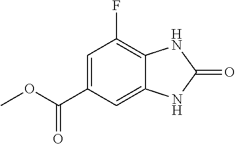 | LC-MS: 211 [M + H]⁺ |
| | P5-S3 | 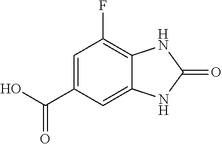 [SMILES: O=C(O)c1cc(F)c2[nH]c(=O)[nH]c2c1] | LC-MS: 197 [M + H]⁺ |
| | E3-S6 | 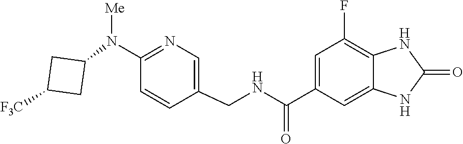 [SMILES: CN(c1ccc(CNC(=O)c2cc(F)c3[nH]c(=O)[nH]c3c2)cn1)[C@H]1C[C@@H](C(F)(F)F)C1] | LC-MS: 438 [M + H]⁺ ¹H-NMR (400 MHz, CD₃OD): δ 8.11-8.10 (d, 1H), 7.61-7.59 (dd, 1H), 7.41-7.38 (dd, 2H), 6.71-6.68 (d, 1H), 4.80-4.76 (m, 1H), 4.44 (s, 2H), 3.00 (s, 3H), 2.85-2.78 (m, 1H), 2.52-2.45 (m, 2H), 2.37-2.29 (m, 2H). |
| | | (Compound 142) | |

TABLE 95

| Ex. | Ref. | Structure | LC-MS, NMR |
|---|---|---|---|
| 143 | E3-S6 | 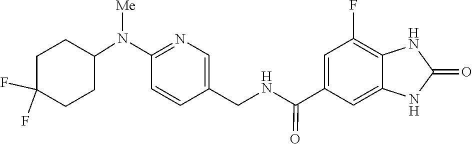 [SMILES: CN(c1ccc(CNC(=O)c2cc(F)c3[nH]c(=O)[nH]c3c2)cn1)C1CCC(F)(F)CC1] | LC-MS: 434 [M + H]⁺ ¹H-NMR (400 MHz, CD₃OD): δ 8.09-8.08 (d, 1H), 7.62-7.60 (dd, 1H), 7.41-7.38 (dd, 2H), 6.72- 6.70 (d, 1H), 4.53 (m, 1H), 4.44 (s, 2H), 2.29 (s, 3H), 2.15-2.12 (m, 2H), 2.03 (m, 1H), 1.95-1.86 (m, 3H), 1.76-1.73 (m, 2H). |
| | | (Compound 143) | |
| 144 | E3-S6 | 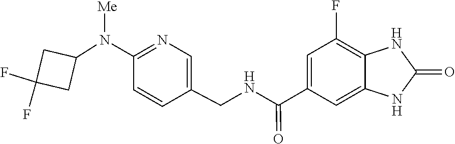 [SMILES: CN(c1ccc(CNC(=O)c2cc(F)c3[nH]c(=O)[nH]c3c2)cn1)C1CC(F)(F)C1] | LC-MS: 406 [M + H]⁺ ¹H-NMR (400 MHz, CD₃OD): δ 8.13-8.12 (d, 1H), 7.63-7.60 (dd, 1H), 7.41-7.38 (dd, 2H), 6.73-6.71 (d, 1H), 4.86 (m, 1H), 4.45 (s, 2H), 3.00 (s, 3H), 2.94-2.88 (m, 2H), 2.78-2.74 (m, 2H). |
| | | (Compound 144) | |

TABLE 96

| Ex. | Ref. | Structure | LC-MS, NMR |
|---|---|---|---|
| 145 | P5-S1 | 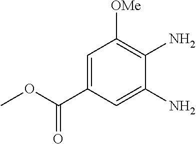 | LC-MS: 197 [M + H]⁺ |
| | P5-S2 | 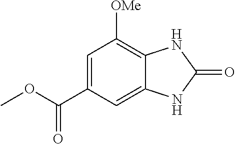 [SMILES: COC(=O)c1cc(OC)c2[nH]c(=O)[nH]c2c1] | LC-MS: 221 [M − H]⁻ |
| | P5-S3 | 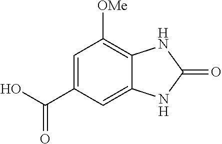 [SMILES: COc1cc(C(=O)O)cc2[nH]c(=O)[nH]c12] | LC-MS: 209 [M + H]⁺ |
| | E3-S6 | 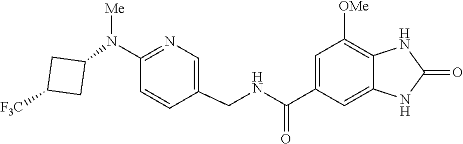 [SMILES: COc1cc(C(=O)NCc2ccc(N(C)[C@H]3C[C@@H](C(F)(F)F)C3)nc2)cc2[nH]c(=O)[nH]c12] (Compound 145) | LC-MS: 450 [M + H]⁺ ¹H-NMR (400 MHz, DMSO-d₆): δ 10.96 (s, 1H), 10.82 (s, 1H), 8.81-8.78 (t, 1H), 8.07-8.06 (d, 1H), 7.52-7.45 (d, 1H), 7.21-7.16 (d, 2H), 6.65-6.63 (d, 1H), 4.92-4.88 (m, 1H), 4.32-4.30 (d, 2H), 3.87 (s, 3H), 2.68 (s, 3H), 2.42-2.35 (m, 3H), 2.34-2.18 (m, 2H). |

TABLE 97

| Ex. | Ref. | Structure | LC-MS, NMR |
|---|---|---|---|
| 146 | E3-S6 | 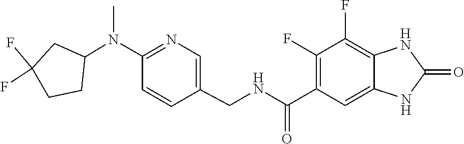 [SMILES: CN(c1ccc(CNC(=O)c2cc3[nH]c(=O)[nH]c3c(F)c2F)cn1)C1CCC(F)(F)C1] | LC-MS: 438 [M + H]⁺ ¹H-NMR (400 MHz, CD₃OD): δ 8.12-8.10 (dd, 1H), 7.96-7.95 (d, 1H), 7.48-7.45 (d, 1H), 7.21-7.19 (d, 1H), 4.85-4.83 (m, 1H), 4.53 (s, 2H), 3.16 (s, 3H), 2.59- 2.57 (m, 1H), 2.39-2.08 (m, 5H). |
| | | (Compound 146) | |
| 147 | E3-S6 | 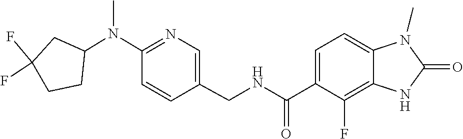 [SMILES: CN(c1ccc(CNC(=O)c2ccc3c([nH]c(=O)n3C)c2F)cn1)C1CCC(F)(F)C1] | LC-MS: 434 [M + H]⁺ ¹H-NMR (400 MHz, CD₃OD): δ 8.12-8.11 (d, 1H), 7.63-7.60 (dd, 1H), 7.50-7.47 (q, 1H), 7.04-7.02 (d, 1H), 6.72-6.70 (d, 1H), 5.28-5.24 (m, 1H), 4.47 (s, 2H), 3.43 (s, 3H), 2.92 (s, 3H), 2.37-2.12 (m, 4H), 2.07-1.96 (m, 2H). |
| | | (Compound 147) | |

### <Biological Assays>

### Example 148 KIT Tyrosine Kinase Assay

An in vitro assay utilizing the recombinant human KIT tyrosine kinase protein was developed to measure compound inhibition of KIT. KIT tyrosine kinase activity was measured by the ADP-Glo Kinase Assay system with poly (Glu4-Tyr) peptide substrate. Kinase Assay was performed in white 384-well plates and the luminescence was measured by Victor X5 Multilabel Counter (Perkin Elmer).

### Materials and Method

Recombinant intracellular domain of human KIT protein was purchased from Carna Biosciences (Kobe, Japan). Poly (Glu4-Tyr) peptide was purchased from Sigma-Aldrich (St. Louis, Missouri, USA). ADP-Glo Kinase Assay reagent was purchased from Promega (Madison, Wisconsin, USA). Each test compound was dissolved in 100% DMSO to prepare 10 mM stock solutions. Stock solutions were diluted to 400 μM, then serially diluted 1:3 in 100% DMSO to make a 9-point serial dilution. To prepare compound working solutions, each serially diluted solution was further diluted 1:8 with Assay Buffer, consisting of 50 mM Tris-HCl pH 7.5, 20 mM MgCl₂, 0.01% Tween-20, 2 mM MnCl₂, 50 μM Dithiothreitol (DTT).

Recombinant KIT protein was diluted to 1 μM with Assay Buffer. Poly (Glu4-Tyr) peptide and ATP were diluted to 300 microgram/mL and 250 μM, respectively, with Assay Buffer, then mixed to make the substrate solution. In a 384-well plate, 2 μL of working compounds and 5 μL of recombinant KIT protein were mixed and incubated for 30 minutes at room temperature. After the preincubation, 3 μL of substrate/ATP mixture was added and incubated for 1 hour at room temperature. The tyrosine phosphorylation by KIT kinase was quantified by ADP-Glo kinase assay kit following the manufacturer's protocol. Briefly, the phosphorylation reaction was terminated by adding 10 μL ADP-Glo reagent and incubated for 60 minutes at room temperature. 20 μL of Kinase Detection Reagent was added to each well and luminescence was measured by plate reader. The assay control for this experiment was 2.5% DMSO and Assay Buffer was used as the blank.

Half maximal inhibitory concentration (IC50) was calculated by a Four Pa-rameters Logistic Regression model using GraphPad Prism (GraphPad Software). The value without recombinant KIT protein and without test compound was used as a negative control (assay background). The value with recombinant KIT protein without test compound was a positive control (100% activity).

The Tables 98 and 99 below list inhibitory effects of representative compounds of the present invention against KIT.

TABLE 98

| Compound No. | KIT IC50 (μM) |
|---|---|
| 1 | 0.057 |
| 2 | 0.45 |
| 3 | 0.026 |
| 4 | 0.021 |
| 5 | 3.2 |
| 6 | 0.12 |
| 6 Peak 1 | 0.036 |
| 6 Peak 2 | 5.8 |
| 7 | 0.040 |
| 8 | 0.19 |
| 9 | 0.034 |
| 10 | 0.042 |
| 11b | 0.096 |
| 12 | 0.036 |
| 13 | 0.040 |
| 14 | 0.063 |
| 15 | 0.067 |
| 16 | 0.061 |
| 17 | 0.022 |
| 18 | 0.057 |
| 19 | 0.060 |
| 20 | 0.038 |
| 21 | 0.66 |
| 22 | 0.46 |
| 23 | 0.078 |
| 24 | 0.34 |
| 25 | 0.36 |
| 26 | 0.27 |
| 27 | 0.074 |
| 28 | 3.6 |
| 29 | 0.056 |
| 30 | 0.067 |
| 31 | 0.070 |
| 32 | 0.029 |
| 33 | 0.078 |
| 34 | 0.058 |
| 35 | 0.083 |
| 36 | 0.050 |
| 37 | 0.31 |
| 38 | 0.060 |
| 39 | 0.035 |
| 40 | 0.039 |
| 41 | 0.036 |
| 42 | 0.027 |
| 43 | 0.14 |
| 44 | 0.12 |
| 45 | 0.13 |
| 46 | 0.027 |
| 47 | 0.056 |
| 48 | 0.55 |
| 49 | 0.95 |
| 50 | 0.17 |
| 51 | 0.21 |
| 52 | 0.024 |
| 53 | 0.079 |
| 54 | 0.033 |
| 55 | 0.037 |
| 56 | 0.029 |
| 57 | 0.227 |
| 58 | 0.021 |
| 59 | 0.014 |
| 60 | 0.14 |
| 61 | 0.056 |
| 62 | 0.17 |
| 63 | 0.037 |
| 64 | 0.095 |
| 65 | 0.035 |
| 66 | 0.033 |
| 67 | 0.025 |
| 68 | 0.042 |
| 69 | 0.096 |
| 70 | 0.061 |
| 71 | 0.060 |
| 72 | 0.084 |
| 73 | 0.027 |
| 74 | 0.025 |
| 75 | 0.061 |
| 76 | 0.086 |
| 77 | 0.029 |
| 78 | 0.026 |
| 79 | 0.21 |
| 80 | 0.031 |
| 81 | 0.15 |
| 82 | 0.075 |
| 82 Peak 1 | 0.18 |
| 82 Peak 2 | 0.029 |
| 83 | 0.041 |
| 84 | 0.016 |
| 85 | 0.15 |
| 86 | 0.015 |
| 87 | 0.068 |
| 88 | 0.098 |
| 89 | 0.054 |
| 90 | 0.048 |
| 91 | 0.12 |
| 92 | 0.059 |
| 93 | 0.27 |
| 94 | 0.12 |
| 95 | 0.024 |
| 96 | 0.060 |
| 97 | 0.034 |
| 98 | 0.071 |
| 99 | 0.050 |
| 100 | 0.053 |
| 101 | 3.3 |

TABLE 99

| Compound No. | KIT IC50 (μM) |
|---|---|
| 102 | 0.055 |
| 103 | 0.042 |
| 104 | 0.051 |
| 105 | 0.021 |
| 106 Isomer I | 0.036 |
| 106 Isomer II | 0.022 |
| 107 Isomer I | 0.018 |
| 107 Isomer II | 0.028 |
| 108 | 0.022 |
| 109 | 0.025 |
| 110 | 0.033 |
| 112 | 59%@10 uM |
| 113 | 1.999 |
| 114a | 0.108 |
| 114b | 0.317 |
| 115 | 0.021 |
| 116 | 0.029 |
| 117 | 0.274 |
| 118 | 0.632 |
| 119 | 0.040 |
| 120 | 0.045 |
| 121 | 0.059 |
| 122 | 0.032 |
| 123 | 0.126 |
| 125 | 0.031 |
| 126 | 0.033 |
| 127 | 0.047 |
| 128 | 0.037 |
| 129 | 0.020 |
| 130 | 0.222 |
| 130 Peak 1 | 0.079 |
| 130 Peak 2 | 1.035 |
| 131 | 0.081 |
| 132 | 0.131 |
| 133 | 0.075 |
| 134 | 0.038 |
| 135 | 0.030 |
| 136 | 0.027 |
| 137 | 0.023 |
| 138 | 0.035 |
| 139 | 0.075 |
| 140 | 0.018 |
| 141 | 0.027 |
| 142 | 0.018 |
| 143 | 0.035 |
| 144 | 0.057 |
| 145 | 0.015 |
| 146 | 0.030 |
| 147 | 0.023 |

Notably, the representative compounds of the present invention showed selective inhibitory activities against KIT while not exhibiting significant inhibition against other kinases.

### Example 149 SCF-Dependent M-07e Cell Proliferation Assay

SCF-dependent cell proliferation assay was developed to measure the inhibitory potency of the compounds in M-07e cells, which express wild-type human KIT protein. The assay was performed in 96-well plates and the IC50 of each KIT inhibitor was determined by CellTiter Glo Assay reagent and Victor X5 Multilabel Counter (Perkin Elmer).

### Materials and Methods

M-07e cells were purchased from AcceGen Biotechnology. Cells were maintained with M-07e Growth medium: RPMI-1640 containing 20% FBS, 100 U/mL penicillin and 100 microgram/mL streptomycin, 10 ng/mL rhGM-CSF, 10 ng/mL rhSCF. M-07e cells were seeded at a density of 5000 cells/90 μL/well in 96-well plates. Test compounds were dissolved in 100% DMSO to make 20 mM stock solution and serially diluted 1:3 with 100% DMSO to make an 11-point dilution series with concen-trations from 15 mM to 0.024 μM. The serially diluted compounds were then each diluted 1:50 with culture medium to obtain assay compound solutions containing 2% DMSO. Ten μL of assay compound solutions were added to the 90 μL of culture cell suspension in the 96-well plate, with a final DMSO concentration of 0.2%. The plates were then incubated in a 5% CO₂ incubator at 37° C. for 3 days. To obtain Day Zero Controls for background calculation, cells were seeded at 5000 cells/100 μL in a new 96-well plate and processed on the same day. Both Day Zero controls and 3 day culture plates were then treated as follows. 100 μL of CellTiter Glo Assay reagent was added to each well and the plate was incubated at room temperature for 15 min on a shaker at 200-300 RPM. Then 100 μL of cell lysate samples were transferred to white wall 96-well plates and luminescence was measured by a plate reader. The luminescence of Day Zero Control was subtracted from the luminescence intensity of 3 day culture readings. The assay control for this experiment was cells treated with 0.2% DMSO in culture medium and wells with 100 μL of culture medium (0.2% DMSO without cells) were used as the blank for data analysis.

The IC50 value of each compound was determined by using % inhibition by nor-malizing inhibition in DMSO treated control wells to 0% using the following formula method:

$$\% inhibition = 100 - [ ( Blanks subtracted L of experimental wells ) / ( Blanks subtracted L of 0.2 \% DMSO control wells ) \times 100 ]$$

*Where L is luminescence

Plot percent inhibition in proliferation of triplicates against the respective concen-trations of compound and fit the dose response curve using the sigmoidal dose response equation to generate the IC50 in GraphPad Prism 8 software.

The Table 100 below lists inhibitory effects of representative compounds of the present invention against the M-07e cells.

TABLE 100

| Compound No. | M-07e IC50 (μM) |
|---|---|
| 3 | 0.18 |
| 4 | 0.38 |
| 6 Peak 1 | 0.68 |
| 9 | 0.61 |
| 10 | 0.24 |
| 11b | 0.22 |
| 12 | 0.038 |
| 14 | 0.56 |
| 15 | 2.2 |
| 17 | 0.39 |
| 18 | 0.21 |
| 19 | 0.16 |
| 20 | 0.40 |
| 36 | 0.87 |
| 39 | 0.14 |
| 48 | 8.9 |
| 57 | 7.3 |
| 58 | 2.4 |
| 59 | 0.19 |
| 61 | 0.76 |
| 62 | 0.29 |
| 64 | 0.61 |
| 65 | 0.27 |
| 66 | 0.079 |
| 67 | 0.22 |
| 68 | 0.13 |
| 69 | 0.52 |
| 70 | 0.14 |
| 71 | 0.31 |
| 72 | 0.80 |
| 73 | 0.098 |
| 74 | 0.11 |
| 75 | 0.50 |
| 76 | 1.6 |
| 77 | 0.11 |
| 78 | 0.15 |
| 80 | 0.24 |
| 82 | 0.73 |
| 82 Peak 1 | 6.3 |
| 82 Peak 2 | 0.62 |
| 83 | 0.30 |
| 84 | 0.18 |
| 86 | 0.12 |
| 89 | 0.25 |
| 90 | 1.8 |
| 92 | 1.9 |
| 95 | 0.19 |
| 96 | 1.2 |
| 97 | 0.24 |
| 98 | 0.99 |
| 99 | 0.54 |
| 100 | 0.25 |
| 102 | 0.27 |
| 103 | 0.819 |
| 104 | 7.661 |
| 105 | 0.013 |
| 106 Isomer I | 0.015 |
| 106 Isomer II | 0.089 |
| 107 Isomer I | 0.047 |
| 107 Isomer II | 0.286 |
| 108 | 0.065 |
| 109 | 0.012 |
| 110 | 0.055 |
| 115 | 0.070 |
| 116 | 0.028 |
| 119 | 0.423 |
| 120 | 0.206 |
| 121 | 1.354 |
| 122 | 0.014 |
| 125 | 0.151 |
| 126 | 0.806 |
| 127 | 1.321 |
| 128 | 2.830 |
| 129 | 1.024 |
| 130 | 1.855 |
| 130 Peak 1 | 0.621 |
| 131 | 0.407 |
| 134 | 0.062 |
| 135 | 0.589 |
| 136 | 0.209 |
| 137 | 0.036 |
| 138 | 4.567 |
| 139 | 0.636 |
| 140 | 0.099 |
| 141 | 0.153 |
| 142 | 0.278 |
| 143 | 1.464 |
| 144 | 3.556 |
| 145 | 0.515 |
| 146 | 0.621 |
| 147 | 0.440 |

### Example 150 GIST Cell Proliferation Assay

Gastrointestinal stromal tumors (GIST) are the most common adult sarcomas and the majority (85-90%) of GIST cases are caused by oncogenic mutations in the receptor tyrosine kinases (RTKs), KIT or PDGFRA, which result in constitutive activation of these receptors. Human GIST-derived cell line GIST-Ti which harboring a mutation in exon 11 of KIT was used for the measurement of inhibitory potency of the compounds.

### Materials and Methods

GIST-T1 cells were purchased from Cosmo Bio (Tokyo, Japan) and cultured in 10% FBS containing DMEM. The assay was performed in 96-well plates and the IC50 of each compound was determined by CellTiter Glo Assay reagent and Victor X5 Multilabel Counter. Cells were seeded at a density of 2000 cells/90 μL/well in 96-well plates. Compound dilution, cell treatment and determination of IC50 were performed as described in the assay methods of M-07e Cell Proliferation Assay.

The Table 101 below lists inhibitory effects of representative compounds of the present invention against the GIST-T1 cells.

TABLE 101

| Compound No. | GIST-T1 IC50 (μM) |
|---|---|
| 1 | 0.057 |
| 2 | 0.45 |
| 28 | 3.6 |
| 29 | 0.056 |
| 30 | 0.067 |
| 31 | 0.070 |
| 32 | 0.029 |
| 33 | 0.078 |
| 34 | 0.058 |
| 35 | 0.083 |

1. A pharmaceutical composition comprising a pharmaceutically acceptable carrier or diluent and a compound represented by the following Formula (I): 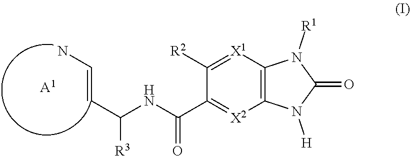
or a pharmaceutically acceptable salt thereof, wherein:
R¹ is hydrogen, C₁₋₃-alkyl, or C₁₋₃-deuteroalkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen, aryl, carboxy, —CO—O—(C₁₋₄-alkyl), or C₁₋₄-alkyl which may be substituted with hydroxy,
a ring A: 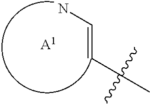
is a group of the formula: 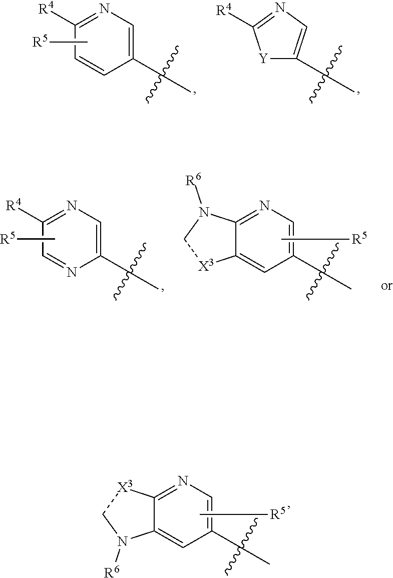
the wavy line indicates a point of attachment to the rest of molecule,
R⁴ is —N(R^{A})(R^{B}), —O—R^{A}, or C₁₋₂-alkyl substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, hydroxy, —O—(C₁₋₄-alkyl), C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, —O—(C₁₋₄-haloalkyl), amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino.

2. The pharmaceutical composition of claim 1, which is a pharmaceutical composition comprising a pharmaceutically acceptable carrier or diluent and a compound represented by the following Formula (I): 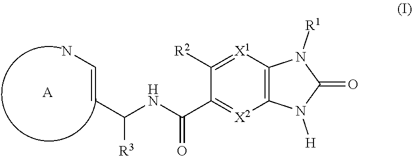
or a pharmaceutically acceptable salt thereof, wherein:
R¹ is hydrogen or C₁₋₃-alkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen or C₁₋₄-alkyl,
a ring A: 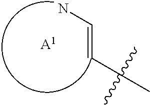
is a group of the formula: 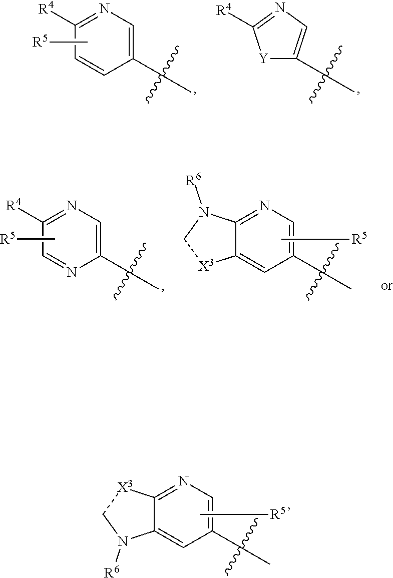
the wavy line indicates a point of attachment to the rest of molecule,
R⁴ is —N(R^{A})(R^{B}), —O—R^{A}, or C₁₋₂-alkyl substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁4-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino.

3. The pharmaceutical composition of claim 2, wherein R¹ is hydrogen, methyl, ethyl, or isopropyl, and R³ is hydrogen or methyl.

4. The pharmaceutical composition of claim 3, wherein the ring A: 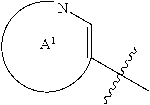
is a group of the formula:

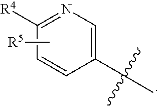

5. The pharmaceutical composition of claim 4, wherein R⁴ is —N(R^{A})(R^{B}), or —O—R^{A}, and R⁵ is hydrogen, in which R^{A} is C₁₋₄-alkyl optionally substituted with one or more groups selected from the group consisting of halogen, C₃₋₆-cycloalkyl, phenyl, tetrahydropyranyl, piperidyl, piperazinyl, pyridyl, pyrimidyl, and indazolyl, in which said C₃₋₆-cycloalkyl, phenyl, tetrahydropyranyl, piperidyl, piperazinyl, pyridyl, pyrimidyl, and indazolyl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; phenyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with tetrahydropyranyl, piperidyl, or piperazinyl, each of which may be further substituted with halogen, cyano, hydroxy, C₁₋₄-alkyl, —O—(C₁₋₄-alkyl), C₁₋₄-haloalkyl, amino, or mono- or di-(C₁₋₄-alkyl)amino; tetrahydropyranyl, piperidyl, or piperazinyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or pyridyl, pyrimidyl, or indazolyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and R^{B} is methyl.

6. The pharmaceutical composition of claim 5, wherein R^{A} is isopropyl; C₃₋₆-cycloalkyl substituted with one or more groups selected from the group consisting of halogen, cyano, C₁₋₄-alkyl, and C₁₋₄-haloalkyl; phenyl substituted with one or more groups selected from the group consisting of halogen, cyano, C₁₋₄-alkyl, and C₁₋₄-haloalkyl; or pyridyl substituted with one or more groups selected from the group consisting of halogen, cyano, C₁₋₄-alkyl, and C₁₋₄-haloalkyl.

7. The pharmaceutical composition of claim 1, wherein the compound is selected from the group consisting of
N-((6-(2-Fluorophenoxy)pyridin-3-yl)methyl)-1-methyl-2-oxo-2,3-dihydro-1H-benzimidazole-5-carboxamide,
N-((6-(4-Fluorophenoxy)pyridin-3-yl)methyl)-1-isopropyl-2-oxo-2,3-dihydro-1H-benzimidazole-5-carboxamide,
6-Fluoro-N-((6-(2-fluorophenoxy)pyridin-3-yl)methyl)-1-methyl-2-oxo-2,3-dihydro-1H-benzimidazole-5-carboxamide,
N-((6-(2,4-Difluorophenoxy)pyridin-3-yl)methyl)-1-methyl-2-oxo-2,3-dihydro-1H-benzimidazole-5-carboxamide,
N-((2-(4-Fluorophenoxy)thiazol-5-yl)methyl)-1-methyl-2-oxo-2,3-dihydro-1H-benzimidazole-5-carboxamide,
N-(1-(6-(4-Fluorophenoxy)pyridin-3-yl)ethyl)-1-methyl-2-oxo-2,3-dihydro-1H-benzimidazole-5-carboxamide,
(S)—N-(1-(6-(3,4-Difluorophenoxy)pyridin-3-yl)ethyl)-1-methyl-2-oxo-2,3-dihydro-1H-benzimidazole-5-carboxamide,
N-((6-((4-Fluorophenyl)amino)pyridin-3-yl)methyl)-2-oxo-2,3-dihydro-1H-benzimidazole-5-carboxamide,
N-((6-((4-Fluorophenyl)(methyl)amino)pyridin-3-yl)methyl)-1-methyl-2-oxo-2,3-dihydro-1H-benzimidazole-5-carboxamide,
N-((6-((4,4-Difluorocyclohexyl)(methyl)amino)pyridin-3-yl)methyl)-1-methyl-2-oxo-2,3-dihydro-1H-benzimidazole-5-carboxamide,
N-((6-(Isopropyl(methyl)amino)pyridin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyridin-3-yl)methyl)-2-oxo-2,3-dihydro-1H-benzimidazole-5-carboxamide,
(S)—N-(1-(6-((4-Fluorophenyl)(methyl)amino)pyridin-3-yl)ethyl)-1-methyl-2-oxo-2,3-dihydro-1H-benzimidazole-5-carboxamide,
(S)-1-Methyl-N-((6-(2-methylpyrrolidin-1-yl)pyridin-3-yl)methyl)-2-oxo-2,3-dihydro-1H-benzimidazole-5-carboxamide,
N-((6-(1-(4-Fluorophenyl)ethyl)pyridin-3-yl)methyl)-1-methyl-2-oxo-2,3-dihydro-1H-benzimidazole-5-carboxamide,
N-((6-(Fluoro(4-fluorophenyl)methyl)pyridin-3-yl)methyl)-1-methyl-2-oxo-2,3-dihydro-1H-benzimidazole-5-carboxamide,
N-((1-Isopropyl-2,3-dihydro-1H-pyrrolo[2,3-b]pyridin-5-yl)methyl)-1-methyl-2-oxo-2,3-dihydro-1H-benzimidazole-5-carboxamide,
N-((1-(4,4-difluorocyclohexyl)-2,3-dihydro-1H-pyrrolo[2,3-b]pyridin-5-yl)methyl)-1-methyl-2-oxo-2,3-dihydro-1H-benzimidazole-5-carboxamide,
N-((1-(2-Fluorophenyl)-2,3-dihydro-1H-pyrrolo[2,3-b]pyridin-5-yl)methyl)-1-methyl-2-oxo-2,3-dihydro-1H-benzimidazole-5-carboxamide,
1-Methyl-2-oxo-N-((1-phenyl-1H-pyrrolo[2,3-b]pyridin-5-yl)methyl)-2,3-dihydro-1H-benzimidazole-5-carboxamide,
N-((3-(2,6-Difluorophenyl)-3H-imidazo[4,5-b]pyridin-6-yl)methyl)-1-methyl-2-oxo-2,3-dihydro-1H-benzimidazol e-5-carboxamide,
N-((6-(3-Cyanophenoxy)pyridin-3-yl)methyl)-1-methyl-2-oxo-2,3-dihydro-1H-benzimidazole-5-carboxamide,
N-((6-(Cyclopropyl(4-fluorophenyl)amino)pyridin-3-yl)methyl)-1-methyl-2-oxo-2,3-dihydro-1H-benzimidazole-5-carboxamide,
1-Methyl-N-((6-(methyl(1-methylpiperidin-4-yl)amino)pyridin-3-yl)methyl)-2-oxo-2,3-dihydro-1H-benzimidazole-5-carboxamide,
N-((2-((4,4-Difluorocyclohexyl)amino)thiazol-5-yl)methyl)-1-methyl-2-oxo-2,3-dihydro-1H-benzimidazole-5-carboxamide,
1-Methyl-N-((6-(methyl(3-((4-methylpiperazin-1-yl)methyl)phenyl)amino)pyridin-3-yl)methyl)-2-oxo-2,3-dihydro-1H-benzimidazole-5-carboxamide,
N-(1-(6-(2,4-Difluorophenoxy)pyridin-3-yl)ethyl)-1-methyl-2-oxo-2,3-dihydro-1H-benzimidazole-5-carboxamide,
N-((6-((4,4-Difluorocyclohexyl)(methyl)amino)pyrazin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyrazin-3-yl)methyl)-2-oxo-2,3-dihydro-1H-benzimidazole-5-carboxamide,
1-Methyl-N-((6-(methyl(spiro[2.3]hexan-5-yl)amino)pyridin-3-yl)methyl)-2-oxo-2,3-dihydro-1H-benzimidazole-5-carboxamide,
(S)—N-((6-((3,3-Difluorocyclopentyl)(methyl)amino)pyridin-3-yl)methyl)-1-methyl-2-oxo-2,3-dihydro-1H-benzimidazole-5-carboxamide,
(R)—N-((6-((3,3-Difluorocyclopentyl)(methyl)amino)pyridin-3-yl)methyl)-1-methyl-2-oxo-2,3-dihydro-1H-benzimidazole-5-carboxamide,
N-((6-((3-(Difluoromethoxy)cyclobutyl)(methyl)amino)pyridin-3-yl)methyl)-1-methyl-2-oxo-2,3-dihydro-1H-benzimidazole-5-carboxamide and its cis- and trans-isomers,
1-(Trideuteromethyl)-N-((6-(methyl(cis-3-(trifluoromethyl)cyclobutyl)amino)pyridin-3-yl)methyl)-2-oxo-2,3-dihydro-1H-benzimidazole-5-carboxamide,
6-Fluoro-N-((6-(methyl(cis-3-(trifluoromethyl)cyclobutyl)amino)pyridin-3-yl)methyl)-2-oxo-2,3-dihydro-1H-benzimidazole-5-carboxamide,
1-Methyl-N-((6-(methyl(3-(trifluoromethoxy)cyclobutyl)amino)pyridin-3-yl)methyl)-2-oxo-2,3-dihydro-1H-benzimidazole-5-carboxamide and its cis- and trans-isomers,
N-((6-(((1S,3R)-3-Fluorocyclopentyl)(methyl)amino)pyridin-3-yl)methyl)-1-methyl-2-oxo-2,3-dihydro-1H-benzimidazole-5-carboxamide,
N-((6-(((1R,3S)-3-Fluorocyclopentyl)(methyl)amino)pyridin-3-yl)methyl)-1-methyl-2-oxo-2,3-dihydro-1H-benzimidazole-5-carboxamide,
N-((6-((6,6-Difluorobicyclo[3.1.0]hexan-3-yl)(methyl)amino)pyridin-3-yl)methyl)-1-methyl-2-oxo-2,3-dihydro-1H-benzimidazole-5-carboxamide, and
N-((6-((4,4-Difluorocyclohexyl)(methyl)amino)pyridin-3-yl)methyl)-6-fluoro-1-methyl-2-oxo-2,3-dihydro-1H-benzimidazole-5-carboxamide,
or a pharmaceutically acceptable salt thereof.

8. A compound represented by the following Formula (Ia): 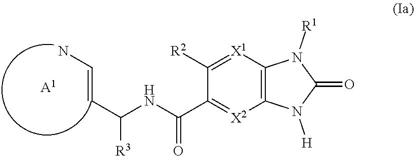
or a pharmaceutically acceptable salt thereof, wherein:
R¹ is hydrogen, C₁-3-alkyl, or C₁-3-deuteroalkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen, aryl, carboxy, —CO—O—(C₁₋₄-alkyl), or C₁₋₄-alkyl which may be substituted with hydroxy,
a ring A¹: 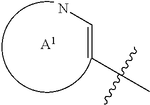
is a group of the formula: 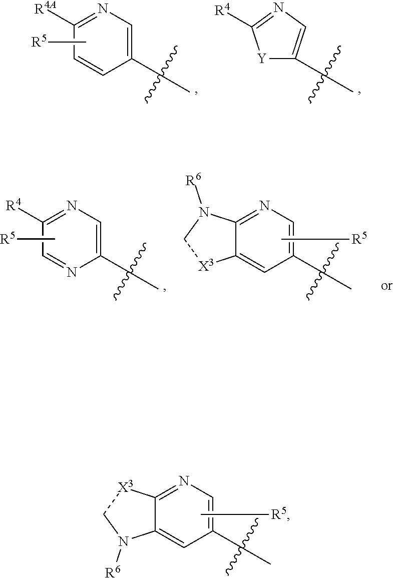
the wavy line indicates a point of attachment to the rest of molecule,
R⁴ is —N(R^{A})(R^{B}), —O—R^{A}, or C₁₋₂-alkyl substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
R^{4A} is —N(R^{A})(R^{B}), or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R^{R} is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, hydroxy, —O—(C₁₋₄-alkyl), C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, —O—(C₁₋₄-haloalkyl), amino, or mono- or di-(C₁₋₄-alkyl)amino; heterocyclyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino,
provided that the compound is not

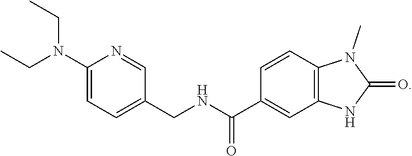 [SMILES: CCN(CC)c1ccc(CNC(=O)c2ccc3c(c2)[nH]c(=O)n3C)cn1]

9. The compound of claim 8, which is a compound represented by the following Formula (Ia): 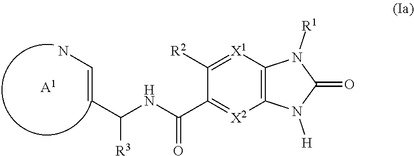
or a pharmaceutically acceptable salt thereof, wherein:
R¹ is hydrogen or C₁₋₃-alkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen or C₁₋₄-alkyl,
a ring A¹: 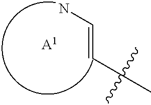
is a group of the formula: 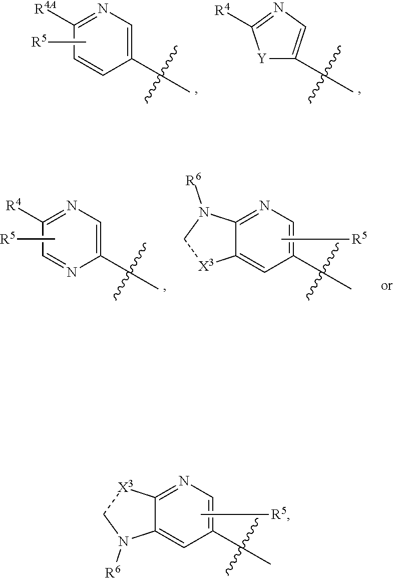
the wavy line indicates a point of attachment to the rest of molecule,
R⁴ is —N(R^{A})(R^{B}), —O—R^{A}, or C₁₋₂-alkyl substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R^{4A} is —N(R^{A})(R^{B}), or C₁₋₂-alkyl optionally substituted with a substituent selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
R⁵ is hydrogen, halogen, or C₁₋₄-alkyl,
R⁶ is R^{A},
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
X³ is N or C(R⁸)ₙ,
R⁸ is hydrogen or halogen,
n is an integer having a value of 1 or 2,
Y is O or S,
a dotted line is a single bond or a double bond, provided that when X³ is N or C(R⁸)ₙ, in which n is an integer having a value of 1, the dotted line is a double bond,
R^{A} is C₁₋₄-alkyl optionally substituted with one or more groups selected from the group consisting of halogen, C₃₋₆-cycloalkyl, aryl, heterocyclyl, and heteroaryl, in which said C₃₋₆-cycloalkyl, aryl, heterocyclyl, and heteroaryl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; aryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with heterocyclyl optionally having halogen, cyano, hydroxy, C₁₋₄-alkyl, —O—(C₁₋₄-alkyl), C₁₋₄-haloalkyl, amino, or mono- or di-(C₁₋₄-alkyl)amino; heterocyclyl optionally substituted with one or more groups selected from the group consisting of halogen, cyano, hydroxy, C₁₋₄-alkyl, —O—(Ci_₄-alkyl), C₁₋₄-haloalkyl, amino, and mono- or di-(C₁₋₄-alkyl)amino; or heteroaryl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and
R^{B} is hydrogen, C₁₋₄-alkyl, or C₃₋₆-cycloalkyl, or
R^{A} and R^{B} at the —N(R^{A})(R^{B}) are taken together with the nitrogen atom to form an N-containing heterocyclic ring optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino,
provided that the compound is not

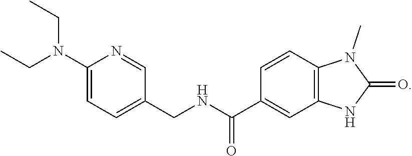 [SMILES: CCN(CC)c1ccc(CNC(=O)c2ccc3c(c2)[nH]c(=O)n3C)cn1]

10. The compound of claim 9, or a pharmaceutically acceptable salt thereof, wherein R¹ is hydrogen, methyl, ethyl, or isopropyl, and R³ is hydrogen or methyl.

11. The compound of claim 10, or a pharmaceutically acceptable salt thereof, wherein X¹ and X² are each independently C—R⁷, in which R⁷ is hydrogen or halogen.

12. The compound of claim 11, or a pharmaceutically acceptable salt thereof, wherein the ring A¹: 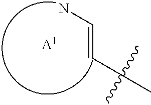
is a group of the formula:

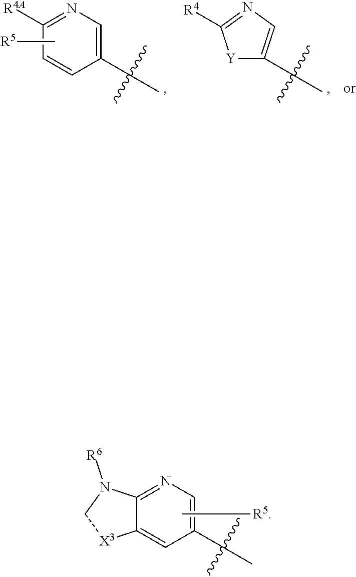

13. The compound of claim 12, or a pharmaceutically acceptable salt thereof, wherein R⁴ and R^{4A} are each —N(R^{A})(R^{B}), and R⁵ is hydrogen, and R⁶ is R^{A}, in which R^{A} is C₁₋₄-alkyl optionally substituted with one or more groups selected from the group consisting of halogen, C₃₋₆-cycloalkyl, phenyl, tetrahydropyranyl, piperidyl, piperazinyl, pyridyl, pyrimidyl, and indazolyl, in which said C₃₋₆-cycloalkyl, phenyl, tetrahydropyranyl, piperidyl, piperazinyl, pyridyl, pyrimidyl, and indazolyl may be further substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; phenyl optionally substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino, in which said C₁₋₄-alkyl may be further substituted with tetrahydropyranyl, piperidyl, or piperazinyl, each of which may be further substituted with halogen, cyano, hydroxy, C₁₋₄-alkyl, —O—(C₁₋₄-alkyl), C₁₋₄-haloalkyl, amino, or mono- or di-(C₁₋₄-alkyl)amino; tetrahydropyranyl, piperidyl, or piperazinyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; or pyridyl, pyrimidyl, or indazolyl, each of which may be substituted with one or more groups selected from the group consisting of halogen, cyano, hydroxy, C₁₋₄-alkyl, —O—(C₁₋₄-alkyl), C₁₋₄-haloalkyl, amino, and mono- or di-(C₁₋₄-alkyl)amino; and R^{B} is methyl.

14. The compound of claim 13, or a pharmaceutically acceptable salt thereof, wherein R^{A} is isopropyl; C₃₋₆-cycloalkyl substituted with one or more groups selected from the group consisting of halogen, cyano, C₁₋₄-alkyl, and C₁₋₄-haloalkyl; phenyl substituted with one or more groups selected from the group consisting of halogen, cyano, C₁₋₄-alkyl, and C₁₋₄-haloalkyl; or pyridyl substituted with one or more groups selected from the group consisting of halogen, cyano, C₁₋₄-alkyl, and C₁₋₄-haloalkyl.

15. The compound of claim 8, or a pharmaceutically acceptable salt thereof, wherein:
R¹ is hydrogen, C₁₋₃-alkyl, or C₁₋₃-deuteroalkyl,
R² is hydrogen or halogen,
R³ is hydrogen or C₁₋₄-alkyl,
a ring A¹: 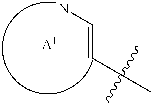
is a group of the formula: 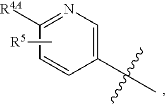
the wavy line indicates a point of attachment to the rest of molecule,
R^{4A} is —N(R^{A})(R^{B}),
R⁵ is hydrogen or halogen,
X¹ and X² are each independently N or C—R⁷,
R⁷ is hydrogen or halogen,
R^{A} is C₃₋₆-cycloalkyl substituted with one or more groups selected from the group consisting of halogen, C₁₋₄-haloalkyl, and —O—(C₁₋₄-haloalkyl), and
R^{B} is hydrogen or C₁₋₄-alkyl.

16. The compound of claim 8, wherein the compound is selected from the group consisting of
N-((2-(4-Fluorophenoxy)thiazol-5-yl)methyl)-1-methyl-2-oxo-2,3-dihydro-1H-benzimidazole-5-carboxamide,
N-((6-((4-Fluorophenyl)amino)pyridin-3-yl)methyl)-2-oxo-2,3-dihydro-1H-benzimidazole-5-carboxamide,
N-((6-((4-Fluorophenyl)(methyl)amino)pyridin-3-yl)methyl)-1-methyl-2-oxo-2,3-dihydro-1H-benzimidazole-5-carboxamide,
N-((6-((4,4-Difluorocyclohexyl)(methyl)amino)pyridin-3-yl)methyl)-1-methyl-2-oxo-2,3-dihydro-1H-benzimidazole-5-carboxamide,
N-((6-(Isopropyl(methyl)amino)pyridin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyridin-3-yl)methyl)-2-oxo-2,3-dihydro-1H-benzimidazole-5-carboxamide,
(S)—N-(1-(6-((4-Fluorophenyl)(methyl)amino)pyridin-3-yl)ethyl)-1-methyl-2-oxo-2,3-dihydro-1H-benzimidazole-5-carboxamide,
(S)-1-Methyl-N-((6-(2-methylpyrrolidin-1-yl)pyridin-3-yl)methyl)-2-oxo-2,3-dihydro-1H-benzimidazole-5-carboxamide,
N-((6-(1-(4-Fluorophenyl)ethyl)pyridin-3-yl)methyl)-1-methyl-2-oxo-2,3-dihydro-1H-benzimidazole-5-carboxamide,
N-((6-(Fluoro(4-fluorophenyl)methyl)pyridin-3-yl)methyl)-1-methyl-2-oxo-2,3-dihydro-1H-benzimidazole-5-carboxamide,
N-((1-Isopropyl-2,3-dihydro-1H-pyrrolo[2,3-b]pyridin-5-yl)methyl)-1-methyl-2-oxo-2,3-dihydro-1H-benzimidazole-5-carboxamide,
N-((1-(4,4-difluorocyclohexyl)-2,3-dihydro-1H-pyrrolo[2,3-b]pyridin-5-yl)methyl)-1-methyl-2-oxo-2,3-dihydro-1H-benzimidazole-5-carboxamide,
N-((1-(2-Fluorophenyl)-2,3-dihydro-1H-pyrrolo[2,3-b]pyridin-5-yl)methyl)-1-methyl-2-oxo-2,3-dihydro-1H-benzimidazole-5-carboxamide,
1-Methyl-2-oxo-N-((1-phenyl-1H-pyrrolo[2,3-b]pyridin-5-yl)methyl)-2,3-dihydro-1H-benzimidazole-5-carboxamide,
N-((3-(2,6-Difluorophenyl)-3H-imidazo[4,5-b]pyridin-6-yl)methyl)-1-methyl-2-oxo-2,3-dihydro-1H-benzimidazole-5-carboxamide,
N-((6-(Cyclopropyl(4-fluorophenyl)amino)pyridin-3-yl)methyl)-1-methyl-2-oxo-2,3-dihydro-1H-benzimidazole-5-carboxamide,
1-Methyl-N-((6-(methyl(1-methylpiperidin-4-yl)amino)pyridin-3-yl)methyl)-2-oxo-2,3-dihydro-1H-benzimidazole-5-carboxamide,
N-((2-((4,4-Difluorocyclohexyl)amino)thiazol-5-yl)methyl)-1-methyl-2-oxo-2,3-dihydro-1H-benzimidazole-5-carboxamide,
1-Methyl-N-((6-(methyl(3-((4-methylpiperazin-1-yl)methyl)phenyl)amino)pyridin-3-yl)methyl)-2-oxo-2,3-dihydro-1H-benzimidazole-5-carboxamide,
N-((6-((4,4-Difluorocyclohexyl)(methyl)amino)pyrazin-3-yl)methyl)-1-methyl-2-oxo-2,3-dihydro-1H-benzimidazole-5-carboxamide,
1-Methyl-N-((6-(methyl(cis-3-(trifluoromethyl)cyclobutyl)amino)pyrazin-3-yl)methyl)-2-oxo-2,3-dihydro-1H-benzimidazole-5-carboxamide,
1-Methyl-N-((6-(methyl(spiro[2.3]hexan-5-yl)amino)pyridin-3-yl)methyl)-2-oxo-2,3-dihydro-1H-benzimidazole-5-carboxamide,
(S)—N-((6-((3,3-Difluorocyclopentyl)(methyl)amino)pyridin-3-yl)methyl)-1-methyl-2-oxo-2,3-dihydro-1H-benzimidazole-5-carboxamide,
(R)—N-((6-((3,3-Difluorocyclopentyl)(methyl)amino)pyridin-3-yl)methyl)-1-methyl-2-oxo-2,3-dihydro-1H-benzimidazole-5-carboxamide,
N-((6-((3-(Difluoromethoxy)cyclobutyl)(methyl)amino)pyridin-3-yl)methyl)-1-methyl-2-oxo-2,3-dihydro-1H-benzimidazole-5-carboxamide and its cis- and trans-isomers,
1-(Trideuteromethyl)-N-((6-(methyl(cis-3-(trifluoromethyl)cyclobutyl)amino)pyridin-3-yl)methyl)-2-oxo-2,3-dihydro-1H-benzimidazole-5-carboxamide,
6-Fluoro-N-((6-(methyl(cis-3-(trifluoromethyl)cyclobutyl)amino)pyridin-3-yl)methyl)-2-oxo-2,3-dihydro-1H-benzimidazole-5-carboxamide,
1-Methyl-N-((6-(methyl(3-(trifluoromethoxy)cyclobutyl)amino)pyridin-3-yl)methyl)-2-oxo-2,3-dihydro-1H-benzimidazole-5-carboxamide and its cis- and trans-isomers,
N-((6-(((1S,3R)-3-Fluorocyclopentyl)(methyl)amino)pyridin-3-yl)methyl)-1-methyl-2-oxo-2,3-dihydro-1H-benzimidazole-5-carboxamide,
N-((6-(((1R,3S)-3-Fluorocyclopentyl)(methyl)amino)pyridin-3-yl)methyl)-1-methyl-2-oxo-2,3-dihydro-1H-benzimidazole-5-carboxamide,
N-((6-((6,6-Difluorobicyclo[3.1.0]hexan-3-yl)(methyl)amino)pyridin-3-yl)methyl)-1-methyl-2-oxo-2,3-dihydro-1H-benzimidazole-5-carboxamide, and
N-((6-((4,4-Difluorocyclohexyl)(methyl)amino)pyridin-3-yl)methyl)-6-fluoro-1-methyl-2-oxo-2,3-dihydro-1H-benzimidazole-5-carboxamide,
or a pharmaceutically acceptable salt thereof.

17. A compound represented by the following Formula (Ib): 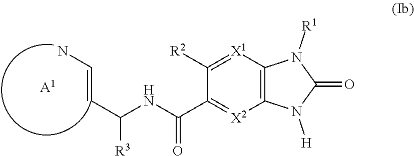
or a pharmaceutically acceptable salt thereof, wherein:
R¹ is hydrogen, C₁₋₃-alkyl, or C₁₋₃-deuteroalkyl,
R² is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R³ is hydrogen, aryl, carboxy, —CO—O—(C₁₋₄-alkyl), or C₁₋₄-alkyl which may be substituted with hydroxy,
a ring A²: 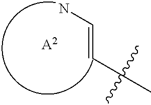
is a group of the formula: 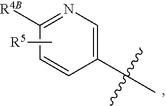
the wavy line indicates a point of attachment to the rest of molecule,
R^{4B} is —O—R^{C},
R⁵ is hydrogen, halogen, or C₁₋₄-alkyl,
X¹ and X² are each independently N or C—R⁷,
R⁷ is hydrogen, halogen, hydroxy, C₁₋₄-alkyl, —O—(C₁₋₄-alkyl), C₁₋₄-haloalkyl, nitro, amino, or mono- or di-(C₁₋₄-alkyl)amino,
R^{C} is C₃₋₆-cycloalkyl optionally substituted with one or more groups selected from the group consisting of halogen, cyano, hydroxy, C₁₋₄-alkyl, —O—(C₁₋₄-alkyl), C₁₋₄-haloalkyl, —O—(C₁₋₄-haloalkyl), amino, and mono- or di-(C₁₋₄-alkyl)amino.

18-22. (canceled)

23. A method of treating a KIT mediated disease or condition in a subject, comprising administering to the subject in need thereof a therapeutically effective amount of one or more the pharmaceutical composition of claim 1.

24. The method of claim 23, wherein the KIT mediated disease or condition is a cancer, an autoimmune disease, an allergic disease, an inflammatory disease, fibrosis, a metabolic disorder, a neurodegenerative disease, bone loss, tumor angiogenesis, interstitial cystitis, pulmonary arterial hypertension (PAH), and primary pulmonary hypertension (PPH).

25. The method of claim 24, wherein:
the cancer is selected from mastocytosis, mastocytoma, solid tumor, gastrointestinal stromal tumor (“GIST”), small cell lung cancer, non-small cell lung cancer, acute myelocytic leukemia, acute lymphocytic leukemia, myelodysplastic syndrome, chronic myelogenous leukemia, colorectal carcinoma, gastric carcinoma, testicular cancer, glioblastoma, astrocytoma, melanoma, mast cell tumor, neuroblastoma, sarcoma, and seminoma;
the autoimmune disease is selected from multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, and proliferative glomerulonephritis;
the allergic disease is selected from asthma, severe asthma, allergic rhinitis, chronic rhinitis, allergic sinusitis, anaphylactic syndrome, urticaria, food allergy, seasonal allergy, angioedema, atopic dermatitis, allergic contact dermatitis, erythema nodosum, erythema multiforme, cutaneous necrotizing venulitis, insect bite skin inflammation, and blood sucking parasitic infestation;
the inflammatory disease is selected from rheumatoid arthritis, conjunctivitis, rheumatoid spondylitis, osteoarthritis, gouty arthritis, an arthritic condition, inflammatory bowel diseases (IBD), irritable bowel syndrome (IBS), and eosinophilic esophagitis;
the fibrosis is selected from pulmonary fibrosis, hepatic fibrosis, cardiac fibrosis, and myelofibrosis;
the metabolic disorder is selected from diabetes mellitus and its chronic complications; obesity; type I diabetes or type II diabetes; hyperlipidemias and dyslipidemias; atherosclerosis; hypertension; and cardiovascular disease; and
the neurodegenerative disease is selected from Alzheimer's disease, Parkinson's disease, Huntington's disease, the prion diseases, motor neuron disease (MND), and amyotrophic lateral sclerosis (ALS).
